SEQUENCE LISTING (25 sequences)

SEQ ID NO: 1 - DNA, 10807 bp, Zika virus
  source (1)..(10807): Zika virus isolate H.sapiens-tc/PHL/2012/ CPC-0740 genome
agttgttgatctgtgtgaatcagactgcgacagttcgagtttgaagcgaaagctagcaacagtatcaacaggttttattttggatttggaaacgagagtttctggtcatgaaaaacccaaaaaagaaatccggaggattccggattgtcaatatgctaaaacgcggagtagcccgtgtgagcccctttgggggcttgaagaggctgccagccggacttctgctgggccatgggcccatcaggatggtcttggcgatactagcctttttgagattcacggcaatcaagccatcactgggtctcatcaatagatggggttcagtggggaaaaaagaggctatggaaataataaagaagttcaagaaagatctggctgccatgctgagaataatcaatgctaggaaggagaagaagagacgaggcgcagatactagcgtcggaattgttggcctcctcctgaccacagccatggcagtagaggtcactagacgtgggagtgcatactatatgtacttggacagaagcgatgctggggaggccatatcttttccaaccacactggggatgaataagtgttacatacaaatcatggatcttggacacatgtgtgatgccaccatgagctatgaatgccctatgttggatgagggggtagaaccagatgacgtcgattgctggtgcaacacgacatcaacttgggttgtgtatggaacctgccaccacaaaaaaggtgaagcacggagatctagaagagctgtgacgctcccctcccattccactaggaagctgcaaacgcggtcgcagacctggttggaatcaagagaatacacaaagcacctgattagagttgaaaattggatattcaggaaccctggcttcgcgttagcagcagctgtcatcgcttggcttttgggaagttcaacgagccaaaaagtcatatatctggtcatgatactgctgattgccccggcatacagcatcaggtgcataggagtcagcaatagggactttgtggaaggtatgtcaggtgggacttgggttgatgttgtcttggaacatggaggttgtgttaccgtaatggcacaggacaaaccgactgtcgacatagagctggttacaacaacagtcagcaacatggcggaggtaagatcctactgctatgaggcatcaatatcggatatggcttcggacagccgctgcccaacacaaggtgaggcctaccttgacaagcagtcagacactcaatatgtctgcaaaagaacgttagtggacagaggctggggaaatggatgtggactttttggcaaagggagcctggtgacatgcgctaagtttgcatgctccaagaaaatgaccgggaagagcatccagccagagaatctggagtaccggataatgctgtcagttcatggctcccagcacagtgggatgatcgttaatgacacaggacatgaaactgatgagaatagagcgaaggttgagataacgcccaattcaccaagagccgaagccaccctggggggttttgggagcctaggacttgattgtgaaccgaggacaggccttgacttttcagatttgtattacctgactatgaataacaagcactggttggttcacaaggagtggttccacgacattccattaccttggcatgctggggcagacactggaactccacattggaacaacaaagaagcactggtagagttcaaggacgcacatgcaaaaaggcaaactgtcgtggttctagggagtcaagaaggagcagttcacacggcccttgctggagctctggaggctgagatggatggagccaagggaaggctgtcctctggccacttgaaatgtcgcctgaaaatggataaacttagattgaagggcgtgtcatactccttgtgcactgcagcgttcacattcaccaagatcccggctgaaacactgcacgggacagtcacagtggaggtacagtacgcagggacagatggaccttgcaaggttccagctcagatggcggtggatatgcaaactctgaccccagttgggaggttgataaccgctaaccctgtaatcactgaaagcaccgagaactctaagatgatgctggaacttgatccaccatttggggactcttacattgtcataggagtcggggagaagaagatcacccatcactggcacaggagtggcagcaccattggaaaagcatttgaagccactgtgagaggtgccaagagaatggcagtcttgggagacacagcctgggactttggatcagttgggggtgctctcaactcattgggcaagggcatccatcaaatttttggagcagctttcaaatcattgttcggaggaatgtcctggttctcacaaattctcattggaacgttgctggtgtggttgggtctgaatacaaagaatggatctatttcccttacgtgcttggccttagggggagtgttgatcttcttatccacagccgtttctgctgatgtggggtgctcggtggacttctcaaagaaggaaacgagatgcggtacaggggtgttcgtctataacgacgttgaagcctggagggacaggtacaagtaccatcctgactcccctcgtagattggcagcagcagtcaagcaagcctgggaagatgggatctgtgggatctcctctgtctcaagaatggaaaacatcatgtggagatcagtagaaggggagctcaacgcaatcctggaagagaatggagttcaactgacggtcgttgtgggatctgtaaaaaaccccatgtggagaggtccacagagattgcccgtgcctgtgaacgagctgccccacggctggaaggcttgggggaaatcgtacttcgtcagagcagcaaagacaaataacagctttgtcgtggatggtgacacactgaaggaatgcccactcaaacatagagcatggaacagctttcttgtggaggatcatgggtttggggtatttcacactagtgtctggctcaaggttagagaagattattcattagagtgtgatccagccgtcattggaacagctgctaagggaaaggaggctgtgcacagcgatctaggctactggattgagagtgagaagaacgacacatggaggctgaagagggcccacctgatcgagatgaaaacatgtgaatggccaaagtcccacacattgtggacagatggagtagaagaaagtgatctgatcatacccaagtctttagctgggccactcagccatcacaacaccagagagggctacaggactcaaatgaaagggccatggcacagtgaagagcttgaaattcggtttgaggaatgcccaggcactaaggtccacgtggaggaaacatgtgggacaagaggaccatccctgagatcaaccactgcaagcggaagggtgatcgaggaatggtgctgcagggaatgcacaatgcccccactgtcgttccgagctaaagatggctgttggtatggaatggagataaggcccaggaaagaaccagaaagtaacttagtaaggtcaatggtgactgcaggatcaactgatcacatggatcacttctctcttggagtgcttgtgattttgctcatggtgcaggaagggctgaagaagagaatgaccacaaagatcatcataagcacatcaatggcagtgctggtagccatgatcctgggaggattttcaatgagtgacctggctaagcttgcaattttgatgggtgccaccttcgcggaaatgaacactggaggagatgtagctcatttggcgctgatagcggcattcaaagtcagacctgcgttgctggtatctttcatcttcagagctaattggacaccccgtgagagcatgctgctggccttggcctcgtgtcttctgcaaactgcgatctccgccttggaaggcgacctgatggttctcatcaatggttttgctttggcctggttggcaatacgagcgatggttgttccacgcactgacaacatcaccttggcaatcctggctgctctgacaccactggcccggggcacactgcttgtggcgtggagagcaggccttgctacttgcggggggttcatgctcctctctctgaaggggaaaggcagtgtgaagaagaacctaccatttgtcatggccttgggactaactgctgtgaggctggtcgaccccatcaacgtggtgggactgctgttgctcacaaggagtgggaagcggagctggccccctagtgaagtactcacagctgttggcctgatatgcgcattggctggagggttcgccaaggcggatatagagatggctgggcccatggccgcggtcggtctgctaattgtcagttacgtggtctcaggaaagagtgtggacatgtacattgaaagagcaggtgacatcacatgggaaaaagatgcggaaatcactggaaacagtccccggctcgatgtggcactagatgagagtggtgatttctccctagtggaggatgatggtccacccatgagagagatcatactcaaagtggtcctgatgaccatctgcggcatgaacccaatagccataccctttgcagctggagcgtggtacgtgtatgtgaagactggaaaaaggagtggtgctctatgggatgtgcctgctcccaaggaagtaaaaaagggggagaccacagatggagtgtacagagtaatgactcgtagactgcttggttcaacacaagttggagtgggagtcatgcaagagggggtcttccacactatgtggcacgtcacaaaaggatccgcgctgagaagcggtgaagggagacttgatccatactggggagatgtcaagcaggatctggtgtcatactgtggtccgtggaagctagacgccgcctgggacgggcacagcgaggtgcagctcttggccgtgccccccggagagagagcgaggaacatccagactctgcccggaacatttaagacaaaggatggggacattggagcagttgcgctggactacccagcaggaacttcaggatctccaatcctagacaagtgtgggagagtgataggactctatggtaatggggtcgtgataaaaaatgggagttatgttagtgccatcacccaagggaggagggaggaagagactcctgttgagtgcttcgagccttcgatgctgaagaagaagcagctaactgtcttagacctgcatcctggagccgggaaaaccaggagagttcttcctgaaatagtccgtgaagccataaaaacaagactccgtactgtgatcttagctccaaccagggtcgtcgctgctgaaatggaggaagcccttagagggcttccagttcgttatatgacaacagcagtcaatgtcacccattctgggacagaaatcgttgacttaatgtgccatgctaccttcacttcacgcctactacaaccaatcagagtccccaactataatttgtatattatggatgaggcccacttcacagatccctcaagtatagcagcaagaggatacatttcaacaagggttgagatgggcgaggcggctgccatcttcatgaccgccacgccaccaggaacccgtgacgcattcccggactccaactcaccaattatggacaccgaggtggaagtcccagagagagcctggagcacaggctttgattgggtgacggatcattctgggaaaacagtctggtttgttccaagcgtgaggaacggcaatgagatcgcagcttgtctgacaaaggctggaaaacgggtcatacagctcagcagaaagacttttgagacagagttccagaaaacgaaaaatcaagagtgggacttcgtcgtgacaaccgacatttcagagatgggcgccaactttaaagctgaccgtgtcatagattccaggagatgcttaaagccggtcatacttgatggcgagagagtcattttggctggacccatgcctgtcacacatgccagcgctgctcagaggagggggcgcataggcaggaatcccaacaaacctggagatgagtatctgtatggaggtgggtgcgcagagactgatgaagatcacgcacactggcttgaagcaagaatgcttcttgacaacatttacctccaagatggcctcatagcttcgctctatcgacctgaggccgacaaagtagcagctattgagggagagttcaagcttaggacggagcaaaggaagacctttgtggaactcatgaaaagaggagatcttccggtttggttggcctatcaggttgcatctgccggaataacctacacagatagaagatggtgctttgatggcatgaccaacaacaccataatggaagacagtgtgccggcagaggtgtggaccagatacggagagaaaagagtgctcaaaccgaggtggatggacgccagagtttgttcagatcatgcggccctgaagtcattcaaagagtttgccgctgggaaaagaggagcggcctttggagtgatagaagccctgggaacactgccaggacacatgacagagagattccaggaagccattgacaacctcgctgtgctcatgcgggcagagactggaagcaggccttacaaagccgcggcggcccaattgccggagaccctagagaccattatgcttttggggttgctgggaacagtctcgctgggaatctttttcgtcttgatgcggaacaagggcatggggaagatgggctttggaatggtgactcttggggccagcgcatggcttatgtggctctcggaaattgagccagccagaattgcatgtgtcctcattgtcgtgttcctattgctggtggtgctcatacctgagccagaaaagcaaagatctcctcaggacaaccaaatggcaatcatcatcatggtagcagtgggtcttctgggcttgattaccgccaatgaactcggatggttggagagaacaaaaagtgacctaagccatctaatgggaaggagagaggagggggcaaccacaggattctcaatggacattgacctgcggccagcctcagcttgggctatctatgctgctctgacaactttcatcaccccagccgtccaacatgcggtgaccacttcatacaacaactactccttaatggcgatggccacgcaagctggggtgttgtttggtatgggcaaagggatgccattctacgcatgggactttggagtcccgctgctaatgatgggttgctactcacaattaacacctctgaccctaatagtggccatcattttgctcgtggcgcactacatgtacttgatcccagggctgcaggcagcagctgcgcgggctgcccagaagagaacggcagctggcatcatgaagaaccctgttgtggatggaatagtggtgactgacattgacacaatgacaattgacccccaagtggaaaaaaagatggggcaggtgctactcatagcagtagccgtctccagcgccatactgtcgcggaccgcctgggggtggggggaggctggggccctgatcacagctgcaacttccaccttgtgggaaggctctccgaacaagtactggaactcctccacagccacttcactgtgtaacatttttaggggaagttacttggctggagcttctctaatctacacagtaacaagaaacgctggcttggtcaagagacgtgggggtggaacgggagagaccctgggagagaaatggaaggcccgcctgaaccagatgtcggccctggagttctactcctacaaaaagtcaggcatcaccgaggtgtgcagagaagaggcccgccgtgccctcaaggacggtgtggcaacaggaggccatgctgtgtcccgaggaagtgcaaagcttagatggctggtggagagaggatacctgcagccctatggaaaggtcattgatcttggatgtggcagagggggctggagttactatgccgccaccatccgcaaagttcaggaagtgaaaggatacacaaaaggaggccctggtcatgaagaacccatgttggtgcaaagctatgggtggaacatagtccgtcttaagagtggggtggacgtctttcacatggcggctgagccgtgtgacactttgctgtgtgatataggtgagtcatcatctagtcctgaagtggaagaagcacggacgctcagagtcctctccatggtgggggattggcttgaaaaaagaccaggagccttttgtataaaagtgttgtgcccatacaccagcactatgatggaaaccctggagcgactgcagcgtaggtatgggggaggactggtcagggtgccactctcccgcaactctacacatgagatgtactgggtctctggagcgaaaagcaacaccataaaaagtgtgtccaccacgagccagctcctcttggggcgcatggacgggcccaggaggccagtgaaatatgaggaggatgtgaatctcggctctggcacgcgggctgtggtaagctgcgctgaagctcccaacatgaagatcattggtaaccgcattgagaggatccgcagtgagcacgcggaaacgtggttctttgacgagaaccacccatataggacatgggcttaccatggaagctatgaggcccctacacaagggtcagcgtcctctctaataaacggggttgtcaggctcctgtcaaaaccctgggatgtggtgactggagtcacaggaatagccatgactgacaccacaccgtatggtcagcaaagagttttcaaggaaaaagtggacactagggtgccagacccccaagaaggcactcgtcaggttatgagcatggtctcttcctggttatggaaggagctaggcaaacacaaacggccacgagtctgtaccaaagaagagttcatcaacaaggttcgtagcaatgcagcattaggggcaatatttgaagaggaaaaagagtggaagactgcagtggaagctgtgaatgatccaaggttctgggctctagtggacaaggaaagagagcatcacctgagaggagagtgtcagagctgtgtgtacaacatgatgggaaaaagagaaaagaaacaaggggaatttggaaaggccaagggcagccgcgccatctggtatatgtggctaggggctagattcctagagttcgaagcccttggattcttgaatgaggatcattggatggggagagagaattcaggaggtggtgttgaaggactgggattacaaagactcggatatgtcctagaagagatgagtcgcataccaggaggaaggatgtatgcagatgatactgctggctgggacacccgcatcagcaggtttgatctggagaatgaagctctaatcaccaaccaaatggagaaagggcacagggccttggcattggccataatcaagtacacataccaaaacaaagtggtaaaggtccttagaccagctgaaaaagggaagacagttatggacattatttcaagacaagaccaaagggggagcggacaagttgtcacttacgctcttaatacattcaccaacctggtggtgcagctcattcggaatatggaggctgaggaagttctagagatgcaagacttgtggctgctgcggaggccagagaaagtgaccaactggttgcaaagcaacggatgggataggctcaaaagaatggcagtcagtggagatgattgcgttgtgaaaccaattgatgataggtttgcacatgccctcaggttcttgaatgatatgggaaaagttaggaaggacacacaagagtggaaaccctcaactggatgggacaactgggaagaagttccgttttgctcccaccacttcaacaaactccatcttaaggacgggaggtccattgtggttccctgccgccaccaagatgaactgattggccgagcccgcgtatcaccaggggcgggatggagcatccgggagactgcttgcctagcaaaatcatatgcgcaaatgtggcagctcctttatttccacagaagggacctccgactgatggccaatgccatttgttcatctgtgccagttgattgggttccaactgggagaactacctggtcaatccatggaaagggagaatggatgaccactgaagacatgcttgtggtatggaacagagtgtggattgaggaaaacgaccacatggaagacaagaccccagttacaaaatggacagacattccctatttgggaaaaagagaagacttgtggtgtggatctctcatagggcacagaccgcgtactacctgggctgagaacatcaaaaatacagtcaacatgatgcgcaggatcataggtgatgaagaaaagtacatggactacctatccacccaggttcgctacttgggtgaagaagggtccacacctggagtgctgtaagcaccaatcttagtgttgtcaggcctgctagtcagccacagcttggggaaagctgtgcagcctgtgacccccccaggagaagctgggaaaccaagcctatagtcaggccgagaacgccatggcacggaagaagccatgctgcctgtgagcccctcagaggacactgagtcaaaaaaccccacgcgcttggaggcgcaggatgggaaaagaaggtggcgaccttccccacccttcaatctggggcctgaactggagatcagctgtggatctccagaagagggactagtggttagaggagaccccccggaaaacgcaaaacagcatattgacgctgggaaagaccagagactccatgagtttccaccacgctggccgccaggcacagatcgccgaatagcggcggccggtgtggggaaatccatgggtct

SEQ ID NO: 2 - DNA, 10648 bp, Zika virus
  source (1)..(10648): Zika virus isolate SSABR1 genome
gacagttcgagtttgaagcgaaagctagcaacagtatcaacaggttttatttggatttggaaacgagagtttctggtcatgaaaaacccaaaaaagaaatccggaggattccggattgtcaatatgctaaaacgcggagtagcccgtgtgagcccctttgggggcttgaagaggctgccagccggacttctgctgggtcatgggcccatcaggatggtcttggcgattctagcctttttgagattcacggcaatcaagccatcactgggtctcatcaatagatggggttcagtggggaaaaaagaggctatggaaataataaagaagttcaagaaagatctggctgccatgctgagaataatcaatgctaggaaggagaagaagagacgaggcgcagatactagtgtcggaattgttggcctcctgctgaccacagctatggcagcggaggtcactagacgtgggagtgcatactatatgtacttggacagaaacgatgctggggaggccatatcttttccaaccacattggggatgaataagtgttatatacagatcatggatcttggacacatgtgtgatgccaccatgagctatgaatgccctatgctggatgagggggtggaaccagatgacgtcgattgttggtgcaacacgacgtcaacttgggttgtgtacggaacctgccatcacaaaaaaggtgaagcacggagatctagaagagctgtgacgctcccctcccattccactaggaagctgcaaacgcggtcgcaaacctggttggaatcaagagaatacacaaagcacttgattagagtcgaaaattggatattcaggaaccctggcttcgcgttagcagcagctgccatcgcttggcttttgggaagctcaacgagccaaaaagtcatatacttggtcatgatactgctgattgccccggcatacagcatcaggtgcataggagtcagcaatagggactttgtggaaggtatgtcaggtgggacctgggttgatgttgtcttggaacatggaggttgtgtcaccgtaatggcacaggacaaaccgactgtcgacatagagctggttacaacaacagtcagcaacatggcggaggtaagatcctactgctatgaggcatcaatatcagacatggcttcggacagccgctgcccaacacaaggtgaagcctaccttgacaagcaatcagacactcaatatgtctgcaaaagaacgttagtggacagaggctggggaaatggatgtggactttttggcaaagggagcctggtgacatgcgctaagtttgcatgctccaagaaaatgaccgggaagagcatccagccagagaatctggagtaccggataatgctgtcagttcatggctcccagcacagtgggatgattgttaatgacacaggacatgaaactgatgagaatagagcgaaagttgagataacgcccaattcaccaagagccgaagccaccctggggggttttggaagcctaggacttgattgtgaaccgaggacaggccttgacttttcagatttgtattacttgactatgaataacaagcactggttggttcacaaggagtggttccacgacattccattaccttggcacgctggggcagacaccggaactccacactggaacaacaaagaagcactggtagagttcaaggacgcacatgccaaaaggcaaactgtcgtggttctagggagtcaagaaggagcagttcacacggcccttgctggagctctggaggctgagatggatggtgcaaagggaaggctgtcctctggccacttgaaatgtcgcctgaaaatggataaacttagattgaagggcgtgtcatactccttgtgtactgcagcgttcacattcaccaagatcccggctgaaacactgcacgggacagtcacagtggaggtacagtacgcagggacagatggaccttgcaaggttccagctcagatggcggtggacatgcaaactctgaccccagttgggaggttgataaccgctaaccccgtaatcactgaaagcactgagaactctaagatgatgctggaacttgatccaccatttggggactcttacattgtcataggagtcggggagaagaagatcacccaccactggcacaggagtggcagcaccattggaaaagcatttgaagccactgtgagaggtgccaagagaatggcagtcttgggagacacagcctgggactttggatcagttggaggcgctctcaactcattgggcaagggcatccatcaaatttttggagcagctttcaaatcattgtttggaggaatgtcctggttctcacaaattctcattggaacgttgctgatgtggttgggtctgaacacaaagaatggatctatttcccttatgtgcttggccttagggggagtgttgatcttcttatccacagccgtctctgctgatgtggggtgctcggtggacttctcaaagaaggagacgagatgcggtacaggggtgttcgtctataacgacgttgaagcctggagggacaggtacaagtaccatcctgactccccccgtagattggcagcagcagtcaagcaagcctgggaagatggtatctgcgggatctcctctgtttcaagaatggaaaacatcatgtggagatcagtagaaggggagctcaacgcaatcctggaagagaatggagttcaactgacggtcgttgtgggatctgtaaaaaaccccatgtggagaggtccacagagattgcccgtgcctgtgaacgagctgccccacggctggaaggcttgggggaaatcgtacttcgtcagagcagcaaagacaaataacagctttgtcgtggatggtgacacactgaaggaatgcccactcaaacatagagcatggaacagctttcttgtggaggatcatgggttcggggtatttcacactagtgtctggctcaaggttagagaagattattcattagagtgtgatccagccgttattggaacagctgttaagggaaaggaggctgtacacagtgatctaggctactggattgagagtgagaagaatgacacatggaggctgaagagggcccatctgatcgagatgaaaacatgtgaatggccaaagtcccacacattgtggacagatggaatagaagagagtgatctgatcatacccaagtctttagctgggccactcagccatcacaataccagagagggctacaggacccaaatgaaagggccatggcacagtgaagagcttgaaattcggtttgaggaatgcccaggcactaaggtccacgtggaggaaacatgtggaacaagaggaccatctctgagatcaaccactgcaagcggaagggtgatcgaggaatggtgctgcagggagtgcacaatgcccccactgtcgttccgggctaaagatggctgttggtatggaatggagataaggcccaggaaagaaccagaaagcaacttagtaaggtcaatggtgactgcaggatcaactgatcacatggaccacttctcccttggagtgcttgtgattctgctcatggtgcaggaagggctgaagaagagaatgaccacaaagatcatcataagcacatcaatggcagtgctggtagctatgatcctgggaggattttcaatgagtgacctggctaagcttgcaattttgatgggtgccaccttcgcggaaatgaacactggaggagatgtagctcatctggcgctgatagcggcattcaaagtcagaccagcgttgctggtatctttcatcttcagagctaattggacaccccgtgaaagcatgctgctggccttggcctcgtgtcttttgcaaactgcgatctccgccttggaaggcgacctgatggttctcatcaatggttttgctttggcctggttggcaatacgagcgatggttgttccacgcactgataacatcaccttggcaatcctggctgctctgacaccactggcccggggcacactgcttgtggcgtggagagcaggccttgctacttgcggggggtttatgctcctctctctgaagggaaaaggcagtgtgaagaagaacttaccatttgtcatggccctgggactaaccgctgtgaggctggtcgaccccatcaacgtggtgggactgctgttgctcacaaggagtgggaagcggagctggccccctagcgaagtactcacagctgttggcctgatatgcgcattggctggagggttcgccaaggcagatatagagatggctgggcccatggccgcggtcggtctgctaattgtcagttacgtggtctcaggaaagagtgtggacatgtacattgaaagagcaggtgacatcacatgggaaaaagatgcggaagtcactggaaacagtccccggctcgatgtggcgctagatgagagtggtgatttctccctggtggaggatgacggtccccccatgagagagatcatactcaaggtggtcctgatgaccatctgtggcatgaacccaatagccataccctttgcagctggagcgtggtacgtatacgtgaagactggaaaaaggagtggtgctctatgggatgtgcctgctcccaaggaagtaaaaaagggggagaccacagatggagtgtacagagtaatgactcgtagactgctaggttcaacacaagttggagtgggagttatgcaagagggggtctttcacactatgtggcacgtcacaaaaggatccgcgctgagaagcggtgaagggagacttgatccatactggggagatgtcaagcaggatctggtgtcatactgtggtccatggaagctagatgccgcctgggacgggcacagcgaggtgcagctcttggccgtgccccccggagagagagcgaggaacatccagactctgcccggaatatttaagacaaaggatggggacattggagcggttgcgctggattacccagcaggaacttcaggatctccaatcctagacaagtgtgggagagtgataggactttatggcaatggggtcgtgatcaaaaatgggagttatgttagtgccatcacccaagggaggagggaggaagagactcctgttgagtgcttcgagccttcgatgctgaagaagaagcagctaactgtcttagacttgcatcctggagctgggaaaaccaggagagttcttcctgaaatagtccgtgaagccataaaaacaagactccgtactgtgatcttagctccaaccagggttgtcgctgctgaaatggaggaggcccttagagggcttccagtgcgttatatgacaacagcagtcaatgtcacccactctggaacagaaatcgtcgacttaatgtgccatgccaccttcacttcacgtctactacagccaatcagagtccccaactataatctgtatattatggatgaggcccacttcacagatccctcaagtatagcagcaagaggatacatttcaacaagggttgagatgggcgaggcggctgccatcttcatgaccgccacgccaccaggaacccgtgacgcatttccggactccaactcaccaattatggacaccgaagtggaagtcccagagagagcctggagctcaggctttgattgggtgacggatcattctggaaaaacagtttggtttgttccaagcgtgaggaacggcaatgagatcgcagcttgtctgacaaaggctggaaaacgggtcatacagctcagcagaaagacttttgagacagagttccagaaaacaaaacatcaagagtgggactttgtcgtgacaactgacatttcagagatgggcgccaactttaaagctgaccgtgtcatagattccaggagatgcctaaagccggtcatacttgatggcgagagagtcattctggctggacccatgcctgtcacacatgccagcgctgcccagaggagggggcgcataggcaggaatcccaacaaacctggagatgagtatctgtatggaggtgggtgcgcagagactgacgaagaccatgcacactggcttgaagcaagaatgctccttgacaatatttacctccaagatggcctcatagcctcgctctatcgacctgaggccgacaaagtagcagccattgagggagagttcaagcttaggacggagcaaaggaagacctttgtggaactcatgaaaagaggagatcttcctgtttggctggcctatcaggttgcatctgccggaataacctacacagatagaagatggtgctttgatggcacgaccaacaacaccataatggaagacagtgtgccggcagaggtgtggaccagacacggagagaaaagagtgctcaaaccgaggtggatggacgccagagtttgttcagatcatgcggccctgaagtcattcaaggagtttgccgctgggaaaagaggagcggcttttggagtgatggaagccctgggaacactgccaggacacatgacagagagattccaggaagccattgacaacctcgctgtgctcatgcgggcagagactggaagcaggccttacaaagccgcggcggcccaattgccggagaccctagagaccattatgcttttggggttgctgggaacagtctcgctgggaatcttcttcgtcttgatgaggaacaagggcatagggaagatgggctttggaatggtgactcttggggccagcgcatggctcatgtggctctcggaaattgagccagccagaattgcatgtgtcctcattgttgtgtttctattgctggtggtgctcatacctgagccagaaaagcaaagatctccccaggacaaccaaatggcaatcatcatcatggtagcagtaggtcttctgggcttgattaccgccaatgaactcggatggttggagagaacaaagagtgacctaagccatctaatgggaaggagagaggagggggcaaccataggattctcaatggacattgacctgcggccagcctcagcttgggccatctatgctgccttgacaactttcattaccccagccgtccaacatgcagtgaccacttcatacaacaactactccttaatggcgatggccacgcaagctggagtgttgtttggtatgggcaaagggatgccattctacgcatgggactttggagtcccgctgctaatgataggttgctactcacaattaacacccctgaccctaatagtggccatcattttgctcgtggcgcactacatgtacttgatcccagggctgcaggcagcagctgcgcgtgctgcccagaagagaacggcagctggcatcatgaagaaccctgttgtggatggaatagtggtgactgacattgacacaatgacaattgacccccaagtggagaaaaagatgggacaggtgctactcatagcagtagccgtctccagcgccatactgtcgcggaccgcctgggggtggggggaggctggggccctgatcacagccgcaacttccactttgtgggaaggctctccgaacaagtactggaactcctctacagccacttcactgtgtaacatttttaggggaagttacttggctggagcttctctaatctacacagtaacaagaaacgctggcttggtcaagagacgtgggggtggaacaggagagaccctgggagagaaatggaaggcccgcttgaaccagatgtcggccctggagttctactcctacaaaaagtcaggcatcaccgaggtgtgcagagaagaggcccgccgcgccctcaaggacggtgtggcaacgggaggccatgctgtgtcccgaggaagtgcaaagctgagatggttggtggagcggggatacctgcagccctatggaaaggtcattgatcttggatgtggcagagggggctggagttactacgccgccaccatccgcaaagttcaagaagtgaaaggatacacaaaaggaggccctggtcatgaagaacccgtgttggtgcaaagctatgggtggaacatagtccgtcttaagagtggggtggacgtctttcatatggcggctgagccgtgtgacacgttgctgtgtgacataggtgagtcatcatctagtcctgaagtggaagaagcacggacgctcagagtcctctccatggtgggggattggcttgaaaaaagaccaggagccttttgtataaaggtgttgtgcccatacaccagcactatgatggaaaccctggagcgactgcagcgtaggtatgggggaggactggtcagagtgccactctcccgcaactctacacatgagatgtattgggtctctggagcgaaaagcaacaccataaaaagtgtgtccaccacgagccagctcctcttggggcgcatggacgggcctaggaggccagtgaaatatgaggaggatgtgaatctcggctctggcacgcgggctgtggtaagctgcgctgaagctcccaacatgaagatcattggtaaccgcattgaaaggatccgcagtgagcacgcggaaacgtggttctttgacgagaaccacccatataggacatgggcttaccatggaagctatgaggcccccacacaagggtcagcgtcctctctaataaacggggttgtcaggctcctgtcaaaaccctgggatgtggtgactggagtcacaggaatagccatgaccgacaccacaccgtatggtcagcaaagagttttcaaggaaaaagtggacactagggtgccagacccccaagaaggcactcgtcaggttatgagcatggtctcttcctggttgtggaaagagctaggcaaacacaaacggccacgagtctgtaccaaagaagagttcatcaacaaggttcgtagcaatgcagcattaggggcaatatttgaagaggaaaaagagtggaagactgcagtggaagctgtgaacgatccaaggttctgggctctagtggataaggaaagagagcaccacctgagaggagagtgccagagttgtgtgtacaacatgatgggaaaaagagaaaagaaacaaggggaatttggaaaggccaagggcagccgcgccatctggtatatgtggctaggggctagatttctagagttcgaagcccttggattcttgaacgaggatcactggatggggagagagaactcaggaggtggtgttgaagggctgggattacaaagactcggatatgtcctagaagagatgagtcgtataccaggaggaaggatgtatgcagatgacactgctggctgggacacccgcatcagcaggtttgatctggagaatgaagctctaatcaccaaccaaatggaaaaagggcacagggccttggcattggccataatcaagtacacataccaaaacaaagtggtaaaggtccttagaccagctgaaaaagggaaaacagttatggacattatttcgagacaagaccaaagggggagcggacaagttgtcacttacgctcttaacacatttaccaacctagtggtgcaactcattcggaatatggaggctgaggaagttctagagatgcaagacttgtggctgctgcggaggtcagagaaagtgaccaactggttgcagagcaacggatgggataggctcaaacgaatggcagtcagtggagatgattgcgttgtgaagccaattgatgataggtttgcacatgccctcaggttcttgaatgatatgggaaaagttaggaaggacacacaagagtggaaaccctcaactggatgggacaactgggaagaagttccgttttgctcccaccacttcaacaagctccatctcaaggacgggaggtccattgtggttccctgccgccaccaagatgaactgattggccgggcccgcgtctctccaggggcgggatggagcatccgggagactgcttgcctagcaaaatcatatgcgcaaatgtggcagctcctttatttccacagaagggacctccgactgatggccaatgccatttgttcatctgtgccagttgactgggttccaactgggagaactacctggtcaatccatggaaagggagaatggatgaccactgaagacatgcttgtggtgtggaacagagtgtggattgaggagaacgaccacatggaagacaagaccccagttacgaaatggacagacatcccctatttgggaaaaagggaagacttgtggtgtggatctctcatagggcacagaccgcgcaccacctgggctgagaacattaaaaacacagtcaacatggtgcgcaggatcataggtgatgaagaaaagtacatggactacctatccacccaagttcgctacttgggtgaagaagggtctacacctggagtgctgtaagcaccagtcttaatgttgtcaggcctgctagtcagccacagcttggggaaagctgtgcagcctgtgacccccccaggagaagctgggaaaccaagcctatagtcaggccgagaacgccatggcacggaagaagccatgctgcctgtgagcccctcagaggacactgagtcaaaaaaccccacgcgcttggaggcgcaggatgggaaaagaaggtggcgaccttccccacccttcaatctggggcctgaactggagatcagctgtggatctccagaagagggactagtggttagaggag

SEQ ID NO: 3 - protein, 3423 aa, Zika virus
MKNPKKKSGGFRIVNMLKRGVARVSPFGGLKRLPAGLLLGHGPIRMVLAILAFLRFTAIKPSLGLINRWGSVGKKEAMEIIKKFKKDLAAMLRIINARKEKKRRGADTSVGIVGLLLTTAMAAEVTRRGSAYYMYLDRNDAGEAISFPTTLGMNKCYIQIMDLGHMCDATMSYECPMLDEGVEPDDVDCWCNTTSTWVVYGTCHHKKGEARRSRRAVTLPSHSTRKLQTRSQTWLESREYTKHLIRVENWIFRNPGFALAAAAIAWLLGSSTSQKVIYLVMILLIAPAYSIRCIGVSNRDFVEGMSGGTWVDVVLEHGGCVTVMAQDKPTVDIELVTTTVSNMAEVRSYCYEASISDMASDSRCPTQGEAYLDKQSDTQYVCKRTLVDRGWGNGCGLFGKGSLVTCAKFACSKKMTGKSIQPENLEYRIMLSVHGSQHSGMIVNDTGHETDENRAKVEITPNSPRAEATLGGFGSLGLDCEPRTGLDFSDLYYLTMNNKHWLVHKEWFHDIPLPWHAGADTGTPHWNNKEALVEFKDAHAKRQTVVVLGSQEGAVHTALAGALEAEMDGAKGRLSSGHLKCRLKMDKLRLKGVSYSLCTAAFTFTKIPAETLHGTVTVEVQYAGTDGPCKVPAQMAVDMQTLTPVGRLITANPVITESTENSKMMLELDPPFGDSYIVIGVGEKKITHHWHRSGSTIGKAFEATVRGAKRMAVLGDTAWDFGSVGGALNSLGKGIHQIFGAAFKSLFGGMSWFSQILIGTLLMWLGLNTKNGSISLMCLALGGVLIFLSTAVSADVGCSVDFSKKETRCGTGVFVYNDVEAWRDRYKYHPDSPRRLAAAVKQAWEDGICGISSVSRMENIMWRSVEGELNAILEENGVQLTVVVGSVKNPMWRGPQRLPVPVNELPHGWKAWGKSYFVRAAKTNNSFVVDGDTLKECPLKHRAWNSFLVEDHGFGVFHTSVWLKVREDYSLECDPAVIGTAVKGKEAVHSDLGYWIESEKNDTWRLKRAHLIEMKTCEWPKSHTLWTDGIEESDLIIPKSLAGPLSHHNTREGYRTQMKGPWHSEELEIRFEECPGTKVHVEETCGTRGPSLRSTTASGRVIEEWCCRECTMPPLSFRAKDGCWYGMEIRPRKEPESNLVRSMVTAGSTDHMDHFSLGVLVILLMVQEGLKKRMTTKIIISTSMAVLVAMILGGFSMSDLAKLAILMGATFAEMNTGGDVAHLALIAAFKVRPALLVSFIFRANWTPRESMLLALASCLLQTAISALEGDLMVLINGFALAWLAIRAMVVPRTDNITLAILAALTPLARGTLLVAWRAGLATCGGFMLLSLKGKGSVKKNLPFVMALGLTAVRLVDPINVVGLLLLTRSGKRSWPPSEVLTAVGLICALAGGFAKADIEMAGPMAAVGLLIVSYVVSGKSVDMYIERAGDITWEKDAEVTGNSPRLDVALDESGDFSLVEDDGPPMREIILKVVLMTICGMNPIAIPFAAGAWYVYVKTGKRSGALWDVPAPKEVKKGETTDGVYRVMTRRLLGSTQVGVGVMQEGVFHTMWHVTKGSALRSGEGRLDPYWGDVKQDLVSYCGPWKLDAAWDGHSEVQLLAVPPGERARNIQTLPGIFKTKDGDIGAVALDYPAGTSGSPILDKCGRVIGLYGNGVVIKNGSYVSAITQGRREEETPVECFEPSMLKKKQLTVLDLHPGAGKTRRVLPEIVREAIKTRLRTVILAPTRVVAAEMEEALRGLPVRYMTTAVNVTHSGTEIVDLMCHATFTSRLLQPIRVPNYNLYIMDEAHFTDPSSIAARGYISTRVEMGEAAAIFMTATPPGTRDAFPDSNSPIMDTEVEVPERAWSSGFDWVTDHSGKTVWFVPSVRNGNEIAACLTKAGKRVIQLSRKTFETEFQKTKHQEWDFVVTTDISEMGANFKADRVIDSRRCLKPVILDGERVILAGPMPVTHASAAQRRGRIGRNPNKPGDEYLYGGGCAETDEDHAHWLEARMLLDNIYLQDGLIASLYRPEADKVAAIEGEFKLRTEQRKTFVELMKRGDLPVWLAYQVASAGITYTDRRWCFDGTTNNTIMEDSVPAEVWTRHGEKRVLKPRWMDARVCSDHAALKSFKEFAAGKRGAAFGVMEALGTLPGHMTERFQEAIDNLAVLMRAETGSRPYKAAAAQLPETLETIMLLGLLGTVSLGIFFVLMRNKGIGKMGFGMVTLGASAWLMWLSEIEPARIACVLIVVFLLLVVLIPEPEKQRSPQDNQMAIIIMVAVGLLGLITANELGWLERTKSDLSHLMGRREEGATIGFSMDIDLRPASAWAIYAALTTFITPAVQHAVTTSYNNYSLMAMATQAGVLFGMGKGMPFYAWDFGVPLLMIGCYSQLTPLTLIVAIILLVAHYMYLIPGLQAAAARAAQKRTAAGIMKNPVVDGIVVTDIDTMTIDPQVEKKMGQVLLIAVAVSSAILSRTAWGWGEAGALITAATSTLWEGSPNKYWNSSTATSLCNIFRGSYLAGASLIYTVTRNAGLVKRRGGGTGETLGEKWKARLNQMSALEFYSYKKSGITEVCREEARRALKDGVATGGHAVSRGSAKLRWLVERGYLQPYGKVIDLGCGRGGWSYYAATIRKVQEVKGYTKGGPGHEEPVLVQSYGWNIVRLKSGVDVFHMAAEPCDTLLCDIGESSSSPEVEEARTLRVLSMVGDWLEKRPGAFCIKVLCPYTSTMMETLERLQRRYGGGLVRVPLSRNSTHEMYWVSGAKSNTIKSVSTTSQLLLGRMDGPRRPVKYEEDVNLGSGTRAVVSCAEAPNMKIIGNRIERIRSEHAETWFFDENHPYRTWAYHGSYEAPTQGSASSLINGVVRLLSKPWDVVTGVTGIAMTDTTPYGQQRVFKEKVDTRVPDPQEGTRQVMSMVSSWLWKELGKHKRPRVCTKEEFINKVRSNAALGAIFEEEKEWKTAVEAVNDPRFWALVDKEREHHLRGECQSCVYNMMGKREKKQGEFGKAKGSRAIWYMWLGARFLEFEALGFLNEDHWMGRENSGGGVEGLGLQRLGYVLEEMSRIPGGRMYADDTAGWDTRISRFDLENEALITNQMEKGHRALALAIIKYTYQNKVVKVLRPAEKGKTVMDIISRQDQRGSGQVVTYALNTFTNLVVQLIRNMEAEEVLEMQDLWLLRRSEKVTNWLQSNGWDRLKRMAVSGDDCVVKPIDDRFAHALRFLNDMGKVRKDTQEWKPSTGWDNWEEVPFCSHHFNKLHLKDGRSIVVPCRHQDELIGRARVSPGAGWSIRETACLAKSYAQMWQLLYFHRRDLRLMANAICSSVPVDWVPTGRTTWSIHGKGEWMTTEDMLVVWNRVWIEENDHMEDKTPVTKWTDIPYLGKREDLWCGSLIGHRPRTTWAENIKNTVNMVRRIIGDEEKYMDYLSTQVRYLGEEGSTPGVL

SEQ ID NO: 4 - DNA, 10649 bp, Dengue virus type 4
  source (1)..(10649): Dengue virus type 4 strain 814669  genome
agttgttagtctgtgtggaccgacaaggacagttccaaatcggaagcttgcttaacacagttctaacagtttgtttgaatagagagcagatctctggaaaaatgaaccaacgaaaaaaggtggttagaccacctttcaatatgctgaaacgcgagagaaaccgcgtatcaacccctcaagggttggtgaagagattctcaaccggacttttttctgggaaaggacccttacggatggtgctagcattcatcacgtttttgcgagtcctttccatcccaccaacagcagggattctgaagagatggggacagttgaagaaaaataaggccatcaagatactgattggattcaggaaggagataggccgcatgctgaacatcttgaacgggagaaaaaggtcaacgataacattgctgtgcttgattcccaccgtaatggcgttttccttgtcaacaagagatggcgaacccctcatgatagtggcaaaacatgaaagggggagacctctcttgtttaagacaacagaggggatcaacaaatgcactctcattgccatggacttgggtgaaatgtgtgaggacactgtcacgtataaatgccccctactggtcaataccgaacctgaagacattgattgctggtgcaacctcacgtctacctgggtcatgtatgggacatgcacccagagcggagaacggagacgagagaagcgctcagtagctttaacaccacattcaggaatgggattggaaacaagagctgagacatggatgtcatcggaaggggcttggaagcatgctcagagagtagagagctggatactcagaaacccaggattcgcgctcttggcaggatttatggcttatatgattgggcaaacaggaatccagcgaactgtcttctttgtcctaatgatgctggtcgccccatcctacggaatgcgatgcgtaggagtaggaaacagagactttgtggaaggagtctcaggtggagcatgggtcgacctggtgctagaacatggaggatgcgtcacaaccatggcccagggaaaaccaaccttggattttgaactgactaagacaacagccaaggaagtggctctgttaagaacctattgcattgaagcctcaatatcaaacataactacggcaacaagatgtccaacgcaaggagagccttatctgaaagaggaacaggaccaacagtacatttgccggagagatgtggtagacagagggtggggcaatggctgtggcttgtttggaaaaggaggagttgtgacatgtgcgaagttttcatgttcggggaagataacaggcaatttggtccaaattgagaaccttgaatacacagtggttgtaacagtccacaatggagacacccatgcagtaggaaatgacacatccaatcatggagttacagccatgataactcccaggtcaccatcggtggaagtcaaattgccggactatggagaactaacactcgattgtgaacccaggtctggaattgactttaatgagatgattctgatgaaaatgaaaaagaaaacatggctcgtgcataagcaatggtttttggatctgcctcttccatggacagcaggagcagacacatcagaggttcactggaattacaaagagagaatggtgacatttaaggttcctcatgccaagagacaggatgtgacagtgctgggatctcaggaaggagccatgcattctgccctcgctggagccacagaagtggactccggtgatggaaatcacatgtttgcaggacatcttaagtgcaaagtccgtatggagaaattgagaatcaagggaatgtcatacacgatgtgttcaggaaagttttcaattgacaaagagatggcagaaacacagcatgggacaacagtggtgaaagtcaagtatgaaggtgctggagctccgtgtaaagtccccatagagataagagatgtaaacaaggaaaaagtggttgggcgtatcatctcatccacccctttggctgagaataccaacagtgtaaccaacatagaattagaacccccctttggggacagctacatagtgataggtgttggaaacagcgcattaacactccattggttcaggaaagggagttccattggcaagatgtttgagtccacatacagaggtgcaaaacgaatggccattctaggtgaaacagcttgggattttggttccgttggtggactgttcacatcattgggaaaggctgtgcaccaggtttttggaagtgtgtatacaaccatgtttggaggagtctcatggatgattagaatcctaattgggttcttagtgttgtggattggcacgaactcaaggaacacttcaatggctatgacgtgcatagctgttggaggaatcactctgtttctgggcttcacagttcaagcagacatgggttgtgtggtgtcatggagtgggaaagaattgaagtgtggaagcggaatttttgtggttgacaacgtgcacacttggacagaacagtacaaatttcaaccagagtccccagcgagactagcgtctgcaatattaaatgcccacaaagatggggtctgtggaattagatcaaccacgaggctggaaaatgtcatgtggaagcaaataaccaacgagctaaactatgttctctgggaaggaggacatgacctcactgtagtggctggggatgtgaagggggtgttgaccaaaggcaagagagcactcacacccccagtgagtgatctgaaatattcatggaagacatggggaaaagcaaaaatcttcaccccagaagcaagaaatagcacatttttaatagacggaccagacacctctgaatgccccaatgaacgaagagcatggaactctcttgaggtggaagactatggatttggcatgttcacgaccaacatatggatgaaattccgagaaggaagttcagaagtgtgtgaccacaggttaatgtcagctgcaattaaagatcagaaagctgtgcatgctgacatgggttattggatagagagctcaaaaaaccagacctggcagatagagaaagcatctcttattgaagtgaaaacatgtctgtggcccaagacccacacactgtggagcaatggagtgctggaaagccagatgctcattccaaaatcatatgcgggccctttttcacagcacaattaccgccagggctatgccacgcaaaccgtgggcccatggcacttaggcaaattagagatagactttggagaatgccccggaacaacagtcacaattcaggaggattgtgaccatagaggcccatctttgaggaccaccactgcatctggaaaactagtcacgcaatggtgctgccgctcctgcacgatgcctcccttaaggttcttgggagaagatgggtgctggtatgggatggagattaggcccttgagtgaaaaagaagagaacatggtcaaatcacaggtgacggccggacagggcacatcagaaactttttctatgggtctgttgtgcctgaccttgtttgtggaagaatgcttgaggagaagagtcactaggaaacacatgatattagttgtggtgatcactctttgtgctatcatcctgggaggcctcacatggatggacttactacgagccctcatcatgttgggggacactatgtctggtagaataggaggacagatccacctagccatcatggcagtgttcaagatgtcaccaggatacgtgctgggtgtgtttttaaggaaactcacttcaagagagacagcactaatggtaataggaatggccatgacaacggtgctttcaattccacatgaccttatggaactcattgatggaatatcactgggactaattttgctaaaaatagtaacacagtttgacaacacccaagtgggaaccttagctctttccttgactttcataagatcaacaatgccattggtcatggcttggaggaccattatggctgtgttgtttgtggtcacactcattcctttgtgcaggacaagctgtcttcaaaaacagtctcattgggtagaaataacagcactcatcctaggagcccaagctctgccagtgtacctaatgactcttatgaaaggagcctcaagaagatcttggcctcttaacgagggcataatggctgtgggtttagttagtctcttaggaagcgctcttttaaagaatgatgtccctttagctggcccaatggtggcaggaggcttacttctggcggcttacgtgatgagtggtagctcagcagatctgtcactagagaaggccgccaatgtgcagtgggatgaaatggcagacataacaggctcaagcccaatcatagaagtgaagcaggatgaagatggctctttctccatacgggacgtcgaggaaaccaatatgataacccttttggtgaaactggcactgataacagtgtcaggtctctaccccttggcaattccagtcacaatgaccttatggtacatgtggcaagtgaaaacacaaagatcaggagccctgtgggacgtcccctcacccgctgccactaaaaaagccgcactgtctgaaggagtgtacaggatcatgcaaagagggttattcgggaaaactcaggttggagtagggatacacatggaaggtgtatttcacacaatgtggcatgtaacaagaggatcagtgatctgccacgagactgggagattggagccatcttgggctgacgtcaggaatgacatgatatcatacggtgggggatggaggcttggagacaaatgggacaaagaagaagacgttcaggtcctcgccatagaaccaggaaaaaatcctaaacatgtccaaacgaaacctggccttttcaagaccctaactggagaaattggagcagtaacattagatttcaaacccggaacgtctggttctcccatcatcaacaggaaaggaaaagtcatcggactctatggaaatggagtagttaccaaatcaggtgattacgtcagtgccataacgcaagccgaaagaattggagagccagattatgaagtggatgaggacatttttcgaaagaaaagattaactataatggacttacaccccggagctggaaagacaaaaagaattcttccatcaatagtgagagaagccttaaaaaggaggctacgaactttgattttagctcccacgagagtggtggcggccgagatggaagaggccctacgtggactgccaatccgttatcagaccccagctgtgaaatcagaacacacaggaagagagattgtagacctcatgtgtcatgcaaccttcacaacaagacttttgtcatcaaccagggttccaaattacaaccttatagtgatggatgaagcacatttcaccgatccttctagtgtcgcggctagaggatacatctcgaccagggtggaaatgggagaggcagcagccatcttcatgaccgcaacccctcccggagcgacagatccctttccccagagcaacagcccaatagaagacatcgagagggaaattccggaaaggtcatggaacacagggttcgactggataacagactaccaagggaaaactgtgtggtttgttcccagcataaaagctggaaatgacattgcaaattgtttgagaaagtcgggaaagaaagttatccagttgagtaggaaaacctttgatacagagtatccaaaaacgaaactcacggactgggactttgtggtcactacagacatatctgaaatgggggccaattttagagccgggagagtgatagaccctagaagatgcctcaagccagttatcctaacagatgggccagagagagtcattttagcaggtcctattccagtgactccagcaagcgctgctcagagaagagggcgaataggaaggaacccagcacaagaagacgaccaatacgttttctccggagacccactaaaaaatgatgaagatcatgcccactggacagaagcaaagatgctgcttgacaatatctacaccccagaagggatcattccaacattgtttggtccggaaagggaaaaaacccaagccattgatggagagtttcgcctcagaggggaacaaaggaagacttttgtggaattaatgaggagaggagaccttccggtgtggctgagctataaggtagcttctgctggcatttcttacaaagatcgggaatggtgcttcacaggggaaagaaataaccaaattttagaagaaaacatggaggttgaaatttggactagagagggagaaaagaaaaagctaaggccaagatggttagatgcacgtgtatacgctgaccccatggctttgaaggatttcaaggagtttgccagtggaaggaagagtataactctcgacatcctaacagagattgccagtttgccaacttacctttcctctagggccaagctcgcccttgataacatagtcatgctccacacaacagaaagaggagggagggcctatcaacacgccctgaacgaacttccggagtcactggaaacactcatgcttgtagctttactaggtgctatgacagcaggcatcttcctgtttttcatgcaagggaaaggaatagggaaattgtcaatgggtttgataaccattgcggtggctagtggcttgctctgggtagcagaaattcaaccccagtggatagcggcctcaatcatactagagttttttctcatggtactgttgataccggaaccagaaaaacaaaggaccccacaagacaatcaattgatctacgtcatattgaccattctcaccatcattggtctaatagcagccaacgagatggggctgattgaaaaaacaaaaacggattttgggttttaccaggtaaaaacagaaaccaccatcctcgatgtggacttgagaccagcttcagcatggacgctctatgcagtagccaccacaattctgactcccatgctgagacacaccatagaaaacacgtcggccaacctatctctagcagccattgccaaccaggcagccgtcctaatggggcttggaaaaggatggccgctccacagaatggacctcggtgtgccgctgttagcaatgggatgctattctcaagtgaacccaacaaccttgacagcatccttagtcatgcttttagtccattatgcaataataggcccaggattgcaggcaaaagccacaagagaggcccagaaaaggacagctgctgggatcatgaaaaatcccacagtggacgggataacagtaatagatctagaaccaatatcctatgacccaaaatttgaaaagcaattagggcaggtcatgctactagtcttgtgtgctggacaactactcttgatgagaacaacatgggctttctgtgaagtcttgactttggccacaggaccaatcttgaccttgtgggagggcaacccgggaaggttttggaacacgaccatagccgtatccaccgccaacattttcaggggaagttacttggcgggagctggactggctttttcactcataaagaatgcacaaacccctaggaggggaactgggaccacaggagagacactgggagagaagtggaagagacagctaaactcattagacagaagagagtttgaagagtataaaagaagtggaatactagaagtggacaggactgaagccaagtctgccctgaaagatgggtctaaaatcaagcatgcagtatctagagggtccagtaagatcagatggattgttgagagagggatggtaaagccaaaagggaaagttgtagatcttggctgtgggagaggaggatggtcttattacatggcgacactcaagaacgtgactgaagtgaaagggtatacaaaaggaggtccaggacatgaagaaccgattcccatggctacttatggttggaatttggtcaaactccattcaggggttgacgtgttctacaaacccacagagcaagtggacaccctgctctgtgatattggggagtcatcttctaatccaacaatagaggaaggaagaacattaagagttttgaagatggtggagccatggctctcttcaaaacctgaattctgcatcaaagtccttaacccctacatgccaacagtcatagaagagctggagaaactgcagagaaaacatggtgggaaccttgtcagatgcccgctgtccaggaactccacccatgagatgtattgggtgtcaggagcgtcgggaaacattgtgagctctgtgaacacaacatcaaagatgttgttgaacaggttcacaacaaggcataggaaacccacttatgagaaggacgtagatcttggggcaggaacgagaagtgtctccactgaaacagaaaaaccagacatgacaatcattgggagaaggcttcagcgattgcaagaagagcacaaagaaacctggcattatgatcaggaaaacccatacagaacctgggcgtatcatggaagctatgaagctccttcgacaggctctgcatcctccatggtgaacggggtagtaaaactgctaacaaaaccctgggatgtgattccaatggtgactcagttagccatgacagatacaaccccttttgggcaacaaagagtgttcaaagagaaggtggataccagaacaccacaaccaaaacccggtacacgaatggttatgaccacgacagccaattggctgtgggccctccttggaaagaagaaaaatcccagactgtgcacaagggaagagttcatctcaaaagttagatcaaacgcagccataggcgcagtctttcaggaagaacagggatggacatcagccagtgaagctgtgaatgacagccggttttgggaactggttgacaaagaaagggccctacaccaggaagggaaatgtgaatcgtgtgtctataacatgatgggaaaacgtgagaaaaagttaggagagtttggcagagccaagggaagccgagcaatctggtacatgtggctgggagcgcggtttctggaatttgaagccctgggttttttgaatgaagatcactggtttggcagagaaaattcatggagtggagtggaaggggaaggtctgcacagattgggatatatcctggaggagatagacaagaaggatggagacctaatgtatgctgatgacacagcaggctgggacacaagaatcactgaggatgaccttcaaaatgaggaactgatcacggaacagatggctccccaccacaagatcctagccaaagccattttcaaactaacctatcaaaacaaagtggtgaaagtcctcagacccacaccgagaggagcggtgatggatatcatatccaggaaagaccaaagaggtagtggacaagttggaacatatggtttgaacacattcaccaacatggaagttcaactcatccgccaaatggaagctgaaggagtcatcacacaagatgacatgcagaacccaaaagggttgaaagaaagagttgagaaatggctgaaagagtgtggtgtcgacaggttaaagaggatggcaatcagtggagacgattgcgtggtgaagcccctagatgagaggtttggcacttccctcctcttcttgaacgacatgggaaaggtgaggaaagacattccgcagtgggaaccatctaagggatggaaaaactggcaagaggttcctttttgctcccaccactttcacaagatctttatgaaggatggccgctcactagttgttccatgtagaaaccaggatgaactgatagggagagccagaatctcgcagggagctggatggagcttaagagaaacagcctgcctgggcaaagcttacgcccagatgtggtcgcttatgtacttccacagaagggatctgcgtttagcctccatggccatatgctcagcagttccaacggaatggtttccaacaagcagaacaacatggtcaatccacgctcatcaccagtggatgaccactgaagatatgctcaaagtgtggaacagagtgtggatagaagacaaccctaatatgactgacaagactccagtccattcgtgggaagatataccttacctagggaaaagagaggatttgtggtgtggatccctgattggactttcttccagagccacctgggcgaagaacattcacacggccataacccaggtcaggaacctgatcggaaaagaggaatacgtggattacatgccagtaatgaaaagatacagtgctccttcagagagtgaaggagttctgtaattaccaacaacaaacaccaaaggctattgaagtcaggccacttgtgccacggtttgagcaaaccgtgctgcctgtagctccgccaataatgggaggcgtaataatccccagggaggccatgcgccacggaagctgtacgcgtggcatattggactagcggttagaggagacccctcccatcactgacaaaacgcagcaaaagggggcccgaagccaggaggaagctgtactcctggtggaaggactagaggttagaggagacccccccaacacaaaaacagcatattgacgctgggaaagaccagagatcctgctgtctctgcaacatcaatccaggcacagagcgccgcaagatggattggtgttgttgatccaacaggttct

SEQ ID NO: 5 - DNA, 10649 bp, Artificial Sequence
  Dengue virus type 4 recombinant clone rDEN4 genome
agttgttagtctgtgtggaccgacaaggacagttccaaatcggaagcttgcttaacacagttctaacagtttgtttgaatagagagcagatctctggaaaaatgaaccaacgaaaaaaggtggttagaccacctttcaatatgctgaaacgcgagagaaaccgcgtatcaacccctcaagggttggtgaagagattctcaaccggacttttttctgggaaaggacccttacggatggtgctagcattcatcacgtttttgcgagtcctttccatcccaccaacagcagggattctgaagagatggggacagttgaagaaaaataaggccatcaagatactgattggattcaggaaggagataggccgcatgctgaacatcttgaacgggagaaaaaggtcaacgataacattgctgtgcttgattcccaccgtaatggcgttttccctcagcacaagagatggcgaacccctcatgatagtggcaaaacatgaaagggggagacctctcttgtttaagacaacagaggggatcaacaaatgcactctcattgccatggacttgggtgaaatgtgtgaggacactgtcacgtataaatgccccctactggtcaataccgaacctgaagacattgattgctggtgcaacctcacgtctacctgggtcatgtatgggacatgcacccagagcggagaacggagacgagagaagcgctcagtagctttaacaccacattcaggaatgggattggaaacaagagctgagacatggatgtcatcggaaggggcttggaagcatgctcagagagtagagagctggatactcagaaacccaggattcgcgctcttggcaggatttatggcttatatgattgggcaaacaggaatccagcgaactgtcttctttgtcctaatgatgctggtcgccccatcctacggaatgcgatgcgtaggagtaggaaacagagactttgtggaaggagtctcaggtggagcatgggtcgacctggtgctagaacatggaggatgcgtcacaaccatggcccagggaaaaccaaccttggattttgaactgactaagacaacagccaaggaagtggctctgttaagaacctattgcattgaagcctcaatatcaaacataactacggcaacaagatgtccaacgcaaggagagccttatctgaaagaggaacaggaccaacagtacatttgccggagagatgtggtagacagagggtggggcaatggctgtggcttgtttggaaaaggaggagttgtgacatgtgcgaagttttcatgttcggggaagataacaggcaatttggtccaaattgagaaccttgaatacacagtggttgtaacagtccacaatggagacacccatgcagtaggaaatgacacatccaatcatggagttacagccatgataactcccaggtcaccatcggtggaagtcaaattgccggactatggagaactaacactcgattgtgaacccaggtctggaattgactttaatgagatgattctgatgaaaatgaaaaagaaaacatggctcgtgcataagcaatggtttttggatctgcctcttccatggacagcaggagcagacacatcagaggttcactggaattacaaagagagaatggtgacatttaaggttcctcatgccaagagacaggatgtgacagtgctgggatctcaggaaggagccatgcattctgccctcgctggagccacagaagtggactccggtgatggaaatcacatgtttgcaggacatcttaagtgcaaagtccgtatggagaaattgagaatcaagggaatgtcatacacgatgtgttcaggaaagttttcaattgacaaagagatggcagaaacacagcatgggacaacagtggtgaaagtcaagtatgaaggtgctggagctccgtgtaaagtccccatagagataagagatgtaaacaaggaaaaagtggttgggcgtatcatctcatccacccctttggctgagaataccaacagtgtaaccaacatagaattagaacccccctttggggacagctacatagtgataggtgttggaaacagcgcattaacactccattggttcaggaaagggagttccattggcaagatgtttgagtccacatacagaggtgcaaaacgaatggccattctaggtgaaacagcttgggattttggttccgttggtggactgttcacatcattgggaaaggctgtgcaccaggtttttggaagtgtgtatacaaccatgtttggaggagtctcatggatgattagaatcctaattgggttcttagtgttgtggattggcacgaactcgaggaacacttcaatggctatgacgtgcatagctgttggaggaatcactctgtttctgggcttcacagttcaagcagacatgggttgtgtggcgtcatggagtgggaaagaattgaagtgtggaagcggaatttttgtggttgacaacgtgcacacttggacagaacagtacaaatttcaaccagagtccccagcgagactagcgtctgcaatattaaatgcccacaaagatggggtctgtggaattagatcaaccacgaggctggaaaatgtcatgtggaagcaaataaccaacgagctaaactatgttctctgggaaggaggacatgacctcactgtagtggctggggatgtgaagggggtgttgaccaaaggcaagagagcactcacacccccagtgagtgatctgaaatattcatggaagacatggggaaaagcaaaaatcttcaccccagaagcaagaaatagcacatttttaatagacggaccagacacctctgaatgccccaatgaacgaagagcatggaactctcttgaggtggaagactatggatttggcatgttcacgaccaacatatggatgaaattccgagaaggaagttcagaagtgtgtgaccacaggttaatgtcagctgcaattaaagatcagaaagctgtgcatgctgacatgggttattggatagagagctcaaaaaaccagacctggcagatagagaaagcatctcttattgaagtgaaaacatgtctgtggcccaagacccacacactgtggagcaatggagtgctggaaagccagatgctcattccaaaatcatatgcgggccctttttcacagcacaattaccgccagggctatgccacgcaaaccgtgggcccatggcacttaggcaaattagagatagactttggagaatgccccggaacaacagtcacaattcaggaggattgtgaccatagaggcccatctttgaggaccaccactgcatctggaaaactagtcacgcaatggtgctgccgctcctgcacgatgcctcccttaaggttcttgggagaagatgggtgctggtatgggatggagattaggcccttgagtgaaaaagaagagaacatggtcaaatcacaggtgacggccggacagggcacatcagaaactttttctatgggtctgttgtgcctgaccttgtttgtggaagaatgcttgaggagaagagtcactaggaaacacatgatattagttgtggtgatcactctttgtgctatcatcctgggaggcctcacatggatggacttactacgagccctcatcatgttgggggacactatgtctggtagaataggaggacagatccacctagccatcatggcagtgttcaagatgtcaccaggatacgtgctgggtgtgtttttaaggaaactcacttcaagagagacagcactaatggtaataggaatggccatgacaacggtgctttcaattccacatgaccttatggaactcattgatggaatatcactgggactaattttgctaaaaatagtaacacagtttgacaacacccaagtgggaaccttagctctttccttgactttcataagatcaacaatgccattggtcatggcttggaggaccattatggctgtgttgtttgtggtcacactcattcctttgtgcaggacaagctgtcttcaaaaacagtctcattgggtagaaataacagcactcatcctaggagcccaagctctgccagtgtacctaatgactcttatgaaaggagcctcaagaagatcttggcctcttaacgagggcataatggctgtgggtttggttagtctcttaggaagcgctcttttaaagaatgatgtccctttagctggcccaatggtggcaggaggcttacttctggcggcttacgtgatgagtggtagctcagcagatctgtcactagagaaggccgccaacgtgcagtgggatgaaatggcagacataacaggctcaagcccaatcgtagaagtgaagcaggatgaagatggctctttctccatacgggacgtcgaggaaaccaatatgataacccttttggtgaaactggcactgataacagtgtcaggtctctaccccttggcaattccagtcacaatgaccttatggtacatgtggcaagtgaaaacacaaagatcaggagccctgtgggacgtcccctcacccgctgccactaaaaaagccgcactgtctgaaggagtgtacaggatcatgcaaagagggttattcgggaaaactcaggttggagtagggatacacatggaaggtgtatttcacacaatgtggcatgtaacaagaggatcagtgatctgccacgagactgggagattggagccatcttgggctgacgtcaggaatgacatgatatcatacggtgggggatggaggcttggagacaaatgggacaaagaagaagacgttcaggtcctcgccatagaaccaggaaaaaatcctaaacatgtccaaacgaaacctggccttttcaagaccctaactggagaaattggagcagtaacattagatttcaaacccggaacgtctggttctcccatcatcaacaggaaaggaaaagtcatcggactctatggaaatggagtagttaccaaatcaggtgattacgtcagtgccataacgcaagccgaaagaattggagagccagattatgaagtggatgaggacatttttcgaaagaaaagattaactataatggacttacaccccggagctggaaagacaaaaagaattcttccatcaatagtgagagaagccttaaaaaggaggctacgaactttgattttagctcccacgagagtggtggcggccgagatggaagaggccctacgtggactgccaatccgttatcagaccccagctgtgaaatcagaacacacaggaagagagattgtagacctcatgtgtcatgcaaccttcacaacaagacttttgtcatcaaccagggttccaaattacaaccttatagtgatggatgaagcacatttcaccgatccttctagtgtcgcggctagaggatacatctcgaccagggtggaaatgggagaggcagcagccatcttcatgaccgcaacccctcccggagcgacagatccctttccccagagcaacagcccaatagaagacatcgagagggaaattccggaaaggtcatggaacacagggttcgactggataacagactaccaagggaaaactgtgtggtttgttcccagcataaaagctggaaatgacattgcaaattgtttgagaaagtcgggaaagaaagttatccagttgagtaggaaaacctttgatacagagtatccaaaaacgaaactcacggactgggactttgtggtcactacagacatatctgaaatgggggccaattttagagccgggagagtgatagaccctagaagatgcctcaagccagttatcctaccagatgggccagagagagtcattttagcaggtcctattccagtgactccagcaagcgctgctcagagaagagggcgaataggaaggaacccagcacaagaagacgaccaatacgttttctccggagacccactaaaaaatgatgaagatcatgcccactggacagaagcaaagatgctgcttgacaatatctacaccccagaagggatcattccaacattgtttggtccggaaagggaaaaaacccaagccattgatggagagtttcgcctcagaggggaacaaaggaagacttttgtggaattaatgaggagaggagaccttccggtgtggctgagctataaggtagcttctgctggcatttcttacgaagatcgggaatggtgcttcacaggggaaagaaataaccaaattttagaagaaaacatggaggttgaaatttggactagagagggagaaaagaaaaagctaaggccaagatggttagatgcacgtgtatacgctgaccccatggctttgaaggatttcaaggagtttgccagtggaaggaagagtataactctcgacatcctaacagagattgccagtttgccaacttacctttcctctagggccaagctcgcccttgataacatagtcatgctccacacaacagaaagaggagggagggcctatcaacacgccctgaacgaacttccggagtcactggaaacactcatgcttgtagctttactaggtgctatgacagcaggcatcttcctgtttttcatgcaagggaaaggaatagggaaattgtcaatgggtttgataaccattgcggtggctagtggcttgctctgggtagcagaaattcaaccccagtggatagcggcctcaatcatactagagttttttctcatggtactgttgataccggaaccagaaaaacaaaggaccccacaagacaatcaattgatctacgtcatattgaccattctcaccatcattggtctaatagcagccaacgagatggggctgattgaaaaaacaaaaacggattttgggttttaccaggtaaaaacagaaaccaccatcctcgatgtggacttgagaccagcttcagcatggacgctctatgcagtagccaccacaattctgactcccatgctgagacacaccatagaaaacacgtcggccaacctatctctagcagccattgccaaccaggcagccgtcctaatggggcttggaaaaggatggccgctccacagaatggacctcggtgtgccgctgttagcaatgggatgctattctcaagtgaacccaacaaccttgacagcatccttagtcatgcttttagtccattatgcaataataggcccaggattgcaggcaaaagccacaagagaggcccagaaaaggacagctgctgggatcatgaaaaatcccacagtggacgggataacagtaatagatctagaaccaatatcctatgacccaaaatttgaaaagcaattagggcaggtcatgctactagtcttgtgtgctggacaactactcttgatgagaacaacatgggctttctgtgaagtcttgactttggccacaggaccaatcttgaccttgtgggagggcaacccgggaaggttttggaacacgaccatagccgtatccaccgccaacattttcaggggaagttacttggcgggagctggactggctttttcactcataaagaatgcacaaacccctaggaggggaactgggaccacaggagagacactgggagagaagtggaagagacagctaaactcattagacagaaaagagtttgaagagtataaaagaagtggaatactagaagtggacaggactgaagccaagtctgccctgaaagatgggtctaaaatcaagcatgcagtatcaagagggtccagtaagatcagatggattgttgagagagggatggtaaagccaaaagggaaagttgtagatcttggctgtgggagaggaggatggtcttattacatggcgacactcaagaacgtgactgaagtgaaagggtatacaaaaggaggtccaggacatgaagaaccgattcccatggctacttatggttggaatttggtcaaactccattcaggggttgacgtgttctacaaacccacagagcaagtggacaccctgctctgtgatattggggagtcatcttctaatccaacaatagaggaaggaagaacattaagagttttgaagatggtggagccatggctctcttcaaaacctgaattctgcatcaaagtccttaacccctacatgccaacagtcatagaagagctggagaaactgcagagaaaacatggtgggaaccttgtcagatgcccgctgtccaggaactccacccatgagatgtattgggtgtcaggagcgtcgggaaacattgtgagctctgtgaacacaacatcaaagatgttgttgaacaggttcacaacaaggcataggaaacccacttatgagaaggacgtagatcttggggcaggaacgagaagtgtctccactgaaacagaaaaaccagacatgacaatcattgggagaaggcttcagcgattgcaagaagagcacaaagaaacctggcattatgatcaggaaaacccatacagaacctgggcgtatcatggaagctatgaagctccttcgacaggctctgcatcctccatggtgaacggggtggtaaaactgctaacaaaaccctgggatgtgattccaatggtgactcagttagccatgacagatacaaccccttttgggcaacaaagagtgttcaaagagaaggtggataccagaacaccacaaccaaaacccggtacacgaatggttatgaccacgacagccaattggctgtgggccctccttggaaagaagaaaaatcccagactgtgcacaagggaagagttcatctcaaaagttagatcaaacgcagccataggcgcagtctttcaggaagaacagggatggacatcagccagtgaagctgtgaatgacagccggttttgggaactggttgacaaagaaagggccctacaccaggaagggaaatgtgaatcgtgtgtctataacatgatgggaaaacgtgagaaaaagttaggagagtttggcagagccaagggaagccgagcaatctggtacatgtggctgggagcgcggtttctggaatttgaagccctgggttttttgaatgaagatcactggtttggcagagaaaattcatggagtggagtggaaggggaaggtctgcacagattgggatatatcctggaggagatagacaagaaggatggagacctaatgtatgctgatgacacagcaggctgggacacaagaatcactgaggatgaccttcaaaatgaggaactgatcacggaacagatggctccccaccacaagatcctagccaaagccattttcaaactaacctatcaaaacaaagtggtgaaagtcctcagacccacaccgcggggagcggtgatggatatcatatccaggaaagaccaaagaggtagtggacaagttggaacatatggtttgaacacattcaccaacatggaagttcaactcatccgccaaatggaagctgaaggagtcatcacacaagatgacatgcagaacccaaaagggttgaaagaaagagttgagaaatggctgaaagagtgtggtgtcgacaggttaaagaggatggcaatcagtggagacgattgcgtggtgaagcccctagatgagaggtttggcacttccctcctcttcttgaacgacatgggaaaggtgaggaaagacattccgcagtgggaaccatctaagggatggaaaaactggcaagaggttcctttttgctcccaccactttcacaagatctttatgaaggatggccgctcactagttgttccatgtagaaaccaggatgaactgatagggagagccagaatctcgcagggagctggatggagcttaagagaaacagcctgcctgggcaaagcttacgcccagatgtggtcgcttatgtacttccacagaagggatctgcgtttagcctccatggccatatgctcagcagttccaacggaatggtttccaacaagcagaacaacatggtcaatccacgctcatcaccagtggatgaccactgaagatatgctcaaagtgtggaacagagtgtggatagaagacaaccctaatatgactgacaagactccagtccattcgtgggaagatataccttacctagggaaaagagaggatttgtggtgtggatccctgattggactttcttccagagccacctgggcgaagaacattcatacggccataacccaggtcaggaacctgatcggaaaagaggaatacgtggattacatgccagtaatgaaaagatacagtgctccttcagagagtgaaggagttctgtaattaccaacaacaaacaccaaaggctattgaagtcaggccacttgtgccacggtttgagcaaaccgtgctgcctgtagctccgccaataatgggaggcgtaataatccccagggaggccatgcgccacggaagctgtacgcgtggcatattggactagcggttagaggagacccctcccatcactgataaaacgcagcaaaagggggcccgaagccaggaggaagctgtactcctggtggaaggactagaggttagaggagacccccccaacacaaaaacagcatattgacgctgggaaagaccagagatcctgctgtctctgcaacatcaatccaggcacagagcgccgcaagatggattggtgttgttgatccaacaggttct

SEQ ID NO: 6 - DNA, 10735 bp, Dengue virus type 1
  source (1)..(10735): Dengue virus type 1 clone 45AZ5 genome
agttgttagtctacgtggaccgacaagaacagtttcgaatcggaagcttgcttaacgtagttctaacagttttttattagagagcagatctctgatgaacaaccaacggaaaaagacgggtcgaccgtctttcaatatgctgaaacgcgcgagaaaccgcgtgtcaactgtttcacagttggcgaagagattctcaaaaggattgctttcaggccaaggacccatgaaattggtgatggcttttatagcattcctaagatttctagccatacctccaacagcaggaattttggctagatggggctcattcaagaagaatggagcgatcaaagtgttacggggtttcaagaaagaaatctcaaacatgttgaacataatgaacaggaggaaaagatctgtgaccatgctcctcatgctgctgcccacagccctggcgttccatctgaccacccgagggggagagccgcacatgatagttagcaagcaggaaagaggaaaatcacttttgtttaagacctctgcaggtgtcaacatgtgcacccttattgcaatggatttgggagagttatgtgaggacacaatgacctacaaatgcccccggatcactgagacggaaccagatgacgttgactgttggtgcaatgccacggagacatgggtgacctatggaacatgttctcaaactggtgaacaccgacgagacaaacgttccgtcgcactggcaccacacgtagggcttggtctagaaacaagaaccgaaacgtggatgtcctctgaaggcgcttggaaacaaatacaaaaagtggagacctgggctctgagacacccaggattcacggtgatagccctttttctagcacatgccataggaacatccatcacccagaaagggatcatttttattttgctgatgctggtaactccatccatggccatgcggtgcgtgggaataggcaacagagacttcgtggaaggactgtcaggagctacgtgggtggatgtggtactggagcatggaagttgcgtcactaccatggcaaaagacaaaccaacactggacattgaactcttgaagacggaggtcacaaaccctgccgtcctgcgcaaactgtgcattgaagctaaaatatcaaacaccaccaccgattcgagatgtccaacacaaggagaagccacgctggtggaagaacaggacacgaactttgtgtgtcgacgaacgttcgtggacagaggctggggcaatggttgtgggctattcggaaaaggtagcttaataacgtgtgctaagtttaagtgtgtgacaaaactggaaggaaagatagtccaatatgaaaacttaaaatattcagtgatagtcaccgtacacactggagaccagcaccaagttggaaatgagaccacagaacatggaacaactgcaaccataacacctcaagctcccacgtcggaaatacagctgacagactacggagctctaacattggattgttcacctagaacagggctagactttaatgagatggtgttgttgacaatgaaaaaaaaatcatggctcgtccacaaacaatggtttctagacttaccactgccttggacctcgggggcttcaacatcccaagagacttggaatagacaagacttgctggtcacatttaagacagctcatgcaaaaaagcaggaagtagtcgtactaggatcacaagaaggagcaatgcacactgcgttgactggagcgacagaaatccaaacgtctggaacgacaacaatttttgcaggacacctgaaatgcagattaaaaatggataaactgattttaaaagggatgtcatatgtaatgtgcacagggtcattcaagttagagaaggaagtggctgagacccagcatggaactgttctagtgcaggttaaatacgaaggaacagatgcaccatgcaagatccccttctcgtcccaagatgagaagggagtaacccagaatgggagattgataacagccaaccccatagtcactgacaaagaaaaaccagtcaacattgaagcggagccaccttttggtgagagctacattgtggtaggagcaggtgaaaaagctttgaaactaagctggttcaagaagggaagcagtatagggaaaatgtttgaagcaactgcccgtggagcacgaaggatggccatcctgggagacactgcatgggacttcggttctataggaggggtgttcacgtctgtgggaaaactgatacaccagatttttgggactgcgtatggagttttgttcagcggtgtttcttggaccatgaagataggaatagggattctgctgacatggctaggattaaactcaaggagcacgtccctttcaatgacgtgtatcgcagttggcatggtcacactgtacctaggagtcatggttcaggcggactcgggatgtgtaatcaactggaaaggcagagaactcaaatgtggaagcggcatttttgtcaccaatgaagtccacacctggacagagcaatataaattccaggccgactcccctaagagactatcagcggccattgggaaggcatgggaggagggtgtgtgtggaattcgatcagccactcgtctcgagaacatcatgtggaagcaaatatcaaatgaattaaaccacatcttacttgaaaatgacatgaaatttacagtggtcgtaggagacgttagtggaatcttggcccaaggaaagaaaatgattaggccacaacccatggaacacaaatactcgtggaaaagctggggaaaagccaaaatcataggagcagatgtacagaataccaccttcatcatcgacggcccaaacaccccagaatgccctgataaccaaagagcatggaacatttgggaagttgaagactatggatttggaattttcacgacaaacatatggttgaaattgcgtgactcctacactcaagtgtgtgaccaccggctaatgtcagctgccatcaaggatagcaaagcagtccatgctgacatggggtactggatagaaagtgaaaagaacgagacttggaagttggcaagagcctccttcatagaagttaagacatgcatctggccaaaatcccacactctatggagcaatggagtcctggaaagtgagatgataatcccaaagatatatggaggaccaatatctcagcacaactacagaccaggatatttcacacaaacagcagggccgtggcacttgggcaagttagaactagattttgatttatgtgaaggtaccactgttgttgtggatgaacattgtggaaatcgaggaccatctcttagaaccacaacagtcacaggaaagacaatccatgaatggtgctgtagatcttgcacgttaccccccctacgtttcaaaggagaagacgggtgctggtacggcatggaaatcagaccagtcaaggagaaggaagagaacctagttaagtcaatggtctctgcagggtcaggagaagtggacagtttttcactaggactgctatgcatatcaataatgatcgaagaggtaatgagatccagatggagcagaaaaatgctgatgactggaacattggctgtgttcctccttctcacaatgggacaattgacatggaatgatctgatcaggctatgtatcatggttggagccaacgcttcagacaagatggggatgggaacaacgtacctagctttgatggccactttcagaatgagaccaatgttcgcagtcgggctactgtttcgcagattaacatctagagaagttcttcttcttacagttggattgagtctggtggcatctgtagaactaccaaattccttagaggagctaggggatggacttgcaatgggcatcatgatgttgaaattactgactgattttcagtcacatcagctatgggctaccttgctgtctttaacatttgtcaaaacaactttttcattgcactatgcatggaagacaatggctatgatactgtcaattgtatctctcttccctttatgcctgtccacgacttctcaaaaaacaacatggcttccggtgttgctgggatctcttggatgcaaaccactaaccatgtttcttataacagaaaacaaaatctggggaaggaaaagctggcctctcaatgaaggaattatggctgttggaatagttagcattcttctaagttcacttctcaagaatgatgtgccactagctggcccactaatagctggaggcatgctaatagcatgttatgtcatatctggaagctcggccgatttatcactggagaaagcggctgaggtctcctgggaagaagaagcagaacactctggtgcctcacacaacatactagtggaggtccaagatgatggaaccatgaagataaaggatgaagagagagatgacacactcaccattctcctcaaagcaactctgctagcaatctcaggggtatacccaatgtcaataccggcgaccctctttgtgtggtatttttggcagaaaaagaaacagagatcaggagtgctatgggacacacccagccctccagaagtggaaagagcagtccttgatgatggcatttatagaattctccaaagaggattgttgggcaggtctcaagtaggagtaggagtttttcaagaaggcgtgttccacacaatgtggcacgtcaccaggggagctgtcctcatgtaccaagggaagagactggaaccaagttgggccagtgtcaaaaaagacttgatctcatatggaggaggttggaggtttcaaggatcctggaacgcgggagaagaagtgcaggtgattgctgttgaaccggggaagaaccccaaaaatgtacagacagcgccgggtaccttcaagacccctgaaggcgaagttggagccatagctctagactttaaacccggcacatctggatctcctatcgtgaacagagagggaaaaatagtaggtctttatggaaatggagtggtgacaacaagtggtacctacgtcagtgccatagctcaagctaaagcatcacaagaagggcctctaccagagattgaggacgaggtgtttaggaaaagaaacttaacaataatggacctacatccaggatcgggaaaaacaagaagataccttccagccatagtccgtgaggccataaaaagaaagctgcgcacgctagtcttagctcccacaagagttgtcgcttctgaaatggcagaggcgctcaagggaatgccaataaggtatcagacaacagcagtgaagagtgaacacacgggaaaggagatagttgaccttatgtgtcacgccactttcactatgcgtctcctgtctcctgtgagagttcccaattataatatgattatcatggatgaagcacattttaccgatccagccagcatagcagccagagggtatatctcaacccgagtgggtatgggtgaagcagctgcgattttcatgacagccactccccccggatcggtggaggcctttccacagagcaatgcagttatccaagatgaggaaagagacattcctgaaagatcatggaactcaggctatgactggatcactgatttcccaggtaaaacagtctggtttgttccaagcatcaaatcaggaaatgacattgccaactgtttaagaaagaatgggaaacgggtggtccaattgagcagaaaaacttttgacactgagtaccagaaaacaaaaaataacgactgggactatgttgtcacaacagacatatccgaaatgggagcaaacttccgagccgacagggtaatagacccgaggcggtgcctgaaaccggtaatactaaaagatggcccagagcgtgtcattctagccggaccgatgccagtgactgtggctagcgccgcccagaggagaggaagaattggaaggaaccaaaataaggaaggcgatcagtatatttacatgggacagcctctaaacaatgatgaggaccacgcccattggacagaagcaaaaatgctccttgacaacataaacacaccagaagggattatcccagccctctttgagccggagagagaaaagagtgcagcaatagacggggaatacagactacggggtgaagcgaggaaaacgttcgtggagctcatgagaagaggagatctacctgtctggctatcctacaaagttgcctcagaaggcttccagtactccgacagaaggtggtgctttgatggggaaaggaacaaccaggtgttggaggagaacatggacgtggagatctggacaaaagaaggagaaagaaagaaactacgaccccgctggctggatgccagaacatactctgacccactggctctgcgcgaattcaaagagttcgcagcaggaagaagaagcgtctcaggtgacctaatattagaaatagggaaacttccacaacatttaacgcaaagggcccagaacgccttggacaatctggttatgttgcacaactctgaacaaggaggaaaagcctatagacacgccatggaagaactaccagacaccatagaaacgttaatgctcctagctttgatagctgtgctgactggtggagtgacgttgttcttcctatcaggaaggggtctaggaaaaacatccattggcctactctgcgtgattgcctcaagtgcactgttatggatggccagtgtggaaccccattggatagcggcctctatcatactggagttctttctgatggtgttgcttattccagagccggacagacagcgcactccacaagacaaccagctagcatacgtggtgataggtctgttattcatgatattgacagtggcagccaatgagatgggattactggaaaccacaaagaaggacctggggattggtcatgcagctgctgaaaaccaccatcatgctgcaatgctggacgtagacctacatccagcttcagcctggactctctatgcagtggccacaacaattatcactcccatgatgagacacacaattgaaaacacaacggcaaatatttccctgacagctattgcaaaccaggcagctatattgatgggacttgacaagggatggccaatatcaaagatggacataggagttccacttctcgccttggggtgctattctcaggtgaacccgctgacgctgacagcggcggtattgatgctagtggctcattatgccataattggacccggactgcaagcaaaagctactagagaagctcaaaaaaggacagcagccggaataatgaaaaacccaactgtcgacgggatcgttgcaatagatttggaccctgtggtttacgatgcaaaatttgaaaaacagctaggccaaataatgttgttgatactttgcacatcacagatcctcctgatgcggaccacatgggccttgtgtgaatccatcacactagccactggacctctgactacgctttgggagggatctccaggaaaattctggaacaccacgatagcggtgtccatggcaaacatttttaggggaagttatctagcaggagcaggtctggccttttcattaatgaaatctctaggaggaggtaggagaggcacgggagcccaaggggaaacactgggagaaaaatggaaaagacagctaaaccaattgagcaagtcagaattcaacacttacaaaaggagtgggattatagaggtggatagatctgaagccaaagaggggttaaaaagaggagaaacgactaaacacgcagtgtcgagaggaacggccaaactgaggtggtttgtggagaggaaccttgtgaaaccagaagggaaagtcatagacctcggttgtggaagaggtggctggtcatattattgcgctgggctgaagaaagtcacagaagtgaaaggatacacgaaaggaggacctggacatgaggaaccaatcccaatggcaacctatggatggaacctagtaaagctatactccgggaaagatgtattctttacaccacctgagaaatgtgacaccctcttgtgtgatattggtgagtcctctccgaacccaactatagaagaaggaagaacgttacgtgttctaaagatggtggaaccatggctcagaggaaaccaattttgcataaaaattctaaatccctatatgccgagtgtggtagaaactttggagcaaatgcaaagaaaacatggaggaatgctagtgcgaaatccactctcaagaaactccactcatgaaatgtactgggtttcatgtggaacaggaaacattgtgtcagcagtaaacatgacatctagaatgctgctaaatcgattcacaatggctcacaggaagccaacatatgaaagagacgtggacttaggcgctggaacaagacatgtggcagtagaaccagaggtggccaacctagatatcattggccagaggatagagaatataaaaaatgaacacaaatcaacatggcattatgatgaggacaatccatacaaaacatgggcctatcatggatcatatgaggtcaagccatcaggatcagcctcatccatggtcaatggtgtggtgagactgctaaccaaaccatgggatgtcattcccatggtcacacaaatagccatgactgacaccacaccctttggacaacagagggtgtttaaagagaaagttgacacgcgtacaccaaaagcgaaacgaggcacagcacaaattatggaggtgacagccaggtggttatggggttttctctctagaaacaaaaaacccagaatctgcacaagagaggagttcacaagaaaagtcaggtcaaacgcagctattggagcagtgttcgttgatgaaaatcaatggaactcagcaaaagaggcagtggaagatgaacggttctgggaccttgtgcacagagagagggagcttcataaacaaggaaaatgtgccacgtgtgtctacaacatgatgggaaagagagagaaaaaattaggagagttcggaaaggcaaaaggaagtcgcgcaatatggtacatgtggttgggagcgcgctttttagagtttgaagcccttggtttcatgaatgaagatcactggttcagcagagagaattcactcagtggagtggaaggagaaggactccacaaacttggatacatactcagagacatatcaaagattccagggggaaatatgtatgcagatgacacagccggatgggacacaagaataacagaggatgatcttcagaatgaggccaaaatcactgacatcatggaacctgaacatgccctattggccacgtcaatctttaagctaacctaccaaaacaaggtagtaagggtgcagagaccagcgaaaaatggaaccgtgatggatgtcatatccagacgtgaccagagaggaagtggacaggttggaacctatggcttaaacaccttcaccaacatggaggcccaactaataagacaaatggagtctgagggaatcttttcacccagcgaattggaaaccccaaatctagccgaaagagtcctcgactggttgaaaaaacatggcaccgagaggctgaaaagaatggcaatcagtggagatgactgtgtggtgaaaccaatcgatgacagatttgcaacagccttaacagctttgaatgacatgggaaaggtaagaaaagacataccgcaatgggaaccttcaaaaggatggaatgattggcaacaagtgcctttctgttcacaccatttccaccagctgattatgaaggatgggagggagatagtggtgccatgccgcaaccaagatgaacttgtaggtagggccagagtatcacaaggcgccggatggagcttgagagaaactgcatgcctaggcaagtcatatgcacaaatgtggcagctgatgtacttccacaggagagacttgagattagcggctaatgctatctgttcagccgttccagttgattgggtcccaaccagccgcaccacctggtcgatccatgcccaccatcaatggatgacaacagaagacatgttgtcagtgtggaatagggtttggatagaggaaaacccatggatggaggacaagactcatgtgtccagttgggaagacgttccatacctaggaaaaagggaagatcaatggtgtggttccctaataggcttaacagcacgagccacctgggccaccaacatacaagtggccataaaccaagtgagaaggctcattgggaatgagaattatctagacttcatgacatcaatgaagagattcaaaaacgagagtgatcccgaaggggcactctggtaagccaactcattcacaaaataaaggaaaataaaaaatcaaacaaggcaagaagtcaggccggattaagccatagcacggtaagagctatgctgcctgtgagccccgtccaaggacgtaaaatgaagtcaggccgaaagccacggttcgagcaagccgtgctgcctgtagctccatcgtggggatgtaaaaacccgggaggctgcaaaccatggaagctgtacgcatggggtagcagactagtggttagaggagacccctcccaagacacaacgcagcagcggggcccaacaccaggggaagctgtaccctggtggtaaggactagaggttagaggagaccccccgcacaacaacaaacagcatattgacgctgggagagaccagagatcctgctgtctctacagcatcattccaggcacagaacgccaaaaaatggaatggtgctgttgaatcaacaggttct

SEQ ID NO: 7 - DNA, 10703 bp, Dengue virus type 1
  misc_feature (1)..(15): n is a, c, g, or t
  source (1)..(10703): Dengue virus type 1 (S1 vaccine strain) genome
nnnnnnnnnnnnnnntggcccgacaaagacagattctttgagggagctgagctcaacgtagttctgactgttttttgattagagagcagatctctgatgaatgaccaacggaaaaaggcgagaaacacgcctttcaatatgctgaaacgcgagagaaaccgcgtgtcaactgtacaacagttgacaaagagattctcacttggaatgctgcagggacgaggaccactaaaattgttcatggccctggtggcattccttcgtttcctaacaatcccaccaacagcagggatattaaaaagatggggaacaattaaaaaatcaaaggctattaatgttctgagaggcttcaggaaagagattggaaggatgctgaatatcttaaacaggagacgtagaactgcaggcatgatcatcatgctgattccaacagtgatggcgtttcatctgaccacacgcaacggagaaccacacatgatcgtcagtagacaagaaaaagggaaaagccttctgtttaagacaaaggacggcacgaacatgtgtaccctcatggccatggaccttggtgagttgtgtgaagacacaatcacgtataaatgtccctttctcaagcagaacgaaccagaagacatagattgttggtgcaactccacgtccacatgggtaacttatgggacatgtaccaccacaggagagcacagaagagaaaaaagatcagtggcgcttgttccacacgtgggaatgggattggagacacgaactgaaacatggatgtcatcagaaggggcctggaaacatgcccagagaattgaaacttggattctgagacatccaggctttaccataatggccgcaatcctggcatacaccataggaacgacgcatttccaaagagtcctgatattcatcctactgacagccatcgctccttcaatgacaatgcgctgcataggaatatcaaatagggactttgtggaaggagtgtcaggagggagttgggttgacatagttttagaacatggaagttgtgtgacgacgatggcaaaaaataaaccaacactggactttgaactgataaaaacagaagccaaacaacccgccaccttaaggaagtactgtatagaggctaaactgaccaacacgacaacagactcgcgctgcccaacacaaggggaacccaccctgaatgaagagcaggacaaaaggtttgtctgcaaacattccatggtagacagaggatggggaaatggatgtggattatttggaaaaggaggcatcgtgacctgtgccatgttcacatgcaaaaagaacatggagggaaaaattgtgcagccagaaaacctggaatacactgtcgttataacacctcattcaggggaagaacatgcagtcggaaatgacacaggaaaacatggtaaagaagtcaagataacaccacagagctccatcacagaggcggaactgacaggctatggcactgttacgatggagtgctctccaagaacgggcctcgacttcaatgagatggtgttgctgcaaatgaaagacaaagcttggctggtgcacagacaatggttcctagacctaccgttgccatggctgcccggagcagacacacaaggatcaaattggatacagaaagagacactggtcaccttcaaaaatccccatgcgaaaaaacaggatgttgttgtcttaggatcccaagagggggccatgcatacagcactcacaggggctacggaaatccagatgtcatcaggaaacctgctgttcacaggacatcttaagtgcaggctgagaatggacaaattacaacttaaagggatgtcatactccatgtgcacaggaaagtttaaagttgtgaaggaaatagcagaaacacaacatggaacaatagtcattagagtacaatatgaaggagacggctctccatgcaagaccccttttgagataatggatctggaaaaaagacatgttttgggccgcctgaccacagtcaacccaattgtaacagaaaaggacagtccagtcaacatagaagcagaacctccattcggagacagctacatcatcataggagtggaaccaggacaattgaagctggactggttcaagaaaggaagttccatcggccaaatgtttgagacaacaatgaggggagcgaaaagaatggccattttgggcgacacagcctgggattttggatctctgggaggagtgttcacatcaataggaaaggctctccaccaggtttttggagcaatctacggggctgctttcagtggggtctcatggactatgaagatcctcataggagttatcatcacatggataggaatgaactcacgtagcacatcactgtctgtgtcactggtattagtgggaatcgtgacactgtacttgggagttatggtgcaggccgatagtggttgcgttgtgagctggaagaacaaagaactaaaatgtggcagtggaatattcgtcacagataacgtgcatacatggacagaacaatacaagttccaaccagaatccccttcaaaactggcttcagccatccagaaagctcatgaagagggcatctgtggaatccgctcagtaacaagactggaaaatcttatgtggaaacaaataacatcagaattgaatcatattctatcagaaaatgaagtgaaactgaccatcatgacaggagacatcaaaggaatcatgcaggtaggaaaacgatctctgcggcctcaacccactgagttgaggtattcatggaaaacatggggtaaagcgaaaatgctctccacagaactccataatcagaccttcctcattgatggtcccgaaacagcagaatgccccaacacaaacagagcttggaattcactagaagttgaggactacggctttggagtattcactaccaatatatggctaagattgagagaaaagcaggatgcattttgtgactcaaaactcatgtcagcggccataaaggacaacagagccgtccatgctgatatgggttattggatagaaagcgcactcaatgatacatggaagatagagaaagcttctttcattgaagtcaaaagttgccactggccaaagtcacacactctatggagtaatggagtgctagaaagcgagatggtaattccaaagaatttcgctggaccagtgtcacaacataataacagaccaggctatcacacacaaacagcaggaccttggcatctaggcaagcttgagatggactttgatttctgcgaagggactacagtggtggtaaccgaggactgtggaaacagagggccctctttaagaacaaccactgcctcaggaaaactcataacggaatggtgttgtcgatcttgcacactaccaccactaagatacagaggtgaggatggatgctggtacgggatggaaatcagaccattgaaagagaaagaagaaaatctggtcagttctctggtcacagccggacatgggcagattgataatttctcattaggaatcttgggaatggcactgttccttgaagaaatgctcaggactcgagtaggaacgaaacatgcaatattactagtcgcagtttctttcgtaacgttaatcacagggaacatgtcttttagagacctgggaagagtgatggttatggtgggtgccaccatgacagatgacataggcatgggtgtgacttatcttgctctactagcagctttcaaagtcagaccaacctttgcagctggactgctcttgagaaaactgacctccaaggaattaatgatgaccaccataggaatcgttcttctctcccagagtagcataccagagaccattcttgaactgaccgatgcgttagctttaggcatgatggtcctcaagatggtgagaaacatggaaaaatatcagctggcagtgaccatcatggctattttgtgcgtcccaaatgctgtgatattacagaacgcatggaaagtgagttgcacaatattggcagtggtgtctgtttcccccctgttcttaacatcctcacaacagaaagcggactggataccattagcgttgacgatcaaaggcctcaatccaacagccatttttctaacaaccctctcaagaaccagcaagaaaaggagctggcctttaaatgaggccatcatggcggttgggatggtgagtatcttggccagctctctcttaaagaatgacacccccatgacaggaccattagtggctggagggcttcttactgtgtgctacgtactaactgggcggtcagccgatctggaactagagagagctaccgatgtcaaatgggatgaccaggcagagatatcaggtagcagtccaattctgtcaataacaatatcagaagatggcagcatgtcaataaagaatgaagaggaagagcaaacattgactatactcattagaacaggattgcttgtgatctcaggactctttccggtatcaataccaattacagcagcagcatggtacctgtgggaagtaaagaaacaacgggctggagtgttgtgggatgtcccctcaccaccacccgtgggaaaggctgaattggaagatggagcctacagaatcaagcaaaaaggaatccttggatattcccagatcggagctggagtttacaaagaaggaacatttcacacaatgtggcacgtcacacgtggcgctgtcctaatgcataaggggaagaggattgaaccatcatgggcggacgtcaagaaagacttaatatcatatggaggaggttggaagctagaaggagaatggaaagaaggagaagaagtccaggtcttggcattggagcctgggaaaaatccaagagccgtccaaacaaaacctggcctttttagaaccaatactggaaccataggtgccgtatctctggacttttcccctgggacgtcaggatctccaatcgtcgacaaaaaaggaaaagttgtaggcctctatggcaatggtgtcgttacaaggagtggagcatatgtgagtgccatagctcagactgaaaaaagcattgaagacaatccagagattgaagatgacatctttcgaaagagaagattgactatcatggatctccacccaggagcaggaaagacaaagagatacctcccggccatagtcagagaggccataaaaagaggcttgagaacactaatcctagcccccactagagtcgtggcagctgaaatggaggaagcccttagaggacttccaataagataccaaactccagctatcagggctgagcacaccgggcgggagattgtggacttaatgtgtcatgccacatttaccatgaggctgctatcaccaatcagggtgccaaattacaacctgatcatcatggacgaagcccattttacagatccagcaagcatagcagctaggggatacatctcaactcgagtggagatgggtgaggcagctggaatttttatgacagccactcctccgggtagcagagatccatttcctcagagtaatgcaccaattatggacgaagaaagagaaatcccggaacgttcatggaactccgggcacgagtgggtcacggattttaaaggaaagactgtctggtttgttccaagcataaaaaccggaaatgacatagcagcctgcctgagaaagaatggaaagagggtgatacaactcagtaggaagacctttgattctgaatatgtcaagactagaaccaatgactgggatttcgtggttacaactgacatctcggaaatgggtgccaactttaaagctgagagggttatagaccccagacgctgcatgaaaccagttatactgacagacggcgaagagcgggtgattctggcaggacccatgccagtgacccactctagtgcagcacaaagaagagggagaataggaaggaatccaaggaatgaaaatgatcaatatatatatatgggggaacccctggaaaatgatgaagactgtgcgcactggaaggaagctaagatgctcctagataacatcaacacacctgaaggaatcattcccagcatgttcgagccagagcgtgaaaaggtggatgccattgacggtgaatatcgcttgagaggagaagcacggaaaacttttgtggacctaatgagaagaggagacctaccagtctggttggcttataaagtggctgctgaaggtatcaactacgcagacagaagatggtgttttgacggaaccagaaacaatcaaatcttggaagaaaatgtggaagtggaaatctggacaaaggaaggggaaaggaaaaaattgaaacctagatggttagatgctaggatctactccgacccactggcgctaaaagaattcgcagccggaagaaagtccctaaccctgaacctaatcacagagatgggcagactcccaacttttatgactcagaaggccagagatgcactagacaacttggcggtgctgcacacggctgaagcgggtggaaaggcatacaatcatgctctcagtgaactaccggagaccctggagacattgcttttgctgacactgttggccacagtcacgggaggaatctttctattcctgatgagcggaaggggtatagggaagatgaccctgggaatgtgctgcataatcacggccagcatcctcttatggtatgcacaaatacaaccacattggatagcagcttcaataatattggagttctttctcatagtcttgctcattccagaaccagaaaagcagaggacaccccaggataaccaattgacttatgtcatcatagccatcctcacagtggtggccgcaaccatggcaaacgaaatgggttttctggaaaaaacaaagaaagacctcggactgggaaacattgcaactcagcaacctgagagcaacattctggacatagatctacgtcctgcatcagcatggacgttgtatgccgtggccacaacatttatcacaccaatgttgagacatagcattgaaaattcctcagtaaatgtgtccctaacagccatagctaaccaagccacagtgctaatgggtctcgggaaaggatggccattgtcaaagatggacattggagttcccctccttgctattgggtgttactcacaagtcaaccctataaccctcacagcggctcttcttttattggtagcacattatgccatcataggaccgggacttcaagccaaagcaaccagagaagctcagaaaagagcagcagcgggcatcatgaaaaacccaactgtggatggaataacagtgatagatctagatccaataccctatgatccaaagtttgaaaagcagttgggacaagtaatgctcctagtcctctgtgtgacccaagtgctgatgatgaggactacgtgggctttgtgtgaagccttaactctagcaaccggacccgtgtccacattgtgggaaggaaatccagggagattctggaacacaaccattgcagtgtcaatggcaaacatctttagagggagttacctggctggagctggacttctcttttctatcatgaagaacacaaccagcacgagaagaggaactggcaacataggagaaacgctaggagagaaatggaaaagcaggctgaacgcattggggaaaagtgaattccagatctataaaaaaagtggaattcaagaagtggacagaaccttagcaaaagaaggcattaaaagaggagaaacggatcatcacgctgtgtcgcgaggctcagcaaaactgagatggttcgttgaaagaaatttggtcacaccagaagggaaagtagtggaccttggttgcggcagagggggctggtcatactattgtggaggattaaagaatgtaagagaagtcaaaggcttaacaaaaggaggaccaggacacgaagaacctatccctatgtcaacatatgggtggaatctagtacgcttacagagcggagttgacgttttttttgttccaccagagaagtgtgacacattgttgtgtgacataggggaatcatcaccaaatcccacggtagaagcgggacgaacactcagagtccttaacctagtggaaaattggttgaacaataacacccaattttgcgtaaaggttcttaacccgtacatgccctcagtcattgaaagaatggaaaccttacaacggaaatacggaggagccttggtgagaaatccactctcacggaattccacacatgagatgtactgggtgtccaatgcttccgggaacatagtgtcatcagtgaacatgatttcaagaatgctgattaacagattcaccatgagacacaagaaggccacctatgagccagatgtcgacctcggaagcggaacccgcaatattggaattgaaagtgagacaccgaacctagacataattgggaaaagaatagaaaaaataaaacaagagcatgaaacgtcatggcactatgaccaagaccacccatacaaaacatgggcttaccatggcagctatgaaacaaaacagactggatcagcatcatccatggtgaacggagtagtcagattgctgacaaaaccctgggacgttgttccaatggtgacacagatggcaatgacagacacaactcctttcggacaacagcgcgtcttcaaagagaaggtggatacgagaacccaagaaccaaaagaaggcacaaaaaaactaatgaaaatcacggcagagtggctctggaaagaactaggaaagaaaaagacacctagaatgtgcaccagagaagaattcacaaaaaaggtgagaagcaatgcagccttgggggccatatttaccgatgagaacaagtggaaatcggcgcgtgaggctgttgaagatagtaggttttgggagctggttgacaaggaaagaaacctccatcttgaagggaaatgtgaaacatgtgtatacaacatgatgggaaaaagagagaaaaaactaggagagtttggtaaagcaaaaggcagcagagccatatggtacatgtggctcggagcacgcttcttagagttcgaagccctaggatttttgaatgaagaccattggttctccagagagaactccctgagtggagtagaaggagaagggctgcataagctaggttacatcttaagagaggtgagcaagaaagaaggaggagcaatgtatgccgatgacaccgcaggctgggacacaagaatcacaatagaggatttaaaaaatgaagaaatgataacgaaccacatggcaggagaacacaagaaacttgccgaggccatttttaaattgacgtatcaaaacaaggtggtgcgtgtgcaaagaccaacaccaagaggcacagtaatggacatcatatcgagaagagaccaaaggggtagtggacaagttggtacctatggcctcaacactttcaccaacatggaagcacaactaattaggcaaatggagggggaaggaatcttcaaaagcatccagcacttgacagcctcagaagaaatcgctgtgcaagattggctagcaagagtagggcgtgaaaggttgtcaagaatggccatcagtggagatgattgtgttgtgaaacctttagatgatagatttgcaagagctctaacagctctaaatgacatgggaaaggttaggaaggacatacagcaatgggagccctcaagaggatggaacgactggacacaggtacccttctgttcacaccattttcacgagttaattatgaaagatggtcgcacactcgtagttccatgcagaaaccaagatgaattgatcggcagagcccgaatttcccagggagctgggtggtctttacgggagacggcctgtttggggaagtcttacgcccaaatgtggagcttgatgtacttccacagacgtgatctcaggctagcggcaaatgccatctgctcggcagtcccgtcacactgggttccaacaagccggacaacctggtccatacacgccagccatgaatggatgacgacggaagacatgttgacagtttggaacaaagtgtggatcctagaaaatccatggatggaagacaaaactccagtggaatcatgggaggaaatcccatacctgggaaaaagagaagaccaatggtgcggctcactgattgggctgacaagcagagccacctgggcgaagaatatccagacagcaataaaccaagtcagatccctcattggcaatgaggaatacacagattacatgccatccatgaaaagattcagaagagaagaggaagaggcaggagttttgtggtagaaaaacatgaaacaaaacagaagtcaggtcggattaagccatagtacgggaaaaactatgctacctgtgagccccgtccaaggacgttaaaagaagtcaggccactttgatgccatagcttgagcaaactgtgcagcctgtagctcacctgagaaggtgtaaaaaatccgggaggccacaaaccatggaagctgtacgcatggcgtagtggactagcggttagaggagacccctcccttacagatcgcagcaacaatgggggcccaaggtgagatgaagctgtagtctcactggaaggactagaggttagaggagacccccccaaaacaaaaaacagcatattgacgctgggaaagaccagagatcctgctgtctcctcagcatcattccaggcacaggacgccagaaaatggaatggtgctgttgaatcaacaggttct

SEQ ID NO: 8 - DNA, 10713 bp, Dengue virus type 2
  source (1)..(10713): Dengue virus type 2 Tonga/73 genomce
agttgttagtctacgtggaccgacaaagacagattctttgagggagctaagctcaacgtagttctaactgttttttgattagagagcagatctctgatgaataaccaacggaaaaaggcgagaaacacgcctttcaatatgctgaaacgcgagagaaaccgcgtgtcaactgtacaacagttgacaaagagattctcacttggaatgctgcagggacgaggaccactaaaattgttcatggccctggtggcattccttcgtttcctaacaatcccaccaacagcagggatattaaaaagatggggaacaattaaaaaatcaaaggctattaatgttctgagaggcttcaggaaagagattggaaggatgctgaatatcttaaacaggagacgtagaactgtaggcatgatcatcatgctgactccaacagtgatggcgtttcatctgaccacacgcaacggagaaccacacatgattgtcagtagacaagaaaaagggaaaagccttctgttcaagacaaaggatggcacgaacatgtgtaccctcatggccatggaccttggtgagttgtgtgaagacacaatcacgtataaatgtccttttctcaagcagaacgaaccagaagacatagattgttggtgcaactccacgtccacatgggtaacttatgggacatgtaccaccacaggagagcacagaagagaaaaaagatcagtggcgcttgttccacacgtgggaatgggattggagacacgaactgaaacatggatgtcatcagaaggggcctggaaacatgcccagagaattgaaacttggattctgagacatccaggctttaccataatggccgcaatcctggcatacaccatagggacgacgcatttccaaagagtcctgatattcatcctactgacagccatcgctccttcaatgacaatgcgctgcataggaatatcaaatagggactttgtggaaggagtgtcaggagggagttgggttgacatagttttagaacatggaagttgtgtgacgacgatggcaaaaaacaaaccaacactggactttgaactgataaaaacagaagccaaacaacctgccaccttaaggaagtactgtatagaggccaaactgaccaacacgacaacagactcgcgctgcccaacacaaggggaacccaccctgaatgaagagcaggacaaaaggtttgtctgcaaacattccatggtagacagaggatggggaaatggatgtggattgtttggaaaaggaggcatcgtgacctgtgctatgttcacatgcaaaaagaacatggaaggaaaaattgtgcagccagaaaacctggaatacactgtcgtgataacacctcattcaggggaagaacatgcagtgggaaatgacacaggaaaacatggtaaagaagtcaagataacaccacagagctccatcacagaggcggaactgacaggctatggcactgttacgatggagtgctctccaagaacgggcctcgacttcaatgagatggtgttgctgcaaatggaagacaaagcctggctggtgcacagacaatggttcctagacctaccgttgccatggctgcccggagcagacacacaaggatcaaattggatacagaaagaaacactggtcaccttcaaaaatccccatgcgaaaaaacaggatgttgttgtcttaggatcccaagagggggccatgcatacagcactcacaggggctacggaaatccagatgtcatcaggaaacctgctgttcacaggacatctcaagtgcaggctgagaatggacaaattacaacttaaagggatgtcatactccatgtgcacaggaaagtttaaaattgtgaaggaaatagcagaaacacaacatggaacaatagtcattagagtacaatatgaaggagacggctctccatgcaagatcccctttgagataatggatctggaaaaaagacatgttttgggccgcctgatcacagtcaacccaattgtaacagaaaaggacagtccagtcaacatagaagcagaacctccattcggagacagctacatcatcataggagtggaaccaggacaattgaagctggactggttcaagaaaggaagttccatcggccaaatgtttgagacaacaatgaggggagcgaaaagaatggccattttgggtgacacagcctgggattttggatctctgggaggagtgttcacatcaataggaaaggctctccaccaggtttttggagcaatctacggggctgctttcagtggggtctcatggactatgaagatcctcataggagttatcatcacatggataggaatgaactcacgtagcacatcactgtctgtgtcactggtgttagtgggaatcgtgacactgtacttgggagttatggtgcaggccgatagtggttgcgttgtgagctggaagaacaaagaactaaaatgtggcagtggaatattcgtcacagataacgtgcatacatggacagaacaatacaagttccaaccagaatccccttcaaaactggcctcagccatccagaaagcgcatgaagagggcatctgtggaatccgctcagtaacaagactggaaaatcttatgtggaaacagataacatcagaattgaatcatattctatcagaaaatgaagtgaaactgaccatcatgacaggagacatcaaaggaatcatgcaggtaggaaaacgatctttgcggcctcaacccactgagttgaggtattcatggaaaacatggggtaaagcgaaaatgctctccacagaactccacaatcagaccttcctcattgatggtcccgaaacagcagaatgccccaacacaaacagagcttggaattcactggaagttgaggactacggctttggagtattcactaccaatatatggctaagattgagagaaaagcaggatgtattttgtgactcaaaactcatgtcagcggccataaaggacaacagagccgtccatgctgatatgggttattggatagaaagcgcactcaatgatacatggaagatagagaaagcttctttcattgaagtcaaaagttgccactggccaaagtcacacaccctatggagtaatggagtgctagaaagcgagatggtcattccaaagaatttcgctggaccagtgtcacaacataataacagaccaggctattacacacaaacagcaggaccttggcatctaggcaagcttgagatggactttgatttctgcgaagggactacagtggtggtaaccgagaactgtggaaacagagggccctctttaagaacaaccactgcctcaggaaaactcataacggaatggtgttgtcgatcttgcacactaccaccactaagatacagaggtgaggatggatgttggtacgggatggaaatcagaccattgaaagagaaagaagaaaatctggtcagttctctggttacagccggacatgggcagattgacaatttctcattaggaatcttgggaatggcactgttccttgaagaaatgctcaggactcgagtaggaacaaaacatgcaatattactagtcgcagtttctttcgtgacgctaatcacagggaacatgtcttttagagacctgggaagagtgatggttatggtgggtgccaccatgacagatgacataggcatgggtgtgacttatctcgctctactagcagcttttagagtcagaccaacctttgcagctggactgctcttgagaaaactgacctccaaggaattaatgatgactaccataggaatcgttcttctctcccagagtagcataccagagaccattcttgaactgaccgacgcgttagctctaggcatgatggtcctcaagatggtgagaaacatggaaaaatatcagctggcagtgaccatcatggctattttgtgcgtcccaaatgctgtgatattacagaacgcatggaaagtgagttgcacaatattggcagtggtgtctgtttcccccctgctcttaacatcctcacaacagaaagcggactggataccattagcgttgacgatcaaaggtcttaatccaacagccatttttctaacaaccctctcaagaaccaacaagaaaaggagctggcctttaaatgaggccatcatggcggttgggatggtgagtatcttggccagctctctcttaaagaatgacatccccatgacaggaccattagtggctggagggctccttactgtgtgctacgtgctaactgggcggtcagccgatctggaattagagagagctaccgatgtcaaatgggatgaccaggcagagatatcaggtagcagtccaatcctgtcaataacaatatcagaagatggcagcatgtcaataaagaatgaagaggaagagcaaacactgactatactcattagaacaggattgcttgtgatctcaggactctttccggtatcaataccaattacagcagcagcatggtacctgtgggaagtaaagaaacaacgggctggagtgctgtgggatgtcccctcaccaccacccgtgggaaaagctgaattggaagatggagcctacagaatcaagcaaaaaggaatccttggatattcccagatcggagctggagtttacaaagaaggaacatttcacacaatgtggcacgtcacacgtggcgctgtcctaatgcataaggggaagaggattgaaccatcatgggcggacgtcaagaaagacttaatatcatatggaggaggttggaagctagaaggagaatggaaagaaggagaagaagtccaggtcttggcattggagccagggaaaaatccaagagccgtccaaacaaagcctggcctttttagaaccaacactggaaccataggtgccgtatctctggacttttcccctgggacgtcaggatctccaatcgtcgacaaaaaaggaaaagttgtaggtctctatggcaatggtgtcgttacaaggagtggagcatatgtgagtgccatagctcagactgaaaaaagcattgaagacaatccagagattgaagatgacatctttcgaaagagaagattgactatcatggatctccacccaggagcaggaaagacaaagagatacctcccggccatagtcagagaggccataaaaagaggcttgagaacactaatcctagcccccactagagtcgtggcagctgaaatggaggaagcccttagaggacttccaataagataccaaactccagctatcagggctgagcacaccgggcgggagattgtagacttaatgtgtcatgccacatttaccatgaggctgctatcaccaatcagggtgccaaattacaacctgatcatcatggacgaagcccattttacagatccagcaagcatagcagctaggggatacatctcaactcgagtggagatgggggaggcagctggaatttttatgacagccactcctccgggtagtagagatccatttcctcagagcaatgcaccaattatggacgaagaaagagaaattccggaacgttcatggaactctgggcacgagtgggtcacggattttaaaggaaagactgtctggtttgttccaagcataaaaaccggaaatgacatagcagcctgcctgagaaagaatggaaagagggtgatacaactcagtaggaagacctttgattctgaatatgtcaagactagaaccaatgactgggatttcgtggttacaactgacatctcggaaatgggcgccaactttaaagctgagagggtcatagaccccagacgctgcatgaaaccagttatattgacagacggcgaagagcgggtgattctggcaggacccatgccagtgacccactctagtgcagcacaaagaagagggagaataggaaggaatccaaggaatgaaaatgatcaatatatatatatgggggaaccactggaaaatgatgaagactgtgcgcactggaaggaagctaagatgctcctagataatatcaacacacctgaaggaatcattcccagcttgttcgagccagagcgtgaaaaggtggatgccattgacggtgaatatcgcttgagaggagaagcacggaaaacttttgtggacctaatgagaagaggagacctaccagtctggttggcttataaagtggcagctgaaggtatcaactacgcagacagaagatggtgttttgacggaaccagaaacaatcaaatcttggaagaaaatgtggaagtggaaatctggacaaaggaaggggaaaggaaaaaattgaaacctagatggttagatgctaggatctactccgacccactggcgctaaaagagttcaaggaatttgcagccggaagaaagtccctaaccctgaacctaattacagagatgggcagactcccaacttttatgactcagaaggccagagatgcactagacaacttggcggtgctgcacacggctgaagcgggtggaaaggcatacaatcatgctctcagtgaattaccggagaccctggagacattgcttttgctgacactgttggccacagtcacgggaggaatcttcctattcctgatgagcggaaggggtatggggaagatgaccctgggaatgtgctgcataatcacggccagcatcctcttatggtatgcacaaatacagccacattggatagcagcctcaataatattggagttctttctcatagtcttgctcattccagaaccagaaaagcagaggacacctcaggataatcaattgacttatgtcatcatagccatcctcacagtggtggccgcaaccatggcaaacgaaatgggttttctggaaaaaacaaagaaagacctcggactgggaaacattgcaactcagcaacctgagagcaacattctggacatagatctacgtcctgcatcagcatggacgttgtatgccgtggctacaacatttatcacaccaatgttgagacatagcattgaaaattcctcagtaaatgtgtccctaacagccatagctaaccaagccacagtgctaatgggtctcggaaaaggatggccattgtcaaagatggacattggagttcccctccttgctattgggtgttactcacaagtcaaccctataaccctcacagcggctcttcttttattggtagcacattatgccatcataggaccgggacttcaagccaaagcaactagagaagctcagaaaagagcagcagcgggcatcatgaaaaacccaactgtggatggaataacagtgatagatctagatccaataccctatgatccaaagtttgaaaagcagttgggacaagtaatgctcctagtcctctgcgtgacccaagtgctgatgatgaggactacgtgggctttgtgtgaagccttaactctagcaactggacccgtgtccacattgtgggaaggaaatccagggagattctggaacacaaccattgcagtgtcaatggcaaacatctttagagggagttacctggctggagctggacttctcttttctatcatgaagaacacaaccagcacgagaagaggaactggcaatataggagaaacgttaggagagaaatggaaaagcagactgaacgcattggggaaaagtgaattccagatctacaaaaaaagtggaattcaagaagtggacagaaccttagcaaaagaaggcattaaaagaggagaaacggatcatcacgctgtgtcgcgaggctcagcaaaactgagatggttcgttgaaaggaatttggtcacaccagaagggaaagtagtggaccttggttgtggcagagggggctggtcatactattgtggaggattaaagaatgtaagagaagttaaaggcttaacaaaaggaggaccaggacacgaagaacctatccctatgtcaacatatgggtggaatctagtacgcttacagagcggagttgatgttttttttgttccaccagagaagtgtgacacattgttgtgtgacataggggaatcatcaccaaatcccacggtagaagcgggacgaacactcagagtcctcaacctagtggaaaattggctgaacaataacacccaattttgcgtaaaggttcttaacccgtacatgccctcagtcattgaaagaatggaaaccttacaacggaaatacggaggagccttggtgagaaatccactctcacggaattccacacatgagatgtactgggtgtccaatgcttccgggaacatagtgtcatcagtgaacatgatttcaagaatgctgatcaacagattcactatgagacacaagaaggccacctatgagccagatgtcgacctcggaagcggaacccgcaatattggaattgaaagtgagacaccgaacctagacataattgggaaaagaatagaaaaaataaaacaagagcatgaaacgtcatggcactatgatcaagaccacccatacaaaacatgggcttaccatggcagctatgaaacaaaacagactggatcagcatcatccatggtgaacggagtagtcagattgctgacaaaaccctgggacgttgttccaatggtgacacagatggcaatgacagacacaactccttttggacaacagcgcgtcttcaaagagaaggtggatacgagaacccaagaaccaaaagaaggcacaaaaaaactaatgaaaatcacggcagagtggctctggaaagaactaggaaagaaaaagacacctagaatgtgtaccagagaagaattcacaaaaaaggtgagaagcaatgcagccttgggggccatattcaccgatgagaacaagtggaaatcggcgcgtgaagccgttgaagatagtaggttttgggagctggttgacaaggaaaggaacctccatcttgaagggaaatgtgaaacatgtgtatacaacatgatggggaaaagagagaaaaaactaggagagtttggtaaagcaaaaggcagcagagccatatggtacatgtggctcggagcacgcttcttagagtttgaagccctaggatttttgaatgaagaccattggttctccagagagaactccctgagtggagtggaaggagaagggctgcataagctaggttacatcttaagagaggtgagcaagaaagaaggaggagcaatgtatgccgatgacaccgcaggctgggacacaagaatcacaatagaggatttgaaaaatgaagaaatgataacgaaccacatggcaggagaacacaagaaacttgccgaggccatttttaaattgacgtaccaaaacaaggtggtgcgtgtgcaaagaccaacaccaagaggcacagtaatggacatcatatcgagaagagaccaaaggggtagtggacaagttggtacctatggcctcaacactttcaccaacatggaagcacaactaattaggcaaatggagggggaaggaatcttcaaaagcatccagcacttgacagcctcagaagaaatcgctgtgcaagattggctagtaagagtagggcgtgaaaggttgtcaagaatggccatcagtggagatgattgtgttgtgaaacctttagatgatagatttgcaagagctctaacagctctaaatgacatgggaaaggttaggaaggacatacagcaatgggagccctcaagaggatggaacgactggacgcaggtgcccttctgttcacaccattttcacgagttaattatgaaagatggtcgcacactcgtagttccatgcagaaaccaagatgaattgatcggcagagcccgaatttcccagggagctgggtggtctttacgggagacggcctgtttggggaagtcttacgcccaaatgtggagcttgatgtacttccacagacgtgatctcaggctagcggcaaatgccatctgctcggcagtcccatcacactggattccaacaagccggacaacctggtccatacacgccagccatgaatggatgacgacggaagacatgttgacagtttggaacagagtgtggatcctagaaaatccatggatggaagacaaaactccagtggaatcatgggaggaaatcccatacctgggaaaaagagaagaccaatggtgcggctcgctgattgggctgacaagcagagccacctgggcgaagaatatccagacagcaataaaccaagtcagatccctcattggcaatgaggaatacacagattacatgccatccatgaaaagattcagaagagaagaggaagaggcaggagttttgtggtagaaaaacatgaaacaaaacagaagtcaggtcggattaagccatagtacgggaaaaactatgctacctgtgagccccgtccaaggacgttaaaagaagtcaggccattttgatgccatagcttgagcaaactgtgcagcctgtagctccacctgagaaggtgtaaaaaatccgggaggccacaaaccatggaagctgtacgcatggcgtagtggactagcggttagaggagacccctcccttacagatcgcagcaacaatgggggcccaaggtgagatgaagctgtagtctcactggaaggactagaggttagaggagacccccccaaaacaaaaaacagcatattgacgctgggaaagaccagagatcctgctgtctcctcagcatcattccaggcacagaacgccagaaaatggaatggtgctgttgaatcaacaggttct

SEQ ID NO: 9 - DNA, 10696 bp, Dengue virus type 3
agttgttagtctacgtggaccgacaagaacagtttcgactcggaagcttgcttaacgtagtgctgacagttttttattagagagcagatctctgatgaacaaccaacggaaaaagacgggaaaaccgtctatcaatatgctgaaacgcgtgagaaaccgtgtgtcaactggatcacagttggcgaagagattctcaagaggattgctgaacggccaaggaccaatgaaattggttatggcgtttatagctttcctcagatttctagccattccaccgacagcaggagtcttggctagatggggtacctttaagaagtcgggggctattaaggtcttaaaaggcttcaagaaggagatctcaaacatgctgagcattatcaacaaacggaaaaagacatcgctctgtctcatgatgatgttaccagcaacacttgctttccacttaacttcacgagatggagagccgcgcatgattgtggggaagaatgaaagaggaaaatccctactttttaagacagcctctggaatcaacatgtgcacactcatagccatggatttgggagagatgtgtgatgacacggtcacttacaaatgcccccacattaccgaagtggagcctgaagacattgactgttggtgcaaccttacatcgacatgggtgacttatggaacatgcaatcaagctggagagcatagacgcgataagagatcagtggcgttagctccccatgtcggcatgggactggacacacgcactcaaacctggatgtcggctgaaggagcttggagacaagtcgagaaggtagagacatgggcccttaggcacccagggtttaccatactagccctatttcttgcccattacataggcacttccttgacccagaaagtggttatttttatactattaatgctggttaccccatccatgacaatgagatgtgtgggagtaggaaacagagattttgtggaaggcctatcgggagctacgtgggttgacgtggtgctcgagcacggtgggtgtgtgactaccatggctaagaacaagcccacgctggacatagagcttcagaagactgaggccactcagctggcgaccctaaggaagctatgcattgagggaaaaattaccaacataacaaccgactcaagatgtcccacccaaggggaagcgattttacctgaggagcaggaccagaactacgtgtgtaagcatacatacgtggacagaggctggggaaacggttgtggtttgtttggcaagggaagcttggtgacatgcgcgaaatttcaatgtttagaatcaatagagggaaaagtggtgcaacatgagaacctcaaatacaccgtcatcatcacagtgcacacaggagaccaacaccaggtgggaaatgaaacgcagggagttacggctgagataacatcccaggcatcaaccgctgaagccattttacctgaatatggaaccctcgggctagaatgctcaccacggacaggtttggatttcaatgaaatgattttattgacaatgaagaacaaagcatggatggtacatagacaatggttctttgacttacccctaccatggacatcaggagctacaacaaaaacaccaacttggaacaggaaagagcttcttgtgacatttaaaaatgcacatgcaaaaaagcaagaagtagttgtccttggatcacaagagggagcaatgcatacagcactgacaggagctacagagatccaaacctcaggaggcacaagtatttttgcggggcacttaaaatgtagactcaagatggacaaattgaaactcaaggggatgagctatgcaatgtgcttgaatacctttgtgttgaagaaagaagtctccgaaacgcagcatgggacaatactcattaaggttgagtacaaaggggaagatgcaccctgcaagattcctttctccacggaggatggacaagggaaagctcacaatggcagactgatcacagccaatccagtggtgaccaagaaggaggagcctgtcaacattgaggctgaacctccttttggggaaagtaatatagtaattggaattggagacaaagccctgaaaatcaactggtacaggaagggaagctcgattgggaagatgttcgaggccactgccagaggtgcaaggcgcatggccatcttgggagacacagcctgggactttggatcagtgggtggtgttttgaattcattagggaaaatggtccaccaaatatttgggagtgcttacacagccctatttagtggagtctcctggataatgaaaattggaataggtgtcctcttaacctggatagggttgaattcaaaaaacacttctatgtcattttcatgcattgcgataggaatcattacactctatctgggggtcgtggtgcaagctgacatggggtgtgtcataaactggaaaggcaaagaactcaaatgtggaagtggaattttcgtcactaatgaggtccacacctggacagagcaatacaaatttcaagcagactcccccaaaagagtggcaacagccattgcaggcgcttgggagaatggagtgtgcggaattaggtcaacaaccagaatggagaacctcttgtggaagcaaatagccaatgaactgaactacatattatgggaaaacgacattaaattaacggtagttgtaggcgacataactggggtcttagagcaagggaaaagaacactaacaccacaacccatggagctaaaatattcttggaaaacatggggtttggcaaaaatagtgacagctgaaacacaaaattcctctttcataatagatgggccaagcacaccggagtgtccaagtgcctcaagagcatggaatgtgtgggaggtggaagattacgggttcggagttttcacaaccaacatatggctgaaactccgagaggtgtacacccaactatgtgaccataggctaatgtcggcagccgtcaaggatgagagggccgtacacgccgacatgggctattggatagaaagccaaaagaatggaagttggaagctagaaaaagcatccctcatagaggtgaaaacctgcacatggccaaaatcacacactctttggagcaatggtgtgctagagagtgacatgattatcccaaagagtctagctggtcccatttcgcaacacaaccacaggcccgggtaccacacccaaacggcaggaccctggcacttaggaaaattggagctggacttcaactattgtgaaggaacaacagttgtcatctcagaaaactgtgggacaagaggcccatcattgagaacaacaacagtgtcagggaagttgatacacgaatggtgttgccgctcgtgcacacttcctcccctgcgatacatgggagaagacggctgctggtatggcatggaaatcagacccattaatgagaaagaagagaacatggtaaagtctctagcctcagcagggagtggaaaggtggacaacttcacaatgggtgtcttgtgtttggcaatcctctttgaagaggtgatgagaggaaaatttgggaaaaaacacatgattgcaggggttctcttcacgtttgtgctcctcctctcagggcaaataacatggagaggcatggcgcacacactcataatgattgggtccaacgcctctgacagaatggggatgggcgtcacttacctagctctaattgcaacatttaaaattcagccattcttggctttgggattcttcctgaggaaactgacatctagagaaaatttattgctgggagttgggttggccatggcagcaacgttacgactgccagaggacattgaacaaatggcgaatggaattgctttggggctcatggctcttaaactgataacacaatttgaaacataccaactatggacggcattagtttccctaacgtgttcaaatacaattttcacgttgactgttgcctggagaacagccactctgattttagccggaatttcgcttttgccagtgtgccagtcttcgagcatgaggaaaacagattggctcccaatgactgtggcagctatgggagttccacccctaccactttttattttcagtctgaaagatacactcaaaaggagaagctggccactgaatgagggggtgatggcagttggacttgtgagcattctagctagttctctccttaggaatgatgtgcccatggctggaccattagtggctgggggcttgctgatagcgtgctacgtcataactggcacgtcagcagacctcactgtagaaaaagcagcagatgtaacatgggaggaagaggccgagcaaacaggagtgtcccacaatttaatgatcacagttgatgatgatggaacaatgagaataaaagatgacgagactgagaacatcttaacagtgcttttaaaaacagcactactaatagtatcaggcatctttccatactccatacccgcaacaatgttggtctggcatacttggcaaaagcaaacccaaagatccggcgtcctatgggacgtacccagccccccagagacacagaaagcggaactggaagaaggggtctataggatcaaacagcaaggaatttttgggaaaacccaagtgggggttggagtacagaaagaaggagttttccacaccatgtggcacgtcacaagaggggcagtgttgacacacaatgggaaaagactggaaccaaactgggctagcgtgaaaaaagatctgatttcatacggaggaggatggagattgagtgcacaatggcaaaagggggaggaggtgcaggttattgccgtagagcctgggaagaacccaaagaactttcaaaccatgccaggcatttttcagacaacaacaggggaaataggagcaattgcactggatttcaagcctggaacttcaggatctcccatcataaacagagagggaaaggtagtgggactgtatggcaatggagtggttacaaagaatggaggctatgttagtggaatagcgcaaacaaatgcagaaccagatggaccgacaccagagttggaagaagagatgttcaaaaagcgaaatctaaccataatggatcttcatcctgggtcaggaaagacgcggaaatatcttccagctattgttagagaggcaatcaagagacgcttaaggactctaattttggcaccaacaagggtagttgcagctgagatggaagaagcaatgaaagggctcccaataaggtatcaaacaactgcaacaaaatctgaacacacaggaagagagattgttgatctaatgtgtcacgcaacgttcacaatgcgcttgctgtcaccagtcagggttccaaactacaacttgataataatggatgaggctcatttcacagacccagccagtatagcggctagagggtacatatcaactcgtgtaggaatgggagaggcagccgcaattttcatgacagcaacaccccctggaacagctgatgcctttcctcagagcaacgctccaattcaagatgaagagagagacataccggaacgctcatggaattcaggcaatgaatggattactgactttgttgggaagacagtgtggtttgtccctagcatcaaagccggaaatgtcatagcaaactgtttgcggaaaaatggaaaaaaggtcattcaactcagcaggaagacctttgacacagaatatcaaaagaccaaactgaatgattgggactttgtggtgacaacagacatttcagaaatgggagccaatttcatagcagatagagtgatcgacccaagaagatgtctcaagccggtgattttgacagatggacccgagcgggtgatcctggctggaccaatgccagtcaccgtagcgagcgctgcgcaaaggagagggagagttggcaggaacccacaaaaagaaaatgaccagtacatattcatgggccagcctctcaacaaagatgaagaccatgctcactggacagaagcaaaaatgctgctggacaacatcaacacaccagaagggattataccagctctctttgaaccagaaagggagaagtcagccgccatagacggcgaataccgcctgaagggtgagtccaggaagactttcgtggaactcatgaggaggggtgacctcccagtttggctagcccataaagtagcatcagaagggatcaaatatacagatagaaaatggtgctttgatggagaacgtaataatcaaattttagaggagaatatggatgtggaaatctggacaaaggaaggagaaaagaaaaaactgagacctaggtggctcgatgcccgcacttattcagatcctttagcactcaaggaattcaaggattttgcagctggcagaaagtcaatcgcccttgatcttgtgacagaaataggaagagtgccttcacacttagcccacagaacgagaaacgccctggacaatttggtgatgctgcacacgtcagaacatggcggtagggcctacaggcatgcagtggaggaactaccagaaacgatggaaacactcttactcctgggactgatgatcttgttaacaggtggagcaatgctcttcttgatatcaggtaaagggattggaaagacttcaataggactcatttgtgtaattgcttccagcggcatgttatggatggctgatgtcccactccaatggatcgcgtcggctatagtcctggagttttttatgatggtgttgctcataccagaaccagaaaagcagagaactccccaagacaaccaactcgcatatgtcgtgataggcatacttacattggctgcaatagtagcggccaatgaaatgggactgttggaaactacaaagagagatttaggaatgtctaaagaaccaggtgttgtttctccaaccagctatttggatgtggacttgcacccagcatcagcctggacattgtacgccgtggccacaacagtaataacaccaatgttgagacacaccatagagaattccacagcaaatgtgtccctggcagccatagctaaccaggcagtggtcctgatgggtttagacaaaggatggccgatatcgaaaatggacttgggcgtaccactattggcactgggttgctattcacaagtgaacccactaactcttatagcggcagtacttttgctagtcacacattatgcaattataggtccaggattgcaggcaaaagccactcgtgaagctcagaaaaggacagctgctggaataatgaagaatccaacggtggatggaataatgacaatagacctagatcctgtaatatatgattcaaaatttgaaaagcaactaggacaggttatgctcctggttctgtgtgcagttcaacttttgttaatgagaacatcatgggccttgtgtgaagttctaaccctagccacaggaccaataacaacactctgggaaggatcacctgggaagttctggaacaccacgatagctgtttccatggcgaacatctttagagggagctatttagcaggagctgggcttgctctttctatcatgaaatcagttggaacaggaaagagaggaacagggtcacaaggtgaaaccttaggagaaaagtggaaaaagaaattaaatcagttatcccggaaagagtttgacctttacaagaaatccggaatcaccgaagtggatagaacagaagccaaagaagggttaaaaagaggagaaataacacaccatgccgtgtccagaggcagcgcaaaacttcaatggttcgtggagagaaacatggtcattcccgaaggaagagtcatagacttaggctgtggaagaggaggctggtcatattactgtgcaggactgaaaaaagttacagaagtgcgaggatacacaaaaggcggcccaggacacgaagaaccagtacctatgtctacatacggatggaacatagtcaagttaatgagtggaaaggatgttttttatctgccacctgaaaagtgtgataccctattgtgtgacattggagaatcttcaccaagcccaacagtggaagaaagcagaaccataagagtcttgaagatggttgaaccatggctaaaaaacaaccagttttgcattaaagtattgaacccatacatgccaactgtgattgagcacttagaaagactacaaaggaaacatggaggaatgcttgtgagaaatccactctcacgaaactccacgcacgaaatgtattggatatccaatggtacaggcaacatcgtctcttcagtcaacatggtatccagattgctactgaacagattcacaatgacacacaggagacccaccatagagaaagatgtggatttaggagcaggaacccgacatgtcaatgcggaaccagaaacacccaacatggatgtcattggggaaagaataaaaaggatcaaagaggagcatagttcaacatggcactatgatgatgaaaatccttacaaaacgtgggcttaccatggatcctatgaagtaaaagccacaggctcagcctcctccatgataaatggagtcgtgaaactcctcacaaaaccatgggatgtggtgcccatggtgacacagatggcaatgacagatacaactccatttggccagcaaagagtttttaaagagaaagtggacaccaggacacctaggcccatgccaggaacaagaaaggttatggagatcacagcggagtggctttggaggaccctgggaaggaacaaaagacccagattatgcacaagggaggagttcacaaagaaggtcagaaccaacgcagctatgggcgctgtcttcacagaagagaaccaatgggacagtgcgagagctgctgttgaggacgaagaattttggaaacttgtggacagagaacgtgaactccacaaactgggcaagtgtggaagctgcgtttacaacatgatgggcaagagagagaaaaaacttggagagtttggtaaagcaaaaggcagtagggctatatggtacatgtggttgggagccaggtaccttgagttcgaggcgctcggattcctcaatgaagaccactggttctcgcgtgaaaactcttacagtggagtagaaggagaaggactgcacaagctgggatacatcttgagagatatttccaagatacccggaggagccatgtatgctgatgacacagccggttgggacacaagaataacagaagatgacctgcacaatgaggaaaaaatcacacagcagatggaccctgaacacaggcagctagcgaacgctatattcaagctcacataccaaaacaaagtggtcaaagtccaacgaccaactccaaagggcacggtaatggacatcatatctaggaaagaccaaagaggcagtggacaggtgggaacttatggtctgaacacattcaccaacatggaagcccagctaatcagacaaatggaaggagaaggcgtgttgtcaaaggcagacctcgagaacccccatccgctagagaagaaaattacacaatggttggaaactaaaggagtggagaggttaaaaagaatggccatcagcggggatgattgcgtagtgaaaccaatcgacgacagattcgccaatgccctgcttgccctgaacgatatgggaaaggttaggaaggacatacctcaatggcagccatcaaagggatggcatgattggcaacaggtccctttctgctcccaccactttcatgaattgatcatgaaagatggaagaaagttggtagttccctgcagaccccaggacgaactaataggaagagcgagaatctctcaaggagcaggatggagccttagagaaactgcatgtctagggaaagcctacgctcaaatgtggactctcatgtattttcacagaagagatcttagactagcatccaacgccatatgttcagcagtaccagtccattgggtccccacgagcagaacgacatggtctattcatgctcaccatcagtggatgactacagaagacatgcttactgtctggaacagggtgtggatagaggacaatccatggatggaagacaaaactccagtcacaacgtgggaagatgttccatatctaggaaagagagaagaccaatggtgcggatcactcataggtctcacttccagagcaacctgggcccagaacatactcacagcaatccaacaggtgagaagcctcataggcaatgaagagtttctggactacatgccttcgatgaagagattcaggaaggaggaggagtcagagggagccatttggtaaaagcaggaggcaaactgtcaggccaccttaagccacagtacggaagaagctgtgcagcctgtgagccccgtccaaggacgttaaaagaagaagtcaggcccaaaagccacggtttgagcaaaccgtgctgcctgtagctccgtcgtggggacgtaaagcctgggaggctgcaaaccgtggaagctgtacgcacggtgtagcagactagtggttagaggagacccctcccatgacacaacgcagcagcggggcccgagcactgagggaagctgtacctccttgcaaaggactagaggttataggagaccccccgcaaacaaaaacagcatattgacgctgggagagaccagagatcctgctgtctcctcagcatcattccaggcacagaacgccagaaaatggaatggtgctgttgaatcaacaggttct

SEQ ID NO: 10 - RNA, 278 nt, Dengue virus
uaaaaacccgggaggcugcaaaccauggaagcuguacgcaugggguagcagacuagugguuagaggagaccccucccaagacacaacgcagcagcggggcccaacaccaggggaagcuguacccuggugguaaggacuagagguuagaggagaccccccgcacaacaacaaacagcauauugacgcugggagagaccagagauccugcugucucuacagcaucauuccaggcacagaacgccagaaaauggaauggugcuguugaaucaacagguucu

SEQ ID NO: 11 - RNA, 248 nt, Artificial Sequence
  Dengue virus construct
uaaaaacccgggaggcugcaaaccauggaagcuguacgcaugggguagcagacuagugguuagaggagaccccucccaagacacaacgcagcagcggggcccaagacuagagguuagaggagaccccccgcacaacaacaaacagcauauugacgcugggagagaccagagauccugcugucucuacagcaucauuccaggcacagaacgccagaaaauggaauggugcuguugaaucaacagguucu

SEQ ID NO: 12 - RNA, 217 nt, Artificial Sequence
  Dengue virus construct
uaaaaacccgggaggcugcaacuagugguuagaggagaccccucccaagacacaacgcagcagcggggcccaagacuagagguuagaggagaccccccgcacaacaacaaacagcauauugacgcugggagagaccagagauccugcugucucuacagcaucauuccaggcacagaacgccagaaaauggaauggugcuguugaaucaacagguucu

SEQ ID NO: 13 - RNA, 194 nt, Artificial Sequence
  Dengue virus construct
uaaaaacccgggaggcugcaaaccauggaagcuguacgcaugggguagcagacuagagguuagaggagaccccccgcacaacaacaaacagcauauugacgcugggagagaccagagauccugcugucucuacagcaucauuccaggcacagaacgccagaaaauggaauggugcuguugaaucaacagguucu

SEQ ID NO: 14 - RNA, 281 nt, Dengue virus
uaaaaaauccgggaggccacaaaccauggaagcuguacgcauggcguaguggacuagcgguuagaggagaccccucccuuacagaucgcagcaacaaugggggcccaaggugagaugaagcuguagucucacuggaaggacuagagguuagaggagacccccccaaaacaaaaaacagcauauugacgcugggaaagaccagagauccugcugucuccucagcaucauuccaggcacaggacgccagaaaauggaauggugcuguugaaucaacagguucu

SEQ ID NO: 15 - RNA, 251 nt, Artificial Sequence
  Dengue virus construct
uaaaaaauccgggaggccacaaaccauggaagcuguacgcauggcguaguggacuagcgguuagaggagaccccucccuuacagaucgcagcaacaaugggggcccaagacuagagguuagaggagacccccccaaaacaaaaaacagcauauugacgcugggaaagaccagagauccugcugucuccucagcaucauuccaggcacaggacgccagaaaauggaauggugcuguugaaucaacagguucu

SEQ ID NO: 16 - RNA, 220 nt, Artificial Sequence
  Dengue virus construct
uaaaaaauccgggaggccacaaauagcgguuagaggagaccccucccuuacagaucgcagcaacaaugggggcccaagacuagagguuagaggagacccccccaaaacaaaaaacagcauauugacgcugggaaagaccagagauccugcugucuccucagcaucauuccaggcacaggacgccagaaaauggaauggugcuguugaaucaacagguucu

SEQ ID NO: 17 - RNA, 196 nt, Artificial Sequence
  Dengue virus construct
uaaaaaauccgggaggccacaaaccauggaagcuguacgcauggcguaguggagacuagagguuagaggagacccccccaaaacaaaaaacagcauauugacgcugggaaagaccagagauccugcugucuccucagcaucauuccaggcacaggacgccagaaaauggaauggugcuguugaaucaacagguucu

SEQ ID NO: 18 - RNA, 276 nt, Dengue virus
uaaaaccugggaggcugcaaacuguggaagcuguacgcacgguguagcagacuagcgguuagaggagaccccucccaugacacaacgcagcagcggggcccgagcucugagggaagcuguaccuccuugcaaaggacuagagguuagaggagaccccccgcaaauaaaaacagcauauugacgcugggagagaccagagauccugcugucuccucagcaucauuccaggcacagaacgccagaaaauggaauggugcuguugaaucaacagguucu

SEQ ID NO: 19 - RNA, 245 nt, Artificial Sequence
  Dengue virus construct
uaaaaccugggaggcugcaaacuguggaagcuguacgcacgguguagcagacuagcgguuagaggagaccccucccaugacacaacgcagcagcggggcccaagacuagagguuagaggagaccccccgcaaauaaaaacagcauauugacgcugggagagaccagagauccugcugucuccucagcaucauuccaggcacagaacgccagaaaauggaauggugcuguugaaucaacagguucu

SEQ ID NO: 20 - RNA, 214 nt, Artificial Sequence
  Dengue virus construct
uaaaaccugggaggcugcgacuagcgguuagaggagaccccucccaugacacaacgcagcagcggggcccaagacuagagguuagaggagaccccccgcaaauaaaaacagcauauugacgcugggagagaccagagauccugcugucuccucagcaucauuccaggcacagaacgccagaaaauggaauggugcuguugaaucaacagguucu

SEQ ID NO: 21 - RNA, 190 nt, Artificial Sequence
  Dengue virus construct
uaaaaccugggaggcugcaaacuguggaagcuguacgcacgguguagcgacuagagguuagaggagaccccccgcaaauaaaaacagcauauugacgcugggagagaccagagauccugcugucuccucagcaucauuccaggcacagaacgccagaaaauggaauggugcuguugaaucaacagguucu

SEQ ID NO: 22 - RNA, 281 nt, Dengue virus
auccccagggaggccaugcgccacggaagcuguacgcguggcauauuggacuagcgguuagaggagaccccucccaucacugacaaaacgcagcaaaagggggcccgaagccaggaggaagcuguacuccugguggaaggacuagagguuagaggagacccccccaacacaaaaacagcauauugacgcugggaaagaccagagauccugcugucucugcaacaucaauccaggcacagagcgccgcaagauggauugguguuguugauccaacagguucu

SEQ ID NO: 23 - RNA, 250 nt, Artificial Sequence
  Dengue virus construct
auccccagggaggccaugcgccacggaagcuguacgcguggcauauuggacuagcgguuagaggagaccccucccaucacugacaaaacgcagcaaaagggggcccaagacuagagguuagaggagacccccccaacacaaaaacagcauauugacgcugggaaagaccagagauccugcugucucugcaacaucaauccaggcacagagcgccgcaagauggauugguguuguugauccaacagguucu

SEQ ID NO: 24 - RNA, 219 nt, Artificial Sequence
  Dengue virus construct
auccccagggaggccaugcgccacgguuagaggagaccccucccaucacugacaaaacgcagcaaaagggggcccaagacuagagguuagaggagacccccccaacacaaaaacagcauauugacgcugggaaagaccagagauccugcugucucugcaacaucaauccaggcacagagcgccgcaagauggauugguguuguugauccaacagguucu

SEQ ID NO: 25 - RNA, 195 nt, Artificial Sequence
  Dengue virus construct
auccccagggaggccaugcgccacggaagcuguacgcguggcauauuggacuagacuagagguuagaggagacccccccaacacaaaaacagcauauugacgcugggaaagaccagagauccugcugucucugcaacaucaauccaggcacagagcgccgcaagauggauugguguuguugauccaacagguucu

(19) United States
(12) Patent Application Publication
(10) Pub. No.: US 2019/0194260 A1
(43) Pub. Date: Jun. 27, 2019

(54) LIVE ATTENUATED ZIKA VIRUS VACCINE

(71) Applicants: The United States of America, as represented by the Secretary, Dept. of Health and Human Services, Bethesda, MD (US); The Johns Hopkins University, Baltimore, MD (US)

(72) Inventors: Stephen S. Whitehead, Montgomery Village, MD (US); Sara E. Woodson, Kensington, MD (US); Anna P. Durbin, Takoma Park, MD (US); Alexander G. Pletnev, Gaithersburg, MD (US); Konstantin A. Tsetsarkin, Gaithersburg, MD (US)

(73) Assignees: The United States of America, as represented by the Secretary, Dept. of Health and Human Services, Bethesda, MD (US); The Johns Hopkins University, Baltimore, MD (US)

(21) Appl. No.: 16/083,652

(22) PCT Filed: Mar. 11, 2017
(86) PCT No.: PCT/US17/21989

§ 371 Date: Sep. 10, 2018

Related U.S. Application Data
(60) Provisional application No. 62/307,170, filed on Mar. 11, 2016.

(51) Int. Cl. C07K 14/005; A61K 39/12

(52) U.S. Cl. CPC C07K 14/005; A61K 2039/5254; A61K 39/12

(57) ABSTRACT

The present disclosure relates to attenuated Zika viruses and vaccines, attenuated chimeric Zika viruses and vaccines, and to multivalent immunogenic compositions comprising Zika vaccines and vaccines to other flaviviruses. The chimeric Zika viruses includes a first nucleotide sequence encoding at least one structural protein from a Zika virus (ZIKV), a second nucleotide sequence encoding at least one nonstructural protein from a first flavivirus, and a third nucleotide sequence of a 3′ untranslated region from a second flavivirus. The multivalent immunogenic compositions comprise an attenuated ZIKV vaccine or an attenuated chimeric ZIKV vaccine (or their combination) together with one or more of a first attenuated virus that is immunogenic against dengue serotype 1, a second attenuated virus that is immunogenic against dengue serotype 2, a third attenuated virus that is immunogenic against dengue serotype 3, and a fourth attenuated virus that is immunogenic against dengue serotype 4. The present disclosure further relates to methods of inducing immune responses, as well as preventing or treating ZIKV infections, and in certain embodiments, the combined prevention or treatment of ZIKV and another flavivirus, e.g., dengue virus.

### CROSS-REFERENCE TO RELATED APPLICATIONS

The present application claims the benefit of U.S. Provisional Application No. 62/307,170, filed 11 Mar. 2016, titled “LIVE ATTENTUATED ZIKA VIRUS VACCINE,” which is incorporated by reference in its entirety for all purposes.

### GOVERNMENT FUNDING

Research supporting this application was carried out by the United States of America as represented by the Secretary, Department of Health and Human Services. The United States Government has certain rights in this invention.

### INCORPORATION BY REFERENCE

In compliance with 37 C.F.R. § 1.52(e)(5), the sequence information contained in electronic file name 1420378_442W02_Sequence_Listing_ST25.txt; size 146 KB; created on: 9 Mar. 2017; using Patent-In 3.5, and Checker 4.4.0 is hereby incorporated herein by reference in its entirety.

### BACKGROUND

### 1. Field of the Disclosure

The present disclosure relates to attenuated, immunogenic Zika viruses and Zika virus (or “ZIKV”) chimeras built on a dengue virus backbone for the production of immunogenic, live, attenuated ZIKV vaccines, and for inclusion in a multivalent (e.g., pentavalent) vaccine composition that is immunogenic against one or more flaviviruses (e.g., a dengue virus and a ZIKV).

### 2. Background

ZIKV belongs to the family Flaviviridae and specifically to the genus Flavivirus, which includes numerous important human pathogenic related viruses, including West Nile virus, dengue virus, and yellow fever virus. Flaviviruses are typically characterized as enveloped viruses having an icosahedral and/or spherical geometry with a diameter of around 50 nm per virion. The flavivirus genomes comprise a linear positive-sense RNA genome (see FIG. 1A) of about 10-11 kilobases in length and containing a single long open reading frame that encodes three major viral structural proteins (capsid (C), premembrane/membrane (prM) and envelope (E) proteins) and at least seven non-structural (NS1, NS2A, NS2B, NS3, NS4A, NS4B, NS5) proteins. The structural and nonstructural proteins are translated as a single polyprotein. The polyprotein is then processed by cellular and viral proteases to form the individual viral polypeptides. In addition, flavivirus genomes also contain conserved 5′ noncoding regions (NCR or untranslated region (5′ UTR)) of about 100 nucleotides (nt) in length and a 3′ UTR of about 400-800 nucleotides in length containing various conserved stem and loop structures that are, in part, involved in virus replication.

Infection by ZIKV has historically only been known to cause mild symptoms in humans. In addition, ZIKV infections were generally observed in limited geographic regions localized near the equator between Africa and Asia. However, the virus is now thought to be linked to infant microcephaly and miscarriage in pregnant women, and has expanded its geographic reach. Zika has now spread to Mexico, Central and South America, and the Caribbean. The Centers for Disease Control (CDC) have now reported that Zika infections in South America have reached pandemic levels

Like other flaviviruses, the ZIKV is primarily transmitted from person-to-person by mosquitoes as a vector. In particular, the ZIKV is transmitted by the female species known as Aedes aegypti, but has been detected in numerous other mosquito species in the Aedes genus, including A. africanus, A. furcifer, and A. hensilli. It is also now believed that Zika infections may also be sexually transmitted

While effective vaccines exist for other flaviviruses, such as dengue, yellow fever, tick-borne encephalitis, and Japanese encephalitis, an effective vaccine against ZIKV is not yet available. Given the current state of global outbreak, there is an immediate need in the art to develop an effective anti-Zika vaccine.

### SUMMARY OF THE INVENTION

The present disclosure relates to attenuated, immunogenic Zika viruses and ZIKV chimeras built on a dengue virus backbone for the production of immunogenic, live, attenuated ZIKV vaccines, and for inclusion in a multivalent (e.g., pentavalent) vaccine that is immunogenic against one or more flaviviruses (e.g., a dengue virus and a ZIKV).

According to an aspect, the present disclosure provides a ZIKV genome modified to contain one or more attenuating mutations (e.g., point mutations, insertions, deletions, inversions, or any combination thereof).

According to a further aspect, the present disclosure provides a chimeric ZIKV genome comprising a portion of a ZIKV genome and a portion of the genome of at least one other flavivirus, such as, but not limited to, dengue virus (e.g., DEN1, DEN2, DEN3, or DEN4), West Nile virus, yellow fever virus, Japanese encephalitis virus, tick-borne encephalitis virus, or combinations thereof.

According to another aspect, the present disclosure provides a ZIKV virion (i.e., virus particle) comprising a ZIKV genome modified to contain one or more attenuating mutations.

According to an additional aspect, the present disclosure provides a ZIKV virion (i.e., virus particle) comprising a chimeric ZIKV genome comprising a portion of a ZIKV genome and a portion of the genome of at least one other flavivirus, such as, but not limited to, dengue virus (e.g., DEN1, DEN2, DEN3, or DEN4), West Nile virus, yellow fever virus, Japanese encephalitis virus, tick-borne encephalitis virus, or combinations thereof.

According to an aspect, the present disclosure provides an immunogenic composition or vaccine comprising an attenuated ZIKV. The attenuation can be the result of the genome of the ZIKV comprising one or more attenuating mutations and/or deletions.

According to a particular aspect, the present disclosure provides an immunogenic composition or vaccine comprising an attenuated chimeric ZIKV. The chimeric ZIKV has a genome comprising a portion of a ZIKV genome and a portion of the genome of at least one other flavivirus, such as, but not limited to, dengue virus (e.g., DEN1, DEN2, DEN3, or DEN4), West Nile virus, yellow fever virus, Japanese encephalitis virus, tick-borne encephalitis virus, or combinations thereof.

According to another aspect, the present disclosure provides a pharmaceutical kit comprising an immunogenic composition or vaccine. The vaccine may comprise an attenuated ZIKV. The attenuation can result from the genome of the ZIKV comprising one or more attenuating mutations and/or deletions, together with a set of instructions for using the composition to vaccinate a subject

According to a further aspect, the present disclosure provides a pharmaceutical kit comprising an immunogenic composition or vaccine. The vaccine comprising an attenuated chimeric ZIKV having a genome comprising a portion of a ZIKV genome and a portion of the genome of at least one other flavivirus, such as, but not limited to, dengue virus (e.g., DEN1, DEN2, DEN3, or DEN4), West Nile virus, yellow fever virus, Japanese encephalitis virus, tick-borne encephalitis virus or combinations thereof, together with a set of instructions for using the composition to vaccinate a subject.

According to an aspect, the present disclosure provides a method for vaccinating a subject to provide immunity against ZIKV. The method comprising administering a pharmaceutically acceptable dose of a ZIKV vaccine. The vaccine comprising an attenuated ZIKV. The attenuation is the result of the genome of the ZIKV comprising one or more attenuating mutations and/or deletions.

According to an aspect, the present disclosure provides a method for vaccinating a subject to provide immunity against ZIKV. The method comprises administering a pharmaceutically acceptable dose of a ZIKV vaccine. The vaccine may comprise an attenuated chimeric ZIKV having a genome comprising a portion of a ZIKV genome and a portion of the genome of at least one other flavivirus, such as, but not limited to, dengue virus (e.g., DEN1, DEN2, DEN3, or DEN4), West Nile virus, yellow fever virus, Japanese encephalitis virus, tick-borne encephalitis virus, or combinations thereof.

According to a further aspect, the present disclosure provides a method for manufacturing a vaccine comprising an attenuated ZIKV, wherein said attenuation is the result of the genome of the ZIKV comprising one or more attenuating mutations and/or deletions. The method for manufacturing comprising introducing at least one attenuating mutation and/or deletions into the genome of a wildtype ZIKV and combining the attenuated ZIKV with one or more pharmaceutical excipients to provide said vaccine.

According to another aspect, the present disclosure provides a method for manufacturing a vaccine comprising an attenuated chimeric ZIKV. The manufacturing comprising combining a portion of a ZIKV genome and a portion of the genome of at least one other flavivirus, such as, but not limited to, dengue virus (e.g., DEN1, DEN2, DEN3, or DEN4), West Nile virus, yellow fever virus, Japanese encephalitis virus, tick-borne encephalitis virus, or combinations thereof, to provide the attenuated chimeric ZIKV, and combining the attenuated chimeric ZIKV with one or more pharmaceutical excipients to provide said vaccine;

According to an additional aspect, the present disclosure provides a pentavalent immunogenic composition. The composition comprising: a first attenuated virus that is immunogenic against dengue serotype 1, a second attenuated virus that is immunogenic against dengue serotype 2, a third attenuated virus that is immunogenic against dengue serotype 3, a fourth attenuated virus that is immunogenic against dengue serotype 4, and a fifth attenuated virus that is immunogenic against ZIKV. In a particular embodiment, the fifth attenuated virus is a Zika nucleic acid chimera in accordance with the present disclosure. In another particular embodiment, the fifth attenuated virus is a ZIKV comprising one or more attenuating mutations in the genome.

According to an aspect of the invention, the present disclosure provides a multivalent immunogenic composition. The composition comprising: one or more first attenuated viruses that are immunogenic against a flavivirus, and a second attenuated virus that is immunogenic against ZIKV. In a particular embodiment, the one or more first attenuated viruses is immunogenic against dengue virus (e.g., serotype 1, 2, 3, 4, or a combination thereof), West Nile virus, yellow fever virus, Japanese encephalitis virus, tick-borne encephalitis virus. In another embodiment, the second attenuated virus is a Zika nucleic acid chimera in accordance with the present disclosure. In another particular embodiment, the second attenuated virus is a ZIKV comprising one or more attenuating mutations and/or deletions in the genome.

According to an additional aspect, the present disclosure provides a method for inducing an immune response against ZIKV. The method comprising administering a pharmaceutically acceptable dose of a ZIKV vaccine comprising an attenuated ZIKV, wherein said attenuation is the result of the genome of the ZIKV comprising one or more attenuating mutations and/or deletions.

According to an additional aspect, the present disclosure provides a method for inducing an immune response against ZIKV. The method comprising administering a pharmaceutically acceptable dose of a ZIKV vaccine comprising an attenuated chimeric ZIKV having a genome comprising a portion of a ZIKV genome and a portion of the genome of at least one other flavivirus, such as, but not limited to, dengue virus (e.g., DEN1, DEN2, DEN3, or DEN4), West Nile virus, yellow fever virus, Japanese encephalitis virus, tick-borne encephalitis virus, or combinations thereof.

According to an aspect, the present disclosure provides a method for inducing an immune response against ZIKV. The method comprising administering a pharmaceutically acceptable dose of the pentavalent immunogenic composition described herein.

According to an additional aspect, the present disclosure provides a method for inducing an immune response against ZIKV. The method comprising administering a pharmaceutically acceptable dose of the multivalent immunogenic composition described herein.

In various embodiments, the attenuating mutations can be introduced into one or more of the genes encoding the three major viral structural proteins (capsid (C), premembrane/membrane (prM) and envelope (E) proteins) or into the genes encoding the at least seven non-structural (NS1, NS2A, NS2B, NS3, NS4A, NS4B, NS5) proteins.

In other embodiments, the attenuating mutations and/or deletions can be introduced into the 5′ UTR.

In still other embodiments, the attenuating mutations and/or deletions can be introduced into the 3′ UTR.

In still further embodiments, the attenuating mutations and/or deletions can be introduced into any of the nonstructural genes, structural genes, the 5′ UTR, or the 3′ UTR, or combinations thereof.

In various embodiments, chimeric flaviviruses that are attenuated and immunogenic are provided. Attenuated Zika viruses are also provided. In certain embodiments, the chimeric Zika viruses contain one or more nonstructural protein genes (e.g., NS1, NS2A, NS2B, NS3, NS4A, NS4B, NS5) of a dengue virus as a backbone, which is combined with one or more of the structural protein genes of a ZIKV (e.g., capsid (C), premembrane/membrane (prM) and envelope (E) protein genes). These chimeric viruses exhibit pronounced immunogenicity in the absence of the accompanying clinical symptoms of viral disease. The attenuated chimeric viruses are effective as immunogens or vaccines and may be combined in a pharmaceutical composition to confer immunity against ZIKV.

According to an aspect, the present disclosure provides a Zika nucleic acid chimera comprising a first nucleotide sequence encoding at least one structural protein from a ZIKV, a second nucleotide sequence encoding at least one nonstructural protein (e.g., NS1, NS2A, NS2B, NS3, NS4A, NS4B, NS5) from a first flavivirus, and a third nucleotide sequence of a 3′ untranslated region from a second flavivirus. In an embodiment, the first flavivirus is a dengue virus. In another embodiment, the first flavivirus is a ZIKV. In yet another embodiment, the second flavivirus is a dengue virus. In a further embodiment, the second flavivirus is a ZIKV. In a particular embodiment, the dengue virus is a dengue serotype 1, serotype 2, serotype 3, or serotype 4. In certain embodiments, the structural protein is pre-membrane (prM), envelope (E), or both.

In certain embodiments, each of the attenuated viruses includes the same attenuating deletion and/or mutation. In other embodiments, the 3′ untranslated region contains one or more deletions in the nucleotide sequence. For example, the deletion may be selected from the group consisting of: a Δ30 deletion, a Δ31 deletion, a Δ30/31 deletion, and a Δ86 deletion. In certain embodiments, the deletion is accompanied by a further attenuating mutation, for example, at a nucleotide that is or corresponds to position 4891 of the NS3 gene of the DEN4 genome and/or a mutation at a nucleotide that is or corresponds with position 4995 of the NS3 gene of the DEN4 genome.

Where applicable or not specifically disclaimed, any one of the embodiments described herein are contemplated to be able to combine with any other one or more embodiments, even though the embodiments are described under different aspects of the invention.

These and other embodiments are disclosed or are contemplated variations from and encompassed by, the following Detailed Description.

### BRIEF DESCRIPTION OF THE DRAWINGS

The above and other features of the present invention will now be described in detail with reference to certain exemplary embodiments thereof illustrated the accompanying drawings which are given herein below by way of illustration only, and thus are not limitative of the present invention, and wherein:

FIG. 1A shows the translation and processing of the flavivirus polyprotein. At the top is depicted the viral genome with the structural and nonstructural protein coding regions, the 5′ cap, and the 5′ and 3′ untranslated regions (UTRs) indicated. Boxes below the genome indicate precursors and mature proteins generated by the proteolytic processing cascade. Mature structural proteins are indicated by shaded boxes and the nonstructural proteins and structural protein precursors by open boxes. Contiguous stretches of uncharged amino acids are shown by black bars. Asterisks denote proteins with N-linked glycans but do not necessarily indicate the position or number of sites utilized. Cleavage sites for host signalase (♦), the viral serine protease (♥), furin or other Golgi-localized protease (♥), or unknown proteases (?) are indicated. Taken from Field's Virology, 2001 Fourth Edition, B. D. Lindenbach and C. M. Rice, page 998, Chapter 32.

FIG. 1B shows a strategy used to replace the genes for prM and E proteins of DEN2 with the corresponding genes of ZIKV to produce Zika/DEN2 chimeras that serve as candidate attenuated vaccine strains.

FIG. 1C shows a strategy used to replace the genes for prM and E proteins of DEN4 with the corresponding genes of ZIKV to produce Zika/DEN4 chimeras that serve as candidate attenuated vaccine strains.

FIG. 2A. Predicted secondary structure of the TL-1, TL-2 and TL-3 region of the 3′-UTR of DEN1 serotype virus. The GenBank accession number of the sequence used for construction of the secondary structure model is indicated. Only the last 278 nucleotides which comprise TL-1, TL-2 and TL-3, are used to avoid circularization of the structure and subsequent misfolding of known and experimentally-verified structural elements. The mfold program constraints specific for each structure model are indicated. Nucleotides that border the principle deletions are circled and numbered, with nucleotide numbering beginning at the 3′ genome end (reverse-direction numbering system) (SEQ ID NO: 10).

FIG. 2B. Δ30 deletion mutation depicted for DEN1. The Δ30 mutation deletes nt 174 to 145 of DEN1, with reverse-direction numbering system. The deleted region is indicated by the A symbol (SEQ ID NO: 11).

FIG. 2C. Δ30/31 deletion mutation depicted for DEN1. In addition to the deletion of the nucleotides comprising the Δ30 mutation, the 431 mutation deletes nt 258 to 228 of DEN1 with reverse-direction numbering system. The deleted region is indicated by the Δ symbol (SEQ ID NO: 12).

FIG. 2D. Δ86 deletion mutation depicted for DEN1. The Δ86 mutation deletes nt 228 to 145 of DEN1 with reverse-direction numbering system. The deleted region is indicated by the Δ symbol (SEQ ID NO: 13).

FIG. 3A. Predicted secondary structure of the TL-1, TL-2 and TL-3 region of the 3′-UTR of DEN2 serotype virus. The GenBank accession number of the sequence used for construction of the secondary structure model is indicated. Only the last 281 nucleotides which comprise TL-1, TL-2 and TL-3, are used to avoid circularization of the structure and subsequent misfolding of known and experimentally-verified structural elements. The mfold program constraints specific for each structure model are indicated. Nucleotides that border the principle deletions are circled and numbered, with nucleotide numbering beginning at the 3′ genome end (reverse-direction numbering system) (SEQ ID NO: 14).

FIG. 3B. Δ30 deletion mutation depicted for DEN2. The Δ30 mutation deletes nt 173 to 144 of DEN2, with reverse-direction numbering system. The deleted region is indicated by the Δ symbol (SEQ ID NO: 15).

FIG. 3C. Δ30/31 deletion mutation depicted for DEN2. In addition to the deletion of the nucleotides comprising the Δ30 mutation, the Δ31 mutation deletes nt 258 to 228 of DEN2 with reverse-direction numbering system. The deleted region is indicated by the A symbol (SEQ ID NO: 16).

FIG. 3D. Δ86 deletion mutation depicted for DEN2. The Δ86 mutation deletes nt 228 to 144 of DEN2 with reverse-direction numbering system. The deleted region is indicated by the A symbol (SEQ ID NO: 17).

FIG. 4A. Predicted secondary structure of the TL-1, TL-2 and TL-3 region of the 3′-UTR of DEN3 serotype virus. The GenBank accession number of the sequence used for construction of the secondary structure model is indicated. Only the last 276 nucleotides which comprise TL-1, TL-2 and TL-3, are used to avoid circularization of the structure and subsequent misfolding of known and experimentally-verified structural elements. The mfold program constraints specific for each structure model are indicated. Nucleotides that border the principle deletions are circled and numbered, with nucleotide numbering beginning at the 3′ genome end (reverse-direction numbering system) (SEQ ID NO: 18).

FIG. 4B. Δ30 deletion mutation depicted for DEN3. The Δ30 mutation deletes nt 173 to 143 of DEN3, with reverse-direction numbering system. The deleted region is indicated by the A symbol (SEQ ID NO: 19).

FIG. 4C. Δ30/31 deletion mutation depicted for DEN3. In addition to the deletion of the nucleotides comprising the Δ30 mutation, the 431 mutation deletes nt 258 to 228 of DEN3 with reverse-direction numbering system. The deleted region is indicated by the symbol (SEQ ID NO: 20).

FIG. 4D. Δ86 deletion mutation depicted for DEN3. The Δ86 mutation deletes nt 228 to 143 of DEN3 with reverse-direction numbering system. The deleted region is indicated by the Δ symbol (SEQ ID NO: 21).

FIG. 5A. Predicted secondary structure of the TL-1, TL-2 and TL-3 region of the 3′-UTR of DEN4 serotype virus. The GenBank accession number of the sequence used for construction of the secondary structure model is indicated. Only the last 281 nucleotides which comprise TL-1, TL-2 and TL-3, are used to avoid circularization of the structure and subsequent misfolding of known and experimentally-verified structural elements. The mfold program constraints specific for each structure model are indicated. Nucleotides that border the principle deletions are circled and numbered, with nucleotide numbering beginning at the 3′ genome end (reverse-direction numbering system) (SEQ ID NO: 22).

FIG. 5B. Δ30 deletion mutation depicted for DEN4. The Δ30 mutation deletes nt 172 to 143 of DEN4, with reverse-direction numbering system. The deleted region is indicated by the Δ symbol (SEQ ID NO: 23).

FIG. 5C. Δ30/31 deletion mutation depicted for DEN4. In addition to the deletion of the nucleotides comprising the Δ30 mutation, the Δ31 mutation deletes nt 258 to 228 of DEN4 with reverse-direction numbering system. The deleted region is indicated by the symbol (SEQ ID NO: 24).

FIG. 5D. Δ86 deletion mutation depicted for DEN4. The Δ86 mutation deletes nt 228 to 143 of DEN4 with reverse-direction numbering system. The deleted region is indicated by the Δ symbol (SEQ ID NO: 25).

FIG. 6. The live attenuated tetravalent dengue virus vaccine contains dengue virus representing each of the 4 serotypes, with each serotype containing its full set of unaltered wild-type structural and non-structural proteins and a shared Δ30 attenuating mutations. The relative location of the Δ30 mutations in the 3′ untranslated region (UTR) of each component is indicated by an arrow. In certain aspects or embodiments as described herein, the live attenuated pentavalent virus vaccine comprises the live attenuated tetravalent dengue virus vaccine combined with an attenuated virus that is immunogenic against ZIKV (not shown).

FIG. 7A. Nucleotide sequence alignment of the TL2 region of DEN1, DEN2, DEN3, and DEN4 and their Δ30 derivatives. Also shown is the corresponding region for each of the four DEN serotypes. Upper case letters indicate sequence homology among all 4 serotypes, underlining indicates nucleotide pairing to form the stem structure.

FIG. 7B. Predicted secondary structure of the TL2 region of each DEN serotype. Nucleotides that are removed by the MO mutations are boxed (DEN1—between nucleotides 10562-10591, DEN2 Tonga/74—between nucleotides 10541-10570, DEN3 Sleman/78—between nucleotides 10535-10565, and DEN4—between nucleotides 10478-10607).

FIG. 8A. Recombinant chimeric dengue viruses were constructed by introducing either the CME or the ME regions of DEN2 (Tonga/74) into the DEN4 genetic background. The relative location of the Δ30 mutation in the 3′ UTR is indicated by an arrow and intertypic junctions 1, 2, and 3 are indicated.

FIG. 8B. Nucleotide and amino acid sequence of the intertypic junction regions. Restriction enzyme recognition sites used in assembly of each chimeric cDNA are indicated.

FIG. 9A. Recombinant chimeric dengue viruses were constructed by introducing either the CME or the ME regions of DEN3 (Sleman/78) into the DEN4 genetic background. The relative location of the Δ30 mutation in the 3′ UTR is indicated by an arrow and intertypic junctions 1, 2, and 3 are indicated. Restriction enzyme recognition sites used in assembly of each chimeric cDNA are indicated.

FIG. 9B. Nucleotide and amino acid sequence of the intertypic junction regions. Restriction enzyme recognition sites used in assembly of each chimeric cDNA are indicated.

FIG. 10A. Recombinant chimeric dengue viruses were constructed by introducing either the CME or the ME regions of DEN1 (Puerto Rico/94) into the DEN4 genetic background. The relative location of the MO mutation in the 3′ UTR is indicated by an arrow and intertypic junctions 1, 2, and 3 are indicated. Restriction enzyme recognition sites used in assembly of each chimeric cDNA are indicated.

FIG. 10B. Nucleotide and amino acid sequence of the intertypic junction regions. Restriction enzyme recognition sites used in assembly of each chimeric cDNA are indicated.

FIG. 11. Plasmid of the Zika/DEN2Δ30 chimera is shown. It should be noted that any of the other dengue virus backbones described below may be substituted for the DEN2Δ30 backbone of FIG. 11.

FIG. 12. Pentavalent DENY and ZIKV vaccine. The depicted chimeric cDNA plasmids replace the prM and E gene regions of either DEN2Δ30 or DEN4Δ30 with those derived from ZIKV-Paraiba/2015 (Brazil). For stability in E. coli, the viral open reading frame was disrupted by intron sequences. To recovery infectious viruses, Vero cells were transfected with the cDNA plasmid and transcription to create the virus genome proceeds from the CMV promoter sequence and is terminated by ribozyme (RBZ) and terminator (TERM) sequences. Intron sequences were removed by the normal RNA splicing process.

FIGS. 13A and 13B. Plasmid maps for DENV-2 (FIG. 13A) and DENV-4 (FIG. 13B) backgrounds.

FIGS. 14A, 14B, and 14C. Illustrate the locations of the intron insertions. The standard intron sequence is the same for each cDNA construct. ZV-D2 contains a single insertion at alanine codon 149 in the NS1 gene region (FIG. 14A). ZV-D4 contains two intron insertions located at alanine codon 148 in NS2A (FIG. 14B) and alanine codon 425 of NS5 (FIG. 14C).

FIGS. 15A and 15B. Virus growth kinetics were evaluated at two different multiplicities of infection (MOI) for the DENV-ZIKV chimeras. Chimeric viruses rZIKV/D2Δ30-710 (DEN2430 background) and rZIKV/D4Δ30-713 (DEN4Δ30 background) were recovered in Vero cells, biologically cloned by two rounds of terminal dilution in Vero cells and then further amplified by passage in Vero cells to generate working seed stocks. Both chimeric viruses replicate to above 6 log₁₀PFU/mL with titers peaking at about day 5. For both viruses, an MOI of 0.01 provided higher yields.

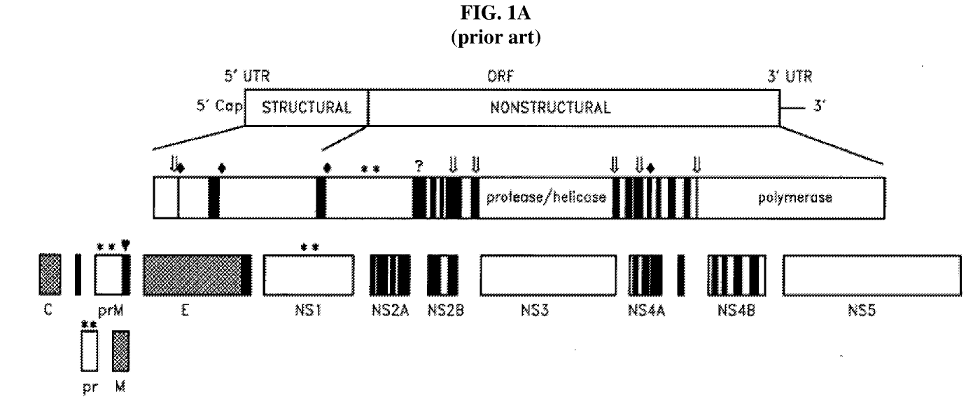

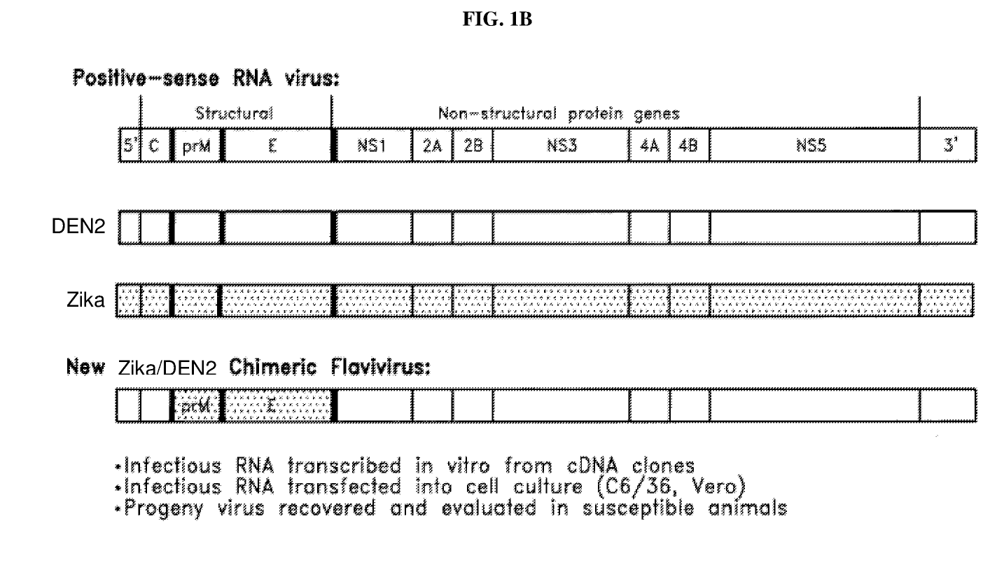

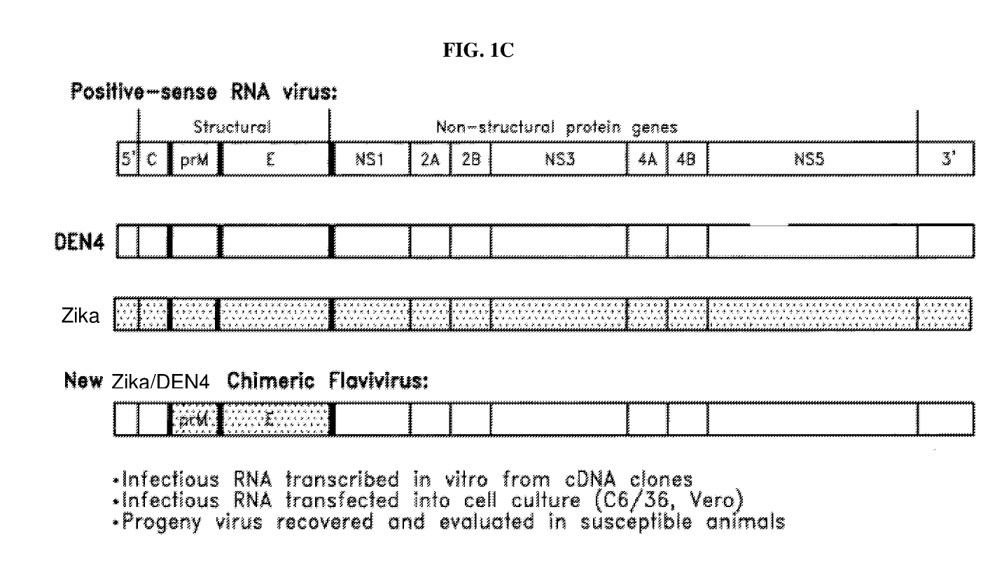

### DETAILED DESCRIPTION OF THE INVENTION

While effective vaccines exist for other flaviviruses, such as dengue, yellow fever, and Japanese encephalitis, an effective vaccine against ZIKV is not yet available. Given the current state of global outbreak, there is an immediate need in the art to develop an effective anti-Zika vaccine. The present disclosure addresses this deficiency in the art by providing a vaccine against ZIKV. The present invention is based, at least in part, on the discovery that immunogenic compositions and vaccines and/or multivalent immunogenic compositions and vaccines including, but not limited to, attenuated dengue virus and ZIKV genomes may generate immune responses and/or provide protection against multiple flaviviruses (e.g., dengue and Zika) in a subject. The disclosure describes various aspects which include: novel attenuated Zika viruses; novel attenuated chimeric Zika viruses; ZIKV genomes comprising one or more attenuating mutations and/or deletions; chimeric ZIKV genomes; immunogenic compositions effective for inducing immunity against ZIKV; anti-Zika vaccines comprising live attenuated Zika virus; methods for vaccinating subjects with an anti-Zika vaccine to protect against Zika infections; methods for manufacturing attenuated ZIKV genomes or chimeric ZIKV genomes; methods for manufacturing attenuated ZIKV vaccines or attenuated chimeric ZIKV vaccines; and pharmaceutical kits comprising attenuated ZIKV vaccines or attenuated chimeric ZIKV vaccines, or multivalent (e.g., pentavalent) vaccines comprising one or more flavivirus vaccines (e.g., one or more dengue virus vaccines) and a Zika vaccine. Other aspects are included and described further herein.

ZIKV and dengue virus are mosquito-borne flavivirus pathogens. The flavivirus genome contains a 5′ untranslated region (5′ UTR), followed by a capsid protein (C) encoding region, followed by a premembrane/membrane protein (prM) encoding region, followed by an envelope protein (E) encoding region, followed by the region encoding the nonstructural proteins (NS1-NS2A-NS2B-NS3-NS4A-NS4B-NS5) and finally a 3′ untranslated region (3′ UTR). The viral structural proteins are C, prM and E, and the nonstructural proteins are NS1-NS5. The structural and nonstructural proteins are translated as a single polyprotein and processed by cellular and viral proteases.

In certain aspects, the present disclosure relates to a nucleic acid chimera comprising a first nucleotide sequence encoding at least one structural protein from a ZIKV, a second nucleotide sequence encoding at least one nonstructural protein from a first flavivirus, and a third nucleotide sequence of a 3′ untranslated region from a second flavivirus. The present disclosure also relates to a pentavalent immunogenic composition comprising: a first attenuated virus that is immunogenic against dengue serotype 1, a second attenuated virus that is immunogenic against dengue serotype 2, a third attenuated virus that is immunogenic against dengue serotype 3, a fourth attenuated virus that is immunogenic against dengue serotype 4, and a fifth attenuated virus that is immunogenic against ZIKV. The fifth attenuated virus can be the nucleic acid chimera in accordance with the present disclosure.

According to an aspect, the present disclosure provides a ZIKV genome modified to contain one or more attenuating mutations (such as point mutations, deletions, insertions, inversions, or combinations thereof) or a chimeric ZIKV genome comprising a portion of a ZIKV genome and a portion of the genome of at least one other flavivirus, such as, but not limited to, dengue virus (e.g., DEN1, DEN2, DEN3, or DEN4), West Nile virus, yellow fever virus, Japanese encephalitis virus, tick-borne encephalitis virus, or combinations thereof.

According to another aspect, the present disclosure provides a ZIKV virion (i.e., virus particle) comprising a ZIKV genome modified to contain one or more attenuating mutations or a chimeric ZIKV genome comprising a portion of a ZIKV genome and a portion of the genome of at least one other flavivirus, such as, but not limited to, dengue virus (e.g., DEN1, DEN2, DEN3, or DEN4), West Nile virus, yellow fever virus, Japanese encephalitis virus, tick-borne encephalitis virus, or combinations thereof.

According to an aspect, the present disclosure provides an immunogenic composition or vaccine comprising an attenuated ZIKV or an attenuated ZIKV. The attenuation of the attenuated ZIKV can be the result of the genome of the ZIKV comprising one or more attenuating mutations and/or deletions. The chimeric ZIKV has a genome comprising a portion of a ZIKV genome and a portion of the genome of at least one other flavivirus, such as, but not limited to, dengue virus (e.g., DEN1, DEN2, DEN3, or DEN4), West Nile virus, yellow fever virus, Japanese encephalitis virus, tick-borne encephalitis virus, or combinations thereof.

According to another aspect, the present disclosure provides a pharmaceutical kit comprising an immunogenic composition or vaccine. In an embodiment, the vaccine comprises an attenuated ZIKV, wherein the attenuation results from the genome of the ZIKV comprising one or more attenuating mutations and/or deletions, together with a set of instructions for using the composition to vaccinate a subject. In another embodiment, the vaccine comprises an attenuated chimeric ZIKV having a genome comprising a portion of a ZIKV genome and a portion of the genome of at least one other flavivirus, such as, but not limited to, dengue virus (e.g., DEN1, DEN2, DEN3, or DEN4), West Nile virus, yellow fever virus, Japanese encephalitis virus, tick-borne encephalitis virus, or combinations thereof, together with a set of instructions for using the composition to vaccinate a subject.

According to an aspect, the present disclosure provides a method for vaccinating a subject to provide immunity against ZIKV. The method comprises administering a pharmaceutically acceptable dose of a ZIKV vaccine. The vaccine comprises either an attenuated ZIKV or an attenuated chimeric ZIKV. The attenuation of the attenuated ZIKV is the result of the genome of the ZIKV comprising one or more attenuating mutations and/or deletions. The attenuated chimeric ZIKV has a genome comprising a portion of a ZIKV genome and a portion of the genome of at least one other flavivirus, such as, but not limited to, dengue virus (e.g., DEN1, DEN2, DEN3, or DEN4), West Nile virus, yellow fever virus, Japanese encephalitis virus, tick-borne encephalitis virus, or combinations thereof.

According to a further aspect, the present disclosure provides a method for manufacturing a vaccine comprising an attenuated ZIKV, wherein said attenuation is the result of the genome of the ZIKV comprising one or more attenuating mutations and/or deletions. In an embodiment, the method for manufacturing comprises introducing at least one attenuating mutation and/or deletions into the genome of a wildtype ZIKV and combining the attenuated ZIKV with one or more pharmaceutical excipients to provide said vaccine.

According to another aspect, the present disclosure provides a method for manufacturing a vaccine comprising an attenuated chimeric ZIKV. In an embodiment, the manufacturing comprises combining a portion of a ZIKV genome and a portion of the genome of at least one other flavivirus, such as, but not limited to, dengue virus (e.g., DEN1, DEN2, DEN3, or DEN4), West Nile virus, yellow fever virus, Japanese encephalitis virus, tick-borne encephalitis virus, or combinations thereof, to provide the attenuated chimeric ZIKV, and combining the attenuated chimeric ZIKV with one or more pharmaceutical excipients to provide said vaccine.

According to an aspect of the invention, the present disclosure provides a multivalent immunogenic composition. The composition comprising: at least one first attenuated viruses that are immunogenic against a flavivirus, and a second attenuated virus that is immunogenic against ZIKV. In a particular embodiment, the one or more first attenuated viruses is immunogenic against dengue virus (e.g., serotype 1, 2, 3, 4, or a combination thereof), West Nile virus, yellow fever virus, Japanese encephalitis virus, tick-borne encephalitis virus. In another embodiment, the second attenuated virus is a Zika nucleic acid chimera in accordance with the present disclosure. In another particular embodiment, the second attenuated virus is a ZIKV comprising one or more attenuating mutations and/or deletions in the genome. In a particular embodiment, the at least one first attenuated viruses include a first attenuated virus that is immunogenic against dengue serotype 1, a second attenuated virus that is immunogenic against dengue serotype 2, a third attenuated virus that is immunogenic against dengue serotype 3, and a fourth attenuated virus that is immunogenic against dengue serotype 4, thereby producing a pentavalent immunogenic composition.

According to an additional aspect, the present disclosure provides a method for inducing an immune response against ZIKV. The method comprising administering a pharmaceutically acceptable dose of a ZIKV vaccine comprising an attenuated ZIKV or an attenuated chimeric ZIKV. In an embodiment, the attenuation of the attenuated ZIKV is the result of the genome of the ZIKV comprising one or more attenuating mutations and/or deletions. In another embodiment, the attenuated chimeric ZIKV has a genome comprising a portion of a ZIKV genome and a portion of the genome of at least one other flavivirus, such as, but not limited to, dengue virus (e.g., DEN1, DEN2, DEN3, or DEN4), West Nile virus, yellow fever virus, Japanese encephalitis virus, tick-borne encephalitis virus, or combinations thereof.

According to an aspect, the present disclosure provides a method for inducing an immune response against ZIKV. The method comprising administering a pharmaceutically acceptable dose of the pentavalent immunogenic composition described herein or the multivalent immunogenic composition described herein.

All publications, patent applications, patents, figures and other references cited or referenced herein and all documents cited or referenced in the herein cited documents, together with any manufacturer's instructions, descriptions, product specifications, and product sheets for any products mentioned herein or in any document incorporated by reference herein, are hereby incorporated by reference, and may be employed in the practice of the invention. For example, the present disclosure is related to U.S. Patent Application Publications 2009/0028900 A1, 2010/0316670 A1, 2005/0010043 A1, 2004/0033594 A, 2005/0100886 A1, 2007/0134256, 2010/0104598 A1, 2007/0009552 A1, WO 2008/157136 A1, WO 2006/036233 A1, WO 03/092592 A1, WO 03/059384 A1, and WO 01/59093 A1, all of which are incorporated by reference for all purposes.

Unless defined otherwise, technical and scientific terms used herein have the same meaning as commonly understood by one of ordinary skill in the art to which this invention belongs. See, e.g., Singleton P and Sainsbury D., Dictionary of Microbiology and Molecular Biology 3rd ed., J. Wiley & Sons, Chichester, New York, 2001, and Fields Virology 4th ed., Knipe D. M. and Howley P. M. eds, Lippincott Williams & Wilkins, Philadelphia 2001.

The term “about” means within 1, 2, or 3 nucleotides.

The articles “a” and “an” as used herein and in the appended claims are used herein to refer to one or to more than one (i.e., to at least one) of the grammatical object of the article unless the context clearly indicates otherwise. By way of example, “an element” means one element or more than one element.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

As used herein in the specification and in the claims, the phrase “at least one,” in reference to a list of one or more elements, should be understood to mean at least one element selected from anyone or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a nonlimiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, in certain methods described herein that include more than one step or act, the order of the steps or acts of the method is not necessarily limited to the order in which the steps or acts of the method are recited unless the context indicates otherwise.

The terms “co-administration” and “co-administering” or “combination therapy” refer to both concurrent administration (administration of two or more therapeutic agents at the same time) and time varied administration (administration of one or more therapeutic agents at a time different from that of the administration of an additional therapeutic agent or agents), as long as the therapeutic agents are present in the patient to some extent, preferably at effective amounts, at the same time. In certain preferred aspects, one or more of the present compounds described herein, are coadministered in combination with at least one additional bioactive agent, especially including an anticancer agent. In particularly preferred aspects, the co-administration of compounds results in synergistic activity and/or therapy, including anticancer activity.

The term “patient” or “subject” is used throughout the specification to describe an animal, preferably a human or a domesticated animal, to whom treatment, including prophylactic treatment, with the compositions according to the present disclosure is provided. For treatment of those infections, conditions or disease states which are specific for a specific animal such as a human patient, the term patient refers to that specific animal, including a domesticated animal such as a dog or cat or a farm animal such as a horse, cow, sheep, etc. In general, in the present disclosure, the term patient refers to a human patient unless otherwise stated or implied from the context of the use of the term.

The term “effective” is used to describe an amount of a compound, composition or component which, when used within the context of its intended use, effects an intended result. The term effective subsumes all other effective amount or effective concentration terms, which are otherwise described or used in the present application.

The term “residue” is used herein to refer to an amino acid (D or L) or an amino acid mimetic that is incorporated into a peptide by an amide bond. As such, the amino acid may be a naturally occurring amino acid or, unless otherwise limited, may encompass known analogs of natural amino acids that function in a manner similar to the naturally occurring amino acids (i.e., amino acid mimetics). Moreover, an amide bond mimetic includes peptide backbone modifications well known to those skilled in the art.

Furthermore, one of skill in the art will recognize that individual substitutions, deletions or additions in the amino acid sequence, or in the nucleotide sequence encoding for the amino acids, which alter, add or delete a single amino acid or a small percentage of amino acids (typically less than 5%, more typically less than 1%) in an encoded sequence are conservatively modified variations, wherein the alterations result in the substitution of an amino acid with a chemically similar amino acid. Conservative substitution tables providing functionally similar amino acids are well known in the art. The following six groups each contain amino acids that are conservative substitutions for one another: (1) Alanine (A), Serine (S), Threonine (T); (2) Aspartic acid (D), Glutamic acid (E); (3) Asparagine (N), Glutamine (Q); (4) Arginine (R), Lysine (K); (5) Isoleucine (I), Leucine (L), Methionine (M), Valine (V); and (6) Phenylalanine (F), Tyrosine (Y), Tryptophan (W). Other terms are defined or otherwise described herein.

Mutant Attenuated Zika Viruses and Chimeric Attenuated Zika Viruses

The invention relates to Zika viruses which are attenuated as a result of (a) the introduction of one or more (e.g., at least 1, 2, 3, 4, or 5) attenuating mutations in the Zika viral genome, or (b) converting a ZIKV to a chimeric virus by modifying a first flavivirus “backbone” genome (e.g., DEN1, DEN2, DEN3, or DEN4, tick-born encephalitis virus, or West Nile virus) to include one or more ZIKV genes encoding immunogenic components (e.g., genes encoding Zika capsid or pre-membrane proteins or both).

In the case of attenuated Zika viruses, the attenuating mutations can include any point mutation, insertion, deletion, or inversion, or any combinations thereof, or any such mutation which reduces or eliminates the virulence of the ZIKV, but which do not block the ability of the virus to replicate and otherwise allow its immunogenic components to be expressed. The attenuating mutations may be introduced anywhere in the genome. For example, mutations may be introduced into one or more nonstructural genes (NS1, NS2A, NS2B, NS3, NS4A, NS4B, NS5 genes), or one or more structural genes (capsid (C), premembrane/membrane (prM) and envelope (E) protein genes), or the ‘5 UTR or the 3’ UTR, or combinations thereof.

In the case of the chimeric Zika viruses, the structure of the chimeric virus may vary considerably. In certain embodiments, a ZIKV genome (e.g., a wildtype strain of ZIKV) can be modified by replacing or substituting one or more genetic components (e.g., a nonstructural gene, a structural gene, a 5′ UTR or a 3′ UTR) in the Zika genome with the same genetic component from another flavivirus (e.g., from DEN1, DEN2, DEN3, or DEN4). In this embodiment, the ZIKV can be considered as a backbone genome into which certain genetic components therein are replaced with corresponding genetic components from another flavivirus to form a chimeric virus. The resulting chimeric viruses are attenuated. In certain other embodiments, a flavivirus genome other than Zika (e.g., DEN1, DEN2, DEN3, or DEN4) can be modified by replacing or substituting one or more genetic components (e.g., a nonstructural gene, a structural gene, a 5′ UTR or a 3′ UTR) in the flavivirus genome with the corresponding genetic component from a ZIKV genome. In this embodiment, the flavivirus genome can be considered as a backbone genome into which certain genetic components therein are replaced with corresponding genetic components from a ZIKV to form a chimeric virus. The resulting chimeric viruses are attenuated.

In one embodiment, the invention provides a chimeric ZIKV constructed from a flavivirus backbone wherein one or more structural genes (flavivirus C, prM, and/or E) therein have been replaced with the corresponding one or more structural genes from a ZIKV.

In another embodiment, the invention provides a chimeric ZIKV constructed from a dengue virus backbone (e.g., DEN1, DEN2, DEN3, or DEN4, or a chimeric thereof) wherein one or more structural genes (dengue C, prM, and/or E) therein have been replaced with the corresponding one or more structural genes from a ZIKV.

In still another embodiment, the invention provides a chimeric ZIKV constructed from a dengue serotype 2 virus backbone, wherein one or more structural genes (dengue serotype 2 C, prM, and/or E) therein have been replaced with the corresponding one or more structural genes from a ZIKV.

In yet another embodiment, the invention provides a chimeric ZIKV constructed from a dengue serotype 4 virus backbone, wherein one or more structural genes (dengue serotype 4 C, prM, and/or E) therein have been replaced with the corresponding one or more structural genes from a ZIKV.

In another embodiment, the invention provides a chimeric ZIKV constructed from a dengue serotype 1 virus backbone, wherein one or more structural genes (dengue serotype 1 C, prM, and/or E) therein have been replaced with the corresponding one or more structural genes from a ZIKV.

In yet another embodiment, the invention provides a chimeric ZIKV constructed from a dengue serotype 3 virus backbone, wherein one or more structural genes (dengue serotype 3 C, prM, and/or E) therein have been replaced with the corresponding one or more structural genes from a ZIKV.

In any of the chimeric ZIKV embodiments, the backbone virus used to form the chimeric ZIKV (e.g., a ZIKV in certain embodiments, or another flavivirus in other embodiments) can comprise, in addition, one or more attenuating mutations as described above. These additional attenuating mutations may be introduced anywhere in the backbone genome. For example, mutations may be introduced into one or more nonstructural genes (NS1, NS2A, NS2B, NS3, NS4A, NS4B, NS5 genes), or one or more structural genes (capsid (C), premembrane/membrane (prM) and envelope (E) protein genes), or the ‘5 UTR or the 3’ UTR, or combinations thereof. For example, a chimeric ZIKV comprising a DEN2 backbone or a DEN4 backbone into which one or more structural protein genes therein were substituted with the corresponding Zika structural protein genes may further comprise a Δ30, Δ30/31, or Δ86, or any other attenuating mutation in the 3′UTR in addition to the Δ30, Δ30/31, or Δ86 mutations.

Immunogenic Zika chimeras and methods for preparing the Zika chimeras are provided herein. The immunogenic ZIKV chimeras are useful, alone or in combination, in a pharmaceutically acceptable carrier as immunogenic compositions to immunize and protect individuals and animals against infection by ZIKV. In certain embodiments, the Zika chimera should induce a humoral (antibody) response to ZIKV, while the non-structural proteins of dengue virus should include a T-cell response.

Zika chimeras of the present disclosure can comprise nucleotide sequences encoding the immunogenic structural proteins of a ZIKV and further nucleotide sequences selected from the backbone of a dengue virus. Zika chimeras of the present disclosure can comprise nucleotides sequences encoding the immunogenic structural proteins and the nonstructural proteins of ZIKV and the 3′UTR of a dengue virus (e.g., serotype 1, serotype 2, serotype 3, or serotype 4). In an embodiment, the 3′ UTR of the dengue virus contains an attenuating deletion. The present disclosure also contemplates an attenuated ZIKV that includes an attenuating deletion or mutations, as described below with regard to dengue virus attenuation. Zika chimeric viruses derived from the nucleotide sequences can be used to induce an immunogenic response against ZIKV.

In another embodiment, the preferred chimera is a Zika nucleic acid chimera comprising a first nucleotide sequence encoding at least one structural protein from a ZIKV, and a second nucleotide sequence encoding nonstructural proteins from a dengue virus. In another embodiment, the dengue virus is attenuated. In another embodiment, the dengue virus is DEN2. In another embodiment, the dengue virus is DEN4. In yet another embodiment, the dengue virus is DEN3. In a further embodiment, the dengue virus is DEN1. In particular embodiments, the structural protein can be the C protein of a ZIKV, the prM protein of a ZIKV, the E protein of a ZIKV, or any combination thereof.

As used herein, the terms “Zika chimera,” “Zika chimeric virus,” and “chimeric ZIKV” means an infectious construct of the invention comprising nucleotide sequences encoding the immunogenicity of a ZIKV and further nucleotide sequences derived from the backbone of a flavivirus, such as, but not limited to, dengue virus, or an attenuated ZIKV.

As used herein, “infectious construct” indicates a virus, a viral construct, a viral chimera, a nucleic acid derived from a virus or any portion thereof, which may be used to infect a cell.

As used herein, “Zika nucleic acid chimera” means a construct as described herein comprising nucleic acid comprising nucleotide sequences encoding the immunogenicity of a ZIKV and further nucleotide sequences derived from the backbone of a flavivirus, such as, but not limited to, dengue virus or an attenuated ZIKV. Correspondingly, any chimeric flavivirus or flavivirus chimera as described herein is to be recognized as an example of a nucleic acid chimera.

As used herein, “structural and nonstructural proteins” can mean or include any protein comprising or any gene encoding the sequence of the complete protein, an epitope of the protein, or any fragment comprising, for example, three or more amino acid residues thereof.

The flavivirus chimeras of the invention are constructs formed by fusing structural protein genes from a ZIKV with non-structural protein genes from a flavivirus, such as, but not limited to, dengue virus, e.g., DEN1, DEN2, DEN3, or DEN4. The use of any strain is contemplated, such as those dengue strains of Table 1.

TABLE 1
Examples of Dengue Virus Strains and their Associated Accession Numbers

| Serotype | Strain | Accession No. |
|---|---|---|
| 1 | 02-20 | AB178040 |
| 1 | 16007 | AF180817 |
| 1 | 16007 PDK-13 | AF180818 |
| 1 | 259par00 | AF514883 |
| 1 | 280par00 | AF514878 |
| 1 | 293arg00 | AY206457 |
| 1 | 295arg00 | AF514885 |
| 1 | 297arg00 | AF514889 |
| 1 | 301arg00 | AF514876 |
| 1 | 98901518 | AB189120 |
| 1 | 98901530 | AB189121 |
| 1 | A88 | AB074761 |
| 1 | Abidjan | AF298807 |
| 1 | ARG0028 | AY277665 |
| 1 | ARG0048 | AY277666 |
| 1 | ARG9920 | AY277664 |
| 1 | BR-90 | AF226685 |
| 1 | BR-01-MR | AF513110 |
| 1 | BR-97-111 | AF311956 |
| 1 | BR-97-233 | AF311958 |
| 1 | BR-97-409 | AF311957 |
| 1 | Cambodia | AF309641 |
| 1 | FGA-89 | AF226687 |
| 1 | FGA-NA d1d | AF226686 |
| 1 | Fj231-04 | DQ193572 |
| 1 | GD05-99 | AY376738 |
| 1 | GD23-95 | AY373427 |
| 1 | GZ-80 | AF350498 |
| 1 | D1-hu-Yap-NIID27-2004 | AB204803 |
| 1 | D1-H-IMTSSA-98-606 | AF298808 |
| 1 | Mochizuki | AB074760 |
| 1 | D1.Myanmar.059-01 | AY708047 |
| 1 | D1.Myanmar.194-01 | AY713474 |
| 1 | D1.Myanmar.206-01 | AY713475 |
| 1 | D1.Myanmar.23819-96 | AY722802 |
| 1 | D1.Myanmar.305-01 | AY713476 |
| 1 | D1.Myanmar.31459-98 | AY726555 |
| 1 | D1.Myanmar.31987-98 | AY726554 |
| 1 | D1.Myanmar.32514-98 | AY722803 |
| 1 | D1.Myanmar.37726-01 | AY726549 |
| 1 | D1.Myanmar.38862-01 | AY726550 |
| 1 | D1.Myanmar.40553-71 | AY713473 |
| 1 | D1.Myanmar.40568-76 | AY722801 |
| 1 | D1.Myanmar.44168-01 | AY726551 |
| 1 | D1.Myanmar.44988-02 | AY726552 |
| 1 | D1.Myamnar.49440-02 | AY726553 |
| 1 | rWestern Pacific-delta30 | AY145123 |
| 1 | Western Pacific rDEN1mutF | AY145122 |
| 1 | S275-90 | A75711 |
| 1 | D1-hu-Seychelles-NIID41-2003 | AB195673 |
| 1 | Singapore 8114-93 | AY762084 |
| 1 | Singapore S275-90 | M87512 |
| 1 | ThD1_0008_81 | AY732483 |
| 1 | ThD1_0049_01 | AY732482 |
| 1 | ThD1_0081_82 | AY732481 |
| 1 | ThD1_0097_94 | AY732480 |
| 1 | ThD1_0102_01 | AY732479 |
| 1 | ThD1_0323_91 | AY732478 |
| 1 | ThD1_0336_91 | AY732477 |
| 1 | ThD1_0442_80 | AY732476 |
| 1 | ThD1_0488_94 | AY732475 |
| 1 | ThD1_0673_80 | AY732474 |
| 1 | Recombinant Western Pacific | AY145121 |
| 1 | Nauru Island Western Pacific 45AZ5 | NC_001477 |
| 1 | Nauru Island, Western Pacific Bethesda | U88535 |
| 1 | Nauru Island Western Pacific 45AZ5-PDK27 | U88537 |
| 2 | 131 | AF100469 |
| 2 | 16681-PDK53 | M84728 |
| 2 | 16681 Blok | M84727 |
| 2 | 16681 Kinney | U87411 |
| 2 | 43 | AF204178 |
| 2 | 44 | AF204177 |
| 2 | 98900663 | AB189122 |
| 2 | 98900665 | AB189123 |
| 2 | 98900666 | AB189124 |
| 2 | BA05i | AY858035 |
| 2 | Bangkok 1974 | AJ487271 |
| 2 | BR64022 | AF489932 |
| 2 | C0166 | AF100463 |
| 2 | C0167 | AF100464 |
| 2 | C0371 | AF100461 |
| 2 | C0390 | AF100462 |
| 2 | China 04 | AF119661 |
| 2 | Cuba115-97 | AY702036 |
| 2 | Cuba13-97 | AY702034 |
| 2 | Cuba165-97 | AY702038 |
| 2 | Cuba205-97 | AY702039 |
| 2 | Cuba58-97 | AY702035 |
| 2 | Cuba89-97 | AY702037 |
| 2 | DR23-01 | AB122020 |
| 2 | DR31-01 | AB122021 |
| 2 | DR59-01 | AB122022 |
| 2 | FJ-10 | AF276619 |
| 2 | FJ11-99 | AF359579 |
| 2 | I348600 | AY702040 |
| 2 | IQT1797 | AF100467 |
| 2 | IQT2913 | AF100468 |
| 2 | Jamaica-N 1409 | M20558 |
| 2 | K0008 | AF100459 |
| 2 | K0010 | AF100460 |
| 2 | Mara4 | AF100466 |
| 2 | DEN2-H-IMTSSA-MART-98-703 | AF208496 |
| 2 | New Guinea C | AF038403 |
| 2 | New Guinea C-PUO-218 hybrid | AF038402 |
| 2 | New Guinea-C | M29095 |
| 2 | PDK-53 | U87412 |
| 2 | S1 vaccine | NC_001474 |
| 2 | TB16i | AY858036 |
| 2 | ThD2_0017_98 | DQ181799 |
| 2 | ThD2_0026_88 | DQ181802 |
| 2 | ThD2_0038_74 | DQ181806 |
| 2 | ThD2_0055_99 | DQ181798 |
| 2 | ThD2_0078_01 | DQ181797 |
| 2 | ThD2_0168_79 | DQ181805 |
| 2 | ThD2_0263_95 | DQ181800 |
| 2 | ThD2_0284_90 | DQ181801 |
| 2 | ThD2_0433_85 | DQ181803 |
| 2 | ThD2_0498_84 | DQ181804 |
| 2 | ThNH-28-93 | AF022435 |
| 2 | ThNH29-93 | AF169678 |
| 2 | ThNH36-93 | AF169679 |
| 2 | ThNH45-93 | AF169680 |
| 2 | ThNH-52-93 | AF022436 |
| 2 | ThNH54-93 | AF169682 |
| 2 | ThNH55-93 | AF169681 |
| 2 | ThNH62-93 | AF169683 |
| 2 | ThNH63-93 | AF169684 |
| 2 | ThNH69-93 | AF169685 |
| 2 | ThNH73-93 | AF169686 |
| 2 | ThNH76-93 | AF169687 |
| 2 | ThNH81-93 | AF169688 |
| 2 | ThNH-p36-93 | AF022441 |
| 2 | ThNH-7-93 | AF022434 |
| 2 | ThNH-p11-93 | AF022437 |
| 2 | ThNH-p12-93 | AF022438 |
| 2 | ThNH-p14-93 | AF022439 |
| 2 | ThNH-p16-93 | AF022440 |
| 2 | Tonga-74 | AY744147 |
| 2 | TSV01 | AY037116 |
| 2 | Taiwan-1008DHF | AY776328 |
| 2 | Ven2 | AF100465 |
| 3 | D3-H-IMTSSA-MART-1999-1243 | AY099337 |
| 3 | D3-H-IMTSSA-SRI-2000-1266 | AY099336 |
| 3 | 80-2 | AF317645 |
| 3 | 98901403 | AB189125 |
| 3 | 98901437 | AB189126 |
| 3 | 98901517 | A6189127 |
| 3 | 98902890 | AB189128 |
| 3 | BA51 | AY858037 |
| 3 | BDH02-1 | AY496871 |
| 3 | BDH02-3 | AY496873 |
| 3 | BDH02-4 | AY496874 |
| 3 | BDH02-7 | AY496877 |
| 3 | BR74886-02 | AY679147 |
| 3 | C0331-94 | AY876494 |
| 3 | C0360-94 | AY923865 |
| 3 | den3_88 | AY858038 |
| 3 | den3_98 | AY858039 |
| 3 | FW01 | AY858040 |
| 3 | FW06 | AY858041 |
| 3 | H87 | NC_001475 |
| 3 | D3-Hu-TL018NIID-2005 | AB214879 |
| 3 | D3-Hu-TL029NIID-2005 | AB214880 |
| 3 | D3-Hu-TL109NIID-2005 | AB214881 |
| 3 | D3-Hu-TL129NIID-2005 | AB214882 |
| 3 | InJ_16_82 | DQ401690 |
| 3 | KJ30i | AY858042 |
| 3 | kJ46 | AY858043 |
| 3 | kJ71 | AY858044 |
| 3 | mutant BDH02_01 | DQ401689 |
| 3 | mutant BDH02_03 | DQ401691 |
| 3 | mutant BDH02_04 | DQ401692 |
| 3 | mutant BDH02_07 | DQ401693 |
| 3 | mutant InJ_I6_82 | DQ401694 |
| 3 | mutant PhMH_J1_97 | DQ401695 |
| 3 | PF89-27643 | AY744677 |
| 3 | PF89-320219 | AY744678 |
| 3 | PF90-3050 | AY744679 |
| 3 | PF90-3056 | AY744680 |
| 3 | PF90-6056 | AY744681 |
| 3 | PF92-2956 | AY744682 |
| 3 | PF92-2986 | AY744683 |
| 3 | PH86 | AY858045 |
| 3 | PhMH-J1-97 | AY496879 |
| 3 | PI64 | AY858046 |
| 3 | Singapore | AY662691 |
| 3 | Singapore 8120-95 | AY766104 |
| 3 | Sleman-78 | AY648961 |
| 3 | TB16 | AY858047 |
| 3 | TB55i | AY858048 |
| 3 | ThD3_0007_87 | AY676353 |
| 3 | ThD3_0010_87 | AY676353 |
| 3 | ThD3_0055_93 | AY676351 |
| 3 | ThD3_0104_93 | AY676350 |
| 3 | ThD3_1283_98 | AY676349 |
| 3 | ThD3_1687_98 | AY676348 |
| 3 | PF92-4190 | AY744684 |
| 3 | PF94-136116 | AY744685 |
| 3 | Taiwan-739079A | AY776329 |
| 4 | 2A | AF375822 |
| 4 | Recombinant clone rDEN4 | AF326825 |
| 4 | 2AdeI30 | AF326826 |
| 4 | 814669 | AF326573 |
| 4 | B5 | AF289029 |
| 4 | rDEN4del30 | AF326827 |
| 4 | H241 | AY947539 |
| 4 | rDEN4 | NC_002640 |
| 4 | Singapore 8976-95 | AY762085 |
| 4 | SW38i | AY858050 |
| 4 | ThD4_0017_97 | AY618989 |
| 4 | ThD4_0087_77 | AY618991 |
| 4 | ThD4_0348_91 | AY618990 |
| 4 | ThD4_0476_97 | AY618988 |
| 4 | ThD4_0485_01 | AY618992 |
| 4 | ThD4_0734_00 | AY618993 |
| 4 | Taiwan-2K0713 | AY776330 |
| 4 | Unknown | M14931 |

The attenuated, immunogenic flavivirus chimeras provided herein contain one or more of the structural protein genes, or antigenic portions thereof, of the ZIKV against which immunogenicity is to be conferred, and the nonstructural protein genes of another flavivirus, e.g., a dengue virus.

In certain aspects, the chimera as described herein contains a dengue virus genome as the backbone, in which the structural protein gene(s) encoding C, prM, or E protein(s) of the dengue genome, or combinations thereof, are replaced with the corresponding structural protein gene(s) from a ZIKV that is to be protected against. The resulting chimeric virus has the properties, by virtue of being chimerized with the dengue virus, of attenuation and is therefore reduced in virulence, but expresses antigenic epitopes of the ZIKV structural gene products and is therefore immunogenic.

The genome of any flavivirus can be used as the backbone in the attenuated chimeras described herein. The backbone can contain mutations that contribute to the attenuation phenotype of the flavivirus or that facilitate replication in the cell substrate used for manufacture, e.g., Vero cells. The mutations can be in the nucleotide sequence encoding nonstructural proteins, the 5′ untranslated region or the 3′ untranslated region. The backbone can also contain further mutations to maintain the stability of the attenuation phenotype and to reduce the possibility that the attenuated virus or chimera might revert back to the virulent wild-type virus.

In certain embodiments, the genome of any dengue virus can be used as the backbone in the attenuated chimeras described herein. The backbone can contain mutations that contribute to the attenuation phenotype of the dengue virus or that facilitate replication in the cell substrate used for manufacture, e.g., Vero cells. The mutations can be in the nucleotide sequence encoding nonstructural proteins, the 5′ untranslated region or the 3′ untranslated region. The backbone can also contain further mutations to maintain the stability of the attenuation phenotype and to reduce the possibility that the attenuated virus or chimera might revert back to the virulent wild-type virus.

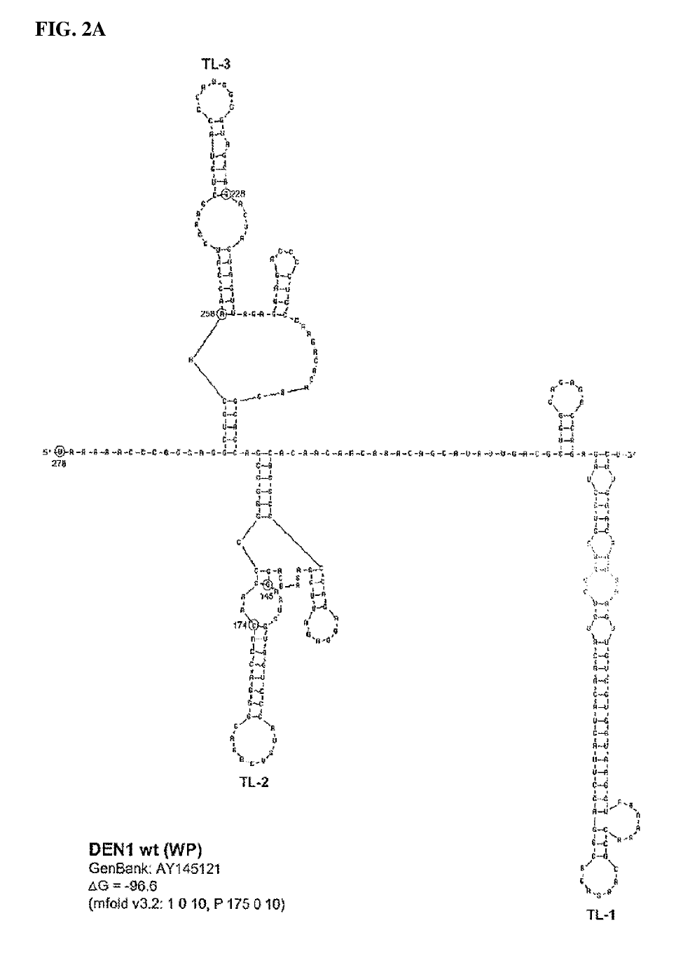

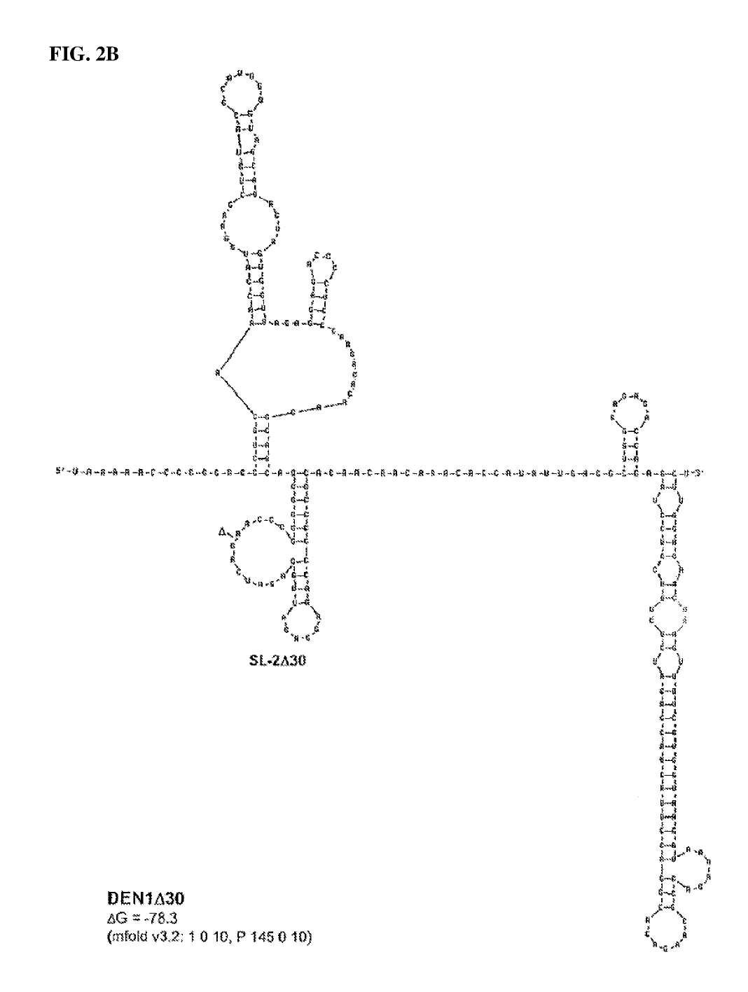

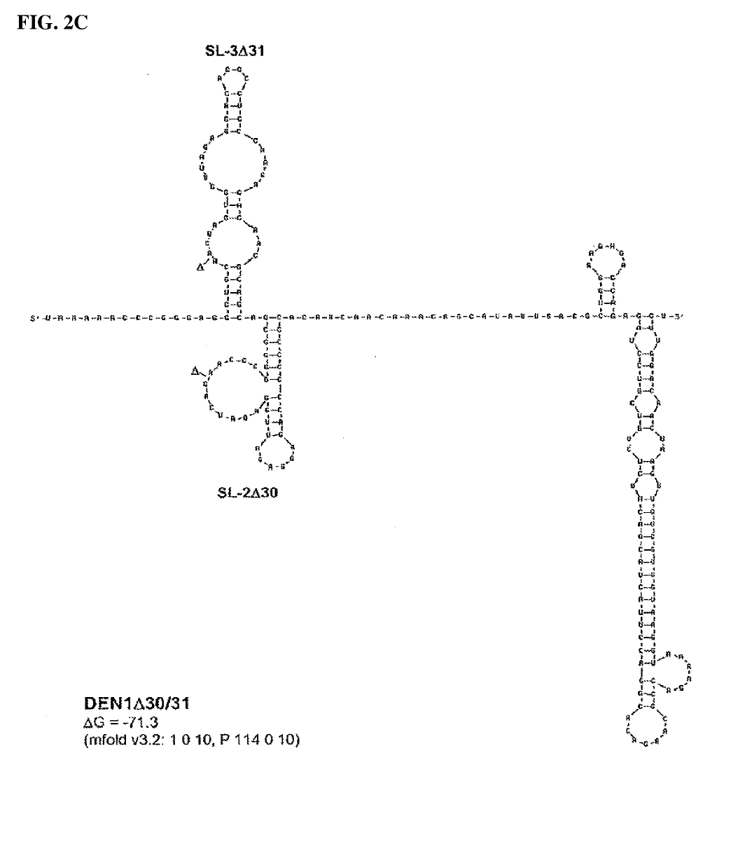

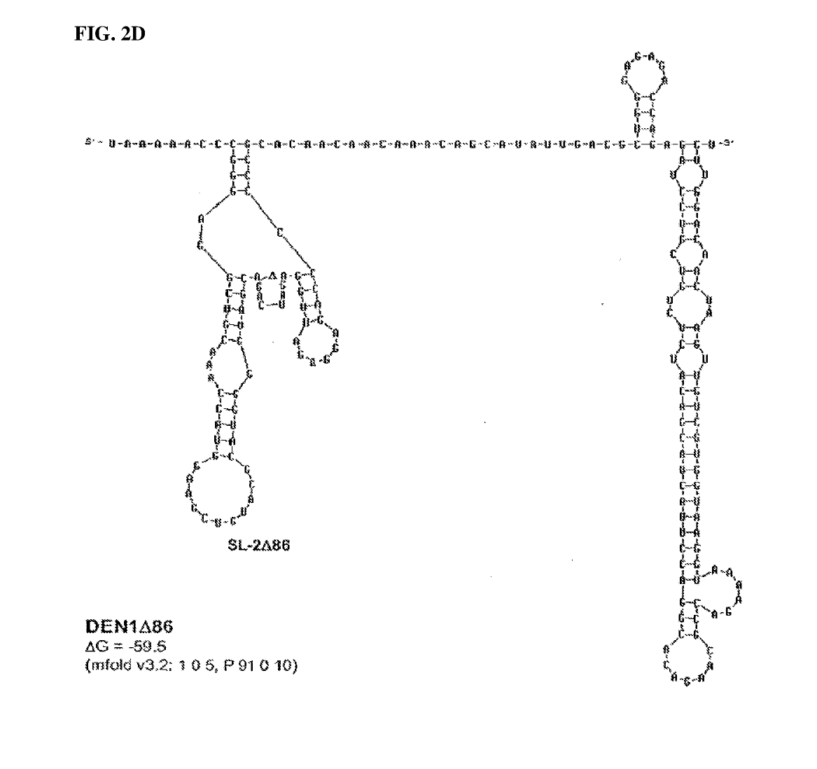

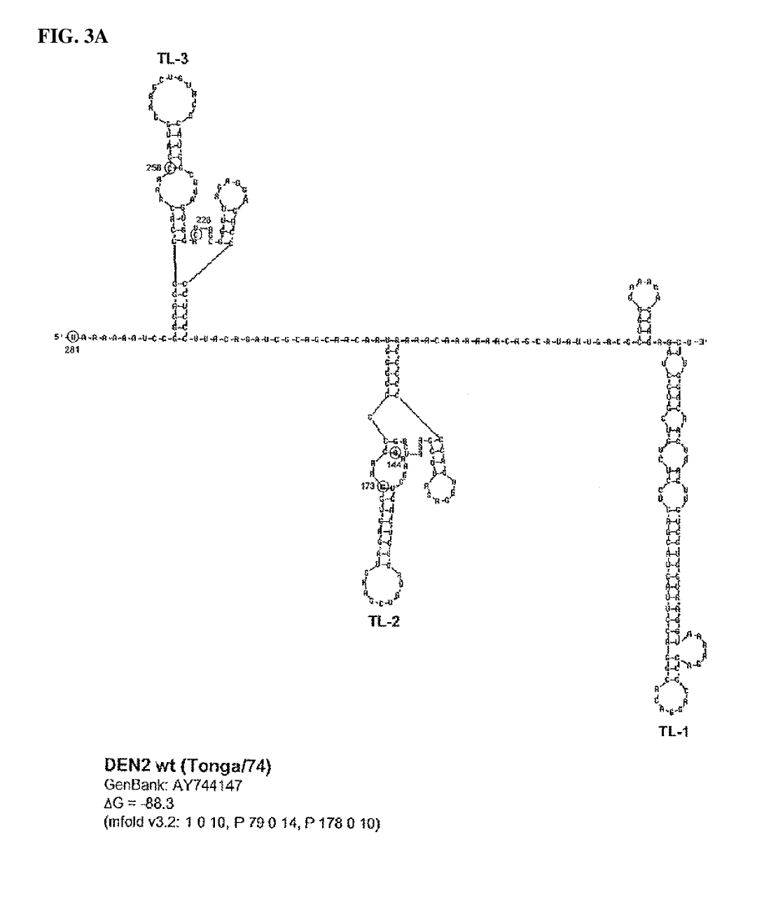

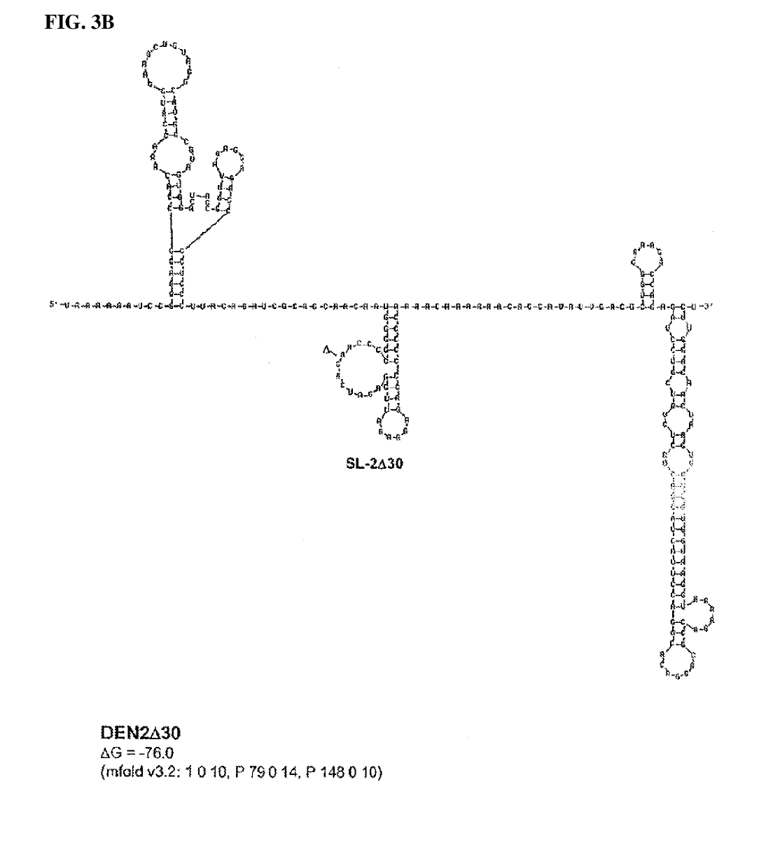

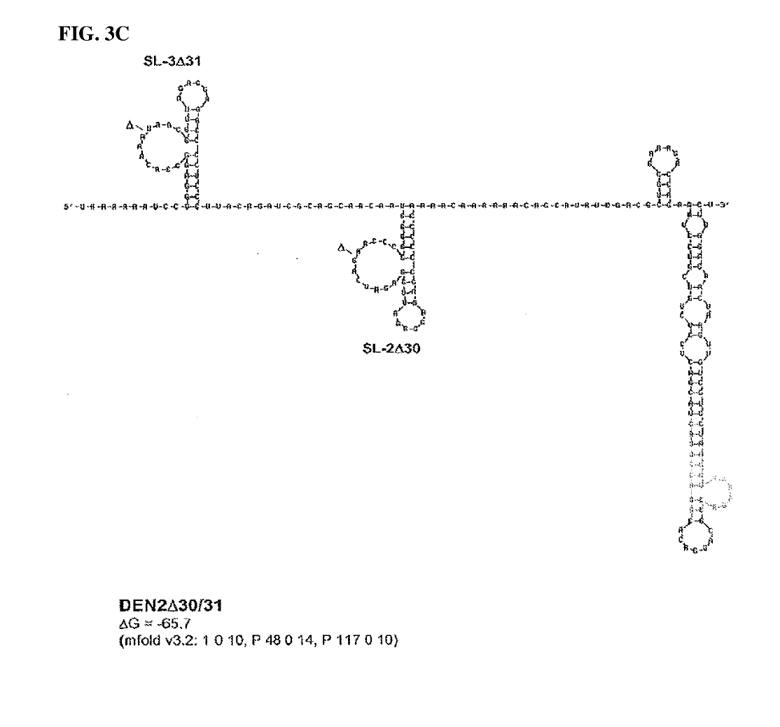

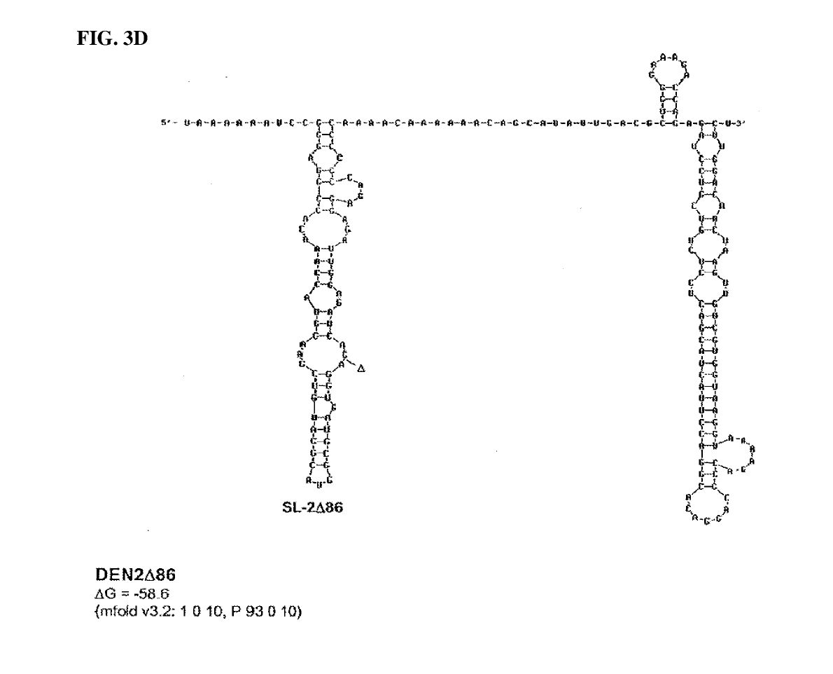

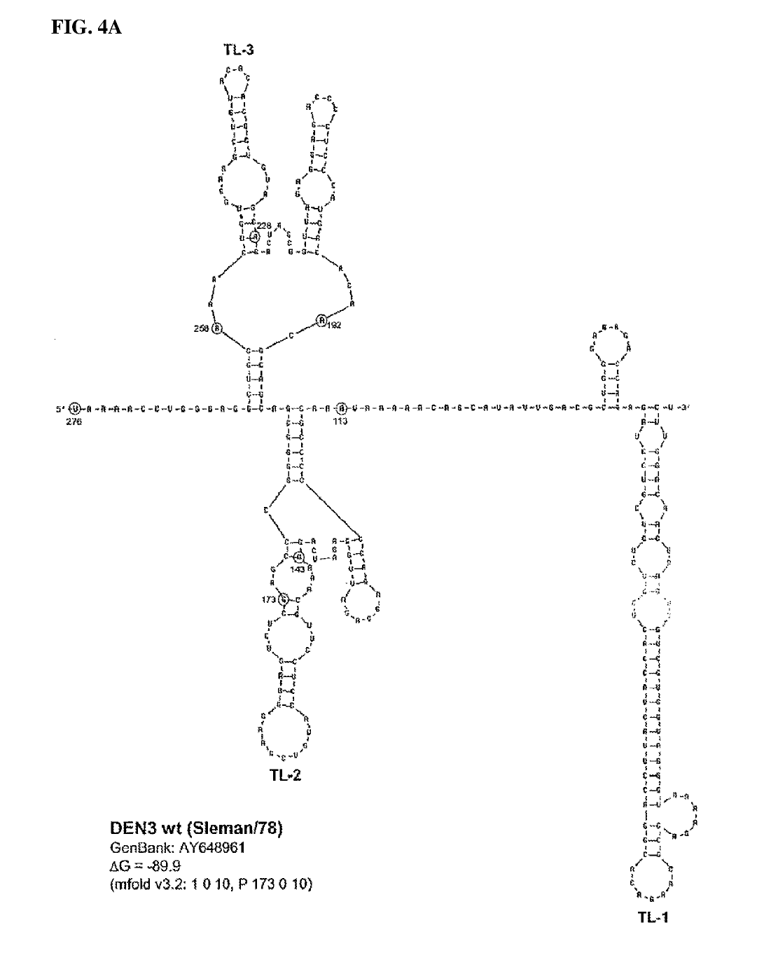

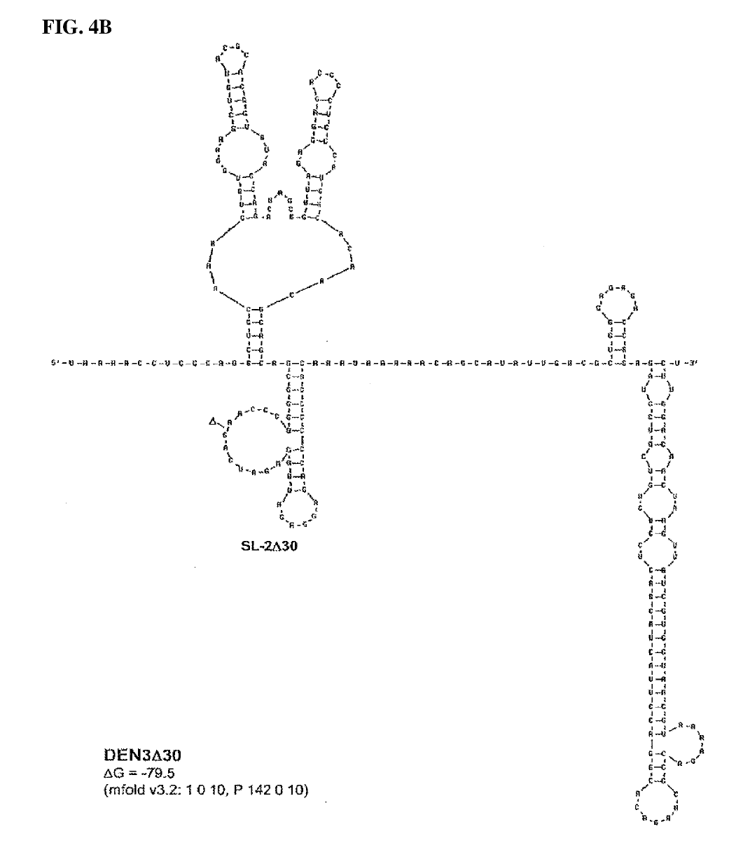

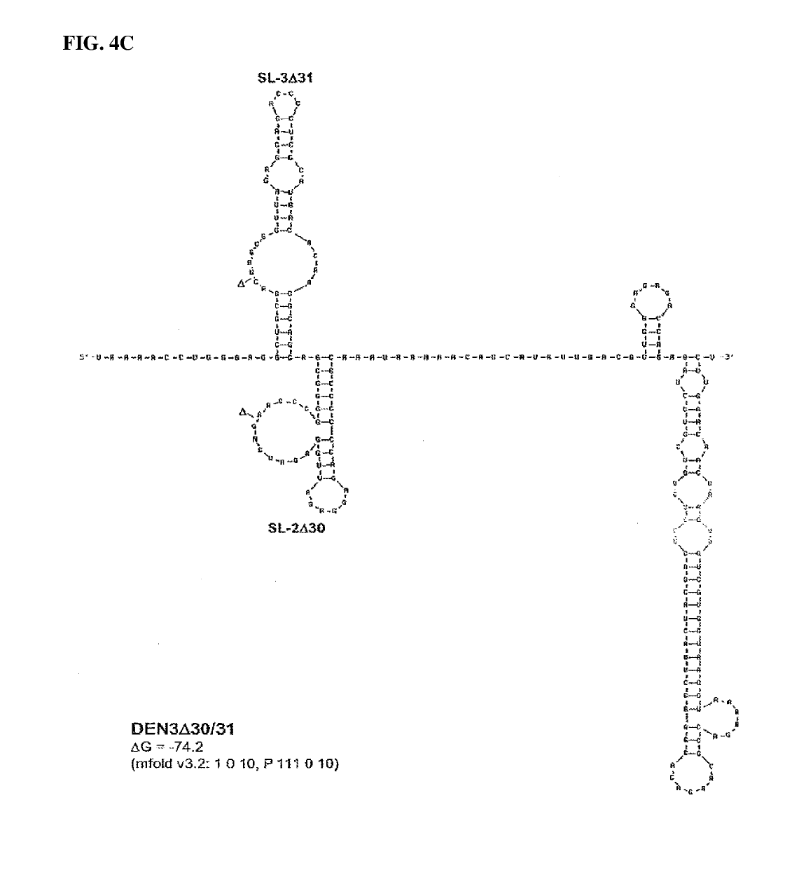

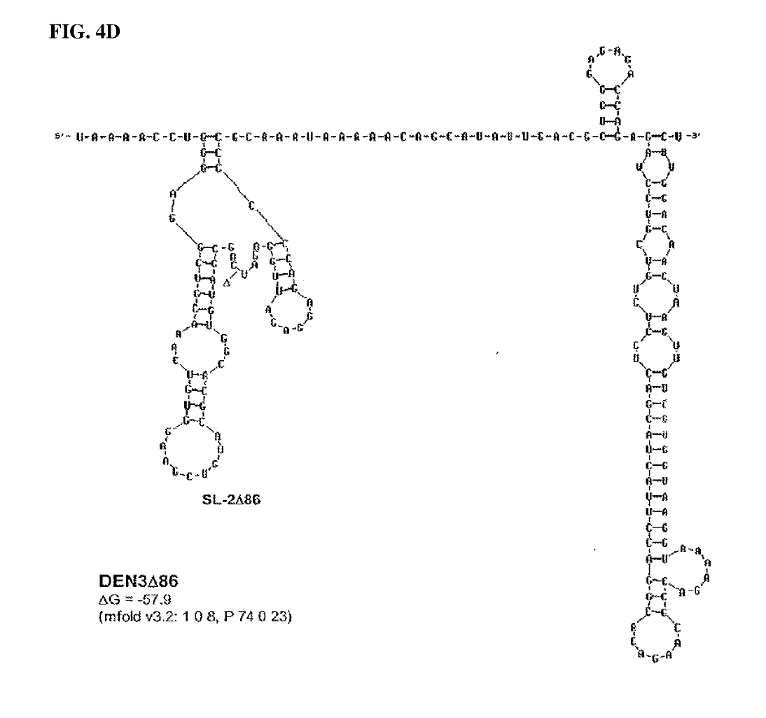

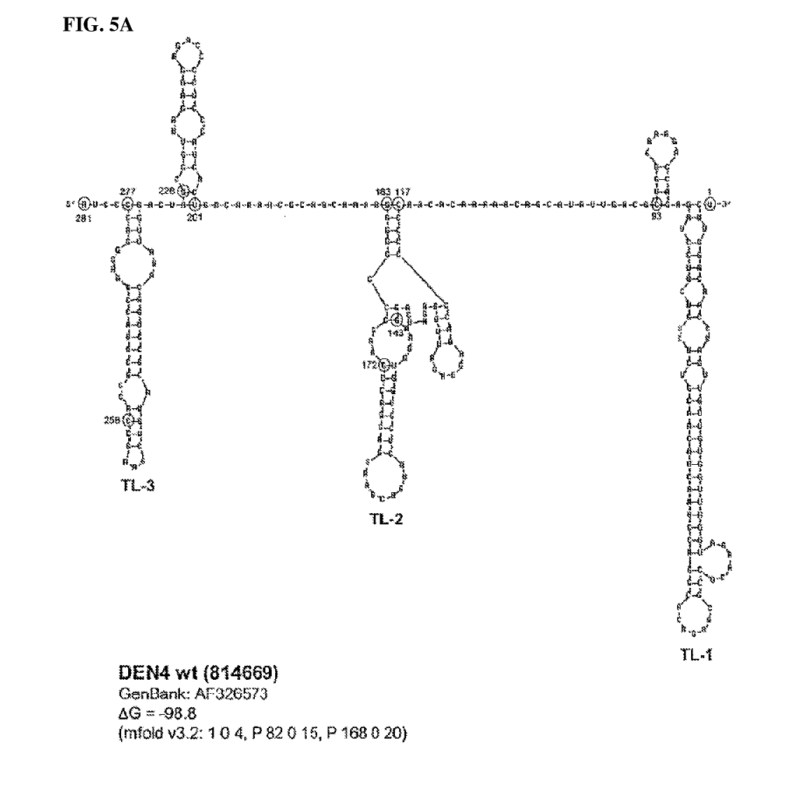

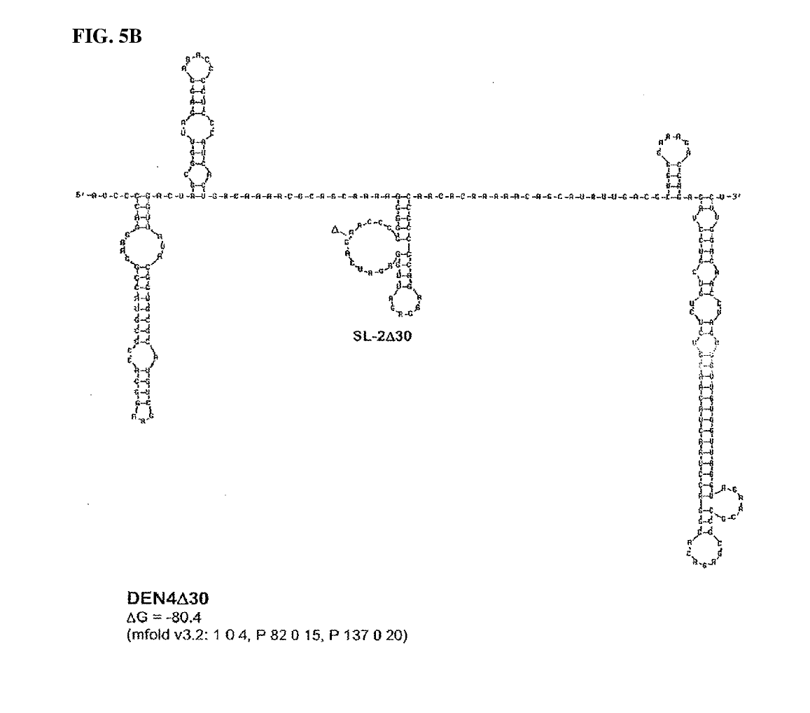

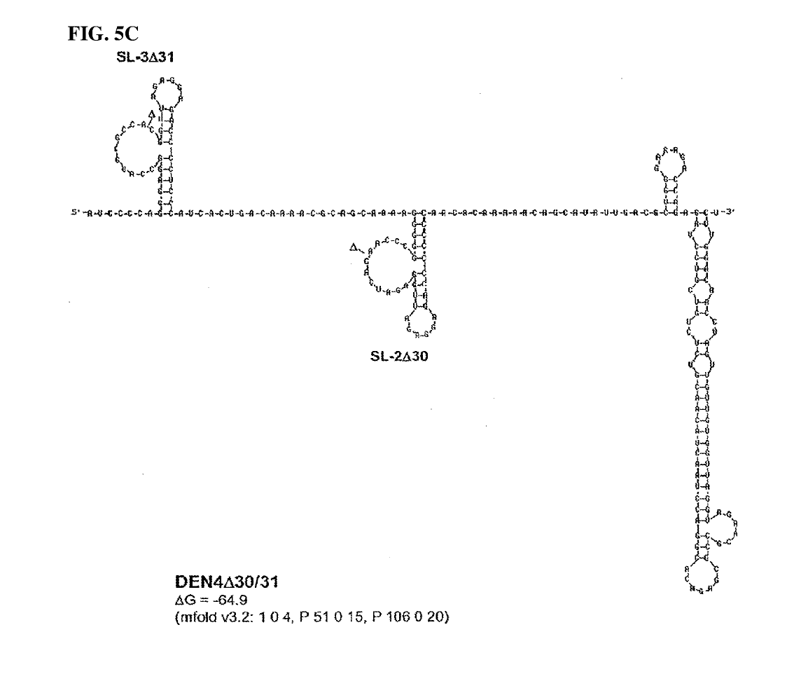

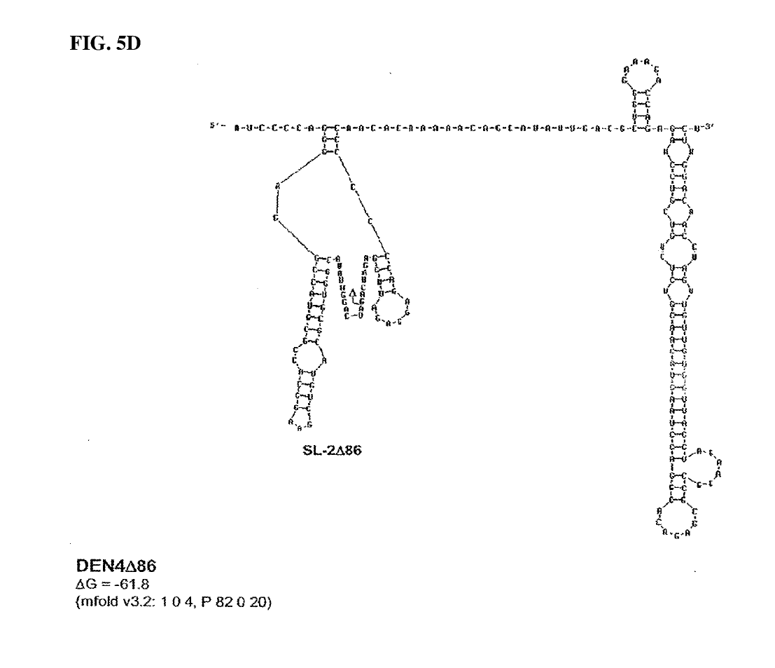

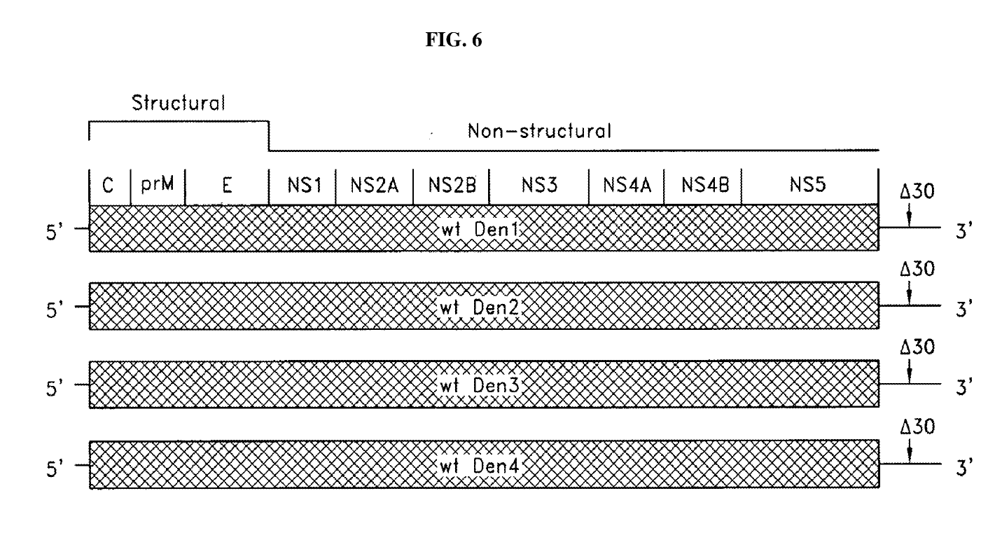

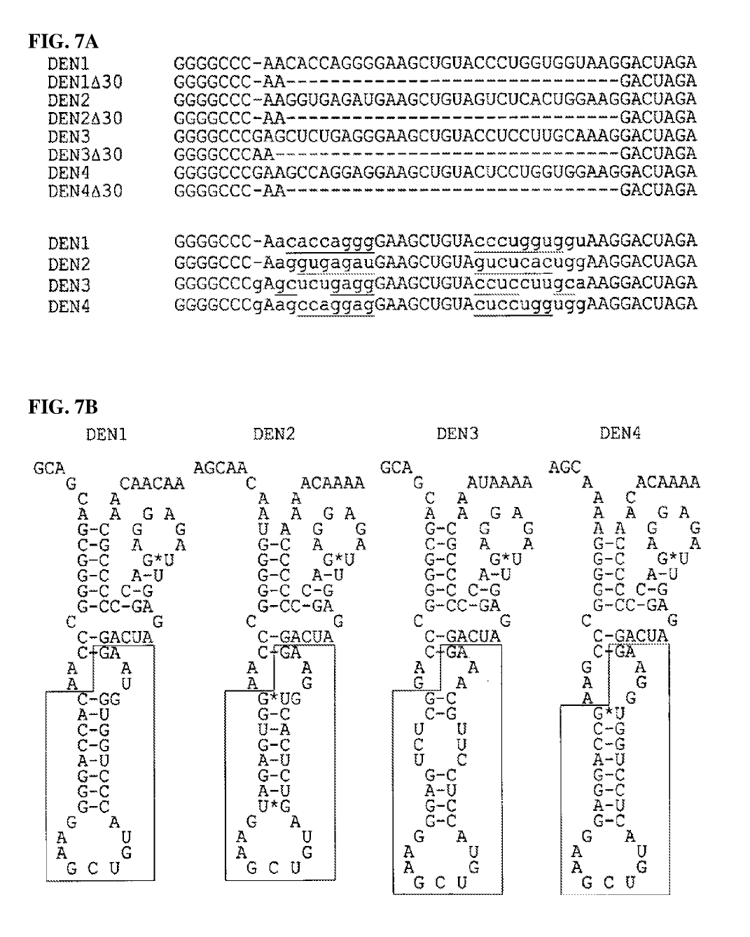

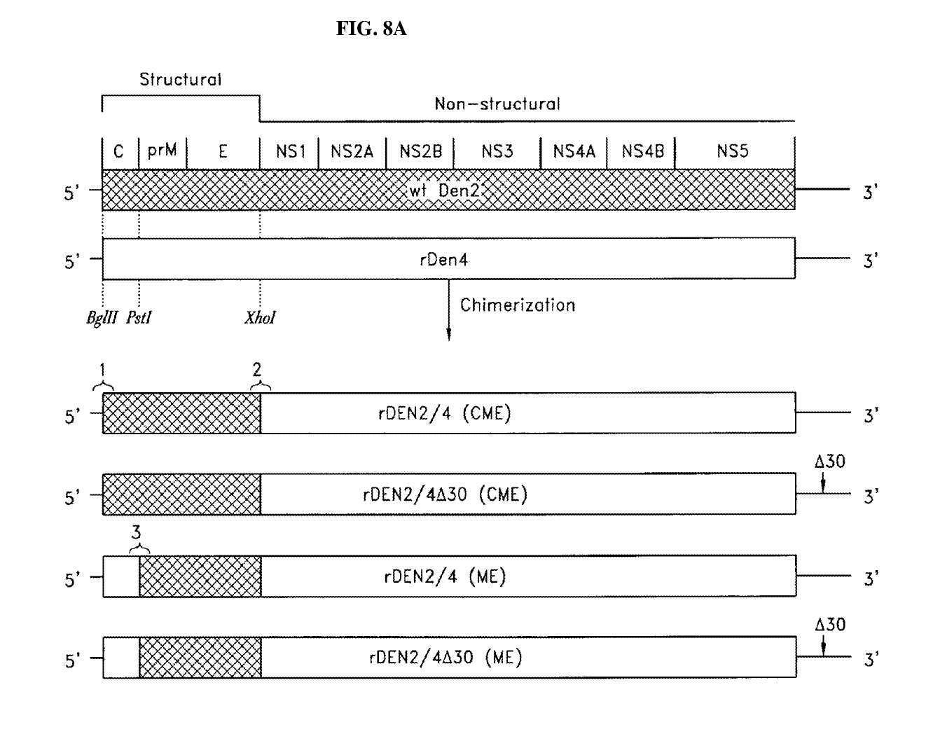

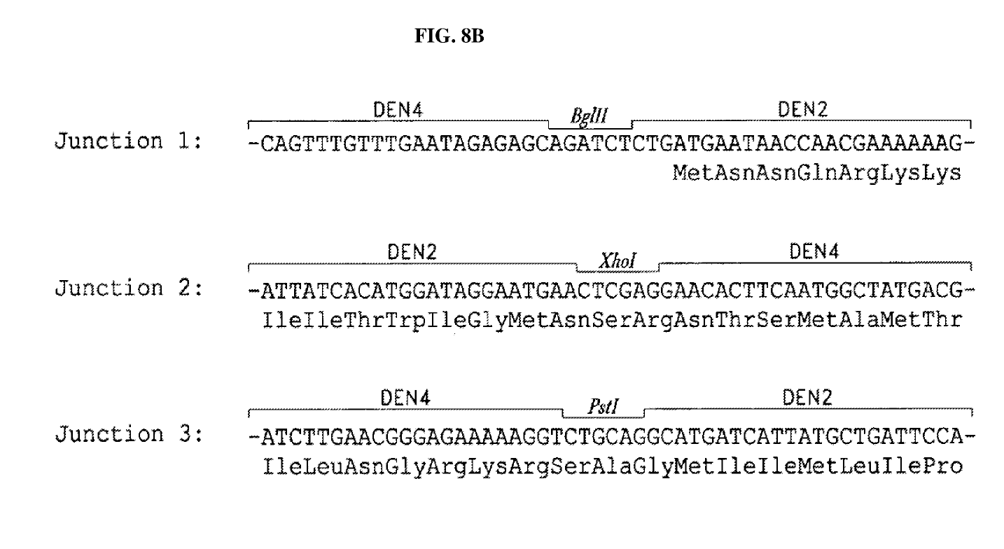

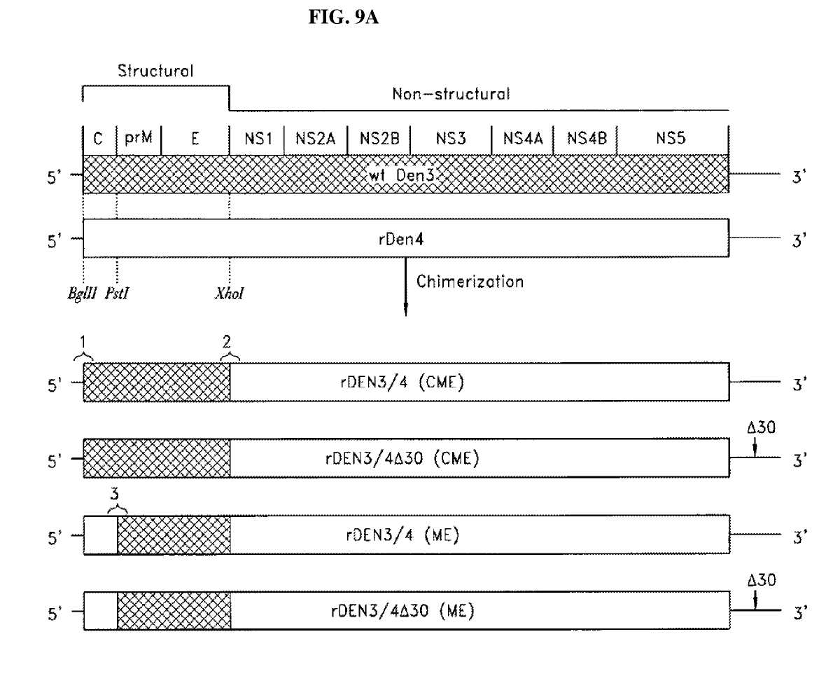

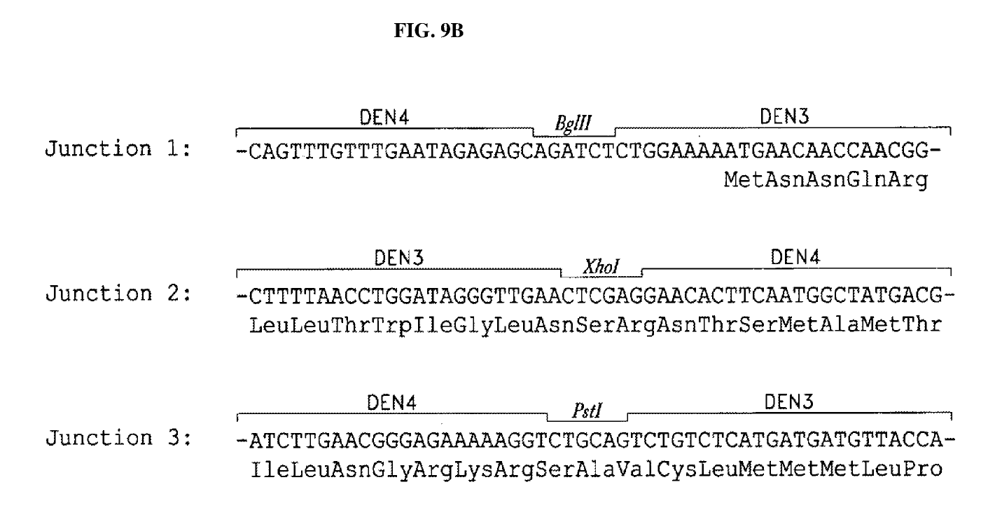

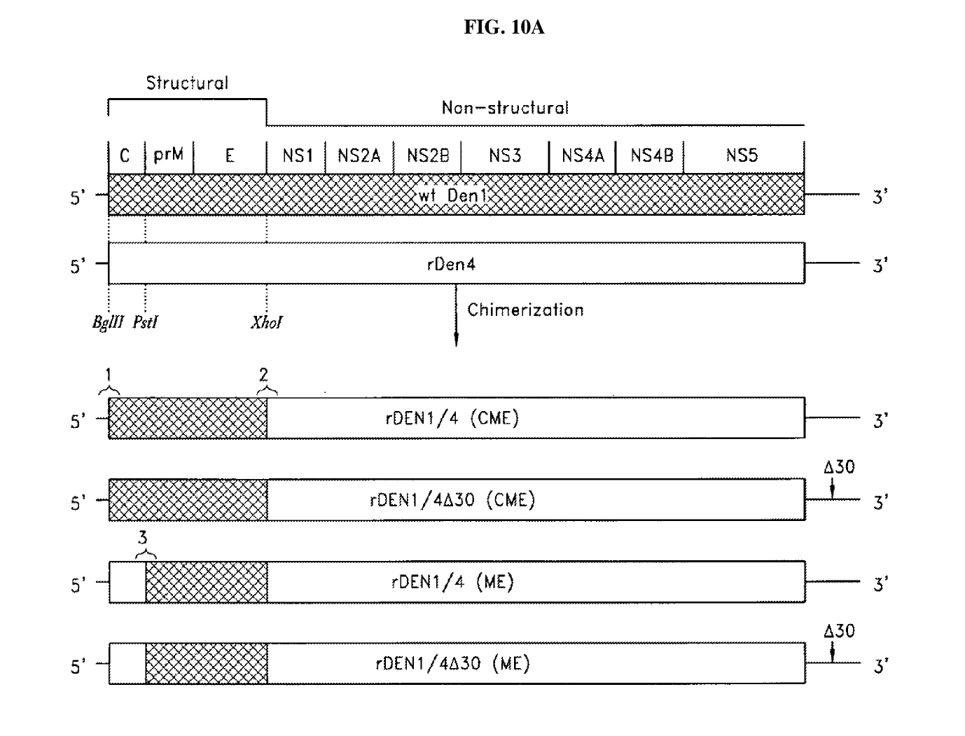

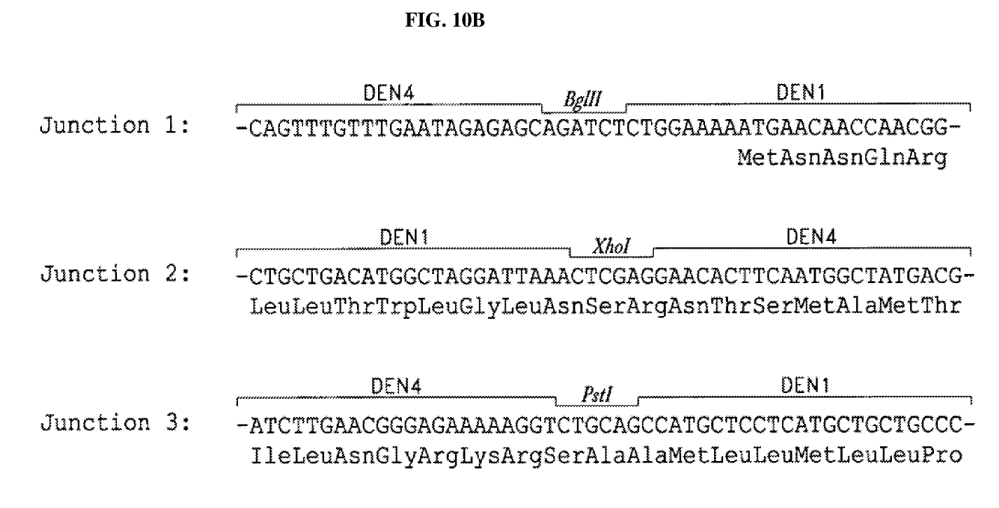

Referring to FIGS. 2A, 3A, 4A, and 5A, using an approach, the 3′ -UTR of dengue virus contains various conserved sequence motifs. The locations of various sequence components in this region are designated with the reverse-direction numbering system. These sequences include the 3′ distal secondary structure (e.g., nucleotides 1-93 in DEN4), predicted to form stem-loop 1 (SL-1), which contains terminal loop 1 (TL-1). Nucleotides 117-183 in DEN4 form step-loop (SL-2) which contains TL-2. Nucleotides 201-277 in DEN4 form a pair of stem-loops (SL-3) which in part contains TL-3. Although the nucleotides spacing between SL-2 and neighboring SL-1 and SL-3 differ among the dengue virus serotypes, the overall structure of SL-2 is well-conserved. In addition, the exposed 9 nucleotides that comprise TL-2 are identical within all 4 dengue serotypes. It is TL-2 and it's supporting stem structure that are removed by a 430 mutation (e.g., about nucleotides 143-172 in DEN4). Removal of these 30 nucleotides results in formation of a new predicted structural element (SL-2Δ30) which has a primary sequence and secondary structure which is identical for each of the dengue virus serotypes.

In particular, a mutation that is a deletion of 30 (“Δ30”) nucleotides from the 3′ untranslated region of the DEN4 genome between nucleotides 10478-10507 results in attenuation of the DEN4 virus. Therefore, the genome of any dengue type 4 virus containing such a mutation at this location can be used as the backbone in the attenuated chimeras described herein. Furthermore, other dengue virus genomes containing an analogous deletion mutation in the 3′ untranslated region of the genomes of other dengue virus serotypes may also be used as the backbone structure of the chimera of the present disclosure. For example, a mutation at this locus can be used in the genome of dengue type 1 (deletion of 30 nucleotides between 10562-10591 of DEN1; DEN1Δ30), dengue type 2 (deletion of 30 nucleotides between 10541-10570 of DEN2 Tonga/74; DEN2Δ30), dengue type 3 (deletion of 30 nucleotides between 10535-10565 of DEN3 Sleman/78; DEN3Δ30), and/or dengue type 4 (deletion of 30 nucleotides between 10478-10507 of DEN4; DEN4Δ30) as a backbone structure of the chimera of the present disclosure. The Δ30 deletion removes the TL-2 homologous structure and sequence up to the TL-3 homologous structure and can be seen in FIGS. 2B, 3B, 4B, and 5B.

In another embodiment, a mutation that is a deletion of 31 (“Δ31”) nucleotides from the TL-3 of the dengue genome attenuates the backbone structure of the chimera of the present invention. FIGS. 2C, 3C, 4C, and 5C illustrate the Δ31 deletions in DEN1, DEN2, DEN3, and DEN4, respectively. Therefore, the genome of any dengue type 2 virus containing such a mutation at this locus can be used as the backbone in the attenuated chimeras described herein. Furthermore, other dengue virus genomes containing an analogous deletion mutation in the TL-3 of the genomes of other dengue virus serotypes may also be used as the backbone structure of the chimera of the present disclosure.

In some embodiments, the dengue backbone structure of the Zika chimera of the present disclosure includes both the Δ30 and Δ31 mutations (i.e., DEN1Δ30/31, DEN2Δ30/314, DEN3Δ30/31, and/or DEN4Δ30/31).

In another embodiment, a mutation that is a deletion of 86 (“Δ86”) nucleotides removes the TL-2 homologous structure and the sequence up to the TL-3 homologous structure of a dengue virus (e.g., DEN1, DEN2, DEN3 and/or DEN4). Therefore, the genome of any dengue type 1, 2, 3, and/or 4 virus containing such a mutation at this locus can be used as the backbone in the attenuated chimeras described herein. FIGS. 2D, 3D, 4D, and 5D illustrate the Δ86 deletions in DEN1, DEN2, DEN2, and DEN4, respectively.

In a particular embodiment, the Zika chimera includes the DEN2Δ30 as the backbone structure of the chimera. In another embodiment, the Zika chimera includes the DEN4Δ30 as the backbone structure of the chimera. In other embodiments, the Zika chimera includes the DEN3Δ30/31 as the backbone structure of the chimera.

In various embodiments, the Zika chimeras of the invention can include mutations and/or deletions in the 3′ UTR and/or 5′ UTR that are in addition to the Δ30,Δ31, and Δ86 deletions, including those described in PCT Application No. PCT/US2007/076004 (DEVELOPMENT OF DENGUE VIRUS VACCINE COMPONENTS), which is incorporated herein by reference.

The mutations described above may be achieved by site-directed mutagenesis using techniques known to those skilled in the art. It will be understood by those skilled in the art that the virulence screening assays, as described herein and as are well known in the art, can be used to distinguish between virulent and attenuated backbone structures. Any of the mutagenesis techniques discussed in PCT Application No. PCT/US2007/076004 (DEVELOPMENT OF DENGUE VIRUS VACCINE COMPONENTS) are contemplated.

Construction of Zika Flavivirus Chimeras

The flavivirus chimeras described herein can be produced by substituting at least one of the structural protein genes of the ZIKV against which immunity is desired into a dengue virus genome backbone, using recombinant engineering techniques well known to those skilled in the art, namely, removing a designated dengue virus gene and replacing it with the desired corresponding gene of ZIKV. Alternatively, using the sequences provided in GenBank, the nucleic acid molecules encoding the flavivirus proteins may be synthesized using known nucleic acid synthesis techniques and inserted into an appropriate vector. Attenuated, immunogenic virus is therefore produced using recombinant engineering techniques known to those skilled in the art.

As mentioned above, the gene to be inserted into the backbone encodes a ZIKV structural protein. Preferably the ZIKV gene to be inserted is a gene encoding a C protein, a prM protein and/or an E protein. The sequence inserted into the dengue virus backbone can encode both the prM and E structural proteins. The sequence inserted into the dengue virus backbone can encode the C, prM and E structural proteins. The dengue virus backbone is the DEN1, DEN2, DEN3, or DEN4 virus genome, or an attenuated dengue virus genome of any of these serotypes, and includes the substituted gene(s) that encode the C, prM and/or E structural protein(s) of a ZIKV or the substituted gene(s) that encode the prM and/or E structural protein(s) of a ZIKV.

Suitable chimeric viruses or nucleic acid chimeras containing nucleotide sequences encoding structural proteins of ZIKV can be evaluated for usefulness as vaccines by screening them for phenotypic markers of attenuation that indicate reduction in virulence with retention of immunogenicity. Antigenicity and immunogenicity can be evaluated using in vitro or in vivo reactivity with Zika antibodies or immunoreactive serum using routine screening procedures known to those skilled in the art.

Flavivirus Vaccines

The preferred chimeric viruses and nucleic acid chimeras provide live, attenuated viruses useful as immunogens or vaccines. In a preferred embodiment, the chimeras exhibit high immunogenicity while at the same time not producing dangerous pathogenic or lethal effects.

The chimeric viruses or nucleic acid chimeras of this invention can comprise the structural genes of a ZIKV in a wild-type or an attenuated dengue virus backbone. For example, the chimera may express the structural protein genes of a ZIKV in either of a dengue virus or an attenuated dengue virus background.

The strategy described herein of using a genetic background that contains nonstructural regions of a dengue virus genome, and, by chimerization, the properties of attenuation, to express the structural protein genes of a ZIKV has led to the development of live, attenuated flavivirus vaccine candidates that express structural protein genes of desired immunogenicity. Thus, vaccine candidates for control of ZIKV pathogens can be designed.

Viruses used in the chimeras described herein are typically grown using techniques known in the art. Virus plaque or focus forming unit (FFU) titrations are then performed and plaques or FFU are counted in order to assess the viability, titer and phenotypic characteristics of the virus grown in cell culture. Wild type viruses are mutagenized to derive attenuated candidate starting materials.

Chimeric infectious clones are constructed from various flavivirus strains. The cloning of virus-specific cDNA fragments can also be accomplished, if desired. The cDNA fragments containing the structural protein or nonstructural protein genes are amplified by reverse transcriptase-polymerase chain reaction (RT-PCR) from flavivirus RNA with various primers. Amplified fragments are cloned into the cleavage sites of other intermediate clones. Intermediate, chimeric flavivirus clones are then sequenced to verify the sequence of the inserted flavivirus-specific cDNA.

Full genome-length chimeric plasmids constructed by inserting the structural or nonstructural protein gene region of flaviviruses into vectors are obtainable using recombinant techniques well known to those skilled in the art.

Multivalent and Pentavalent Flavivirus Chimera Vaccine

The present disclosure not only relates to Zika and Zika chimeric viruses for use as vaccines and to said vaccines themselves, but also to multivalent vaccines comprising the combination of at least two different vaccines, wherein at least one vaccine is a vaccine against ZIKV. In other words, the disclosure contemplates combining one or more Zika vaccines (e.g., an attenuated ZIKV, a chimeric attenuated ZIKV, or both) with one or more additional vaccines to other pathogens. In certain embodiments, the one or more additional vaccines are flavivirus vaccines. The one or more additional vaccines can be selected from any flavivirus vaccine, such as, but not limited to, a dengue vaccine (against DEN1, DEN2, DEN3, DEN4, or combinations thereof), yellow fever virus vaccine, JEV vaccine, TBEV vaccine, West Nile virus vaccine, or combinations thereof.

In certain embodiments, a multivalent vaccine comprises:

(a) one or more Zika vaccines (e.g., attenuated ZIKV or chimeric attenuated ZIKV), combined with one, two, three, four, or five additional flavivirus vaccines;

(b) one or more Zika vaccines (e.g., attenuated ZIKV or chimeric attenuated ZIKV), combined with one, two, three, four, or five additional dengue vaccines (against DEN1, DEN2, DEN3, DEN4, chimeras thereof, or combinations thereof);

(c) a chimeric attenuated ZIKV vaccine combined one or more dengue virus vaccines, said dengue virus vaccines each comprising at least one of a DEN1, DEN2, DEN3, or DEN4 virus, or chimerics thereof, or combinations thereof; and

(d) a chimeric attenuated ZIKV vaccine combined a DEN1 virus vaccine, a DEN2 virus vaccine, a DEN3 virus vaccine, and a DEN4 virus vaccine, or chimerics thereof, i.e., to provide a pentavalent vaccine.

In any embodiments relating to multivalent and/or pentavalent vaccines, the one or more additional flavivirus vaccines may comprise flaviviruses which comprise one or more attenuating mutations, including deletions and/or mutations in the 3′UTR, e.g., Δ30, Δ30/31, and Δ86 attenuating mutations.

The description provides a set of type-specific, live attenuated flavivirus vaccine components (e.g., dengue virus) that can be formulated into a safe, effective, and economical multivalent flavivirus vaccine (e.g., bivalent, trivalent, tetravalent, or pentavalent) with an attenuated ZIKV or Zika chimera. The Δ30 mutation attenuates DEN2 and DEN4 in rhesus monkeys. The Δ30 mutation removes a homologous structure (TL-2) in each of the dengue virus serotypes 1, 2, 3, and 4 (FIGS. 2B, 3B, 4B, and 5B). However, the Δ30 mutation was found to not attenuate DEN3 to the same extent as in DEN2 and DEN4 in rhesus monkeys. In contrast, the Δ30 mutation was found to attenuate DEN1 to a greater extent than DEN2 and DEN4.

In certain embodiments, the description provides flavivirus (e.g., dengue viruses) and chimeric flaviviruses (e.g., dengue viruses) having one or more mutations that result in attenuation, methods of making such dengue viruses, and methods for using these flaviviruses to prevent or treat flavivirus infection (e.g., dengue virus infection). The mutation (or mutations) in the dengue virus of the invention is present in the 3′ untranslated region (3′-UTR) formed by the most downstream approximately 384 nucleotides of the viral RNA, which have been shown to play a role in determining attenuation. The viruses and methods of the invention are described further, as follows. A molecular approach is used to develop a genetically stable live attenuated multivalent (e.g., pentavalent) Zika, flavivirus virus immunogenic composition or vaccine. The multivalent immunogenic composition comprising: at least one first attenuated viruses that are immunogenic against a flavivirus, and a second attenuated virus that is immunogenic against ZIKV. In a particular embodiment, the first attenuated virus is immunogenic against a virus selected from the group consisting of: dengue virus (e.g., DEN1, DEN2, DEN3, DEN4, or a combination thereof), West Nile virus, yellow fever virus, Japanese encephalitis virus, tick-borne encephalitis virus, or combinations thereof. In another embodiment, the second attenuated virus is a Zika nucleic acid chimera in accordance with the present disclosure. In another particular embodiment, the second attenuated virus is a ZIKV comprising one or more attenuating mutations in the genome. Each component of the multivalent vaccine must be attenuated, genetically stable and immunogenic.

For example, each component of the pentavalent vaccine, e.g., DEN1, DEN2, DEN3, DEN4, and ZIKV, must be attenuated, genetically stable, and immunogenic. The pentavalent vaccine will ensure simultaneous protection against each of the four dengue viruses, thereby precluding the possibility of developing the more serious illnesses dengue hemorrhagic fever/dengue shock syndrome (DHF/DSS), which occur in humans during secondary infection with a heterotypic wild-type dengue virus. Since dengue viruses may undergo genetic recombination in nature, the pentavalent vaccine will be genetically incapable of undergoing a recombination event between its five virus components that could lead to the generation of viruses lacking attenuating mutations. Previous approaches to develop a tetravalent dengue virus vaccine have been based on independently deriving each of the four virus components through separate mutagenic procedures, such as passage in tissue culture cells derived from a heterologous host. This strategy has yielded attenuated vaccine candidates previously. However, it is possible that gene exchanges among the four components of these independently derived tetravalent vaccines could occur in vaccinees, possibly creating a virulent recombinant virus. Virulent polioviruses derived from recombination have been generated in vaccinees following administration of a trivalent poliovirus vaccine.

In certain aspects, the present disclosure provides for a pentavalent vaccine that can include: (1) attenuated Zika chimera according to the present disclosure, rDEN4Δ30 and rDEN1Δ30, rDEN2Δ30, and rDEN3Δ30 recombinant viruses containing a 30 nucleotide deletion (Δ30) in a section of the 3′ untranslated region (UTR) that is homologous to that in the rDEN4Δ30 recombinant virus; (2) attenuated nucleic acid Zika chimera according to the present disclosure, rDEN1Δ30, rDEN2Δ30, rDEN3Δ30, and rDEN4Δ30; (3) attenuated antigenic chimeric viruses, rDEN1/4Δ30, rDEN2/4Δ30, and rDEN3/4Δ30, for which the CME, ME, or E gene regions of rDEN4Δ30 have been replaced with those derived from DEN1, DEN2, or DENS, rDEN4Δ30, and Zika chimera; alternatively rDEN1/3Δ30, rDEN2/3Δ30, and rDEN4/3Δ30 for which CME, ME, or E gene regions of rDEN3Δ30 have been replaced with those derived from DEN1, 2, or 4, rDEN3Δ30, and Zika chimera; alternatively rDEN1/2Δ30, rDEN3/2Δ30, and rDEN4/2Δ30 for which CME, ME, or E gene regions of rDEN2Δ30 have been replaced with those derived from DEN1, 3, or, 4, rDEN2Δ30, and Zika chimera; and alternatively rDEN2/1Δ30, rDEN3/1Δ30, and rDEN4/1Δ30 for which CME, ME, or E gene regions of rDEN1Δ30 have been replaced with those derived from DEN2, 3, or 4, rDEN1Δ30, and Zika chimera; and (4) attenuated rDEN1/4Δ30, rDEN2/4Δ30, rDEN3/4Δ30, rDEN4Δ30, and Zika chimera; alternatively rDEN1/3Δ30, rDEN2/3Δ30, rDEN4/3Δ30, rDEN3Δ30, and Zika chimera; alternatively rDEN1/2Δ30, rDEN3/2Δ30, rDEN4/2Δ30, rDEN2Δ30, and Zika chimera; and alternatively rDEN2/1Δ30, rDEN3/1Δ30, rDEN4/1Δ30, rDEN1Δ30, and Zika chimera. These pentavalent vaccines are unique since they contain a common shared attenuating mutation which eliminates the possibility of generating a virulent wild-type virus in a vaccinee since each component of the vaccine possesses the same Δ30 attenuating deletion mutation. In addition, the rDEN1Δ30, rDEN2Δ30, rDEN3Δ30, rDEN4Δ30, Zika chimera pentavalent vaccine is the first to combine the stability of the Δ30 mutation with broad antigenicity. Alternatively, utilizing the same scheme, the Δ31, Δ30/31 or Δ86 deletions of the 3′UTR may be utilized in the chimera schemes described above, or within DEN1, DEN2, DEN3, DEN4, and Zika chimera. Since the Δ30, Δ31, Δ30/31, and Δ86 deletion mutation is in the 3′ UTR of each virus, all of the proteins of the five component viruses are available to induce a protective immune response. Thus, the method provides a mechanism of attenuation that maintains each of the proteins of DEN1, DEN2, DEN3, DEN4, and Zika chimera viruses in a state that preserves the full capability of each of the proteins of the five viruses to induce humoral and cellular immune responses against all of the structural and non-structural proteins present in each dengue virus serotype and ZIKV.

As previously described, the DEN4 recombinant virus, rDEN4Δ30 (previously referred to as 2AΔ30), was engineered to contain a 30 nucleotide deletion in the 3′ UTR of the viral genome (Durbin, A. P. et al. 2001 Am J Trop Med Hyg 65:405-13; Men, R. et al. 1996 J Virol 70:3930-7). Evaluation in rhesus monkeys showed the virus to be significantly attenuated relative to wild-type parental virus, yet highly immunogenic and completely protective. Also, a phase I clinical trial with adult human volunteers showed the rDEN4Δ30 recombinant virus to be safe and satisfactorily immunogenic (Durbin, A. P. et al. 2001 Am J Trop Med Hyg 65:405-13). To develop a pentavalent vaccine bearing a shared attenuating mutation in a untranslated region, the Δ30, Δ31, Δ30/31, or Δ86 deletion to attenuate wild-type dengue viruses of serotypes 1, 2, and 3 since it attenuated wild-type DEN4 virus for rhesus monkeys and was safe in humans, may be used. U.S. Patent Publication Application 2007/0009552.

According to an aspect, the present disclosure provides for a pentavalent immunogenic composition comprising: a first attenuated virus that is immunogenic against dengue serotype 1 (DEN1), a second attenuated virus that is immunogenic against dengue serotype 2 (DEN2), a third attenuated virus that is immunogenic against dengue serotype 3 (DEN3), a fourth attenuated virus that is immunogenic against dengue serotype 4 (DEN4), and a fifth attenuated virus that is immunogenic against ZIKV. In a particular embodiment, the fifth attenuated virus is the Zika nucleic acid chimera in accordance with the present disclosure.

In an embodiment, the first, second, third, and fourth attenuated viruses are selected from the group consisting of: (1) rDEN1Δ30, rDEN2Δ30, rDEN3Δ30, rDEN4Δ30, (2) rDEN1Δ30, rDEN2Δ30, rDEN3Δ30, rDEN4/1Δ30, (3) rDEN1Δ30, rDEN2Δ30, rDEN3Δ30, rDEN4/2Δ30, (4) rDEN1Δ30, rDEN2Δ30, rDEN3Δ30, rDEN4/3Δ30, (5) rDEN1Δ30, rDEN2Δ30, rDEN3/1Δ30, rDEN4Δ30, (6) rDEN1Δ30, rDEN2Δ30, rDEN3/1Δ30, rDEN4/1Δ30, (7) rDEN1Δ30, rDEN2Δ30, rDEN3/1Δ30, rDEN4/2Δ30, (8) rDEN1Δ30, rDEN2Δ30, rDEN3/1Δ30, rDEN4/3Δ30, (9) rDEN1Δ30, rDEN2Δ30, rDEN3/2Δ30, rDEN4Δ30, (10) rDEN1Δ30, rDEN2Δ30, rDEN3/2Δ30, rDEN4/1Δ30, (11) rDEN1Δ30, rDEN2Δ30, rDEN3/2Δ30, rDEN4/2Δ30, (12) rDEN1Δ30, rDEN2Δ30, rDEN3/2Δ30, rDENΔ/3Δ30, (13) rDEN1Δ30, rDEN2Δ30, rDEN3/4Δ30, rDEN4Δ30, (14) rDEN1Δ30, rDEN2Δ30, rDEN3/4Δ30, rDENΔ/1Δ30, (15) rDEN1Δ30, rDEN2Δ30, rDEN3/4Δ30, rDEN4/2Δ30, (16) rDEN1Δ30, rDEN2Δ30, rDEN3/4Δ30, rDEN4/3Δ30, (17) rDEN1Δ30, rDEN2/1Δ30, rDEN3Δ30, rDEN4Δ30, (18) rDEN1Δ30, rDEN2/1Δ30, rDEN3Δ30, rDENΔ/1Δ30, (19) rDEN1Δ30, rDEN2/1Δ30, rDEN3Δ30, rDENΔ/2Δ30, (20) rDEN1Δ30, rDEN2/1Δ30, rDEN3Δ30, rDEN4/3Δ30, (21) rDEN1Δ30, rDEN2/1Δ30, rDEN3/1Δ30, rDEN4Δ30, (22) rDEN1Δ30, rDEN2/1Δ30, rDEN3/1Δ30, rDENΔ/1Δ30, (23) rDEN1Δ30, rDEN2/1Δ30, rDEN3/1Δ30, rDENΔ/2Δ30, (24) rDEN1Δ30, rDEN2/1Δ30, rDEN3/1Δ30, rDENΔ/3Δ30, (25) rDEN1Δ30, rDEN2/1Δ30, rDEN3/2Δ30, rDEN4Δ30, (26) rDEN1Δ30, rDEN2/1Δ30, rDEN3/2Δ30, rDENΔ/1Δ30, (27) rDEN1Δ30, rDEN2/1Δ30, rDEN3/2Δ30, rDENΔ/2Δ30, (28) rDEN1Δ30, rDEN2/1Δ30, rDEN3/2Δ30, rDEN4/3Δ30, (29) rDEN1Δ30, rDEN2/1Δ30, rDEN3/4Δ30, rDEN4Δ30, (30) rDEN1Δ30, rDEN2/1Δ30, rDEN3/4Δ30, rDENΔ/1Δ30, (31) rDEN1Δ30, rDEN2/1Δ30, rDEN3/4Δ30, rDEN4/2Δ30, (32) rDEN1Δ30, rDEN2/1Δ30, rDEN3/4Δ30, rDEN4/3Δ30, (33) rDEN1Δ30, rDEN2/3Δ30, rDEN3Δ30, rDEN4Δ30, (34) rDEN1Δ30, rDEN2/3Δ30, rDEN3Δ30, rDEN4/1Δ30, (35) rDEN1Δ30, rDEN2/3Δ30, rDEN3Δ30, rDENΔ/2Δ30, (36) rDEN1Δ30, rDEN2/3Δ30, rDEN3Δ30, rDEN4/3Δ30, (37) rDEN1Δ30, rDEN2/3Δ30, rDEN3/1Δ30, rDEN4Δ30, (38) rDEN1Δ30, rDEN2/3Δ30, rDEN3/1Δ30, rDEN4/1Δ30, (39) rDEN1Δ30, rDEN2/3Δ30, rDEN3/1Δ30, rDENΔ/2Δ30, (40) rDEN1Δ30, rDEN2/3Δ30, rDEN3/1Δ30, rDEN4/3Δ30, (41) rDEN1Δ30, rDEN2/3Δ30, rDEN3/2Δ30, rDEN4Δ30, (42) rDEN1Δ30, rDEN2/3Δ30, rDEN3/2Δ30, rDENΔ/1Δ30, (43) rDEN1Δ30, rDEN2/3Δ30, rDEN3/2Δ30, rDEN4/2Δ30, (44) rDEN1Δ30, rDEN2/3Δ30, rDEN3/2Δ30, rDEN4/3Δ30, (45) rDEN1Δ30, rDEN2/3Δ30, rDEN3/4Δ30, rDEN4Δ30, (46) rDEN1Δ30, rDEN2/3Δ30, rDEN3/4Δ30, rDEN4/1Δ30, (47) rDEN1Δ30, rDEN2/3Δ30, rDEN3/4Δ30, rDEN4/2Δ30, (48) rDEN1Δ30, rDEN2/3Δ30, rDEN3/4Δ30, rDEN4/3Δ30, (49) rDEN1Δ30, rDEN2/4Δ30, rDEN3Δ30, rDEN4Δ30, (50) rDEN1Δ30, rDEN2/4Δ30, rDEN3Δ30, rDEN4/1Δ30, (51) rDEN1Δ30, rDEN2/4Δ30, rDEN3Δ30, rDEN4/2Δ30, (52) rDEN1Δ30, rDEN2/4Δ30, rDEN3Δ30, rDEN4/3Δ30, (53) rDEN1Δ30, rDEN2/4Δ30, rDEN3/1Δ30, rDEN4Δ30, (54) rDEN1Δ30, rDEN2/4Δ30, rDEN3/1Δ30, rDEN4/1Δ30, (55) rDEN1Δ30, rDEN2/4Δ30, rDEN3/1Δ30, rDEN4/2Δ30, (56) rDEN1Δ30, rDEN2/4Δ30, rDEN3/1Δ30, rDEN4/3Δ30, (57) rDEN1Δ30, rDEN2/4Δ30, rDEN3/2Δ30, rDEN4Δ30, (58) rDEN1Δ30, rDEN2/4Δ30, rDEN3/2Δ30, rDEN4/1Δ30, (59) rDEN1Δ30, rDEN2/4Δ30, rDEN3/2Δ30, rDEN4/2Δ30, (60) rDEN1Δ30, rDEN2/4Δ30, rDEN3/2Δ30, rDEN4/3Δ30, (61) rDEN1Δ30, rDEN2/4Δ30, rDEN3/4Δ30, rDEN4Δ30, (62) rDEN1Δ30, rDEN2/4Δ30, rDEN3/4Δ30, rDEN4/1Δ30, (63) rDEN1Δ30, rDEN2/4Δ30, rDEN3/4Δ30, rDEN4/2Δ30, (64) rDEN1Δ30, rDEN2/4Δ30, rDEN3/4Δ30, rDEN4/3Δ30, (65) rDEN1/2Δ30, rDEN2Δ30, rDEN3Δ30, rDEN4Δ30, (66) rDEN1/2Δ30, rDEN2Δ30, rDEN3Δ30, rDEN4/1Δ30, (67) rDEN1/2Δ30, rDEN2Δ30, rDEN3Δ30, rDEN4/2Δ30, (68) rDEN1/2Δ30, rDEN2Δ30, rDEN3Δ30, rDEN4/3Δ30, (69) rDEN1/2Δ30, rDEN2Δ30, rDEN3/1Δ30, rDEN4Δ30, (70) rDEN1/2Δ30, rDEN2Δ30, rDEN3/1Δ30, rDEN4/1Δ30, (71) rDEN1/2Δ30, rDEN2Δ30, rDEN3/1Δ30, rDEN4/2Δ30, (72) rDEN1/2Δ30, rDEN2Δ30, rDEN3/1Δ30, rDEN4/3Δ30, (73) rDEN1/2Δ30, rDEN2Δ30, rDEN3/2Δ30, rDEN4Δ30, (74) rDEN1/2Δ30, rDEN2Δ30, rDEN3/2Δ30, rDEN4/1Δ30, (75) rDEN1/2Δ30, rDEN2Δ30, rDEN3/2Δ30, rDEN4/2Δ30, (76) rDEN1/2Δ30, rDEN2Δ30, rDEN3/2Δ30, rDEN4/3Δ30, (77) rDEN1/2Δ30, rDEN2Δ30, rDEN3/4Δ30, rDEN4Δ30, (78) rDEN1/2Δ30, rDEN2Δ30, rDEN3/4Δ30, rDEN4/1Δ30, (79) rDEN1/2Δ30, rDEN2Δ30, rDEN3/4Δ30, rDEN4/2Δ30, (80) rDEN1/2Δ30, rDEN2Δ30, rDEN3/4Δ30, rDEN4/3Δ30, (81) rDEN1/2Δ30, rDEN2/1Δ30, rDEN3Δ30, rDEN4Δ30, (82) rDEN1/2Δ30, rDEN2/1Δ30, rDEN3Δ30, rDEN4/1Δ30, (83) rDEN1/2Δ30, rDEN2/1Δ30, rDEN3Δ30, rDEN4/2Δ30, (84) rDEN1/2Δ30, rDEN2/1Δ30, rDEN3Δ30, rDEN4/3Δ30, (85) rDEN1/2Δ30, rDEN2/1Δ30, rDEN3/1Δ30, rDEN4Δ30, (86) rDEN1/2Δ30, rDEN2/1Δ30, rDEN3/1Δ30, rDEN4/1Δ30, (87) rDEN1/2Δ30, rDEN2/1Δ30, rDEN3/1Δ30, rDEN4/2Δ30, (88) rDEN1/2Δ30, rDEN2/1Δ30, rDEN3/1Δ30, rDEN4/3Δ30, (89) rDEN1/2Δ30, rDEN2/1Δ30, rDEN3/2Δ30, rDEN4Δ30, (90) rDEN1/2Δ30, rDEN2/1Δ30, rDEN3/2Δ30, rDEN4/1Δ30, (91) rDEN1/2Δ30, rDEN2/1Δ30, rDEN3/2Δ30, rDEN4/2Δ30, (92) rDEN1/2Δ30, rDEN2/1Δ30, rDEN3/2Δ30, rDEN4/3Δ30, (93) rDEN1/2Δ30, rDEN2/1Δ30, rDEN3/4Δ30, rDEN4Δ30, (94) rDEN1/2Δ30, rDEN2/1Δ30, rDEN3/4Δ30, rDEN4/1Δ30, (95) rDEN1/2Δ30, rDEN2/1Δ30, rDEN3/4Δ30, rDEN4/2Δ30, (96) rDEN1/2Δ30, rDEN2/1Δ30, rDEN3/4Δ30, rDEN4/3Δ30, (97) rDEN1/2Δ30, rDEN2/3Δ30, rDEN3Δ30, rDEN4Δ30, (98) rDEN1/2Δ30, rDEN2/3Δ30, rDEN3Δ30, rDEN4/1Δ30, (99) rDEN1/2Δ30, rDEN2/3Δ30, rDEN3Δ30, rDEN4/2Δ30, (100) rDEN1/2Δ30, rDEN2/3Δ30, rDEN3Δ30, rDEN4/3Δ30, (101) rDEN1/2Δ30, rDEN2/3Δ30, rDEN3/1Δ30, rDEN4Δ30, (102) rDEN1/2Δ30, rDEN2/3Δ30, rDEN3/1Δ30, rDEN4/1Δ30, (103) rDEN1/2Δ30, rDEN2/3Δ30, rDEN3/1Δ30, rDEN4/2Δ30, (104) rDEN1/2Δ30, rDEN2/3Δ30, rDEN3/1Δ30, rDEN4/3Δ30, (105) rDEN1/2Δ30, rDEN2/3Δ30, rDEN3/2Δ30, rDEN4Δ30, (106) rDEN1/2Δ30, rDEN2/3Δ30, rDEN3/2Δ30, rDEN4/1Δ30, (107) rDEN1/2Δ30, rDEN2/3Δ30, rDEN3/2Δ30, rDEN4/2Δ30, (108) rDEN1/2Δ30, rDEN2/3Δ30, rDEN3/2Δ30, rDEN4/3Δ30, (109) rDEN1/2Δ30, rDEN2/3Δ30, rDEN3/4Δ30, rDEN4Δ30, (110) rDEN1/2Δ30, rDEN2/3Δ30, rDEN3/4Δ30, rDEN4/1Δ30, (111) rDEN1/2Δ30, rDEN2/3Δ30, rDEN3/4Δ30, rDEN4/2Δ30, (112) rDEN1/2Δ30, rDEN2/3Δ30, rDEN3/4Δ30, rDEN4/3Δ30, (113) rDEN1/2Δ30, rDEN2/4Δ30, rDEN3Δ30, rDEN4Δ30, (114) rDEN1/2Δ30, rDEN2/4Δ30, rDEN3Δ30, rDEN4/1Δ30, (115) rDEN1/2Δ30, rDEN2/4Δ30, rDEN3Δ30, rDEN4/2Δ30, (116) rDEN1/2Δ30, rDEN2/4Δ30, rDEN3Δ30, rDEN4/3Δ30, (117) rDEN1/2Δ30, rDEN2/4Δ30, rDEN3/1Δ30, rDEN4Δ30, (118) rDEN1/2Δ30, rDEN2/4Δ30, rDEN3/1Δ30, rDEN4/1Δ30, (119) rDEN1/2Δ30, rDEN2/4Δ30, rDEN3/1Δ30, rDEN4/2Δ30, (120) rDEN1/2Δ30, rDEN2/4Δ30, rDEN3/1Δ30, rDEN4/3Δ30, (121) rDEN1/2Δ30, rDEN2/4Δ30, rDEN3/2Δ30, rDEN4Δ30, (122) rDEN1/2Δ30, rDEN2/4Δ30, rDEN3/2Δ30, rDEN4/1Δ30, (123) rDEN1/2Δ30, rDEN2/4Δ30, rDEN3/2Δ30, rDEN4/2Δ30, (124) rDEN1/2Δ30, rDEN2/4Δ30, rDEN3/2Δ30, rDEN4/3Δ30, (125) rDEN1/2Δ30, rDEN2/4Δ30, rDEN3/4Δ30, rDEN4Δ30, (126) rDEN1/2Δ30, rDEN2/4Δ30, rDEN3/4Δ30, rDEN4/1Δ30, (127) rDEN1/2Δ30, rDEN2/4Δ30, rDEN3/4Δ30, rDEN4/2Δ30, (128) rDEN1/2Δ30, rDEN2/4Δ30, rDEN3/4Δ30, rDEN4/3Δ30, (129) rDEN1/3Δ30, rDEN2Δ30, rDEN3Δ30, rDEN4Δ30, (130) rDEN1/3Δ30, rDEN2Δ30, rDEN3Δ30, rDEN4/1Δ30, (131) rDEN1/3Δ30, rDEN2Δ30, rDEN3Δ30, rDEN4/2Δ30, (132) rDEN1/3Δ30, rDEN2Δ30, rDEN3Δ30, rDEN4/3Δ30, (133) rDEN1/3Δ30, rDEN2Δ30, rDEN3/1Δ30, rDEN4Δ30, (134) rDEN1/3Δ30, rDEN2Δ30, rDEN3/1Δ30, rDEN4/1Δ30, (135) rDEN1/3Δ30, rDEN2Δ30, rDEN3/1Δ30, rDEN4/2Δ30, (136) rDEN1/3Δ30, rDEN2Δ30, rDEN3/1Δ30, rDEN4/3Δ30, (137) rDEN1/3Δ30, rDEN2Δ30, rDEN3/2Δ30, rDEN4Δ30, (138) rDEN1/3Δ30, rDEN2Δ30, rDEN3/2Δ30, rDEN4/1Δ30, (139) rDEN1/3Δ30, rDEN2Δ30, rDEN3/2Δ30, rDEN4/2Δ30, (140) rDEN1/3Δ30, rDEN2Δ30, rDEN3/2Δ30, rDEN4/3Δ30, (141) rDEN1/3Δ30, rDEN2Δ30, rDEN3/4Δ30, rDEN4Δ30, (142) rDEN1/3Δ30, rDEN2Δ30, rDEN3/4Δ30, rDEN4/1Δ30, (143) rDEN1/3Δ30, rDEN2Δ30, rDEN3/4Δ30, rDEN4/2Δ30, (144) rDEN1/3Δ30, rDEN2Δ30, rDEN3/4Δ30, rDEN4/3Δ30, (145) rDEN1/3Δ30, rDEN2/1Δ30, rDEN3Δ30, rDEN4Δ30, (146) rDEN1/3Δ30, rDEN2/1Δ30, rDEN3Δ30, rDEN4/1Δ30, (147) rDEN1/3Δ30, rDEN2/1Δ30, rDEN3Δ30, rDEN4/2Δ30, (148) rDEN1/3Δ30, rDEN2/1Δ30, rDEN3Δ30, rDEN4/3Δ30, (149) rDEN1/3Δ30, rDEN2/1Δ30, rDEN3/1Δ30, rDEN4Δ30, (150) rDEN1/3Δ30, rDEN2/1Δ30, rDEN3/1Δ30, rDEN4/1Δ30, (151) rDEN1/3Δ30, rDEN2/1Δ30, rDEN3/1Δ30, rDEN4/2Δ30, (152) rDEN1/3Δ30, rDEN2/1Δ30, rDEN3/1Δ30, rDEN4/3Δ30, (153) rDEN1/3Δ30, rDEN2/1Δ30, rDEN3/2Δ30, rDEN4Δ30, (154) rDEN1/3Δ30, rDEN2/1Δ30, rDEN3/2Δ30, rDEN4/1Δ30, (155) rDEN1/3Δ30, rDEN2/1Δ30, rDEN3/2Δ30, rDEN4/2Δ30, (156) rDEN1/3Δ30, rDEN2/1Δ30, rDEN3/2Δ30, rDEN4/3Δ30, (157) rDEN1/3Δ30, rDEN2/1Δ30, rDEN3/4Δ30, rDEN4Δ30, (158) rDEN1/3Δ30, rDEN2/1Δ30, rDEN3/4Δ30, rDEN4/1Δ30, (159) rDEN1/3Δ30, rDEN2/1Δ30, rDEN3/4Δ30, rDEN4/2Δ30, (160) rDEN1/3Δ30, rDEN2/1Δ30, rDEN3/4Δ30, rDEN4/3Δ30, (161) rDEN1/3Δ30, rDEN2/3Δ30, rDEN3Δ30, rDEN4Δ30, (162) rDEN1/3Δ30, rDEN2/3Δ30, rDEN3Δ30, rDEN4/1Δ30, (163) rDEN1/3Δ30, rDEN2/3Δ30, rDEN3Δ30, rDEN4/2A30, (164) rDEN1/3Δ30, rDEN2/3Δ30, rDEN3Δ30, rDEN4/3Δ30, (165) rDEN1/3Δ30, rDEN2/3Δ30, rDEN3/1Δ30, rDEN4Δ30, (166) rDEN1/3Δ30, rDEN2/3Δ30, rDEN3/1Δ30, rDEN4/1Δ30, (167) rDEN1/3Δ30, rDEN2/3Δ30, rDEN3/1Δ30, rDEN4/2Δ30, (168) rDEN1/3Δ30, rDEN2/3Δ30, rDEN3/1Δ30, rDEN4/3Δ30, (169) rDEN1/3Δ30, rDEN2/3Δ30, rDEN3/2Δ30, rDEN4Δ30, (170) rDEN1/3Δ30, rDEN2/3Δ30, rDEN3/2A30, rDEN4/1Δ30, (171) rDEN1/3Δ30, rDEN2/3Δ30, rDEN3/2Δ30, rDEN4/2Δ30, (172) rDEN1/3Δ30, rDEN2/3Δ30, rDEN3/2Δ30, rDEN4/3Δ30, (173) rDEN1/3Δ30, rDEN2/3Δ30, rDEN3/4Δ30, rDEN4Δ30, (174) rDEN1/3Δ30, rDEN2/3Δ30, rDEN3/4Δ30, rDEN4/1Δ30, (175) rDEN1/3Δ30, rDEN2/3Δ30, rDEN3/4Δ30, rDEN4/2Δ30, (176) rDEN1/3Δ30, rDEN2/3Δ30, rDEN3/4Δ30, rDEN4/3Δ30, (177) rDEN1/3Δ30, rDEN2/4Δ30, rDEN3Δ30, rDEN4Δ30, (178) rDEN1/3Δ30, rDEN2/4Δ30, rDEN3Δ30, rDEN4/1Δ30, (179) rDEN1/3Δ30, rDEN2/4Δ30, rDEN3Δ30, rDEN4/2Δ30, (180) rDEN1/3Δ30, rDEN2/4Δ30, rDEN3Δ30, rDEN4/3Δ30, (181) rDEN1/3Δ30, rDEN2/4Δ30, rDEN3/1Δ30, rDEN4Δ30, (182) rDEN1/3Δ30, rDEN2/4Δ30, rDEN3/1Δ30, rDEN4/1Δ30, (183) rDEN1/3Δ30, rDEN2/4Δ30, rDEN3/1Δ30, rDEN4/2Δ30, (184) rDEN1/3Δ30, rDEN2/4Δ30, rDEN3/1Δ30, rDEN4/3Δ30, (185) rDEN1/3Δ30, rDEN2/4Δ30, rDEN3/2Δ30, rDEN4Δ30, (186) rDEN1/3Δ30, rDEN2/4Δ30, rDEN3/2Δ30, rDEN4/1Δ30, (187) rDEN1/3Δ30, rDEN2/4Δ30, rDEN3/2Δ30, rDEN4/2Δ30, (188) rDEN1/3Δ30, rDEN2/4Δ30, rDEN3/2Δ30, rDEN4/3Δ30, (189) rDEN1/3Δ30, rDEN2/4Δ30, rDEN3/4Δ30, rDEN4Δ30, (190) rDEN1/3Δ30, rDEN2/4Δ30, rDEN3/4Δ30, rDEN4/1Δ30, (191) rDEN1/3Δ30, rDEN2/4Δ30, rDEN3/4Δ30, rDEN4/2Δ30, (192) rDEN1/3Δ30, rDEN2/4Δ30, rDEN3/4Δ30, rDEN4/3Δ30, (193) rDEN1/4Δ30, rDEN2Δ30, rDEN3Δ30, rDEN4Δ30, (194) rDEN1/4Δ30, rDEN2Δ30, rDEN3Δ30, rDEN4/1Δ30, (195) rDEN1/4Δ30, rDEN2Δ30, rDEN3Δ30, rDEN4/2Δ30, (196) rDEN1/4Δ30, rDEN2Δ30, rDEN3Δ30, rDEN4/3Δ30, (197) rDEN1/4Δ30, rDEN2A30, rDEN3/1Δ30, rDEN4Δ30, (198) rDEN1/4Δ30, rDEN2Δ30, rDEN3/1Δ30, rDEN4/1Δ30, (199) rDEN1/4Δ30, rDEN2Δ30, rDEN3/1Δ30, rDEN4/2Δ30, (200) rDEN1/4Δ30, rDEN2Δ30, rDEN3/1Δ30, rDEN4/3Δ30, (201) rDEN1/4Δ30, rDEN2Δ30, rDEN3/2Δ30, rDEN4Δ30, (202) rDEN1/4Δ30, rDEN2Δ30, rDEN3/2Δ30, rDEN4/1Δ30, (203) rDEN1/4Δ30, rDEN2Δ30, rDEN3/2Δ30, rDEN4/2Δ30, (204) rDEN1/4Δ30, rDEN2A30, rDEN3/2Δ30, rDEN4/3Δ30, (205) rDEN1/4Δ30, rDEN2Δ30, rDEN3/4Δ30, rDEN4Δ30, (206) rDEN1/4Δ30, rDEN2Δ30, rDEN3/4Δ30, rDEN4/1Δ30, (207) rDEN1/4Δ30, rDEN2Δ30, rDEN3/4Δ30, rDEN4/2Δ30, (208) rDEN1/4Δ30, rDEN2Δ30, rDEN3/4Δ30, rDEN4/3Δ30, (209) rDEN1/4Δ30, rDEN2/1Δ30, rDEN3Δ30, rDEN4A30, (210) rDEN1/4Δ30, rDEN2/1Δ30, rDEN3Δ30, rDEN4/1Δ30, (211) rDEN1/4Δ30, rDEN2/1Δ30, rDEN3Δ30, rDEN4/2Δ30, (212) rDEN1/4Δ30, rDEN2/1Δ30, rDEN3Δ30, rDEN4/3Δ30, (213) rDEN1/4Δ30, rDEN2/1Δ30, rDEN3/1Δ30, rDEN4Δ30, (214) rDEN1/4Δ30, rDEN2/1Δ30, rDEN3/1Δ30, rDEN4/1Δ30, (215) rDEN1/4Δ30, rDEN2/1Δ30, rDEN3/1Δ30, rDEN4/2Δ30, (216) rDEN1/4Δ30, rDEN2/1Δ30, rDEN3/1Δ30, rDEN4/3Δ30, (217) rDEN1/4Δ30, rDEN2/1Δ30, rDEN3/2Δ30, rDEN4A30, (218) rDEN1/4Δ30, rDEN2/1Δ30, rDEN3/2Δ30, rDEN4/1Δ30, (219) rDEN1/4Δ30, rDEN2/1Δ30, rDEN3/2Δ30, rDEN4/2Δ30, (220) rDEN1/4Δ30, rDEN2/1Δ30, rDEN3/2Δ30, rDEN4/3Δ30, (221) rDEN1/4Δ30, rDEN2/1Δ30, rDEN3/4Δ30, rDEN4Δ30, (222) rDEN1/4Δ30, rDEN2/1Δ30, rDEN3/4Δ30, rDEN4/1Δ30, (223) rDEN1/4Δ30, rDEN2/1Δ30, rDEN3/4Δ30, rDEN4/2Δ30, (224) rDEN1/4Δ30, rDEN2/1Δ30, rDEN3/4Δ30, rDEN4/3Δ30, (225) rDEN1/4Δ30, rDEN2/3Δ30, rDEN3Δ30, rDEN4Δ30, (226) rDEN1/4Δ30, rDEN2/3Δ30, rDEN3Δ30, rDEN4/1Δ30, (227) rDEN1/4Δ30, rDEN2/3Δ30, rDEN3Δ30, rDEN4/2Δ30, (228) rDEN1/4Δ30, rDEN2/3Δ30, rDEN3Δ30, rDEN4/3Δ30, (229) rDEN1/4Δ30, rDEN2/3Δ30, rDEN3/1Δ30, rDEN4A30, (230) rDEN1/4Δ30, rDEN2/3Δ30, rDEN3/1Δ30, rDEN4/1Δ30, (231) rDEN1/4Δ30, rDEN2/3Δ30, rDEN3/1Δ30, rDEN4/2Δ30, (232) rDEN1/4Δ30, rDEN2/3Δ30, rDEN3/1Δ30, rDEN4/3Δ30, (233) rDEN1/4Δ30, rDEN2/3Δ30, rDEN3/2Δ30, rDEN4Δ30, (234) rDEN1/4Δ30, rDEN2/3Δ30, rDEN3/2Δ30, rDEN4/1Δ30, (235) rDEN1/4Δ30, rDEN2/3Δ30, rDEN3/2Δ30, rDEN4/2Δ30, (236) rDEN1/4Δ30, rDEN2/3Δ30, rDEN3/2Δ30, rDEN4/3Δ30, (237) rDEN1/4Δ30, rDEN2/3Δ30, rDEN3/4Δ30, rDEN4Δ30, (238) rDEN1/4Δ30, rDEN2/3Δ30, rDEN3/4Δ30, rDEN4/1Δ30, (239) rDEN1/4Δ30, rDEN2/3Δ30, rDEN3/4Δ30, rDEN4/2Δ30, (240) rDEN1/4Δ30, rDEN2/3Δ30, rDEN3/4Δ30, rDEN4/3Δ30, (241) rDEN1/4Δ30, rDEN2/4Δ30, rDEN3Δ30, rDEN4Δ30, (242) rDEN1/4Δ30, rDEN2/4Δ30, rDEN3Δ30, rDEN4/1Δ30, (243) rDEN1/4Δ30, rDEN2/4Δ30, rDEN3Δ30, rDEN4/2Δ30, (244) rDEN1/4Δ30, rDEN2/4Δ30, rDEN3Δ30, rDEN4/3Δ30, (245) rDEN1/4Δ30, rDEN2/4Δ30, rDEN3/1Δ30, rDEN4Δ30, (246) rDEN1/4Δ30, rDEN2/4Δ30, rDEN3/1Δ30, rDEN4/1Δ30, (247) rDEN1/4Δ30, rDEN2/4Δ30, rDEN3/1Δ30, rDEN4/2Δ30, (248) rDEN1/4Δ30, rDEN2/4Δ30, rDEN3/1Δ30, rDEN4/3Δ30, (249) rDEN1/4Δ30, rDEN2/4Δ30, rDEN3/2Δ30, rDEN4Δ30, (250) rDEN1/4Δ30, rDEN2/4Δ30, rDEN3/2Δ30, rDEN4/1Δ30, (251) rDEN1/4Δ30, rDEN2/4Δ30, rDEN3/2Δ30, rDEN4/2Δ30, (252) rDEN1/4Δ30, rDEN2/4Δ30, rDEN3/2Δ30, rDEN4/3Δ30, (253) rDEN1/4Δ30, rDEN2/4Δ30, rDEN3/4Δ30, rDEN4Δ30, (254) rDEN1/4Δ30, rDEN2/4Δ30, rDEN3/4Δ30, rDEN4/1Δ30, (255) rDEN1/4Δ30, rDEN2/4Δ30, rDEN3/4Δ30, rDEN4/2Δ30, and (256) rDEN1/4Δ30, rDEN2/4Δ30, rDEN3/4Δ30, rDEN4/3Δ30. In another embodiment, the fifth attenuated virus of the pentavalent immunogenic composition of any of the combinations of the first, second, third and fourth attenuated viruses described above is a Zika chimera of the present disclosure, as described in greater detail above. In an embodiment, each of the attenuated viruses comprises the same dengue backbone. In another embodiment, the fifth attenuated virus comprises a different dengue virus backbone than the first, second, third, and fourth attenuated viruses.

In an embodiment, the first, second, third, and fourth attenuated viruses are selected from the group consisting of: (1) rDEN1Δ31, rDEN2Δ31, rDEN3431, rDEN4Δ31, (2) rDEN1Δ31, rDEN2Δ31, rDEN3431, rDEN4/1Δ31, (3) rDEN1Δ31, rDEN2Δ31, rDEN3431, rDEN4/2Δ31, (4) rDEN1Δ31, rDEN2Δ31, rDEN3431, rDEN4/3431, (5) rDEN1Δ31, rDEN2Δ31, rDEN3/1Δ31, rDEN4Δ31, (6) rDEN1Δ31, rDEN2Δ31, rDEN3/1Δ31, rDEN4/1Δ31, (7) rDEN1Δ31, rDEN2Δ31, rDEN3/1Δ31, rDEN4/2Δ31, (8) rDEN1Δ31, rDEN2Δ31, rDEN3/1Δ31, rDEN4/3Δ31, (9) rDEN1Δ31, rDEN2Δ31, rDEN3/2Δ31, rDEN4Δ31, (10) rDEN1Δ31, rDEN2Δ31, rDEN3/2Δ31, rDEN4/1Δ31, (11) rDEN1Δ31, rDEN2Δ31, rDEN3/2Δ31, rDEN4/2Δ31, (12) rDEN1Δ31, rDEN2Δ31, rDEN3/2Δ31, rDEN4/3Δ31, (13) rDEN1Δ31, rDEN2Δ31, rDEN3/4Δ31, rDEN4Δ31, (14) rDEN1Δ31, rDEN2Δ31, rDEN3/4Δ31, rDEN4/1Δ31, (15) rDEN1Δ31, rDEN2Δ31, rDEN3/4Δ31, rDEN4/2Δ31, (16) rDEN1Δ31, rDEN2Δ31, rDEN3/4Δ31, rDEN4/3Δ31, (17) rDEN1Δ31, rDEN2/1Δ31, rDEN3Δ31, rDEN4Δ31, (18) rDEN1Δ31, rDEN2/1Δ31, rDEN3Δ31, rDEN4/1Δ31, (19) rDEN1Δ31, rDEN2/1Δ31, rDEN3Δ31, rDEN4/2Δ31, (20) rDEN1Δ31, rDEN2/1Δ31, rDEN3Δ31, rDEN4/3Δ31, (21) rDEN1Δ31, rDEN2/1Δ31, rDEN3/1Δ31, rDEN4Δ31, (22) rDEN1Δ31, rDEN2/1Δ31, rDEN3/1Δ31, rDEN4/1Δ31, (23) rDEN1Δ31, rDEN2/1Δ31, rDEN3/1Δ31, rDEN4/2Δ31, (24) rDEN1Δ31, rDEN2/1Δ31, rDEN3/1Δ31, rDEN4/3Δ31, (25) rDEN1Δ31, rDEN2/1Δ31, rDEN3/2Δ31, rDEN4Δ31, (26) rDEN1Δ31, rDEN2/1Δ31, rDEN3/2Δ31, rDEN4/1Δ31, (27) rDEN1Δ31, rDEN2/1Δ31, rDEN3/2Δ31, rDEN4/2Δ31, (28) rDEN1Δ31, rDEN2/1Δ31, rDEN3/2A31, rDEN4/3Δ31, (29) rDEN1Δ31, rDEN2/1Δ31, rDEN3/4Δ31, rDEN4Δ31, (30) rDEN1Δ31, rDEN2/1Δ31, rDEN3/4Δ31, rDEN4/1Δ31, (31) rDEN1Δ31, rDEN2/1Δ31, rDEN3/4Δ31, rDEN4/2A31, (32) rDEN1Δ31, rDEN2/1Δ31, rDEN3/4Δ31, rDEN4/3Δ31, (33) rDEN1Δ31, rDEN2/3Δ31, rDEN3Δ31, rDEN4Δ31, (34) rDEN1Δ31, rDEN2/3Δ31, rDEN3Δ31, rDEN4/1Δ31, (35) rDEN1Δ31, rDEN2/3Δ31, rDEN3Δ31, rDEN4/2Δ31, (36) rDEN1Δ31, rDEN2/3Δ31, rDEN3Δ31, rDEN4/3Δ31, (37) rDEN1Δ31, rDEN2/3Δ31, rDEN3/1Δ31, rDEN4Δ31, (38) rDEN1Δ31, rDEN2/3Δ31, rDEN3/1Δ31, rDEN4/1Δ31, (39) rDEN1Δ31, rDEN2/3Δ31, rDEN3/1Δ31, rDEN4/2Δ31, (40) rDEN1Δ31, rDEN2/3Δ31, rDEN3/1Δ31, rDEN4/3Δ31, (41) rDEN1Δ31, rDEN2/3Δ31, rDEN3/2Δ31, rDEN4Δ31, (42) rDEN1Δ31, rDEN2/3Δ31, rDEN3/2Δ31, rDEN4/1Δ31, (43) rDEN1Δ31, rDEN2/3Δ31, rDEN3/2Δ31, rDEN4/2Δ31, (44) rDEN1Δ31, rDEN2/3Δ31, rDEN3/2Δ31, rDEN4/3Δ31, (45) rDEN1Δ31, rDEN2/3Δ31, rDEN3/4Δ31, rDEN4Δ31, (46) rDEN1Δ31, rDEN2/3Δ31, rDEN3/4Δ31, rDEN4/1Δ31, (47) rDEN1Δ31, rDEN2/3Δ31, rDEN3/4Δ31, rDEN4/2Δ31, (48) rDEN1Δ31, rDEN2/3Δ31, rDEN3/4Δ31, rDEN4/3Δ31, (49) rDEN1Δ31, rDEN2/4Δ31, rDEN3Δ31, rDEN4Δ31, (50) rDEN1Δ31, rDEN2/4Δ31, rDEN3Δ31, rDEN4/1Δ31, (51) rDEN1Δ31, rDEN2/4Δ31, rDEN3Δ31, rDEN4/2Δ31, (52) rDEN1Δ31, rDEN2/4Δ31, rDEN3Δ31, rDEN4/3Δ31, (53) rDEN1Δ31, rDEN2/4Δ31, rDEN3/1Δ31, rDEN4Δ31, (54) rDEN1Δ31, rDEN2/4Δ31, rDEN3/1Δ31, rDEN4/1Δ31, (55) rDEN1Δ31, rDEN2/4Δ31, rDEN3/1Δ31, rDEN4/2Δ31, (56) rDEN1Δ31, rDEN2/4Δ31, rDEN3/1Δ31, rDEN4/3Δ31, (57) rDEN1Δ31, rDEN2/4Δ31, rDEN3/2Δ31, rDEN4Δ31, (58) rDEN1Δ31, rDEN2/4Δ31, rDEN3/2Δ31, rDEN4/1Δ31, (59) rDEN1Δ31, rDEN2/4Δ31, rDEN3/2Δ31, rDEN4/2Δ31, (60) rDEN1Δ31, rDEN2/4Δ31, rDEN3/2Δ31, rDEN4/3Δ31, (61) rDEN1Δ31, rDEN2/4Δ31, rDEN3/4Δ31, rDEN4Δ31, (62) rDEN1Δ31, rDEN2/4Δ31, rDEN3/4Δ31, rDEN4/1Δ31, (63) rDEN1Δ31, rDEN2/4Δ31, rDEN3/4Δ31, rDEN4/2Δ31, (64) rDEN1Δ31, rDEN2/4Δ31, rDEN3/4Δ31, rDEN4/3Δ31, (65) rDEN1/2Δ31, rDEN2Δ31, rDEN3Δ31, rDEN4Δ31, (66) rDEN1/2Δ31, rDEN2Δ31, rDEN3Δ31, rDEN4/1Δ31, (67) rDEN1/2Δ31, rDEN2Δ31, rDEN3Δ31, rDEN4/2Δ31, (68) rDEN1/2Δ31, rDEN2Δ31, rDEN3Δ31, rDEN4/3Δ31, (69) rDEN1/2Δ31, rDEN2Δ31, rDEN3/1Δ31, rDEN4Δ31, (70) rDEN1/2Δ31, rDEN2Δ31, rDEN3/1Δ31, rDEN4/1Δ31, (71) rDEN1/2Δ31, rDEN2Δ31, rDEN3/1Δ31, rDEN4/2Δ31, (72) rDEN1/2Δ31, rDEN2Δ31, rDEN3/1Δ31, rDEN4/3Δ31, (73) rDEN1/2Δ31, rDEN2Δ31, rDEN3/2Δ31, rDEN4Δ31, (74) rDEN1/2Δ31, rDEN2Δ31, rDEN3/2Δ31, rDEN4/1Δ31, (75) rDEN1/2Δ31, rDEN2Δ31, rDEN3/2Δ31, rDEN4/2Δ31, (76) rDEN1/2Δ31, rDEN2Δ31, rDEN3/2Δ31, rDEN4/3Δ31, (77) rDEN1/2Δ31, rDEN2Δ31, rDEN3/4Δ31, rDEN4Δ31, (78) rDEN1/2Δ31, rDEN2Δ31, rDEN3/4Δ31, rDEN4/1Δ31, (79) rDEN1/2Δ31, rDEN2Δ31, rDEN3/4Δ31, rDEN4/2Δ31, (80) rDEN1/2Δ31, rDEN2Δ31, rDEN3/4Δ31, rDEN4/3Δ31, (81) rDEN1/2Δ31, rDEN2/1Δ31, rDEN3Δ31, rDEN4Δ31, (82) rDEN1/2Δ31, rDEN2/1Δ31, rDEN3Δ31, rDEN4/1Δ31, (83) rDEN1/2Δ31, rDEN2/1Δ31, rDEN3Δ31, rDEN4/2Δ31, (84) rDEN1/2Δ31, rDEN2/1Δ31, rDEN3Δ31, rDEN4/3Δ31, (85) rDEN1/2Δ31, rDEN2/1Δ31, rDEN3/1Δ31, rDEN4Δ31, (86) rDEN1/2Δ31, rDEN2/1Δ31, rDEN3/1Δ31, rDEN4/1Δ31, (87) rDEN1/2Δ31, rDEN2/1Δ31, rDEN3/1Δ31, rDEN4/2Δ31, (88) rDEN1/2Δ31, rDEN2/1Δ31, rDEN3/1Δ31, rDEN4/3Δ31, (89) rDEN1/2Δ31, rDEN2/1Δ31, rDEN3/2Δ31, rDEN4Δ31, (90) rDEN1/2Δ31, rDEN2/1Δ31, rDEN3/2Δ31, rDEN4/1Δ31, (91) rDEN1/2Δ31, rDEN2/1Δ31, rDEN3/2Δ31, rDEN4/2Δ31, (92) rDEN1/2Δ31, rDEN2/1Δ31, rDEN3/2Δ31, rDEN4/3Δ31, (93) rDEN1/2Δ31, rDEN2/1Δ31, rDEN3/4Δ31, rDEN4Δ31, (94) rDEN1/2Δ31, rDEN2/1Δ31, rDEN3/4Δ31, rDEN4/1Δ31, (95) rDEN1/2Δ31, rDEN2/1Δ31, rDEN3/4Δ31, rDEN4/2Δ31, (96) rDEN1/2Δ31, rDEN2/1Δ31, rDEN3/4Δ31, rDEN4/3Δ31, (97) rDEN1/2Δ31, rDEN2/3Δ31, rDEN3Δ31, rDEN4Δ31, (98) rDEN1/2Δ31, rDEN2/3Δ31, rDEN3Δ31, rDEN4/1Δ31, (99) rDEN1/2Δ31, rDEN2/3Δ31, rDEN3Δ31, rDEN4/2Δ31, (100) rDEN1/2Δ31, rDEN2/3Δ31, rDEN3Δ31, rDEN4/3Δ31, (101) rDEN1/2Δ31, rDEN2/3Δ31, rDEN3/1Δ31, rDEN4Δ31, (102) rDEN1/2Δ31, rDEN2/3Δ31, rDEN3/1Δ31, rDEN4/1Δ31, (103) rDEN1/2Δ31, rDEN2/3Δ31, rDEN3/1Δ31, rDEN4/2Δ31, (104) rDEN1/2Δ31, rDEN2/3Δ31, rDEN3/1Δ31, rDEN4/3Δ31, (105) rDEN1/2Δ31, rDEN2/3Δ31, rDEN3/2Δ31, rDEN4Δ31, (106) rDEN1/2Δ31, rDEN2/3Δ31, rDEN3/2Δ31, rDEN4/1Δ31, (107) rDEN1/2Δ31, rDEN2/3Δ31, rDEN3/2Δ31, rDEN4/2Δ31, (108) rDEN1/2Δ31, rDEN2/3Δ31, rDEN3/2Δ31, rDEN4/3Δ31, (109) rDEN1/2Δ31, rDEN2/3Δ31, rDEN3/4Δ31, rDEN4Δ31, (110) rDEN1/2Δ31, rDEN2/3Δ31, rDEN3/4Δ31, rDEN4/1Δ31, (111) rDEN1/2Δ31, rDEN2/3Δ31, rDEN3/4Δ31, rDEN4/2Δ31, (112) rDEN1/2Δ31, rDEN2/3Δ31, rDEN3/4Δ31, rDEN4/3Δ31, (113) rDEN1/2Δ31, rDEN2/4Δ31, rDEN3Δ31, rDEN4Δ31, (114) rDEN1/2Δ31, rDEN2/4Δ31, rDEN3Δ31, rDEN4/1Δ31, (115) rDEN1/2Δ31, rDEN2/4Δ31, rDEN3Δ31, rDEN4/2Δ31, (116) rDEN1/2Δ31, rDEN2/4Δ31, rDEN3Δ31, rDEN4/3Δ31, (117) rDEN1/2Δ31, rDEN2/4Δ31, rDEN3/1Δ31, rDEN4Δ31, (118) rDEN1/2Δ31, rDEN2/4Δ31, rDEN3/1Δ31, rDEN4/1Δ31, (119) rDEN1/2Δ31, rDEN2/4Δ31, rDEN3/1Δ31, rDEN4/2Δ31, (120) rDEN1/2Δ31, rDEN2/4Δ31, rDEN3/1Δ31, rDEN4/3Δ31, (121) rDEN1/2Δ31, rDEN2/4Δ31, rDEN3/2Δ31, rDEN4Δ31, (122) rDEN1/2Δ31, rDEN2/4Δ31, rDEN3/2Δ31, rDEN4/1Δ31, (123) rDEN1/2Δ31, rDEN2/4Δ31, rDEN3/2Δ31, rDEN4/2Δ31, (124) rDEN1/2Δ31, rDEN2/4Δ31, rDEN3/2Δ31, rDEN4/3Δ31, (125) rDEN1/2Δ31, rDEN2/4Δ31, rDEN3/4Δ31, rDEN4Δ31, (126) rDEN1/2Δ31, rDEN2/4Δ31, rDEN3/4Δ31, rDEN4/1Δ31, (127) rDEN1/2Δ31, rDEN2/4Δ31, rDEN3/4Δ31, rDEN4/2Δ31, (128) rDEN1/2Δ31, rDEN2/4Δ31, rDEN3/4Δ31, rDEN4/3Δ31, (129) rDEN1/3Δ31, rDEN2Δ31, rDEN3Δ31, rDEN4Δ31, (130) rDEN1/3Δ31, rDEN2Δ31, rDEN3Δ31, rDEN4/1Δ31, (131) rDEN1/3Δ31, rDEN2Δ31, rDEN3Δ31, rDEN4/2Δ31, (132) rDEN1/3Δ31, rDEN2Δ31, rDEN3Δ31, rDEN4/3Δ31, (133) rDEN1/3Δ31, rDEN2Δ31, rDEN3/1Δ31, rDEN4Δ31, (134) rDEN1/3Δ31, rDEN2Δ31, rDEN3/1Δ31, rDEN4/1Δ31, (135) rDEN1/3Δ31, rDEN2Δ31, rDEN3/1Δ31, rDEN4/2Δ31, (136) rDEN1/3Δ31, rDEN2Δ31, rDEN3/1Δ31, rDEN4/3Δ31, (137) rDEN1/3Δ31, rDEN2Δ31, rDEN3/2Δ31, rDEN4Δ31, (138) rDEN1/3Δ31, rDEN2Δ31, rDEN3/2Δ31, rDEN4/1Δ31, (139) rDEN1/3Δ31, rDEN2Δ31, rDEN3/2Δ31, rDEN4/2Δ31, (140) rDEN1/3Δ31, rDEN2Δ31, rDEN3/2Δ31, rDEN4/3Δ31, (141) rDEN1/3Δ31, rDEN2Δ31, rDEN3/4Δ31, rDEN4Δ31, (142) rDEN1/3Δ31, rDEN2Δ31, rDEN3/4Δ31, rDEN4/1Δ31, (143) rDEN1/3Δ31, rDEN2Δ31, rDEN3/4Δ31, rDEN4/2Δ31, (144) rDEN1/3Δ31, rDEN2Δ31, rDEN3/4Δ31, rDEN4/3Δ31, (145) rDEN1/3Δ31, rDEN2/1Δ31, rDEN3Δ31, rDEN4Δ31, (146) rDEN1/3Δ31, rDEN2/1Δ31, rDEN3Δ31, rDEN4/1Δ31, (147) rDEN1/3Δ31, rDEN2/1Δ31, rDEN3Δ31, rDEN4/2Δ31, (148) rDEN1/3Δ31, rDEN2/1Δ31, rDEN3Δ31, rDEN4/3Δ31, (149) rDEN1/3Δ31, rDEN2/1Δ31, rDEN3/1Δ31, rDEN4Δ31, (150) rDEN1/3Δ31, rDEN2/1Δ31, rDEN3/1Δ31, rDEN4/1Δ31, (151) rDEN1/3Δ31, rDEN2/1Δ31, rDEN3/1Δ31, rDEN4/2Δ31, (152) rDEN1/3Δ31, rDEN2/1Δ31, rDEN3/1Δ31, rDEN4/3Δ31, (153) rDEN1/3Δ31, rDEN2/1Δ31, rDEN3/2Δ31, rDEN4Δ31, (154) rDEN1/3Δ31, rDEN2/1Δ31, rDEN3/2Δ31, rDEN4/1Δ31, (155) rDEN1/3Δ31, rDEN2/1Δ31, rDEN3/2Δ31, rDEN4/2Δ31, (156) rDEN1/3Δ31, rDEN2/1Δ31, rDEN3/2Δ31, rDEN4/3Δ31, (157) rDEN1/3Δ31, rDEN2/1Δ31, rDEN3/4Δ31, rDEN4Δ31, (158) rDEN1/3Δ31, rDEN2/1Δ31, rDEN3/4Δ31, rDEN4/1Δ31, (159) rDEN1/3Δ31, rDEN2/1Δ31, rDEN3/4Δ31, rDEN4/2Δ31, (160) rDEN1/3Δ31, rDEN2/1Δ31, rDEN3/4Δ31, rDEN4/3Δ31, (161) rDEN1/3Δ31, rDEN2/3Δ31, rDEN3Δ31, rDEN4Δ31, (162) rDEN1/3Δ31, rDEN2/3Δ31, rDEN3Δ31, rDEN4/1Δ31, (163) rDEN1/3Δ31, rDEN2/3Δ31, rDEN3Δ31, rDEN4/2Δ31, (164) rDEN1/3Δ31, rDEN2/3Δ31, rDEN3Δ31, rDEN4/3Δ31, (165) rDEN1/3Δ31, rDEN2/3Δ31, rDEN3/1Δ31, rDEN4Δ31, (166) rDEN1/3Δ31, rDEN2/3Δ31, rDEN3/1Δ31, rDEN4/1Δ31, (167) rDEN1/3Δ31, rDEN2/3Δ31, rDEN3/1Δ31, rDEN4/2Δ31, (168) rDEN1/3Δ31, rDEN2/3Δ31, rDEN3/1Δ31, rDEN4/3Δ31, (169) rDEN1/3Δ31, rDEN2/3Δ31, rDEN3/2Δ31, rDEN4Δ31, (170) rDEN1/3Δ31, rDEN2/3Δ31, rDEN3/2Δ31, rDEN4/1Δ31, (171) rDEN1/3Δ31, rDEN2/3Δ31, rDEN3/2Δ31, rDEN4/2Δ31, (172) rDEN1/3Δ31, rDEN2/3Δ31, rDEN3/2Δ31, rDEN4/3Δ31, (173) rDEN1/3Δ31, rDEN2/3Δ31, rDEN3/4Δ31, rDEN4Δ31, (174) rDEN1/3Δ31, rDEN2/3Δ31, rDEN3/4Δ31, rDEN4/1Δ31, (175) rDEN1/3Δ31, rDEN2/3Δ31, rDEN3/4Δ31, rDEN4/2Δ31, (176) rDEN1/3Δ31, rDEN2/3Δ31, rDEN3/4Δ31, rDEN4/3Δ31, (177) rDEN1/3Δ31, rDEN2/4Δ31, rDEN3Δ31, rDEN4Δ31, (178) rDEN1/3Δ31, rDEN2/4Δ31, rDEN3Δ31, rDEN4/1Δ31, (179) rDEN1/3Δ31, rDEN2/4Δ31, rDEN3Δ31, rDEN4/2Δ31, (180) rDEN1/3Δ31, rDEN2/4Δ31, rDEN3Δ31, rDEN4/3Δ31, (181) rDEN1/3Δ31, rDEN2/4Δ31, rDEN3/1Δ31, rDEN4Δ31, (182) rDEN1/3Δ31, rDEN2/4Δ31, rDEN3/1Δ31, rDEN4/1Δ31, (183) rDEN1/3Δ31, rDEN2/4Δ31, rDEN3/1Δ31, rDEN4/2Δ31, (184) rDEN1/3Δ31, rDEN2/4Δ31, rDEN3/1Δ31, rDEN4/3Δ31, (185) rDEN1/3Δ31, rDEN2/4Δ31, rDEN3/2Δ31, rDEN4Δ31, (186) rDEN1/3Δ31, rDEN2/4Δ31, rDEN3/2Δ31, rDEN4/1Δ31, (187) rDEN1/3Δ31, rDEN2/4Δ31, rDEN3/2Δ31, rDEN4/2Δ31, (188) rDEN1/3Δ31, rDEN2/4Δ31, rDEN3/2Δ31, rDEN4/3Δ31, (189) rDEN1/3Δ31, rDEN2/4Δ31, rDEN3/4Δ31, rDEN4Δ31, (190) rDEN1/3Δ31, rDEN2/4Δ31, rDEN3/4Δ31, rDEN4/1Δ31, (191) rDEN1/3Δ31, rDEN2/4Δ31, rDEN3/4Δ31, rDEN4/2Δ31, (192) rDEN1/3Δ31, rDEN2/4Δ31, rDEN3/4Δ31, rDEN4/3Δ31, (193) rDEN1/4Δ31, rDEN2Δ31, rDEN3Δ31, rDEN4Δ31, (194) rDEN1/4Δ31, rDEN2Δ31, rDEN3Δ31, rDEN4/1Δ31, (195) rDEN1/4Δ31, rDEN2Δ31, rDEN3Δ31, rDEN4/2Δ31, (196) rDEN1/4Δ31, rDEN2Δ31, rDEN3Δ31, rDEN4/3Δ31, (197) rDEN1/4Δ31, rDEN2Δ31, rDEN3/1Δ31, rDEN4Δ31, (198) rDEN1/4Δ31, rDEN2Δ31, rDEN3/1Δ31, rDEN4/1Δ31, (199) rDEN1/4Δ31, rDEN2Δ31, rDEN3/1Δ31, rDEN4/2Δ31, (200) rDEN1/4Δ31, rDEN2Δ31, rDEN3/1Δ31, rDEN4/3Δ31, (201) rDEN1/4Δ31, rDEN2Δ31, rDEN3/2Δ31, rDEN4Δ31, (202) rDEN1/4Δ31, rDEN2Δ31, rDEN3/2Δ31, rDEN4/1Δ31, (203) rDEN1/4Δ31, rDEN2Δ31, rDEN3/2Δ31, rDEN4/2Δ31, (204) rDEN1/4Δ31, rDEN2Δ31, rDEN3/2Δ31, rDEN4/3Δ31, (205) rDEN1/4Δ31, rDEN2Δ31, rDEN3/4Δ31, rDEN4Δ31, (206) rDEN1/4Δ31, rDEN2Δ31, rDEN3/4Δ31, rDEN4/1Δ31, (207) rDEN1/4Δ31, rDEN2Δ31, rDEN3/4Δ31, rDEN4/2Δ31, (208) rDEN1/4Δ31, rDEN2Δ31, rDEN3/4Δ31, rDEN4/3Δ31, (209) rDEN1/4Δ31, rDEN2/1Δ31, rDEN3Δ31, rDEN4Δ31, (210) rDEN1/4Δ31, rDEN2/1Δ31, rDEN3Δ31, rDEN4/1Δ31, (211) rDEN1/4Δ31, rDEN2/1Δ31, rDEN3Δ31, rDEN4/2Δ31, (212) rDEN1/4Δ31, rDEN2/1Δ31, rDEN3Δ31, rDEN4/3Δ31, (213) rDEN1/4Δ31, rDEN2/1Δ31, rDEN3/1Δ31, rDEN4Δ31, (214) rDEN1/4Δ31, rDEN2/1Δ31, rDEN3/1Δ31, rDEN4/1Δ31, (215) rDEN1/4Δ31, rDEN2/1Δ31, rDEN3/1Δ31, rDEN4/2Δ31, (216) rDEN1/4Δ31, rDEN2/1Δ31, rDEN3/1Δ31, rDEN4/3Δ31, (217) rDEN1/4Δ31, rDEN2/1Δ31, rDEN3/2Δ31, rDEN4Δ31, (218) rDEN1/4Δ31, rDEN2/1Δ31, rDEN3/2Δ31, rDEN4/1Δ31, (219) rDEN1/4Δ31, rDEN2/1Δ31, rDEN3/2Δ31, rDEN4/2Δ31, (220) rDEN1/4Δ31, rDEN2/1Δ31, rDEN3/2Δ31, rDEN4/3Δ31, (221) rDEN1/4Δ31, rDEN2/1Δ31, rDEN3/4Δ31, rDEN4Δ31, (222) rDEN1/4Δ31, rDEN2/1Δ31, rDEN3/4Δ31, rDEN4/1Δ31, (223) rDEN1/4Δ31, rDEN2/1Δ31, rDEN3/4Δ31, rDEN4/2Δ31, (224) rDEN1/4Δ31, rDEN2/1Δ31, rDEN3/4Δ31, rDEN4/3Δ31, (225) rDEN1/4Δ31, rDEN2/3Δ31, rDEN3Δ31, rDEN4Δ31, (226) rDEN1/4Δ31, rDEN2/3Δ31, rDEN3Δ31, rDEN4/1Δ31, (227) rDEN1/4Δ31, rDEN2/3Δ31, rDEN3Δ31, rDEN4/2Δ31, (228) rDEN1/4Δ31, rDEN2/3Δ31, rDEN3Δ31, rDEN4/3Δ31, (229) rDEN1/4Δ31, rDEN2/3Δ31, rDEN3/1Δ31, rDEN4Δ31, (230) rDEN1/4Δ31, rDEN2/3Δ31, rDEN3/1Δ31, rDEN4/1Δ31, (231) rDEN1/4Δ31, rDEN2/3Δ31, rDEN3/1Δ31, rDEN4/2Δ31, (232) rDEN1/4Δ31, rDEN2/3Δ31, rDEN3/1Δ31, rDEN4/3Δ31, (233) rDEN1/4Δ31, rDEN2/3Δ31, rDEN3/2Δ31, rDEN4Δ31, (234) rDEN1/4Δ31, rDEN2/3Δ31, rDEN3/2Δ31, rDEN4/1Δ31, (235) rDEN1/4Δ31, rDEN2/3Δ31, rDEN3/2Δ31, rDEN4/2Δ31, (236) rDEN1/4Δ31, rDEN2/3Δ31, rDEN3/2Δ31, rDEN4/3Δ31, (237) rDEN1/4Δ31, rDEN2/3Δ31, rDEN3/4Δ31, rDEN4Δ31, (238) rDEN1/4Δ31, rDEN2/3Δ31, rDEN3/4Δ31, rDEN4/1Δ31, (239) rDEN1/4Δ31, rDEN2/3Δ31, rDEN3/4Δ31, rDEN4/2Δ31, (240) rDEN1/4Δ31, rDEN2/3Δ31, rDEN3/4Δ31, rDEN4/3Δ31, (241) rDEN1/4Δ31, rDEN2/4Δ31, rDEN3Δ31, rDEN4Δ31, (242) rDEN1/4Δ31, rDEN2/4Δ31, rDEN3Δ31, rDEN4/1Δ31, (243) rDEN1/4Δ31, rDEN2/4Δ31, rDEN3Δ31, rDEN4/2Δ31, (244) rDEN1/4Δ31, rDEN2/4Δ31, rDEN3Δ31, rDEN4/3Δ31, (245) rDEN1/4Δ31, rDEN2/4Δ31, rDEN3/1Δ31, rDEN4Δ31, (246) rDEN1/4Δ31, rDEN2/4Δ31, rDEN3/1Δ31, rDEN4/1Δ31, (247) rDEN1/4Δ31, rDEN2/4Δ31, rDEN3/1Δ31, rDEN4/2Δ31, (248) rDEN1/4Δ31, rDEN2/4Δ31, rDEN3/1Δ31, rDEN4/3Δ31, (249) rDEN1/4Δ31, rDEN2/4Δ31, rDEN3/2Δ31, rDEN4Δ31, (250) rDEN1/4Δ31, rDEN2/4Δ31, rDEN3/2Δ31, rDEN4/1Δ31, (251) rDEN1/4Δ31, rDEN2/4Δ31, rDEN3/2Δ31, rDEN4/2Δ31, (252) rDEN1/4Δ31, rDEN2/4Δ31, rDEN3/2Δ31, rDEN4/3Δ31, (253) rDEN1/4Δ31, rDEN2/4Δ31, rDEN3/4Δ31, rDEN4Δ31, (254) rDEN1/4Δ31, rDEN2/4Δ31, rDEN3/4Δ31, rDEN4/1Δ31, (255) rDEN1/4Δ31, rDEN2/4Δ31, rDEN3/4Δ31, rDEN4/2Δ31, and (256) rDEN1/4Δ31, rDEN2/4Δ31, rDEN3/4Δ31, rDEN4/3Δ31. In another embodiment, the fifth attenuated virus of the pentavalent immunogenic composition of any of the combinations of the first, second, third and four attenuated viruses described above is a Zika chimera of the present disclosure, as described in greater detail above. In an embodiment, each of the attenuated viruses comprises the same dengue backbone. In another embodiment, the fifth attenuated virus comprises a different dengue virus backbone than the first, second, third, and fourth attenuated viruses.

In an embodiment, the first, second, third, and fourth attenuated viruses are selected from the group consisting of: (1) rDEN1Δ30/31, rDEN2Δ30/31, rDEN3Δ30/31, rDEN4Δ30/31, (2) rDEN1Δ30/31, rDEN2Δ30/31, rDEN3Δ30/31, rDEN4/1Δ30/31, (3) rDEN1Δ30/31, rDEN2Δ30/31, rDEN3Δ30/31, rDEN4/2Δ30/31, (4) rDEN1Δ30/31, rDEN2Δ30/31, rDEN3Δ30/31, rDEN4/3Δ30/31, (5) rDEN1Δ30/31, rDEN2Δ30/31, rDEN3/1Δ30/31, rDEN4Δ30/31, (6) rDEN1Δ30/31, rDEN2Δ30/31, rDEN3/1Δ30/31, rDEN4/1Δ30/31, (7) rDEN1Δ30/31, rDEN2Δ30/31, rDEN3/1Δ30/31, rDEN4/2Δ30/31, (8) rDEN1Δ30/31, rDEN2Δ30/31, rDEN3/1Δ30/31, rDEN4/3Δ30/31, (9) rDEN1Δ30/31, rDEN2Δ30/31, rDEN3/2Δ30/31, rDEN4Δ30/31, (10) rDEN1Δ30/31, rDEN2Δ30/31, rDEN3/2Δ30/31, rDEN4/1Δ30/31, (11) rDEN1Δ30/31, rDEN2Δ30/31, rDEN3/2Δ30/31, rDEN4/2Δ30/31, (12) rDEN1Δ30/31, rDEN2Δ30/31, rDEN3/2Δ30/31, rDEN4/3Δ30/31, (13) rDEN1Δ30/31, rDEN2Δ30/31, rDEN3/4Δ30/31, rDEN4Δ30/31, (14) rDEN1Δ30/31, rDEN2Δ30/31, rDEN3/4Δ30/31, rDEN4/1Δ30/31, (15) rDEN1Δ30/31, rDEN2Δ30/31, rDEN3/4Δ30/31, rDEN4/2Δ30/31, (16) rDEN1Δ30/31, rDEN2Δ30/31, rDEN3/4Δ30/31, rDEN4/3Δ30/31, (17) rDEN1Δ30/31, rDEN2/1Δ30/31, rDEN3Δ30/31, rDEN4Δ30/31, (18) rDEN1Δ30/31, rDEN2/1Δ30/31, rDEN3Δ30/31, rDEN4/1Δ30/31, (19) rDEN1Δ30/31, rDEN2/1Δ30/31, rDEN3Δ30/31, rDEN4/2Δ30/31, (20) rDEN1Δ30/31, rDEN2/1Δ30/31, rDEN3Δ30/31, rDEN4/3Δ30/31, (21) rDEN1Δ30/31, rDEN2/1Δ30/31, rDEN3/1Δ30/31, rDEN4Δ30/31, (22) rDEN1Δ30/31, rDEN2/1Δ30/31, rDEN3/1Δ30/31, rDEN4/1Δ30/31, (23) rDEN1Δ30/31, rDEN2/1Δ30/31, rDEN3/1Δ30/31, rDEN4/2Δ30/31, (24) rDEN1Δ30/31, rDEN2/1Δ30/31, rDEN3/1Δ30/31, rDEN4/3Δ30/31, (25) rDEN1Δ30/31, rDEN2/1Δ30/31, rDEN3/2Δ30/31, rDEN4Δ30/31, (26) rDEN1Δ30/31, rDEN2/1Δ30/31, rDEN3/2Δ30/31, rDEN4/1Δ30/31, (27) rDEN1Δ30/31, rDEN2/1Δ30/31, rDEN3/2Δ30/31, rDEN4/2Δ30/31, (28) rDEN1Δ30/31, rDEN2/1Δ30/31, rDEN3/2Δ30/31, rDEN4/3Δ30/31, (29) rDEN1Δ30/31, rDEN2/1Δ30/31, rDEN3/4Δ30/31, rDEN4Δ30/31, (30) rDEN1Δ30/31, rDEN2/1Δ30/31, rDEN3/4Δ30/31, rDEN4/1Δ30/31, (31) rDEN1Δ30/31, rDEN2/1Δ30/31, rDEN3/4Δ30/31, rDEN4/2Δ30/31, (32) rDEN1Δ30/31, rDEN2/1Δ30/31, rDEN3/4Δ30/31, rDEN4/3Δ30/31, (33) rDEN1Δ30/31, rDEN2/3Δ30/31, rDEN3Δ30/31, rDEN4Δ30/31, (34) rDEN1Δ30/31, rDEN2/3Δ30/31, rDEN3Δ30/31, rDEN4/1Δ30/31, (35) rDEN1Δ30/31, rDEN2/3Δ30/31, rDEN3Δ30/31, rDEN4/2Δ30/31, (36) rDEN1Δ30/31, rDEN2/3Δ30/31, rDEN3Δ30/31, rDEN4/3Δ30/31, (37) rDEN1Δ30/31, rDEN2/3Δ30/31, rDEN3/1Δ30/31, rDEN4Δ30/31, (38) rDEN1Δ30/31, rDEN2/3Δ30/31, rDEN3/1Δ30/31, rDEN4/1Δ30/31, (39) rDEN1Δ30/31, rDEN2/3Δ30/31, rDEN3/1Δ30/31, rDEN4/2Δ30/31, (40) rDEN1Δ30/31, rDEN2/3Δ30/31, rDEN3/1Δ30/31, rDEN4/3Δ30/31, (41) rDEN1Δ30/31, rDEN2/3Δ30/31, rDEN3/2Δ30/31, rDEN4Δ30/31, (42) rDEN1Δ30/31, rDEN2/3Δ30/31, rDEN3/2Δ30/31, rDEN4/1Δ30/31, (43) rDEN1M 0/31, rDEN2/3Δ30/31, rDEN3/2Δ30/31, rDEN4/2Δ30/31, (44) rDEN1M 0/31, rDEN2/3Δ30/31, rDEN3/2Δ30/31, rDEN4/3Δ30/31, (45) rDEN1Δ30/31, rDEN2/3Δ30/31, rDEN3/4Δ30/31, rDEN4Δ30/31, (46) rDEN1M0/31, rDEN2/3Δ30/31, rDEN3/4Δ30/31, rDEN4/1Δ30/31, (47) rDEN1Δ30/31, rDEN2/3Δ30/31, rDEN3/4Δ30/31, rDEN4/2Δ30/31, (48) rDEN1Δ30/31, rDEN2/3Δ30/31, rDEN3/4Δ30/31, rDEN4/3Δ30/31, (49) rDEN1Δ30/31, rDEN2/4Δ30/31, rDEN3Δ30/31, rDEN4Δ30/31, (50) rDEN1Δ30/31, rDEN2/4Δ30/31, rDEN3Δ30/31, rDEN4/1Δ30/31, (51) rDEN1Δ30/31, rDEN2/4Δ30/31, rDEN3Δ30/31, rDEN4/2Δ30/31, (52) rDEN1Δ30/31, rDEN2/4Δ30/31, rDEN3Δ30/31, rDEN4/3Δ30/31, (53) rDEN1Δ30/31, rDEN2/4Δ30/31, rDEN3/1Δ30/31, rDEN4Δ30/31, (54) rDEN1Δ30/31, rDEN2/4Δ30/31, rDEN3/1Δ30/31, rDEN4/1Δ30/31, (55) rDEN1Δ30/31, rDEN2/4Δ30/31, rDEN3/1Δ30/31, rDEN4/2Δ30/31, (56) rDEN1Δ30/31, rDEN2/4Δ30/31, rDEN3/1Δ30/31, rDEN4/3Δ30/31, (57) rDEN1Δ30/31, rDEN2/4Δ30/31, rDEN3/2Δ30/31, rDEN4Δ30/31, (58) rDEN1Δ30/31, rDEN2/4Δ30/31, rDEN3/2Δ30/31, rDEN4/1Δ30/31, (59) rDEN1Δ30/31, rDEN2/4Δ30/31, rDEN3/2Δ30/31, rDEN4/2Δ30/31, (60) rDEN1Δ30/31, rDEN2/4Δ30/31, rDEN3/2Δ30/31, rDEN4/3Δ30/31, (61) rDEN1Δ30/31, rDEN2/4Δ30/31, rDEN3/4Δ30/31, rDEN4Δ30/31, (62) rDEN1Δ30/31, rDEN2/4Δ30/31, rDEN3/4Δ30/31, rDEN4/1Δ30/31, (63) rDEN1Δ30/31, rDEN2/4Δ30/31, rDEN3/4Δ30/31, rDEN4/2Δ30/31, (64) rDEN1M 0/31, rDEN2/4Δ30/31, rDEN3/4Δ30/31, rDEN4/3Δ30/31, (65) rDEN1/2Δ30/31, rDEN2Δ30/31, rDEN3Δ30/31, rDEN4Δ30/31, (66) rDEN1/2Δ30/31, rDEN2Δ30/31, rDEN3Δ30/31, rDEN4/1Δ30/31, (67) rDEN1/2Δ30/31, rDEN2Δ30/31, rDEN3Δ30/31, rDEN4/2Δ30/31, (68) rDEN1/2Δ30/31, rDEN2Δ30/31, rDEN3Δ30/31, rDEN4/3Δ30/31, (69) rDEN1/2Δ30/31, rDEN2Δ30/31, rDEN3/1Δ30/31, rDEN4Δ30/31, (70) rDEN1/2Δ30/31, rDEN2Δ30/31, rDEN3/1Δ30/31, rDEN4/1Δ30/31, (71) rDEN1/2Δ30/31, rDEN2Δ30/31, rDEN3/1Δ30/31, rDEN4/2Δ30/31, (72) rDEN1/2Δ30/31, rDEN2Δ30/31, rDEN3/1Δ30/31, rDEN4/3Δ30/31, (73) rDEN1/2Δ30/31, rDEN2Δ30/31, rDEN3/2Δ30/31, rDEN4Δ30/31, (74) rDEN1/2Δ30/31, rDEN2Δ30/31, rDEN3/2Δ30/31, rDEN4/1Δ30/31, (75) rDEN1/2Δ30/31, rDEN2Δ30/31, rDEN3/2Δ30/31, rDEN4/2Δ30/31, (76) rDEN1/2Δ40/31, rDEN2Δ30/31, rDEN3/2Δ30/31, rDEN4/3Δ30/31, (77) rDEN1/2Δ40/31, rDEN2Δ30/31, rDEN3/4Δ30/31, rDEN4Δ30/31, (78) rDEN1/2Δ40/31, rDEN2Δ30/31, rDEN3/4Δ30/31, rDEN4/1Δ30/31, (79) rDEN1/2Δ40/31, rDEN2Δ30/31, rDEN3/4Δ30/31, rDEN4/2Δ30/31, (80) rDEN1/2Δ30/31, rDEN2Δ30/31, rDEN3/4Δ30/31, rDEN4/3Δ30/31, (81) rDEN1/2Δ30/31, rDEN2/1Δ30/31, rDEN3Δ30/31, rDEN4Δ30/31, (82) rDEN1/2Δ30/31, rDEN2/1Δ30/31, rDEN3Δ30/31, rDEN4/1Δ30/31, (83) rDEN1/2Δ30/31, rDEN2/1Δ30/31, rDEN3Δ30/31, rDEN4/2Δ30/31, (84) rDEN1/2Δ30/31, rDEN2/1Δ30/31, rDEN3Δ30/31, rDEN4/3Δ30/31, (85) rDEN1/2Δ30/31, rDEN2/1Δ30/31, rDEN3/1Δ30/31, rDEN4Δ30/31, (86) rDEN1/2Δ30/31, rDEN2/1Δ30/31, rDEN3/1Δ30/31, rDEN4/1Δ30/31, (87) rDEN1/2Δ30/31, rDEN2/1Δ30/31, rDEN3/1Δ30/31, rDEN4/2Δ30/31, (88) rDEN1/2Δ30/31, rDEN2/1Δ30/31, rDEN3/1Δ30/31, rDEN4/3Δ30/31, (89) rDEN1/2Δ30/31, rDEN2/1Δ30/31, rDEN3/2Δ30/31, rDEN4Δ30/31, (90) rDEN1/2Δ30/31, rDEN2/1Δ30/31, rDEN3/2Δ30/31, rDEN4/1Δ30/31, (91) rDEN1/2Δ30/31, rDEN2/1Δ30/31, rDEN3/2Δ30/31, rDEN4/2Δ30/31, (92) rDEN1/2Δ30/31, rDEN2/1Δ30/31, rDEN3/2Δ30/31, rDEN4/3Δ30/31, (93) rDEN1/2Δ30/31, rDEN2/1Δ30/31, rDEN3/4Δ30/31, rDEN4Δ30/31, (94) rDEN1/2Δ30/31, rDEN2/1Δ30/31, rDEN3/4Δ30/31, rDEN4/1Δ30/31, (95) rDEN1/2Δ30/31, rDEN2/1Δ30/31, rDEN3/4Δ30/31, rDEN4/2Δ30/31, (96) rDEN1/2Δ30/31, rDEN2/1Δ30/31, rDEN3/4Δ30/31, rDEN4/3Δ30/31, (97) rDEN1/2Δ30/31, rDEN2/3Δ30/31, rDEN3Δ30/31, rDEN4Δ30/31, (98) rDEN1/2Δ30/31, rDEN2/3Δ30/31, rDEN3Δ30/31, rDEN4/1Δ30/31, (99) rDEN1/2Δ30/31, rDEN2/3Δ30/31, rDEN3Δ30/31, rDEN4/2Δ30/31, (100) rDEN1/2Δ30/31, rDEN2/3Δ30/31, rDEN3Δ30/31, rDEN4/3Δ30/31, (101) rDEN1/2Δ30/31, rDEN2/3Δ30/31, rDEN3/1Δ30/31, rDEN4Δ30/31, (102) rDEN1/2Δ30/31, rDEN2/3Δ30/31, rDEN3/1Δ30/31, rDEN4/1Δ30/31, (103) rDEN1/2Δ30/31, rDEN2/3Δ30/31, rDEN3/1Δ30/31, rDEN4/2Δ30/31, (104) rDEN1/2Δ30/31, rDEN2/3Δ30/31, rDEN3/1Δ30/31, rDEN4/3Δ30/31, (105) rDEN1/2Δ30/31, rDEN2/3Δ30/31, rDEN3/2Δ30/31, rDEN4Δ30/31, (106) rDEN1/2Δ30/31, rDEN2/3Δ30/31, rDEN3/2Δ30/31, rDEN4/1Δ30/31, (107) rDEN1/2Δ30/31, rDEN2/3Δ30/31, rDEN3/2Δ30/31, rDEN4/2Δ30/31, (108) rDEN1/2Δ30/31, rDEN2/3Δ30/31, rDEN3/2Δ30/31, rDEN4/3Δ30/31, (109) rDEN1/2Δ30/31, rDEN2/3Δ30/31, rDEN3/4Δ30/31, rDEN4Δ30/31, (110) rDEN1/2Δ30/31, rDEN2/3Δ30/31, rDEN3/4Δ30/31, rDEN4/1Δ30/31, (111) rDEN1/2Δ30/31, rDEN2/3Δ30/31, rDEN3/4Δ30/31, rDEN4/2Δ30/31, (112) rDEN1/2Δ30/31, rDEN2/3Δ30/31, rDEN3/4Δ30/31, rDEN4/3Δ30/31, (113) rDEN1/2Δ30/31, rDEN2/4Δ30/31, rDEN3Δ30/31, rDEN4Δ30/31, (114) rDEN1/2Δ30/31, rDEN2/4Δ30/31, rDEN3Δ30/31, rDEN4/1Δ30/31, (115) rDEN1/2Δ30/31, rDEN2/4Δ30/31, rDEN3Δ30/31, rDEN4/2Δ30/31, (116) rDEN1/2Δ30/31, rDEN2/4Δ30/31, rDEN3Δ30/31, rDEN4/3Δ30/31, (117) rDEN1/2Δ30/31, rDEN2/4Δ30/31, rDEN3/1Δ30/31, rDEN4Δ30/31, (118) rDEN1/2Δ30/31, rDEN2/4Δ30/31, rDEN3/1Δ30/31, rDEN4/1Δ30/31, (119) rDEN1/2Δ30/31, rDEN2/4Δ30/31, rDEN3/1Δ30/31, rDEN4/2Δ30/31, (120) rDEN1/2Δ30/31, rDEN2/4Δ30/31, rDEN3/1Δ30/31, rDEN4/3Δ30/31, (121) rDEN1/2Δ30/31, rDEN2/4Δ30/31, rDEN3/2Δ30/31, rDEN4Δ30/31, (122) rDEN1/2Δ30/31, rDEN2/4Δ30/31, rDEN3/2Δ30/31, rDEN4/1Δ30/31, (123) rDEN1/2Δ30/31, rDEN2/4Δ30/31, rDEN3/2Δ30/31, rDEN4/2Δ30/31, (124) rDEN1/2Δ30/31, rDEN2/4Δ30/31, rDEN3/2Δ30/31, rDEN4/3Δ30/31, (125) rDEN1/2Δ30/31, rDEN2/4Δ30/31, rDEN3/4Δ30/31, rDEN4Δ30/31, (126) rDEN1/2Δ30/31, rDEN2/4Δ30/31, rDEN3/4Δ30/31, rDEN4/1Δ30/31, (127) rDEN1/2Δ30/31, rDEN2/4Δ30/31, rDEN3/4Δ30/31, rDEN4/2Δ30/31, (128) rDEN1/2Δ30/31, rDEN2/4Δ30/31, rDEN3/4Δ30/31, rDEN4/3Δ30/31, (129) rDEN1/3Δ30/31, rDEN2Δ30/31, rDEN3Δ30/31, rDEN4Δ30/31, (130) rDEN1/3Δ30/31, rDEN2Δ30/31, rDEN3Δ30/31, rDEN4/1Δ30/31, (131) rDEN1/3Δ30/31, rDEN2Δ30/31, rDEN3Δ30/31, rDEN4/2Δ30/31, (132) rDEN1/3Δ30/31, rDEN2Δ30/31, rDEN3Δ30/31, rDEN4/3Δ30/31, (133) rDEN1/3Δ30/31, rDEN2Δ30/31, rDEN3/1Δ30/31, rDEN4Δ30/31, (134) rDEN1/3Δ30/31, rDEN2Δ30/31, rDEN3/1Δ30/31, rDEN4/1Δ30/31, (135) rDEN1/3Δ30/31, rDEN2Δ30/31, rDEN3/1Δ30/31, rDEN4/2Δ30/31, (136) rDEN1/3Δ30/31, rDEN2Δ30/31, rDEN3/1Δ30/31, rDEN4/3Δ30/31, (137) rDEN1/3Δ30/31, rDEN2Δ30/31, rDEN3/2Δ30/31, rDEN4Δ30/31, (138) rDEN1/3Δ30/31, rDEN2Δ30/31, rDEN3/2Δ30/31, rDEN4/1Δ30/31, (139) rDEN1/3Δ30/31, rDEN2Δ30/31, rDEN3/2Δ30/31, rDEN4/2Δ30/31, (140) rDEN1/3Δ30/31, rDEN2Δ30/31, rDEN3/2Δ30/31, rDEN4/3Δ30/31, (141) rDEN1/3Δ30/31, rDEN2Δ30/31, rDEN3/4Δ30/31, rDEN4Δ30/31, (142) rDEN1/3Δ30/31, rDEN2Δ30/31, rDEN3/4Δ30/31, rDEN4/1Δ30/31, (143) rDEN1/3Δ30/31, rDEN2Δ30/31, rDEN3/4Δ30/31, rDEN4/2Δ30/31, (144) rDEN1/3Δ30/31, rDEN2Δ30/31, rDEN3/4Δ30/31, rDEN4/3Δ30/31, (145) rDEN1/3Δ30/31, rDEN2/1Δ30/31, rDEN3Δ30/31, rDEN4Δ30/31, (146) rDEN1/3Δ30/31, rDEN2/1Δ30/31, rDEN3Δ30/31, rDEN4/1Δ30/31, (147) rDEN1/3Δ30/31, rDEN2/1Δ30/31, rDEN3Δ30/31, rDEN4/2Δ30/31, (148) rDEN1/3Δ30/31, rDEN2/1Δ30/31, rDEN3Δ30/31, rDEN4/3Δ30/31, (149) rDEN1/3Δ30/31, rDEN2/1Δ30/31, rDEN3/1Δ30/31, rDEN4Δ30/31, (150) rDEN1/3Δ30/31, rDEN2/1Δ30/31, rDEN3/1Δ30/31, rDEN4/1Δ30/31, (151) rDEN1/3Δ30/31, rDEN2/1Δ30/31, rDEN3/1Δ30/31, rDEN4/2Δ30/31, (152) rDEN1/3Δ30/31, rDEN2/1Δ30/31, rDEN3/1Δ30/31, rDEN4/3Δ30/31, (153) rDEN1/3Δ30/31, rDEN2/1Δ30/31, rDEN3/2Δ30/31, rDEN4Δ30/31, (154) rDEN1/3Δ30/31, rDEN2/1Δ30/31, rDEN3/2Δ30/31, rDEN4/1Δ30/31, (155) rDEN1/3Δ30/31, rDEN2/1Δ30/31, rDEN3/2Δ30/31, rDEN4/2Δ30/31, (156) rDEN1/3Δ30/31, rDEN2/1Δ30/31, rDEN3/2Δ30/31, rDEN4/3Δ30/31, (157) rDEN1/3Δ30/31, rDEN2/1Δ30/31, rDEN3/4Δ30/31, rDEN4Δ30/31, (158) rDEN1/3Δ30/31, rDEN2/1Δ30/31, rDEN3/4Δ30/31, rDEN4/1Δ30/31, (159) rDEN1/3Δ30/31, rDEN2/1Δ30/31, rDEN3/4Δ30/31, rDEN4/2Δ30/31, (160) rDEN1/3Δ30/31, rDEN2/1Δ30/31, rDEN3/4Δ30/31, rDEN4/3Δ30/31, (161) rDEN1/3Δ30/31, rDEN2/3Δ30/31, rDEN3Δ30/31, rDEN4Δ30/31, (162) rDEN1/3Δ30/31, rDEN2/3Δ30/31, rDEN3Δ30/31, rDEN4/1Δ30/31, (163) rDEN1/3Δ30/31, rDEN2/3Δ30/31, rDEN3Δ30/31, rDEN4/2Δ30/31, (164) rDEN1/3Δ30/31, rDEN2/3Δ30/31, rDEN3Δ30/31, rDEN4/3Δ30/31, (165) rDEN1/3Δ30/31, rDEN2/3Δ30/31, rDEN3/1Δ30/31, rDEN4Δ30/31, (166) rDEN1/3Δ30/31, rDEN2/3Δ30/31, rDEN3/1Δ30/31, rDEN4/1Δ30/31, (167) rDEN1/3Δ30/31, rDEN2/3Δ30/31, rDEN3/1Δ30/31, rDEN4/2Δ30/31, (168) rDEN1/3Δ30/31, rDEN2/3Δ30/31, rDEN3/1Δ30/31, rDEN4/3Δ30/31, (169) rDEN1/3Δ30/31, rDEN2/3Δ30/31, rDEN3/2Δ30/31, rDEN4Δ30/31, (170) rDEN1/3Δ30/31, rDEN2/3Δ30/31, rDEN3/2Δ30/31, rDEN4/1Δ30/31, (171) rDEN1/3Δ30/31, rDEN2/3Δ30/31, rDEN3/2Δ30/31, rDEN4/2Δ30/31, (172) rDEN1/3Δ30/31, rDEN2/3Δ30/31, rDEN3/2Δ30/31, rDEN4/3Δ30/31, (173) rDEN1/3Δ30/31, rDEN2/3Δ30/31, rDEN3/4Δ30/31, rDEN4Δ30/31, (174) rDEN1/3Δ30/31, rDEN2/3Δ30/31, rDEN3/4Δ30/31, rDEN4/1Δ30/31, (175) rDEN1/3Δ30/31, rDEN2/3Δ30/31, rDEN3/4Δ30/31, rDEN4/2Δ30/31, (176) rDEN1/3Δ30/31, rDEN2/3Δ30/31, rDEN3/4Δ30/31, rDEN4/3Δ30/31, (177) rDEN1/3Δ30/31, rDEN2/4Δ30/31, rDEN3Δ30/31, rDEN4Δ30/31, (178) rDEN1/3Δ30/31, rDEN2/4Δ30/31, rDEN3Δ30/31, rDEN4/1Δ30/31, (179) rDEN1/3Δ30/31, rDEN2/4Δ30/31, rDEN3Δ30/31, rDEN4/2Δ30/31, (180) rDEN1/3Δ30/31, rDEN2/4Δ30/31, rDEN3Δ30/31, rDEN4/3Δ30/31, (181) rDEN1/3Δ30/31, rDEN2/4Δ30/31, rDEN3/1Δ30/31, rDEN4Δ30/31, (182) rDEN1/3Δ30/31, rDEN2/4Δ30/31, rDEN3/1Δ30/31, rDEN4/1Δ30/31, (183) rDEN1/3Δ30/31, rDEN2/4Δ30/31, rDEN3/1Δ30/31, rDEN4/2Δ30/31, (184) rDEN1/3Δ30/31, rDEN2/4Δ30/31, rDEN3/1Δ30/31, rDEN4/3Δ30/31, (185) rDEN1/3Δ30/31, rDEN2/4Δ30/31, rDEN3/2Δ30/31, rDEN4Δ30/31, (186) rDEN1/3Δ30/31, rDEN2/4Δ30/31, rDEN3/2Δ30/31, rDEN4/1Δ30/31, (187) rDEN1/3Δ30/31, rDEN2/4Δ30/31, rDEN3/2Δ30/31, rDEN4/2Δ30/31, (188) rDEN1/3Δ30/31, rDEN2/4Δ30/31, rDEN3/2Δ30/31, rDEN4/3Δ30/31, (189) rDEN1/3Δ30/31, rDEN2/4Δ30/31, rDEN3/4Δ30/31, rDEN4Δ30/31, (190) rDEN1/3Δ30/31, rDEN2/4Δ30/31, rDEN3/4Δ30/31, rDEN4/1Δ30/31, (191) rDEN1/3Δ30/31, rDEN2/4Δ30/31, rDEN3/4Δ30/31, rDEN4/2Δ30/31, (192) rDEN1/3Δ30/31, rDEN2/4Δ30/31, rDEN3/4Δ30/31, rDEN4/3Δ30/31, (193) rDEN1/4Δ30/31, rDEN2Δ30/31, rDEN3Δ30/31, rDEN4Δ30/31, (194) rDEN1/4Δ30/31, rDEN2Δ30/31, rDEN3Δ30/31, rDEN4/1Δ30/31, (195) rDEN1/4Δ30/31, rDEN2Δ30/31, rDEN3Δ30/31, rDEN4/2Δ30/31, (196) rDEN1/4Δ30/31, rDEN2Δ30/31, rDEN3Δ30/31, rDEN4/3Δ30/31, (197) rDEN1/4Δ30/31, rDEN2Δ30/31, rDEN3/1Δ30/31, rDEN4Δ30/31, (198) rDEN1/4Δ30/31, rDEN2Δ30/31, rDEN3/1Δ30/31, rDEN4/1Δ30/31, (199) rDEN1/4Δ30/31, rDEN2Δ30/31, rDEN3/1Δ30/31, rDEN4/2Δ30/31, (200) rDEN1/4Δ30/31, rDEN2Δ30/31, rDEN3/1Δ30/31, rDEN4/3Δ30/31, (201) rDEN1/4Δ30/31, rDEN2Δ30/31, rDEN3/2Δ30/31, rDEN4Δ30/31, (202) rDEN1/4Δ30/31, rDEN2Δ30/31, rDEN3/2Δ30/31, rDEN4/1Δ30/31, (203) rDEN1/4Δ30/31, rDEN2Δ30/31, rDEN3/2Δ30/31, rDEN4/2Δ30/31, (204) rDEN1/4Δ30/31, rDEN2Δ30/31, rDEN3/2Δ30/31, rDEN4/3Δ30/31, (205) rDEN1/4Δ30/31, rDEN2Δ30/31, rDEN3/4Δ30/31, rDEN4Δ30/31, (206) rDEN1/4Δ30/31, rDEN2Δ30/31, rDEN3/4Δ30/31, rDEN4/1Δ30/31, (207) rDEN1/4Δ30/31, rDEN2Δ30/31, rDEN3/4Δ30/31, rDEN4/2Δ30/31, (208) rDEN1/4Δ30/31, rDEN2Δ30/31, rDEN3/4Δ30/31, rDEN4/3Δ30/31, (209) rDEN1/4Δ30/31, rDEN2/1Δ30/31, rDEN3Δ30/31, rDEN4Δ30/31, (210) rDEN1/4Δ30/31, rDEN2/1Δ30/31, rDEN3Δ30/31, rDEN4/1Δ30/31, (211) rDEN1/4Δ30/31, rDEN2/1Δ30/31, rDEN3Δ30/31, rDEN4/2Δ30/31, (212) rDEN1/4Δ30/31, rDEN2/1Δ30/31, rDEN3Δ30/31, rDEN4/3Δ30/31, (213) rDEN1/4Δ30/31, rDEN2/1Δ30/31, rDEN3/1Δ30/31, rDEN4Δ30/31, (214) rDEN1/4Δ30/31, rDEN2/1Δ30/31, rDEN3/1Δ30/31, rDEN4/1Δ30/31, (215) rDEN1/4Δ30/31, rDEN2/1Δ30/31, rDEN3/1Δ30/31, rDEN4/2Δ30/31, (216) rDEN1/4Δ30/31, rDEN2/1Δ30/31, rDEN3/1Δ30/31, rDEN4/3Δ30/31, (217) rDEN1/4Δ30/31, rDEN2/1Δ30/31, rDEN3/2Δ30/31, rDEN4Δ30/31, (218) rDEN1/4Δ30/31, rDEN2/1Δ30/31, rDEN3/2Δ30/31, rDEN4/1Δ30/31, (219) rDEN1/4Δ30/31, rDEN2/1Δ30/31, rDEN3/2Δ30/31, rDEN4/2Δ30/31, (220) rDEN1/4Δ30/31, rDEN2/1Δ30/31, rDEN3/2Δ30/31, rDEN4/3Δ30/31, (221) rDEN1/4Δ30/31, rDEN2/1Δ30/31, rDEN3/4Δ30/31, rDEN4Δ30/31, (222) rDEN1/4Δ30/31, rDEN2/1Δ30/31, rDEN3/4Δ30/31, rDEN4/1Δ30/31, (223) rDEN1/4Δ30/31, rDEN2/1Δ30/31, rDEN3/4Δ30/31, rDEN4/2Δ30/31, (224) rDEN1/4Δ30/31, rDEN2/1Δ30/31, rDEN3/4Δ30/31, rDEN4/3Δ30/31, (225) rDEN1/4Δ30/31, rDEN2/3Δ30/31, rDEN3Δ30/31, rDEN4Δ30/31, (226) rDEN1/4Δ30/31, rDEN2/3Δ30/31, rDEN3Δ30/31, rDEN4/1Δ30/31, (227) rDEN1/4Δ30/31, rDEN2/3Δ30/31, rDEN3Δ30/31, rDEN4/2Δ30/31, (228) rDEN1/4Δ30/31, rDEN2/3Δ30/31, rDEN3Δ30/31, rDEN4/3Δ30/31, (229) rDEN1/4Δ30/31, rDEN2/3Δ30/31, rDEN3/1Δ30/31, rDEN4Δ30/31, (230) rDEN1/4Δ30/31, rDEN2/3Δ30/31, rDEN3/1Δ30/31, rDEN4/1Δ30/31, (231) rDEN1/4Δ30/31, rDEN2/3Δ30/31, rDEN3/1Δ30/31, rDEN4/2Δ30/31, (232) rDEN1/4Δ30/31, rDEN2/3Δ30/31, rDEN3/1Δ30/31, rDEN4/3Δ30/31, (233) rDEN1/4Δ30/31, rDEN2/3Δ30/31, rDEN3/2Δ30/31, rDEN4Δ30/31, (234) rDEN1/4Δ30/31, rDEN2/3Δ30/31, rDEN3/2Δ30/31, rDEN4/1Δ30/31, (235) rDEN1/4Δ30/31, rDEN2/3Δ30/31, rDEN3/2Δ30/31, rDEN4/2Δ30/31, (236) rDEN1/4Δ30/31, rDEN2/3Δ30/31, rDEN3/2Δ30/31, rDEN4/3Δ30/31, (237) rDEN1/4Δ30/31, rDEN2/3Δ30/31, rDEN3/4Δ30/31, rDEN4Δ30/31, (238) rDEN1/4Δ30/31, rDEN2/3Δ30/31, rDEN3/4Δ30/31, rDEN4/1Δ30/31, (239) rDEN1/4Δ30/31, rDEN2/3Δ30/31, rDEN3/4Δ30/31, rDEN4/2Δ30/31, (240) rDEN1/4Δ30/31, rDEN2/3Δ30/31, rDEN3/4Δ30/31, rDEN4/3Δ30/31, (241) rDEN1/4Δ30/31, rDEN2/4Δ30/31, rDEN3Δ30/31, rDEN4Δ30/31, (242) rDEN1/4Δ30/31, rDEN2/4Δ30/31, rDEN3Δ30/31, rDEN4/1Δ30/31, (243) rDEN1/4Δ30/31, rDEN2/4Δ30/31, rDEN3Δ30/31, rDEN4/2Δ30/31, (244) rDEN1/4Δ30/31, rDEN2/4Δ30/31, rDEN3Δ30/31, rDEN4/3Δ30/31, (245) rDEN1/4Δ30/31, rDEN2/4Δ30/31, rDEN3/1Δ30/31, rDEN4Δ30/31, (246) rDEN1/4Δ30/31, rDEN2/4Δ30/31, rDEN3/1Δ30/31, rDEN4/1Δ30/31, (247) rDEN1/4Δ30/31, rDEN2/4Δ30/31, rDEN3/1Δ30/31, rDEN4/2Δ30/31, (248) rDEN1/4Δ30/31, rDEN2/4Δ30/31, rDEN3/1Δ30/31, rDEN4/3Δ30/31, (249) rDEN1/4Δ30/31, rDEN2/4Δ30/31, rDEN3/2Δ30/31, rDEN4Δ30/31, (250) rDEN1/4Δ30/31, rDEN2/4Δ30/31, rDEN3/2Δ30/31, rDEN4/1Δ30/31, (251) rDEN1/4Δ30/31, rDEN2/4Δ30/31, rDEN3/2Δ30/31, rDEN4/2Δ30/31, (252) rDEN1/4Δ30/31, rDEN2/4Δ30/31, rDEN3/2Δ30/31, rDEN4/3Δ30/31, (253) rDEN1/4Δ30/31, rDEN2/4Δ30/31, rDEN3/4Δ30/31, rDEN4Δ30/31, (254) rDEN1/4Δ30/31, rDEN2/4Δ30/31, rDEN3/4Δ30/31, rDEN4/1Δ30/31, (255) rDEN1/4Δ30/31, rDEN2/4Δ30/31, rDEN3/4Δ30/31, rDEN4/2Δ30/31, and (256) rDEN1/4Δ30/31, rDEN2/4Δ30/31, rDEN3/4Δ30/31, rDEN4/3Δ30/31. In another embodiment, the fifth attenuated virus of the pentavalent immunogenic composition of any of the combinations of the first, second, third and four attenuated viruses described above is a Zika chimera of the present disclosure, as described in greater detail above. In an embodiment, each of the attenuated viruses comprises the same dengue backbone. In another embodiment, the fifth attenuated virus comprises a different dengue virus backbone than the first, second, third, and fourth attenuated viruses.

In an embodiment, the first, second, third, and four attenuated viruses are selected from the group consisting of: (1) rDEN1Δ86, rDEN2Δ86, rDEN3Δ86, rDEN4Δ86, (2) rDEN1Δ86, rDEN2Δ86, rDEN3Δ86, rDEN4/1Δ86, (3) rDEN1Δ86, rDEN2Δ86, rDEN3Δ86, rDEN4/2Δ86, (4) rDEN1Δ86, rDEN2Δ86, rDEN3Δ86, rDEN4/3Δ86, (5) rDEN1Δ86, rDEN2Δ86, rDEN3/1Δ86, rDEN4Δ86, (6) rDEN1Δ86, rDEN2Δ86, rDEN3/1Δ86, rDEN4/1Δ86, (7) rDEN1Δ86, rDEN2Δ86, rDEN3/1Δ86, rDEN4/2Δ86, (8) rDEN1Δ86, rDEN2Δ86, rDEN3/1Δ86, rDEN4/3Δ86, (9) rDEN1Δ86, rDEN2Δ86, rDEN3/2Δ86, rDEN4Δ86, (10) rDEN1Δ86, rDEN2Δ86, rDEN3/2Δ86, rDEN4/1Δ86, (11) rDEN1Δ86, rDEN2Δ86, rDEN3/2Δ86, rDEN4/2Δ86, (12) rDEN1Δ86, rDEN2Δ86, rDEN3/2Δ86, rDEN4/3Δ86, (13) rDEN1Δ86, rDEN2Δ86, rDEN3/4Δ86, rDEN4Δ86, (14) rDEN1Δ86, rDEN2Δ86, rDEN3/4Δ86, rDEN4/1Δ86, (15) rDEN1Δ86, rDEN2Δ86, rDEN3/4Δ86, rDEN4/2Δ86, (16) rDEN1Δ86, rDEN2Δ86, rDEN3/4Δ86, rDEN4/3Δ86, (17) rDEN1Δ86, rDEN2/1Δ86, rDEN3Δ86, rDEN4Δ86, (18) rDEN1Δ86, rDEN2/1Δ86, rDEN3Δ86, rDEN4/1Δ86, (19) rDEN1Δ86, rDEN2/1Δ86, rDEN3Δ86, rDEN4/2Δ86, (20) rDEN1Δ86, rDEN2/1Δ86, rDEN3Δ86, rDEN4/3Δ86, (21) rDEN1Δ86, rDEN2/1Δ86, rDEN3/1Δ86, rDEN4Δ86, (22) rDEN1Δ86, rDEN2/1Δ86, rDEN3/1Δ86, rDEN4/1Δ86, (23) rDEN1Δ86, rDEN2/1Δ86, rDEN3/1Δ86, rDEN4/2Δ86, (24) rDEN1Δ86, rDEN2/1Δ86, rDEN3/1Δ86, rDEN4/3Δ86, (25) rDEN1Δ86, rDEN2/1Δ86, rDEN3/2Δ86, rDEN4Δ86, (26) rDEN1Δ86, rDEN2/1Δ86, rDEN3/2Δ86, rDEN4/1Δ86, (27) rDEN1Δ86, rDEN2/1Δ86, rDEN3/2Δ86, rDEN4/2Δ86, (28) rDEN1Δ86, rDEN2/1Δ86, rDEN3/2Δ86, rDEN4/3Δ86, (29) rDEN1Δ86, rDEN2/1Δ86, rDEN3/4Δ86, rDEN4Δ86, (30) rDEN1Δ86, rDEN2/1Δ86, rDEN3/4Δ86, rDEN4/1Δ86, (31) rDEN1Δ86, rDEN2/1Δ86, rDEN3/4Δ86, rDEN4/2Δ86, (32) rDEN1Δ86, rDEN2/1Δ86, rDEN3/4Δ86, rDEN4/3Δ86, (33) rDEN1Δ86, rDEN2/3Δ86, rDEN3Δ86, rDEN4Δ86, (34) rDEN1Δ86, rDEN2/3Δ86, rDEN3Δ86, rDEN4/1Δ86, (35) rDEN1Δ86, rDEN2/3Δ86, rDEN3Δ86, rDEN4/2Δ86, (36) rDEN1Δ86, rDEN2/3Δ86, rDEN3Δ86, rDEN4/3Δ86, (37) rDEN1Δ86, rDEN2/3Δ86, rDEN3/1Δ86, rDEN4Δ86, (38) rDEN1Δ86, rDEN2/3Δ86, rDEN3/1Δ86, rDEN4/1Δ86, (39) rDEN1Δ86, rDEN2/3Δ86, rDEN3/1Δ86, rDEN4/2Δ86, (40) rDEN1Δ86, rDEN2/3Δ86, rDEN3/1Δ86, rDEN4/3Δ86, (41) rDEN1Δ86, rDEN2/3Δ86, rDEN3/2Δ86, rDEN4Δ86, (42) rDEN1Δ86, rDEN2/3Δ86, rDEN3/2Δ86, rDEN4/1Δ86, (43) rDEN1Δ86, rDEN2/3Δ86, rDEN3/2Δ86, rDEN4/2Δ86, (44) rDEN1Δ86, rDEN2/3Δ86, rDEN3/2Δ86, rDEN4/3Δ86, (45) rDEN1Δ86, rDEN2/3Δ86, rDEN3/4Δ86, rDEN4Δ86, (46) rDEN1Δ86, rDEN2/3Δ86, rDEN3/4Δ86, rDEN4/1Δ86, (47) rDEN1Δ86, rDEN2/3Δ86, rDEN3/4Δ86, rDEN4/2Δ86, (48) rDEN1Δ86, rDEN2/3Δ86, rDEN3/4Δ86, rDEN4/3Δ86, (49) rDEN1Δ86, rDEN2/4Δ86, rDEN3Δ86, rDEN4Δ86, (50) rDEN1Δ86, rDEN2/4Δ86, rDEN3Δ86, rDEN4/1Δ86, (51) rDEN1Δ86, rDEN2/4Δ86, rDEN3Δ86, rDEN4/2Δ86, (52) rDEN1Δ86, rDEN2/4Δ86, rDEN3Δ86, rDEN4/3Δ86, (53) rDEN1Δ86, rDEN2/4Δ86, rDEN3/1Δ86, rDEN4Δ86, (54) rDEN1Δ86, rDEN2/4Δ86, rDEN3/1Δ86, rDEN4/1Δ86, (55) rDEN1Δ86, rDEN2/4Δ86, rDEN3/1Δ86, rDEN4/2Δ86, (56) rDEN1Δ86, rDEN2/4Δ86, rDEN3/1Δ86, rDEN4/3Δ86, (57) rDEN1Δ86, rDEN2/4Δ86, rDEN3/2Δ86, rDEN4Δ86, (58) rDEN1Δ86, rDEN2/4Δ86, rDEN3/2Δ86, rDEN4/1Δ86, (59) rDEN1Δ86, rDEN2/4Δ86, rDEN3/2Δ86, rDEN4/2Δ86, (60) rDEN1Δ86, rDEN2/4Δ86, rDEN3/2Δ86, rDEN4/3Δ86, (61) rDEN1Δ86, rDEN2/4Δ86, rDEN3/4Δ86, rDEN4Δ86, (62) rDEN1Δ86, rDEN2/4Δ86, rDEN3/4Δ86, rDEN4/1Δ86, (63) rDEN1Δ86, rDEN2/4Δ86, rDEN3/4Δ86, rDEN4/2Δ86, (64) rDEN1Δ86, rDEN2/4Δ86, rDEN3/4Δ86, rDEN4/3Δ86, (65) rDEN1/2Δ86, rDEN2Δ86, rDEN3Δ86, rDEN4Δ86, (66) rDEN1/2Δ86, rDEN2Δ86, rDEN3Δ86, rDEN4/1Δ86, (67) rDEN1/2Δ86, rDEN2Δ86, rDEN3Δ86, rDEN4/2Δ86, (68) rDEN1/2Δ86, rDEN2Δ86, rDEN3Δ86, rDEN4/3Δ86, (69) rDEN1/2Δ86, rDEN2Δ86, rDEN3/1Δ86, rDEN4Δ86, (70) rDEN1/2Δ86, rDEN2Δ86, rDEN3/1Δ86, rDEN4/1Δ86, (71) rDEN1/2Δ86, rDEN2Δ86, rDEN3/1Δ86, rDEN4/2Δ86, (72) rDEN1/2Δ86, rDEN2Δ86, rDEN3/1Δ86, rDEN4/3Δ86, (73) rDEN1/2Δ86, rDEN2Δ86, rDEN3/2Δ86, rDEN4Δ86, (74) rDEN1/2Δ86, rDEN2Δ86, rDEN3/2Δ86, rDEN4/1Δ86, (75) rDEN1/2Δ86, rDEN2Δ86, rDEN3/2Δ86, rDEN4/2Δ86, (76) rDEN1/2Δ86, rDEN2Δ86, rDEN3/2Δ86, rDEN4/3Δ86, (77) rDEN1/2Δ86, rDEN2Δ86, rDEN3/4Δ86, rDEN4Δ86, (78) rDEN1/2Δ86, rDEN2Δ86, rDEN3/4Δ86, rDEN4/1Δ86, (79) rDEN1/2Δ86, rDEN2Δ86, rDEN3/4Δ86, rDEN4/2Δ86, (80) rDEN1/2Δ86, rDEN2Δ86, rDEN3/4Δ86, rDEN4/3Δ86, (81) rDEN1/2Δ86, rDEN2/1Δ86, rDEN3Δ86, rDEN4Δ86, (82) rDEN1/2Δ86, rDEN2/1Δ86, rDEN3Δ86, rDEN4/1Δ86, (83) rDEN1/2Δ86, rDEN2/1Δ86, rDEN3Δ86, rDEN4/2Δ86, (84) rDEN1/2Δ86, rDEN2/1Δ86, rDEN3Δ86, rDEN4/3Δ86, (85) rDEN1/2Δ86, rDEN2/1Δ86, rDEN3/1Δ86, rDEN4Δ86, (86) rDEN1/2Δ86, rDEN2/1Δ86, rDEN3/1Δ86, rDEN4/1Δ86, (87) rDEN1/2Δ86, rDEN2/1Δ86, rDEN3/1Δ86, rDEN4/2Δ86, (88) rDEN1/2Δ86, rDEN2/1Δ86, rDEN3/1Δ86, rDEN4/3Δ86, (89) rDEN1/2Δ86, rDEN2/1Δ86, rDEN3/2Δ86, rDEN4Δ86, (90) rDEN1/2Δ86, rDEN2/1Δ86, rDEN3/2Δ86, rDEN4/1Δ86, (91) rDEN1/2Δ86, rDEN2/1Δ86, rDEN3/2Δ86, rDEN4/2Δ86, (92) rDEN1/2Δ86, rDEN2/1Δ86, rDEN3/2Δ86, rDEN4/3Δ86, (93) rDEN1/2Δ86, rDEN2/1Δ86, rDEN3/4Δ86, rDEN4Δ86, (94) rDEN1/2Δ86, rDEN2/1Δ86, rDEN3/4Δ86, rDEN4/1Δ86, (95) rDEN1/2Δ86, rDEN2/1Δ86, rDEN3/4Δ86, rDEN4/2Δ86, (96) rDEN1/2Δ86, rDEN2/1Δ86, rDEN3/4Δ86, rDEN4/3Δ86, (97) rDEN1/2Δ86, rDEN2/3Δ86, rDEN3Δ86, rDEN4Δ86, (98) rDEN1/2Δ86, rDEN2/3Δ86, rDEN3Δ86, rDEN4/1Δ86, (99) rDEN1/2Δ86, rDEN2/3Δ86, rDEN3Δ86, rDEN4/2Δ86, (100) rDEN1/2Δ86, rDEN2/3Δ86, rDEN3Δ86, rDEN4/3Δ86, (101) rDEN1/2Δ86, rDEN2/3Δ86, rDEN3/1Δ86, rDEN4Δ86, (102) rDEN1/2Δ86, rDEN2/3Δ86, rDEN3/1Δ86, rDEN4/1Δ86, (103) rDEN1/2Δ86, rDEN2/3Δ86, rDEN3/1Δ86, rDEN4/2Δ86, (104) rDEN1/2Δ86, rDEN2/3Δ86, rDEN3/1Δ86, rDEN4/3Δ86, (105) rDEN1/2Δ86, rDEN2/3Δ86, rDEN3/2Δ86, rDEN4Δ86, (106) rDEN1/2Δ86, rDEN2/3Δ86, rDEN3/2Δ86, rDEN4/1Δ86, (107) rDEN1/2Δ86, rDEN2/3Δ86, rDEN3/2Δ86, rDEN4/2Δ86, (108) rDEN1/2Δ86, rDEN2/3Δ86, rDEN3/2Δ86, rDEN4/3Δ86, (109) rDEN1/2Δ86, rDEN2/3Δ86, rDEN3/4Δ86, rDEN4Δ86, (110) rDEN1/2Δ86, rDEN2/3Δ86, rDEN3/4Δ86, rDEN4/1Δ86, (111) rDEN1/2Δ86, rDEN2/3Δ86, rDEN3/4Δ86, rDEN4/2Δ86, (112) rDEN1/2Δ86, rDEN2/3Δ86, rDEN3/4Δ86, rDEN4/3Δ86, (113) rDEN1/2Δ86, rDEN2/4Δ86, rDEN3Δ86, rDEN4Δ86, (114) rDEN1/2Δ86, rDEN2/4Δ86, rDEN3Δ86, rDEN4/1Δ86, (115) rDEN1/2Δ86, rDEN2/4Δ86, rDEN3Δ86, rDEN4/2Δ86, (116) rDEN1/2Δ86, rDEN2/4Δ86, rDEN3Δ86, rDEN4/3Δ86, (117) rDEN1/2Δ86, rDEN2/4Δ86, rDEN3/1Δ86, rDEN4Δ86, (118) rDEN1/2Δ86, rDEN2/4Δ86, rDEN3/1Δ86, rDEN4/1Δ86, (119) rDEN1/2Δ86, rDEN2/4Δ86, rDEN3/1Δ86, rDEN4/2Δ86, (120) rDEN1/2Δ86, rDEN2/4Δ86, rDEN3/1Δ86, rDEN4/3Δ86, (121) rDEN1/2Δ86, rDEN2/4Δ86, rDEN3/2Δ86, rDEN4Δ86, (122) rDEN1/2Δ86, rDEN2/4Δ86, rDEN3/2Δ86, rDEN4/1Δ86, (123) rDEN1/2Δ86, rDEN2/4Δ86, rDEN3/2Δ86, rDEN4/2Δ86, (124) rDEN1/2Δ86, rDEN2/4Δ86, rDEN3/2Δ86, rDEN4/3Δ86, (125) rDEN1/2Δ86, rDEN2/4Δ86, rDEN3/4Δ86, rDEN4Δ86, (126) rDEN1/2Δ86, rDEN2/4Δ86, rDEN3/4Δ86, rDEN4/1Δ86, (127) rDEN1/2Δ86, rDEN2/4Δ86, rDEN3/4Δ86, rDEN4/2Δ86, (128) rDEN1/2Δ86, rDEN2/4Δ86, rDEN3/4Δ86, rDEN4/3Δ86, (129) rDEN1/3Δ86, rDEN2Δ86, rDEN3Δ86, rDEN4Δ86, (130) rDEN1/3Δ86, rDEN2Δ86, rDEN3Δ86, rDEN4/1Δ86, (131) rDEN1/3Δ86, rDEN2Δ86, rDEN3Δ86, rDEN4/2Δ86, (132) rDEN1/3Δ86, rDEN2Δ86, rDEN3Δ86, rDEN4/3Δ86, (133) rDEN1/3Δ86, rDEN2Δ86, rDEN3/1Δ86, rDEN4Δ86, (134) rDEN1/3Δ86, rDEN2Δ86, rDEN3/1Δ86, rDEN4/1Δ86, (135) rDEN1/3Δ86, rDEN2Δ86, rDEN3/1Δ86, rDEN4/2Δ86, (136) rDEN1/3Δ86, rDEN2Δ86, rDEN3/1Δ86, rDEN4/3Δ86, (137) rDEN1/3Δ86, rDEN2Δ86, rDEN3/2Δ86, rDEN4Δ86, (138) rDEN1/3Δ86, rDEN2Δ86, rDEN3/2Δ86, rDEN4/1Δ86, (139) rDEN1/3Δ86, rDEN2Δ86, rDEN3/2Δ86, rDEN4/2Δ86, (140) rDEN1/3Δ86, rDEN2Δ86, rDEN3/2Δ86, rDEN4/3Δ86, (141) rDEN1/3Δ86, rDEN2Δ86, rDEN3/4Δ86, rDEN4Δ86, (142) rDEN1/3Δ86, rDEN2Δ86, rDEN3/4Δ86, rDEN4/1Δ86, (143) rDEN1/3Δ86, rDEN2Δ86, rDEN3/4Δ86, rDEN4/2Δ86, (144) rDEN1/3Δ86, rDEN2Δ86, rDEN3/4Δ86, rDEN4/3Δ86, (145) rDEN1/3Δ86, rDEN2/1Δ86, rDEN3Δ86, rDEN4Δ86, (146) rDEN1/3Δ86, rDEN2/1Δ86, rDEN3Δ86, rDEN4/1Δ86, (147) rDEN1/3Δ86, rDEN2/1Δ86, rDEN3Δ86, rDEN4/2Δ86, (148) rDEN1/3Δ86, rDEN2/1Δ86, rDEN3Δ86, rDEN4/3Δ86, (149) rDEN1/3Δ86, rDEN2/1Δ86, rDEN3/1Δ86, rDEN4Δ86, (150) rDEN1/3Δ86, rDEN2/1Δ86, rDEN3/1Δ86, rDEN4/1Δ86, (151) rDEN1/3Δ86, rDEN2/1Δ86, rDEN3/1Δ86, rDEN4/2Δ86, (152) rDEN1/3Δ86, rDEN2/1Δ86, rDEN3/1Δ86, rDEN4/3Δ86, (153) rDEN1/3Δ86, rDEN2/1Δ86, rDEN3/2Δ86, rDEN4Δ86, (154) rDEN1/3Δ86, rDEN2/1Δ86, rDEN3/2Δ86, rDEN4/1Δ86, (155) rDEN1/3Δ86, rDEN2/1Δ86, rDEN3/2Δ86, rDEN4/2Δ86, (156) rDEN1/3Δ86, rDEN2/1Δ86, rDEN3/2Δ86, rDEN4/3Δ86, (157) rDEN1/3Δ86, rDEN2/1Δ86, rDEN3/4Δ86, rDEN4Δ86, (158) rDEN1/3Δ86, rDEN2/1Δ86, rDEN3/4Δ86, rDEN4/1Δ86, (159) rDEN1/3Δ86, rDEN2/1Δ86, rDEN3/4Δ86, rDEN4/2Δ86, (160) rDEN1/3Δ86, rDEN2/1Δ86, rDEN3/4Δ86, rDEN4/3Δ86, (161) rDEN1/3Δ86, rDEN2/3Δ86, rDEN3Δ86, rDEN4Δ86, (162) rDEN1/3Δ86, rDEN2/3Δ86, rDEN3Δ86, rDEN4/1Δ86, (163) rDEN1/3Δ86, rDEN2/3Δ86, rDEN3Δ86, rDEN4/2Δ86, (164) rDEN1/3Δ86, rDEN2/3Δ86, rDEN3Δ86, rDEN4/3Δ86, (165) rDEN1/3Δ86, rDEN2/3Δ86, rDEN3/1Δ86, rDEN4Δ86, (166) rDEN1/3Δ86, rDEN2/3Δ86, rDEN3/1Δ86, rDEN4/1Δ86, (167) rDEN1/3Δ86, rDEN2/3Δ86, rDEN3/1Δ86, rDEN4/2Δ86, (168) rDEN1/3Δ86, rDEN2/3Δ86, rDEN3/1Δ86, rDEN4/3Δ86, (169) rDEN1/3Δ86, rDEN2/3Δ86, rDEN3/2Δ86, rDEN4Δ86, (170) rDEN1/3Δ86, rDEN2/3Δ86, rDEN3/2Δ86, rDEN4/1Δ86, (171) rDEN1/3Δ86, rDEN2/3Δ86, rDEN3/2Δ86, rDEN4/2Δ86, (172) rDEN1/3Δ86, rDEN2/3Δ86, rDEN3/2Δ86, rDEN4/3Δ86, (173) rDEN1/3Δ86, rDEN2/3Δ86, rDEN3/4Δ86, rDEN4Δ86, (174) rDEN1/3Δ86, rDEN2/3Δ86, rDEN3/4Δ86, rDEN4/1Δ86, (175) rDEN1/3Δ86, rDEN2/3Δ86, rDEN3/4Δ86, rDEN4/2Δ86, (176) rDEN1/3Δ86, rDEN2/3Δ86, rDEN3/4Δ86, rDEN4/3Δ86, (177) rDEN1/3Δ86, rDEN2/4Δ86, rDEN3Δ86, rDEN4Δ86, (178) rDEN1/3Δ86, rDEN2/4Δ86, rDEN3Δ86, rDEN4/1Δ86, (179) rDEN1/3Δ86, rDEN2/4Δ86, rDEN3Δ86, rDEN4/2Δ86, (180) rDEN1/3Δ86, rDEN2/4Δ86, rDEN3Δ86, rDEN4/3Δ86, (181) rDEN1/3Δ86, rDEN2/4Δ86, rDEN3/1Δ86, rDEN4Δ86, (182) rDEN1/3Δ86, rDEN2/4Δ86, rDEN3/1Δ86, rDEN4/1Δ86, (183) rDEN1/3Δ86, rDEN2/4Δ86, rDEN3/1Δ86, rDEN4/2Δ86, (184) rDEN1/3Δ86, rDEN2/4Δ86, rDEN3/1Δ86, rDEN4/3Δ86, (185) rDEN1/3Δ86, rDEN2/4Δ86, rDEN3/2Δ86, rDEN4Δ86, (186) rDEN1/3Δ86, rDEN2/4Δ86, rDEN3/2Δ86, rDEN4/1Δ86, (187) rDEN1/3Δ86, rDEN2/4Δ86, rDEN3/2Δ86, rDEN4/2Δ86, (188) rDEN1/3Δ86, rDEN2/4Δ86, rDEN3/2Δ86, rDEN4/3Δ86, (189) rDEN1/3Δ86, rDEN2/4Δ86, rDEN3/4Δ86, rDEN4Δ86, (190) rDEN1/3Δ86, rDEN2/4Δ86, rDEN3/4Δ86, rDEN4/1Δ86, (191) rDEN1/3Δ86, rDEN2/4Δ86, rDEN3/4Δ86, rDEN4/2Δ86, (192) rDEN1/3Δ86, rDEN2/4Δ86, rDEN3/4Δ86, rDEN4/3Δ86, (193) rDEN1/4Δ86, rDEN2Δ86, rDEN3Δ86, rDEN4Δ86, (194) rDEN1/4Δ86, rDEN2Δ86, rDEN3Δ86, rDEN4/1Δ86, (195) rDEN1/4Δ86, rDEN2Δ86, rDEN3Δ86, rDEN4/2Δ86, (196) rDEN1/4Δ86, rDEN2Δ86, rDEN3Δ86, rDEN4/3Δ86, (197) rDEN1/4Δ86, rDEN2Δ86, rDEN3/1Δ86, rDEN4Δ86, (198) rDEN1/4Δ86, rDEN2Δ86, rDEN3/1Δ86, rDEN4/1Δ86, (199) rDEN1/4Δ86, rDEN2Δ86, rDEN3/1Δ86, rDEN4/2Δ86, (200) rDEN1/4Δ86, rDEN2Δ86, rDEN3/1Δ86, rDEN4/3Δ86, (201) rDEN1/4Δ86, rDEN2Δ86, rDEN3/2Δ86, rDEN4Δ86, (202) rDEN1/4Δ86, rDEN2Δ86, rDEN3/2Δ86, rDEN4/1Δ86, (203) rDEN1/4Δ86, rDEN2Δ86, rDEN3/2Δ86, rDEN4/2Δ86, (204) rDEN1/4Δ86, rDEN2Δ86, rDEN3/2Δ86, rDEN4/3Δ86, (205) rDEN1/4Δ86, rDEN2Δ86, rDEN3/4Δ86, rDEN4Δ86, (206) rDEN1/4Δ86, rDEN2Δ86, rDEN3/4Δ86, rDEN4/1Δ86, (207) rDEN1/4Δ86, rDEN2Δ86, rDEN3/4Δ86, rDEN4/2Δ86, (208) rDEN1/4Δ86, rDEN2Δ86, rDEN3/4Δ86, rDEN4/3Δ86, (209) rDEN1/4Δ86, rDEN2/1Δ86, rDEN3Δ86, rDEN4Δ86, (210) rDEN1/4Δ86, rDEN2/1Δ86, rDEN3Δ86, rDEN4/1Δ86, (211) rDEN1/4Δ86, rDEN2/1Δ86, rDEN3Δ86, rDEN4/2Δ86, (212) rDEN1/4Δ86, rDEN2/1Δ86, rDEN3Δ86, rDEN4/3Δ86, (213) rDEN1/4Δ86, rDEN2/1Δ86, rDEN3/1Δ86, rDEN4Δ86, (214) rDEN1/4Δ86, rDEN2/1Δ86, rDEN3/1Δ86, rDEN4/1Δ86, (215) rDEN1/4Δ86, rDEN2/1Δ86, rDEN3/1Δ86, rDEN4/2Δ86, (216) rDEN1/4Δ86, rDEN2/1Δ86, rDEN3/1Δ86, rDEN4/3Δ86, (217) rDEN1/4Δ86, rDEN2/1Δ86, rDEN3/2Δ86, rDEN4Δ86, (218) rDEN1/4Δ86, rDEN2/1Δ86, rDEN3/2Δ86, rDEN4/1Δ86, (219) rDEN1/4Δ86, rDEN2/1Δ86, rDEN3/2Δ86, rDEN4/2Δ86, (220) rDEN1/4Δ86, rDEN2/1Δ86, rDEN3/2Δ86, rDEN4/3Δ86, (221) rDEN1/4Δ86, rDEN2/1Δ86, rDEN3/4Δ86, rDEN4Δ86, (222) rDEN1/4Δ86, rDEN2/1Δ86, rDEN3/4Δ86, rDEN4/1Δ86, (223) rDEN1/4Δ86, rDEN2/1Δ86, rDEN3/4Δ86, rDEN4/2Δ86, (224) rDEN1/4Δ86, rDEN2/1Δ86, rDEN3/4Δ86, rDEN4/3Δ86, (225) rDEN1/4Δ86, rDEN2/3Δ86, rDEN3Δ86, rDEN4Δ86, (226) rDEN1/4Δ86, rDEN2/3Δ86, rDEN3Δ86, rDEN4/1Δ86, (227) rDEN1/4Δ86, rDEN2/3Δ86, rDEN3Δ86, rDEN4/2Δ86, (228) rDEN1/4Δ86, rDEN2/3Δ86, rDEN3Δ86, rDEN4/3Δ86, (229) rDEN1/4Δ86, rDEN2/3Δ86, rDEN3/1Δ86, rDEN4Δ86, (230) rDEN1/4Δ86, rDEN2/3Δ86, rDEN3/1Δ86, rDEN4/1Δ86, (231) rDEN1/4Δ86, rDEN2/3Δ86, rDEN3/1Δ86, rDEN4/2Δ86, (232) rDEN1/4Δ86, rDEN2/3Δ86, rDEN3/1Δ86, rDEN4/3Δ86, (233) rDEN1/4Δ86, rDEN2/3Δ86, rDEN3/2Δ86, rDEN4Δ86, (234) rDEN1/4Δ86, rDEN2/3Δ86, rDEN3/2Δ86, rDEN4/1Δ86, (235) rDEN1/4Δ86, rDEN2/3Δ86, rDEN3/2Δ86, rDEN4/2Δ86, (236) rDEN1/4Δ86, rDEN2/3Δ86, rDEN3/2Δ86, rDEN4/3Δ86, (237) rDEN1/4Δ86, rDEN2/3Δ86, rDEN3/4Δ86, rDEN4Δ86, (238) rDEN1/4Δ86, rDEN2/3Δ86, rDEN3/4Δ86, rDEN4/1Δ86, (239) rDEN1/4Δ86, rDEN2/3Δ86, rDEN3/4Δ86, rDEN4/2Δ86, (240) rDEN1/4Δ86, rDEN2/3Δ86, rDEN3/4Δ86, rDEN4/3Δ86, (241) rDEN1/4Δ86, rDEN2/4Δ86, rDEN3Δ86, rDEN4Δ86, (242) rDEN1/4Δ86, rDEN2/4Δ86, rDEN3Δ86, rDEN4/1Δ86, (243) rDEN1/4Δ86, rDEN2/4Δ86, rDEN3Δ86, rDEN4/2Δ86, (244) rDEN1/4Δ86, rDEN2/4Δ86, rDEN3Δ86, rDEN4/3Δ86, (245) rDEN1/4Δ86, rDEN2/4Δ86, rDEN3/1Δ86, rDEN4Δ86, (246) rDEN1/4Δ86, rDEN2/4Δ86, rDEN3/1Δ86, rDEN4/1Δ86, (247) rDEN1/4Δ86, rDEN2/4Δ86, rDEN3/1Δ86, rDEN4/2Δ86, (248) rDEN1/4Δ86, rDEN2/4Δ86, rDEN3/1Δ86, rDEN4/3Δ86, (249) rDEN1/4Δ86, rDEN2/4Δ86, rDEN3/2Δ86, rDEN4Δ86, (250) rDEN1/4Δ86, rDEN2/4Δ86, rDEN3/2Δ86, rDEN4/1Δ86, (251) rDEN1/4Δ86, rDEN2/4Δ86, rDEN3/2Δ86, rDEN4/2Δ86, (252) rDEN1/4Δ86, rDEN2/4Δ86, rDEN3/2Δ86, rDEN4/3Δ86, (253) rDEN1/4Δ86, rDEN2/4Δ86, rDEN3/4Δ86, rDEN4Δ86, (254) rDEN1/4Δ86, rDEN2/4Δ86, rDEN3/4Δ86, rDEN4/1Δ86, (255) rDEN1/4Δ86, rDEN2/4Δ86, rDEN3/4Δ86, rDEN4/2Δ86, and (256) rDEN1/4Δ86, rDEN2/4Δ86, rDEN3/4Δ86, rDEN4/3Δ86. In another embodiment, the fifth attenuated virus of the pentavalent immunogenic composition of any of the combinations of the first, second, third and four attenuated viruses described above is a Zika chimera of the present disclosure, as described in greater detail above. In an embodiment, each of the attenuated viruses comprises the same dengue backbone. In another embodiment, the fifth attenuated virus comprises a different dengue virus backbone than the first, second, third, and fourth attenuated viruses.

In other embodiments, the first, second, third and four attenuated viruses are selected independently from the first, second, third and four attenuated viruses articulated in paragraphs [0150] through [0153], and the fifth attenuated virus is an attenuated ZIKV or chimeric ZIKV of the present disclosure, as described in greater detail above. For example, in an embodiment, the first attenuate virus is rDEN1Δ30 (from paragraph [0148]), the second attenuated virus is rDEN2/4Δ30 (from paragraph [0148]), the third attenuated virus is rDEN3Δ30/31 (from paragraph [0150]), and the fourth attenuated virus is rDEN4Δ30 (from paragraph [0148]). In an embodiment, the first attenuate virus is rDEN1Δ30, the second attenuated virus is rDEN2/4Δ30, the third attenuated virus is rDEN3Δ30/31, the fourth attenuated virus is rDEN4Δ30, and the fifth attenuated virus is ZIKV/DEN2Δ30 or ZIKV/DEN3Δ30.

In certain embodiments, each of the attenuated viruses includes the same attenuating deletion and/or mutation. In a particular embodiment, the deletion can be a deletion in nucleotide sequence of the 3′ untranslated region. For example, the deletion is selected from the group consisting of: a Δ30 deletion, a Δ31 deletion, a Δ30/31 deletion, and a Δ86 deletion. In another embodiment, the mutation is at nucleotide 4891 of the NS3 gene and/or at nucleotide 4995 of the NS3 gene. It should be noted that, that the same attenuating deletion and/or mutation utilized in each of the attenuated viruses can be on at least two different dengue backbones (i.e., each of the attenuated viruses can have the same and/or different dengue backbones that contain the same type of attenuating deletion and/or mutation).

Immunogenic Dengue Chimeras and Methods for their Preparation

Immunogenic dengue chimeras and methods for preparing the dengue chimeras are provided herein. The immunogenic dengue chimeras are useful with the Zika chimeras of the present disclosure, alone or in combination, in a pharmaceutically acceptable carrier as immunogenic compositions to minimize, inhibit, or immunize individuals and animals against infection by dengue virus and ZIKV.

The dengue chimeras comprise nucleotide sequences encoding the immunogenicity of a dengue virus of one serotype and further nucleotide sequences selected from the backbone of a dengue virus of a different serotype. These chimeras can be used to induce an immunogenic response against dengue virus.

In another embodiment, the preferred dengue chimera is a nucleic acid chimera comprising a first nucleotide sequence encoding at least one structural protein from a dengue virus of a first serotype, and a second nucleotide sequence encoding nonstructural proteins from a dengue virus of a second serotype different from the first. In another embodiment the dengue virus of the second serotype is DEN4. In another embodiment, the structural protein can be the C protein of a dengue virus of the first serotype, the prM protein of a dengue virus of the first serotype, the E protein of a dengue virus of the first serotype, or any combination thereof.

Furthermore, one of skill in the art will recognize that individual substitutions, deletions or additions in the amino acid sequence, or in the nucleotide sequence encoding for the amino acids, which alter, add or delete a single amino acid or a small percentage of amino acids (typically less than 5%, more typically less than 1%) in an encoded sequence are conservatively modified variations, wherein the alterations result in the substitution of an amino acid with a chemically similar amino acid. Conservative substitution tables providing functionally similar amino acids are well known in the art. The following six groups each contain amino acids that are conservative substitutions for one another: [0030] 1) Alanine (A), Serine (S), Threonine (T); [0031] 2) Aspartic acid (D), Glutamic acid (E); [0032] 3) Asparagine (N), Glutamine (Q); [0033] 4) Arginine (R), Lysine (K); [0034] 5) Isoleucine (I), Leucine (L), Methionine (M), Valine (V); and [0035] 6) Phenylalanine (F), Tyrosine (Y), Tryptophan (W).

As used herein, the terms “dengue chimera” and “chimeric dengue virus” means an infectious construct of the invention comprising nucleotide sequences encoding the immunogenicity of a dengue virus of one serotype and further nucleotide sequences derived from the backbone of a dengue virus of a different serotype.

As used herein, “infectious construct” indicates a virus, a viral construct, a viral chimera, a nucleic acid derived from a virus or any portion thereof, which may be used to infect a cell.

As used herein, “dengue nucleic acid chimera” means a construct of the invention comprising nucleic acid comprising nucleotide sequences encoding the immunogenicity of a dengue virus of one serotype and further nucleotide sequences derived from the backbone of a dengue virus of a different serotype. Correspondingly, any chimeric virus or virus chimera of the invention is to be recognized as an example of a nucleic acid chimera.

The structural and nonstructural proteins of the invention are to be understood to include any protein comprising or any gene encoding the sequence of the complete protein, an epitope of the protein, or any fragment comprising, for example, three or more amino acid residues thereof.

Dengue Chimeras

The dengue chimeras of the invention are constructs formed by fusing structural protein genes from a dengue virus of one serotype, e.g. DEN1, DEN2, DEN3, or DEN4, with non-structural protein genes from a dengue virus of a different serotype, e.g., DEN1, DEN2, DEN3, or DEN4.

The attenuated, immunogenic dengue chimeras provided herein contain one or more of the structural protein genes, or antigenic portions thereof, of the dengue virus of one serotype against which immunogenicity is to be conferred, and the nonstructural protein genes of a dengue virus of a different serotype.

The dengue chimeras contain a dengue virus genome of one serotype as the backbone, in which the structural protein gene(s) encoding C, prM, or E protein(s) of the dengue genome, or combinations thereof, are replaced with the corresponding structural protein gene(s) from a dengue virus of a different serotype that is to be protected against. The resulting viral dengue chimera has the properties, by virtue of being chimerized with a dengue virus of another serotype, of attenuation and is therefore reduced in virulence, but expresses antigenic epitopes of the structural gene products and is therefore immunogenic.

The genome of any dengue virus can be used as the backbone in the attenuated chimeras (dengue and Zika) described herein. The backbone can contain mutations that contribute to the attenuation phenotype of the dengue virus or that facilitate replication in the cell substrate used for manufacture, e.g., Vero cells. The mutations can be in the nucleotide sequence encoding nonstructural proteins, the 5′ untranslated region or the 3′ untranslated region, as described above with regard to the Zika chimera. The backbone can also contain further mutations to maintain the stability of the attenuation phenotype and to reduce the possibility that the attenuated virus or chimera might revert back to the virulent wild-type virus. For example, a first mutation in the 3′ untranslated region and a second mutation in the 5′ untranslated region will provide additional attenuation phenotype stability, if desired.

Such mutations may be achieved by site-directed mutagenesis using techniques known to those skilled in the art. It will be understood by those skilled in the art that the virulence screening assays, as described herein and as are well known in the art, can be used to distinguish between virulent and attenuated backbone structures.

Construction of Dengue Chimeras

The dengue virus chimeras described herein can be produced by substituting at least one of the structural protein genes of the dengue virus of one serotype against which immunity is desired into a dengue virus genome backbone of a different serotype, using recombinant engineering techniques well known to those skilled in the art, namely, removing a designated dengue virus gene of one serotype and replacing it with the desired corresponding gene of dengue virus of a different serotype. Alternatively, using the sequences provided in GenBank, the nucleic acid molecules encoding the dengue proteins may be synthesized using known nucleic acid synthesis techniques and inserted into an appropriate vector. Attenuated, immunogenic virus is therefore produced using recombinant engineering techniques known to those skilled in the art.

As mentioned above, the gene to be inserted into the backbone encodes a dengue structural protein of one serotype. The dengue gene of a different serotype to be inserted is a gene encoding a C protein, a prM protein and/or an E protein. The sequence inserted into the dengue virus backbone can encode both the prM and E structural proteins of the other serotype. The sequence inserted into the dengue virus backbone can encode the C, prM and E structural proteins of the other serotype. The dengue virus backbone is the DEN1, DEN2, DEN3, or DEN4 virus genome, or an attenuated dengue virus genome of any of these serotypes, and includes the substituted gene(s) that encode the C, prM and/or E structural protein(s) of a dengue virus of a different serotype, or the substituted gene(s) that encode the prM and/or E structural protein(s) of a dengue virus of a different serotype.

Suitable chimeric viruses or nucleic acid chimeras containing nucleotide sequences encoding structural proteins of dengue virus of any of the serotypes can be evaluated for usefulness as vaccines by screening them for phenotypic markers of attenuation that indicate reduction in virulence with retention of immunogenicity. Antigenicity and immunogenicity can be evaluated using in vitro or in vivo reactivity with dengue antibodies or immunoreactive serum using routine screening procedures known to those skilled in the art.

Dengue and Zika Vaccines

The preferred dengue and Zika chimeric viruses and nucleic acid chimeras provide live, attenuated viruses useful as immunogens or vaccines. In a preferred embodiment, the chimeras exhibit high immunogenicity while at the same time not producing dangerous pathogenic or lethal effects.

The dengue chimeric viruses or nucleic acid chimeras of the present disclosure can comprise the structural genes of a dengue virus of one serotype in a wild-type or an attenuated dengue virus backbone of a different serotype, while the Zika-dengue chimeric viruses or nucleic acid chimeras of the present disclosure comprise the structural genes of a ZIKV in a wilde-type or an attenuated dengue virus backbone. For example, the dengue chimera may express the structural protein genes of a dengue virus of one serotype in either of a dengue virus or an attenuated dengue virus background of a different serotype.

The strategy described herein of using a genetic background that contains nonstructural regions of a dengue virus genome of one serotype, and, by chimerization, the properties of attenuation, to express the structural protein genes of a dengue virus of a different serotype and a ZIKV has led to the development of live, attenuated Zika, dengue vaccine candidates that express structural protein genes of desired immunogenicity. Thus, vaccine candidates for control of dengue and Zika pathogens can be designed.

Viruses used in the chimeras described herein are typically grown using techniques known in the art. Virus plaque or focus forming unit (FFU) titrations are then performed and plaques or FFU are counted in order to assess the viability, titer and phenotypic characteristics of the virus grown in cell culture. Wild type viruses are mutagenized to derive attenuated candidate starting materials.

Chimeric infectious clones are constructed from various dengue serotypes. The cloning of virus-specific cDNA fragments can also be accomplished, if desired. The cDNA fragments containing the structural protein or nonstructural protein genes are amplified by reverse transcriptase-polymerase chain reaction (RT-PCR) from dengue RNA with various primers. Amplified fragments are cloned into the cleavage sites of other intermediate clones. Intermediate, chimeric dengue clones are then sequenced to verify the sequence of the inserted dengue-specific cDNA.

Full genome-length chimeric plasmids constructed by inserting the structural or nonstructural protein gene region of dengue viruses into vectors are obtainable using recombinant techniques well known to those skilled in the art.

Method of Administration

The viral chimeras described herein are individually or jointly combined with a pharmaceutically acceptable carrier or vehicle for administration as an immunogen or vaccine to humans or animals. The terms “pharmaceutically acceptable carrier” or “pharmaceutically acceptable vehicle” are used herein to mean any composition or compound including, but not limited to, water or saline, a gel, salve, solvent, diluent, fluid ointment base, liposome, micelle, giant micelle, and the like, which is suitable for use in contact with living animal or human tissue without causing adverse physiological responses, and which does not interact with the other components of the composition in a deleterious manner.

The immunogenic or vaccine formulations may be conveniently presented in viral plaque forming unit (PFU) unit or focus forming unit (FFU) dosage form and prepared by using conventional pharmaceutical techniques. Such techniques include the step of bringing into association the active ingredient and the pharmaceutical carrier(s) or excipient(s). In general, the formulations are prepared by uniformly and intimately bringing into association the active ingredient with liquid carriers. Formulations suitable for parenteral administration include aqueous and non-aqueous sterile injection solutions which may contain anti-oxidants, buffers, bacteriostats and solutes which render the formulation isotonic with the blood of the intended recipient, and aqueous and non-aqueous sterile suspensions which may include suspending agents and thickening agents. The formulations may be presented in unit-dose or multi-dose containers, for example, sealed ampoules and vials, and may be stored in a freeze-dried (lyophilized) condition requiring only the addition of the sterile liquid carrier, for example, water for injections, immediately prior to use. Extemporaneous injection solutions and suspensions may be prepared from sterile powders, granules and tablets commonly used by one of ordinary skill in the art.

Preferred unit dosage formulations are those containing a dose or unit, or an appropriate fraction thereof, of the administered ingredient. It should be understood that in addition to the ingredients particularly mentioned above, the formulations of the present invention may include other agents commonly used by one of ordinary skill in the art.

The immunogenic or vaccine composition may be administered through different routes, such as oral or parenteral, including, but not limited to, buccal and sublingual, rectal, aerosol, nasal, intramuscular, subcutaneous, intradermal, and topical. The composition may be administered in different forms, including, but not limited to, solutions, emulsions and suspensions, microspheres, particles, microparticles, nanoparticles and liposomes. It is expected that from about 1 to about 5 doses may be required per immunization schedule. Initial doses may range from about 100 to about 100,000 PFU or FFU, with a preferred dosage range of about 500 to about 20,000 PFU or FFU, a more preferred dosage range of from about 750 to about 12,000 PFU or FFU and a most preferred dosage range of about 750 to about 4000 PFU or FFU. Booster injections may range in dosage from about 100 to about 20,000 PFU or FFU, with a preferred dosage range of about 500 to about 15,000, a more preferred dosage range of about 500 to about 10,000 PFU or FFU, and a most preferred dosage range of about 500 to about 5000 PFU or FFU. For example, the volume of administration will vary depending on the route of administration. Intramuscular injections may range in volume from about 0.1 ml to 1.0 ml.

The composition may be stored at temperatures of from about −100° C. to about 4° C. The composition may also be stored in a lyophilized state at different temperatures including room temperature. The composition may be sterilized through conventional means known to one of ordinary skill in the art. Such means include, but are not limited to, filtration. The composition may also be combined with bacteriostatic agents to inhibit bacterial growth.

Administration Schedule

The immunogenic or vaccine composition described herein may be administered to humans or domestic animals, such as horses or birds, especially individuals travelling to regions where ZIKV infection is present, and also to inhabitants of those regions. The optimal time for administration of the composition is about one to three months before the initial exposure to the ZIKV. However, the composition may also be administered after initial infection to ameliorate disease progression, or after initial infection to treat the disease.

Adjuvants

A variety of adjuvants known to one of ordinary skill in the art may be administered in conjunction with the chimeric virus in the immunogen or vaccine composition of this invention. Such adjuvants include, but are not limited to, the following: polymers, co-polymers such as polyoxyethylene-polyoxypropylene copolymers, including block co-polymers, polymer p 1005, Freund's complete adjuvant (for animals), Freund's incomplete adjuvant; sorbitan monooleate, squalene, CRL-8300 adjuvant, alum, QS 21, muramyl dipeptide, CpG oligonucleotide motifs and combinations of CpG oligonucleotide motifs, trehalose, bacterial extracts, including mycobacterial extracts, detoxified endotoxins, membrane lipids, or combinations thereof.

Nucleic Acid Sequences

Nucleic acid sequences of ZIKV and dengue virus are useful for designing nucleic acid probes and primers for the detection of ZIKV and dengue virus chimeras in a sample or specimen with high sensitivity and specificity. Probes or primers corresponding to ZIKV and dengue virus can be used to detect the presence of a vaccine virus. The nucleic acid and corresponding amino acid sequences are useful as laboratory tools to study the organisms and diseases and to develop therapies and treatments for the diseases.

Nucleic acid probes and primers selectively hybridize with nucleic acid molecules encoding ZIKV and dengue virus or complementary sequences thereof. By “selective” or “selectively” is meant a sequence which does not hybridize with other nucleic acids to prevent adequate detection of the ZIKV sequence and dengue virus sequence. Therefore, in the design of hybridizing nucleic acids, selectivity will depend upon the other components present in the sample. The hybridizing nucleic acid should have at least 70% complementarity with the segment of the nucleic acid to which it hybridizes. As used herein to describe nucleic acids, the term “selectively hybridizes” excludes the occasional randomly hybridizing nucleic acids, and thus has the same meaning as “specifically hybridizing.” The selectively hybridizing nucleic acid probes and primers of this invention can have at least 70%, 80%, 85%, 90%, 95%, 97%, 98% and 99% complementarity with the segment of the sequence to which it hybridizes, preferably 85% or more.

The present invention also contemplates sequences, probes and primers that selectively hybridize to the encoding nucleic acid or the complementary, or opposite, strand of the nucleic acid. Specific hybridization with nucleic acid can occur with minor modifications or substitutions in the nucleic acid, so long as functional species-species hybridization capability is maintained. By “probe” or “primer” is meant nucleic acid sequences that can be used as probes or primers for selective hybridization with complementary nucleic acid sequences for their detection or amplification, which probes or primers can vary in length from about 5 to about 100 nucleotides, or preferably from about 10 to about 50 nucleotides, or most preferably about 18 to about 24 nucleotides. Isolated nucleic acids are provided herein that selectively hybridize with the species-specific nucleic acids under stringent conditions and should have at least five nucleotides complementary to the sequence of interest as described in Molecular Cloning: A Laboratory Manual, 2^{nd} ed., Sambrook, Fritsch and Maniatis, Cold Spring Harbor Laboratory, Cold Spring Harbor, N.Y., 1989.

If used as primers, the composition preferably includes at least two nucleic acid molecules which hybridize to different regions of the target molecule so as to amplify a desired region. Depending on the length of the probe or primer, the target region can range between 70% complementary bases and full complementarity and still hybridize under stringent conditions. For example, for the purpose of detecting the presence of ZIKV and dengue virus, the degree of complementarity between the hybridizing nucleic acid (probe or primer) and the sequence to which it hybridizes is at least enough to distinguish hybridization with a nucleic acid from other organisms.

The nucleic acid sequences encoding ZIKV and dengue virus can be inserted into a vector, such as a plasmid, and recombinantly expressed in a living organism to produce recombinant ZIKV and dengue virus peptide and/or polypeptides.

The nucleic acid sequences of the invention include a diagnostic probe that serves to report the detection of a cDNA amplicon amplified from the viral genomic RNA template by using a reverse-transcription/polymerase chain reaction (RT-PCR), as well as forward and reverse amplimers that are designed to amplify the cDNA amplicon. In certain instances, one of the amplimers is designed to contain a vaccine virus-specific mutation at the 3′-terminal end of the amplimer, which effectively makes the test even more specific for the vaccine strain because extension of the primer at the target site, and consequently amplification, will occur only if the viral RNA template contains that specific mutation.

Automated PCR-based nucleic acid sequence detection systems and TaqMan assays (Applied Biosystems) are widely used. A more recently developed strategy for diagnostic genetic testing makes use of molecular beacons (Tyagi and Kramer 1996 Nature Biotechnology 14:303-308). Molecular beacon assays employ quencher and reporter dyes that differ from those used in the TaqMan assay. These and other detection systems may be used by one skilled in the art.

Flavivirus Chimera Production

As described herein, live attenuated flavivirus vaccines are developed using recombinant DNA technology. The techniques herein are facilitated by the conservation among flaviviruses of genome organization, number of viral proteins, replicative strategy, gene expression, virion structure and morphogenesis. All flaviviruses have a positive sense non-segmented RNA genome that encodes a single long polyprotein that is processed to yield capsid (C), premembrane (prM) and envelope glycoprotein (E) structural proteins followed by nonstructural proteins NS1, NS2A, NS2B, NS3, NS4A, NS4B, and NS5 in that order. Due to these shared properties viable chimeric viruses are produced by replacing the genes for the viral structural proteins in a full-length infectious cDNA clone of a flavivirus with the corresponding viral genes (in cDNA form) of another flavivirus. When tested, this strategy was successful for chimeras that contained the sequence for viral structural proteins prM and E of tick-borne encephalitis virus (TBEV) or tick-borne Langat virus (LGT), while all other sequences were derived from the full-length infectious cDNA of mosquito-borne dengue type 4 virus (DEN4). This indicated that viral structural proteins of a disparate flavivirus, TBEV or LGT, could function in the context of cis-acting 5′ and 3′ sequences and nonstructural proteins of DEN4. Significantly, both chimeras proved to be highly attenuated in mice with respect to peripheral virulence, namely, the ability of a virus to spread to the CNS from a peripheral site of inoculation and cause encephalitis. Nonetheless, the chimeras proved to be immunogenic and able to induce resistance in mice against challenge with TBEV or LGT. It appeared that a favorable balance between reduction in virus replication in vivo (attenuation) and induction of protective immunity had been achieved. This is interpreted to mean that tick-borne flavivirus prM and E can interact in the context of DEN4 nonstructural proteins and cis-acting 5′ and 3′ sequences at a level sufficient for infectivity and induction of immunity but not sufficient for full expression of virulence that requires a high level of replication in vivo and ability to spread into the CNS. Recently, a West Nile virus and dengue virus chimeras containing structural proteins from the West Nile virus and an attenuated dengue virus backbone was shown to be effective as immunogens or vaccines. See U.S. Patent Application Publication 2005/0100886A1, which is incorporated herein by reference.

Further Attenuation of Zika/Dengue Chimeras by Introduction of Additional Mutations in the Genes for the Non-Structural Proteins of Dengue virus that Serve as a Component of these Vaccine Candidates. An increase in the level of attenuation of the candidate vaccine Zika/DEN or Zika/DEN-Δ30 chimera (serotype 1, 2, 3, or 4) is effectuated by adding one or more attenuation mutations to the dengue component of the chimeras. For example, a large set of mutations that attenuate DEN4 in mice have been identified in the part of the DEN4 genome included in the WN/DEN4 chimeric viruses described in U.S. Patent Application Publication 2005/0100886 A1. Members from this set of attenuating mutations can be introduced in the Zika/Dengue (serotype 1, 2, 3, or 4) chimeric virus to further attenuate these viruses. It might be necessary to further attenuate the Zika/DEN4 virus. The feasibility of this approach to achieve further attenuation is exemplified by introducing a viable mutation that specifies a temperature sensitive phenotype as well as a phenotype of growth restriction in suckling mouse brain into the non-structural protein 3 (NS3) of the Dengue component of the Zika/Dengue chimera. Mutation 4891 (isoleucine>threonine) had previously been identified at nucleotide 4891 of the NS3 gene of DEN4. Mutation 4891 specified two desirable phenotypes, i.e., temperature sensitivity and growth restriction in brain tissue. Similarly, mutation 4995 (serine >proline), also in NS3, specified the same two desirable phenotypes. The 4891 and 4995 mutations also increase replication fitness of DEN4 in Vero cells, i.e., they are Vero cell adaptation mutations. The wild type amino acid residue at DEN4 4891 (isoleucine) is conserved in DEN2 Tonga/74 and DEN3 Sleman/78, but not DEN1 West Pacific. The wild type amino acid residue at DEN4 4995 (serine) is conserved in DEN1 West Pacific, DEN2 Tonga/74, but not DEN3 Sleman. One or both of these mutations may also be included in a Zika/DEN1, 2, or 3 chimera. Thus, their inclusion in Zika/DEN4 virus is contemplated as achieving an increase in replication of the virus in Vero cells or the genetic stability of the mutation during manufacture in Vero cells.

Although prM and E proteins of distantly related tick-borne and mosquito-borne flaviviruses are highly divergent, it was demonstrated that these proteins could be interchanged in some instances without loss of virus viability. This approach has been used to create new chimeric flaviviruses.

In addition, viable tick-borne/DEN4 chimeras were constructed and recovered. In these instances, the tick-borne flavivirus parent was tick-borne encephalitis virus, a highly virulent virus, or Langat virus, a naturally attenuated tick-borne virus. Thus, the two components of these chimeras had disparate vector hosts, namely ticks, and in the case of DEN4, mosquitoes. Decreased efficiency of gene product interactions in the chimeras was thought to be the basis for the marked attenuation exhibited by these hybrid viruses. Nonetheless, although highly attenuated in mice, the TBEV/DEN4 and LGT/DEN4 chimeras were immunogenic and provided considerable protection against their parental tick-borne flavivirus.

Viable WN/DEN4 chimeras that contained a DEN4 genome whose genes for structural prM and E proteins were replaced by the corresponding genes of WN strain NY99 were also constructed. U.S. Patent Application Publication 2005/0100886 A1. The parent viruses of the WN/DEN4 chimeras are transmitted by mosquitoes. However, vector preference differs, Aedes for DEN4 and Culex for WN. Although highly attenuated, the WN/DEN4 chimeras stimulated a moderate to high level of serum neutralizing antibodies against WN NY99. There was a strong correlation between the level of neutralizing antibodies to WN induced by immunization and resistance to subsequent lethal WN challenge.

### EXAMPLES

Construction of an Attenuated VIKV. An attenuated ZIKV is constructed that contains the MO mutation (rZIKVΔ30) or a ZIKV containing the 3′UTR from rDEN4Δ30 (rZIKV-3′D4Δ30).

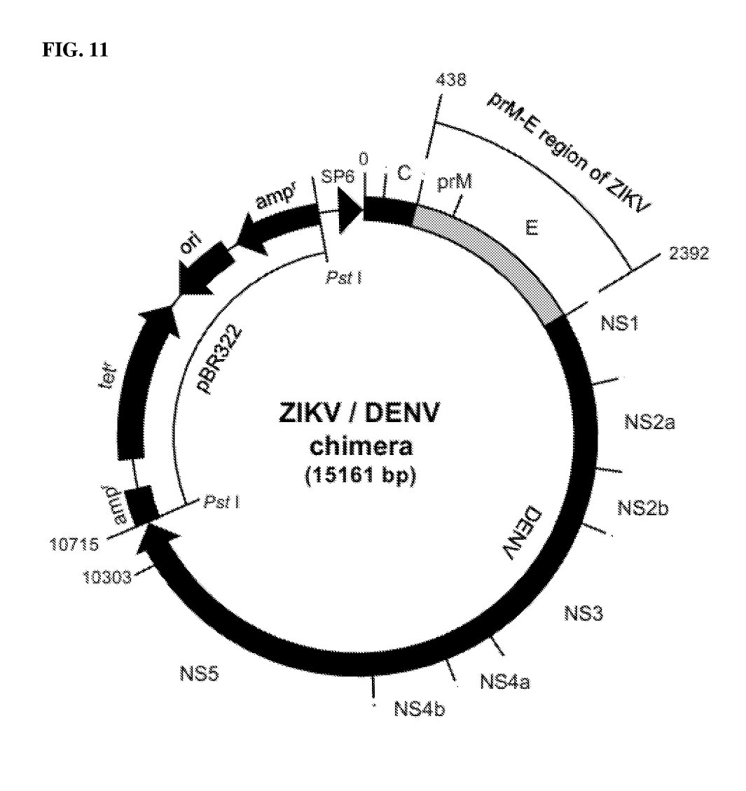

Construction of an Attenuated Chimeric ZIKV. An attenuated ZIKV is constructed by introducing ZIKV prM and E into DEN2Δ30, as shown in FIG. 11. The new chimeric virus would be termed rZIKV/D2Δ30. Similarly, ZIKV prM and E can be introduced into DEN3Δ30.

Manufacturing of a Multivalent Vaccine. A multivalent vaccine is constructed by combining attenuated viruses, such as, rDEN1Δ30, rDEN2/4Δ30, rDEN3Δ30/31, rDEN4Δ30, and rZIKV/D2Δ30.

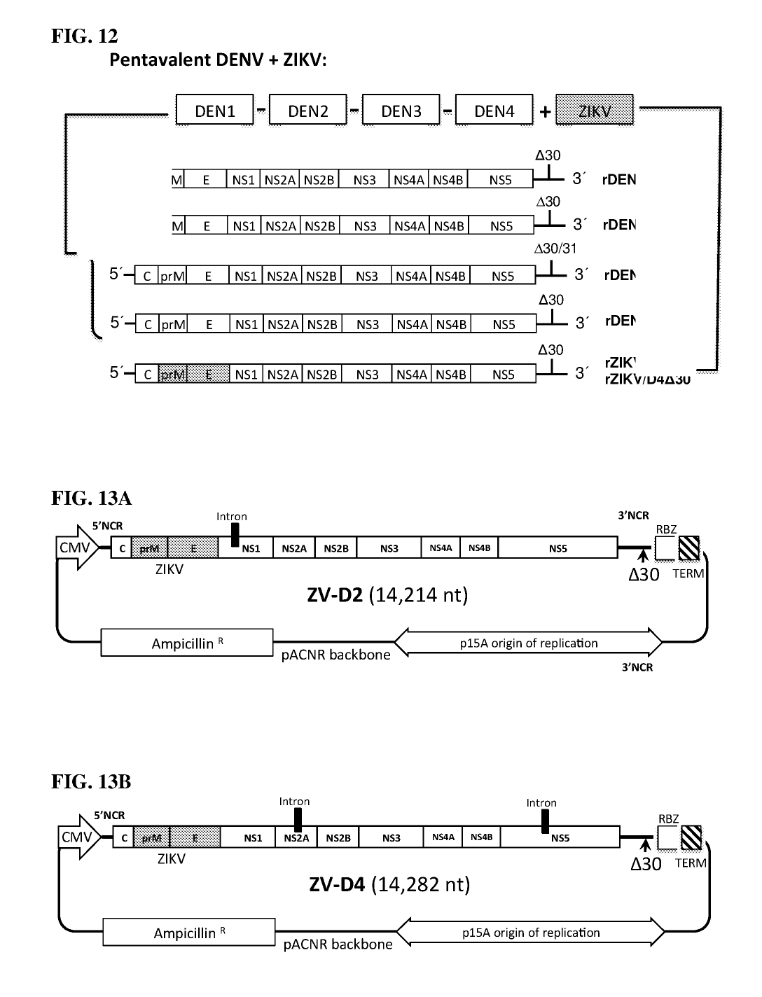

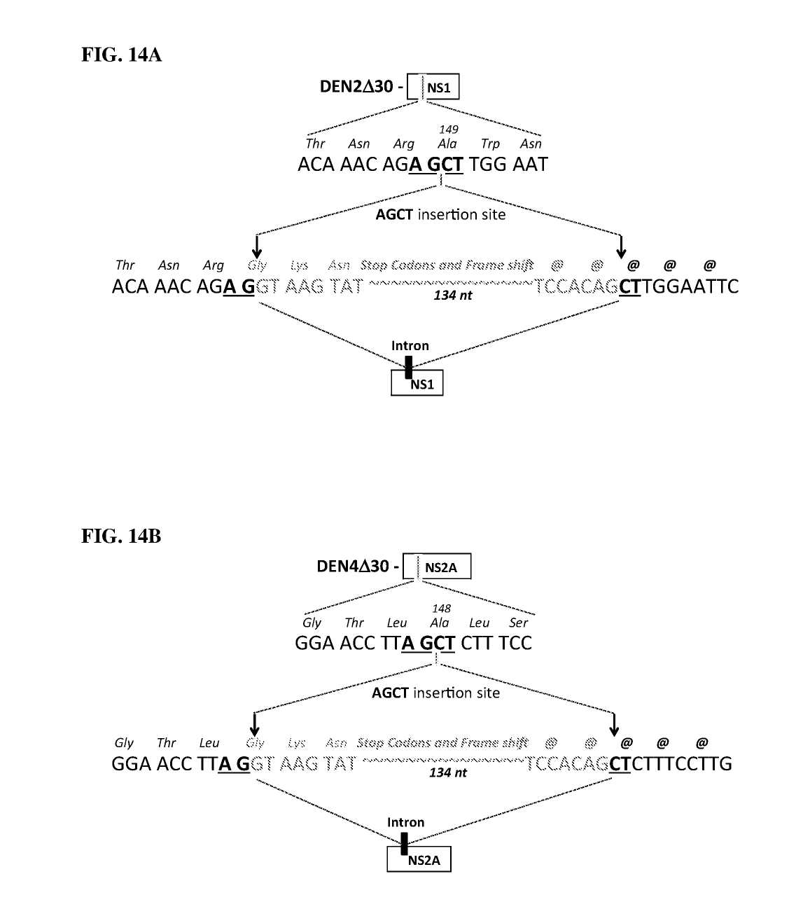

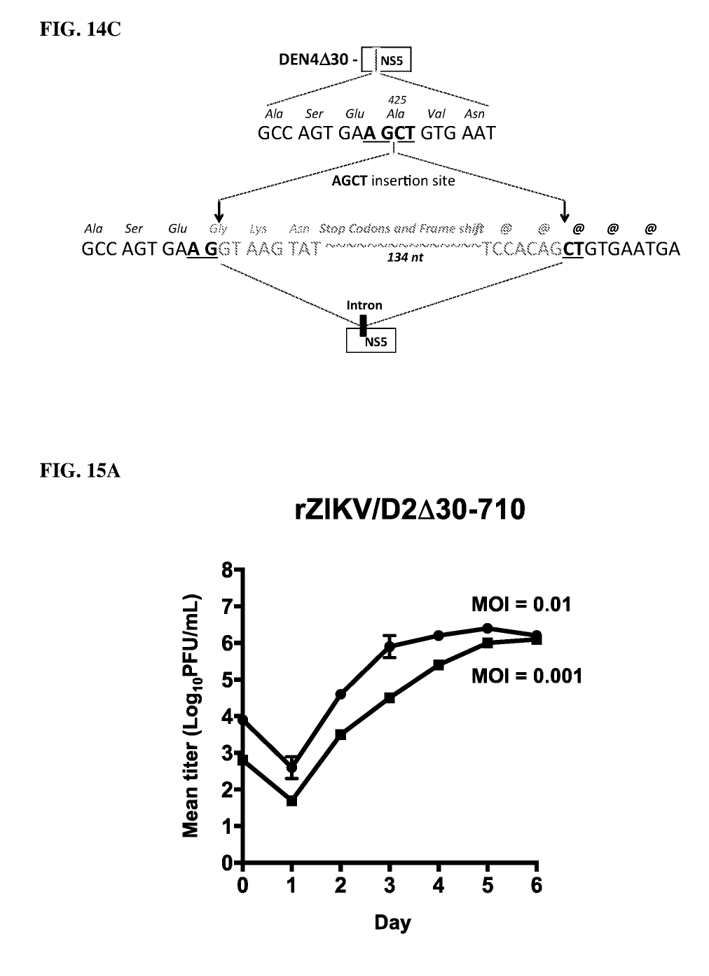

Pentavalent DENV-ZIKV Vaccine Development. The chimeric cDNA plasmids depicted in FIG. 12 replaces the prM and E gene regions of either DEN2Δ30 or DEN4Δ30 with those derived from ZIKV-Paraiba/2015 (Brazil). For stability in E. coli, it was determined that the viral open reading frame had to be disrupted by the insertion of intron sequences, which is shown in greater detail in FIGS. 14A-14C and discussed below. To recovery infectious viruses, Vero cells were transfected with the cDNA plasmid. Transcription proceeds from the CMV promoter sequence and is terminated by ribozyme (RBZ) and terminator (TERM) sequences to create the virus genome. Intron sequence is removed by the normal RNA splicing process. Plasmid maps for the DENV-2 and DENV-4 backgrounds are shown in FIGS. 13A and 13B.

As discussed above, intron sequences were required to stabilize the chimeric DENV-ZIKV constructs. The same standard intron sequence was used for each cDNA construct. ZV-D2 contains a single insertion at alanine codon 149 in the NS1 gene region (FIG. 14A). ZV-D4 contains two intron insertions located at alanine codon 148 in NS2A (FIG. 14B) and alanine codon 425 of NS5 (FIG. 14C).

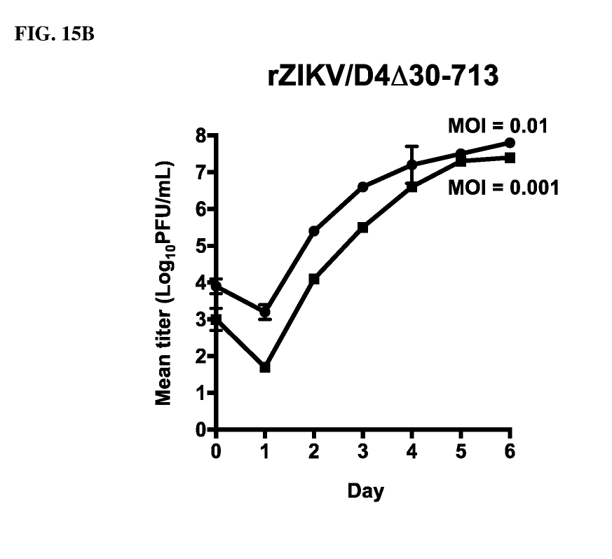

Evaluation of replication kinetics and peak titers of DENV-ZIKV chimeric viruses in tissue culture cells. Chimeric viruses rZIKV/D2Δ30-710 (DEN2Δ30 background) and rZIKV/D4Δ30-713 (DEN4Δ30 background) were produced in and recovered from Vero cells, biologocally cloned by two rounds of terminal dilution in Vero cells and then further amplified by passage in Vero cells to generate working seed stocks. The virus growth kinetics were evaluated at two different multiplicites of infection (MOI). As shown in FIG. 15A, both viruses replicate to above 6 log₁₀PFU/mL with titers peaking at about day 5. For both viruses, an MOI of 0.01 provided higher yields (FIG. 15B). Both viruses were deemed suitable for further evaluation and manufacture.

The level of attenuation is likely to be slightly different for the two backgrounds. Examination of attentuation will be determined by studies in non-human primates and Phase I evaluation in human subjects. Down-selection to a final vaccine candidate will be based on safety, infectivity, and immunogenity in human subjects.

Evaluation of the Attentuation of DENV-ZIKV Chimeric Viruses. Attenuation of the vaccine candidates was accessed via nonhuman primate studies, where virus replication and immunogenicity of the vaccine candidates was compared to wildtype ZIKV. Rhesus monkeys were inoculated subcutaneously with 10⁴ pfu (4.0 log ₁₀PFU) of either wildtype virus (ZIKV-SJRP/2016, ZIKV-Nicaragua/2016, or ZIKV-Paraiba/2015) or chimeric ZIKV vaccine candidates (rZIKVD2Δ30-710 or rZIKVD4Δ30-713). Serum was collected daily from 2 to 8 days post inoculation. The collected serum was assayed on Vero cells for infectious virus. Table 1 below shows the results of the viremia evaluation, and Table 2 shows the mean titers of the viremia evaluation.

The wildtype ZIKV replicated to titers of about 2 log₁₀ PFU/mL for 3-4 days. Replication of the chimeric vaccine candidates was below the level of detection (<0.7 log ₁₀PFU/mL), thereby confirming their attenuated phenotype compared to wildtype ZIKV. The detected attenuation is likely due to chimerization and the presence of the Δ30 mutation.

TABLE 2
Mean titer of wildtype ZIKV or chimeric ZIKV vaccine candidates.

| Mean titer (by day): | Day 2 | Day 3 | Day 4 | Day 5 | Day 6 |
|---|---|---|---|---|---|
| ZIKV-SJRP | 1.2 | 1.6 | 1.8 | 0.8 | <0.7 |
| ZIKV-Nicaragua | 1.9 | 2.3 | 1.9 | 1.0 | <0.7 |
| ZIKV-Paraiba | 1.7 | 2.0 | 1.8 | 1.3 | <0.7 |
| rZIKVD2Δ30 | <0.7 | <0.7 | <0.7 | <0.7 | <0.7 |
| rZIKVD4Δ30 | <0.7 | <0.7 | <0.7 | <0.7 | <0.7 |

### Specific Embodiments

According to an aspect, the present disclosure provides a Zika nucleic acid chimera that comprises: a first nucleotide sequence encoding at least one structural protein from a Zika virus (ZIKV), a second nucleotide sequence encoding at least one nonstructural protein from a first flavivirus, and a third nucleotide sequence of a 3′ untranslated region from a second flavivirus.

In any aspect or embodiment described herein, the first flavivirus is a dengue virus.

In any aspect or embodiment described herein, the first flavivirus is a ZIKV.

In any aspect or embodiment described herein, the second flavivirus is a dengue virus.

In any aspect or embodiment described herein, the second flavivirus is a ZIKV.

In any aspect or embodiment described herein, the dengue virus is a dengue serotype 1.

In any aspect or embodiment described herein, the dengue virus is a dengue serotype 2.

In any aspect or embodiment described herein, the dengue virus is a dengue serotype 3.

In any aspect or embodiment described herein, the dengue virus is a dengue serotype 4.

In any aspect or embodiment described herein, the 3′ untranslated region contains a deletion in the nucleotide sequence.

In any aspect or embodiment described herein, the deletion is selected from the group consisting of: a Δ30 deletion, a Δ31 deletion, a Δ30/31 deletion, and a Δ86 deletion.

In any aspect or embodiment described herein, the Zika nucleic acid chimera further comprises a mutation at nucleotide 4891 of the NS3 gene and/or at nucleotide 4995 of the NS3 gene.

In any aspect or embodiment described herein, the at least one structural protein is pre-membrane (prM), envelope (E), or both.

According to a further aspect, the present disclosure provides a pentavalent immunogenic composition that comprises: a first attenuated virus that is immunogenic against dengue serotype 1, a second attenuated virus that is immunogenic against dengue serotype 2, a third attenuated virus that is immunogenic against dengue serotype 3, a fourth attenuated virus that is immunogenic against dengue serotype 4, and a fifth attenuated virus that is immunogenic against ZIKV.

In any aspect or embodiment described herein, the fifth attenuated virus is the Zika nucleic acid chimera of the present disclosure.

In any aspect or embodiment described herein, each of the attenuated viruses includes the same attenuating deletion or mutation.

In any aspect or embodiment described herein, the deletion is a deletion in nucleotide sequence of the 3′ untranslated region.

In any aspect or embodiment described herein, the deletion is selected from the group consisting of: a Δ30 deletion, a Δ31 deletion, a Δ30/31 deletion, and a Δ86 deletion.

In any aspect or embodiment described herein, wherein the pentavalent immunogenic composition further comprising a mutation is at nucleotide 4891 of the NS3 gene and/or at nucleotide 4995 of the NS3 gene.

In any aspect or embodiment described herein, where the pentavalent immunogenic composition of the present disclosure further comprises an adjuvant.

According to an additional aspect, the present disclosure provides a multivalent immunogenic composition that comprises: at least one first attenuated virus that is immunogenic against a flavivirus, and a second attenuated virus that is immunogenic against ZIKV.

In any aspect or embodiment described herein, the flavivirus is at least one of dengue virus serotype 1, dengue virus serotype 2, dengue virus serotype 3, dengue virus serotype 4, West Nile virus, yellow fever virus, Japanese encephalitis virus, and tick-borne encephalitis virus, or a combination thereof.

In any aspect or embodiment described herein, the second attenuated virus is a Zika nucleic acid chimera of the present disclosure.

In any aspect or embodiment described herein, the second attenuated virus is a ZIKV comprising one or more attenuating mutations and/or deletions in the genome.

According to another aspect, the present disclosure provides a method of inducing an immune response in a subject. The method comprises administering an effective amount of the composition of the present disclosure.

According to yet a further aspect, the present disclosure provides a method of preventing or treating a ZIKV infection in a subject. The method comprises administering to the subject an effective amount of the Zika nucleic acid chimera of the present disclosure or an effective amount of the immunogenic composition of the present disclosure.

While the present invention has been described in some detail for purposes of clarity and understanding, one skilled in the art will appreciate that various changes in form and detail can be made without departing from the true scope of the invention. All figures, tables, appendices, patents, patent applications and publications, referred to above, are hereby incorporated by reference.

### REFERENCES

1. Bhamarapravati, N. and Sutee, Y. 2000 Vaccine 18:44-7

2. Blaney, et al. 2001 J Virol 75:9731-9740;

3. Blaney, et al. 2002 Virology 300:125-139;

4. Blaney et al., Vaccine (2008) 26, 817-828).

5. Blaney et al, BMC Infectious Diseases 2004, 4:39.

6. Bray, M., Men, R. & Lai, C.-J. 1996 J. Virol. 70:4162-4166;

7. Burke, D. S. & Monath, T. P. 2001 in Fields Virology, eds. Knipe, D. M. & Howley, P. M. Lippincott Williams and Wilkins, Philadelphia, 4-th ed., pp. 1043-1125; Hayes, C. G. 1989 in The Arboviruses: Epidemiology and Ecology, ed. Monath T. P. Boca Raton, F. L.: CRC Press, Volume V, pp. 59-88

8. Caufour, P. S. et al. 2001 Virus Res 79:1-14

9. Chambers, T. J. et al. 1999 J Virol 73:3095-3101;

10. M. Chastain, “National Institutes of Health: Zika Virus is a ‘Pandemic’”, Jan. 30, 2016, Breitbart online news source, http://www.breitbart.com/national-security/2016/01/30/zika-virus-re aches-pandemic-levels.

11. Durbin et al. 2001 Am J Trop Med 65:405-413

12. Guillot, S. et al. 2000 J Virol 74:8434-43

13. Guirakhoo, F. et al. 2000 J Virol 74:5477-5485;

14. Hanley, et al. 2002 J Virol 76:525-31

15. Huang, C. Y. et al. 2000 J Virol 74:3020-3028;

16. Lindenbach, B. D. & Rice, C. M. 2001 in: Fields Virology, eds. Knipe, D. M. & Howley, P. M. Lippincott Williams and Wilkins, Philadelphia, 4-th ed., pp. 1043-1125

17. Men et al. 1996 J Virol 70:3930-3937;

18. Oster et al., “Interim Guidelines for Prevention of Sexual Transmission of Zika Virus—United States, 2016”, Morbity and Mortality Weekly Report 2016; 65; 1-2

19. Pletnev, A. G., Bray, M. & Lai, C.-J. 1993 J Virol 67:4956-4963

20. Pletnev, A. G. et al. 1992 PNAS USA 89:10532-10536;

21. Pletnev, A. G. & Men, R. 1998 PNAS USA 95:1746-1751;

22. Pletnev, A. G. et al. 2000 Virology 274:26-31;

23. Pletnev, A. G. et al. 2001 J Virol 75:8259-8267

24. Worobey, M. et al. 1999 PNAS USA 96:7352-7

25. Van Der Most, R. G. et al. 2000 J Virol 74:8094-8101;

All publications, patent applications, patents, figures and other references cited or referenced herein and all documents cited or referenced in the herein cited documents, together with any manufacturer's instructions, descriptions, product specifications, and product sheets for any products mentioned herein or in any document incorporated by reference herein, are hereby incorporated by reference, and may be employed in the practice of the invention.

1. A Zika nucleic acid chimera comprising:
a first nucleotide sequence encoding at least one structural protein from a Zika virus (ZIKV),
a second nucleotide sequence encoding at least one nonstructural protein from a first flavivirus, and
a third nucleotide sequence of a 3′ untranslated region from a second flavivirus.

2. The Zika nucleic acid chimera of claim 1, wherein the first flavivirus is a dengue virus.

3. The Zika nucleic acid chimera of claim 1, wherein the first flavivirus is a ZIKV.

4. The Zika nucleic acid chimera of claim 1, wherein the second flavivirus is a dengue virus.

5. The Zika nucleic acid chimera of claim 1, wherein the second flavivirus is a ZIKV.

6. The Zika nucleic acid chimera of claim 2, wherein the dengue virus is a dengue serotype 1, a dengue serotype 2, a dengue serotype 3, or a dengue serotype 4.

7-9. (canceled)

10. The Zika nucleic acid chimera of claim 4, wherein the dengue virus is a dengue serotype 1, a dengue serotype 2, a dengue serotype 3, a dengue serotype 4.

11-13. (canceled)

14. The Zika nucleic acid chimera of claim 1, wherein the 3′ untranslated region contains a deletion in the nucleotide sequence.

15. The Zika nucleic acid chimera of claim 14, wherein the deletion is selected from the group consisting of: a Δ30 deletion, a Δ31 deletion, a Δ30/31 deletion, and a Δ86 deletion.

16. The Zika nucleic acid chimera of claim 14, further comprising a mutation at nucleotide 4891 of the NS3 gene and/or at nucleotide 4995 of the NS3 gene.

17. The Zika nucleic acid chimera of claim 1, wherein the at least one structural protein is pre-membrane (prM), envelope (E), or both.

18. A pentavalent immunogenic composition comprising:
a first attenuated virus that is immunogenic against dengue serotype 1,
a second attenuated virus that is immunogenic against dengue serotype 2,
a third attenuated virus that is immunogenic against dengue serotype 3,
a fourth attenuated virus that is immunogenic against dengue serotype 4, and
a fifth attenuated virus that is immunogenic against ZIKV.

19. The pentavalent immunogenic composition of claim 18, wherein the fifth attenuated virus is a Zika nucleic acid chimera comprising:
a first nucleotide sequence encoding at least one structural protein from a Zika virus (ZIKV),
a second nucleotide sequence encoding at least one nonstructural protein from a first flavivirus, and
a third nucleotide sequence of a 3′ untranslated region from a second flavivirus.

20. The pentavalent immunogenic composition of claim 18, wherein each of the attenuated viruses includes the same attenuating deletion or mutation.

21. The pentavalent immunogenic composition of claim 20, wherein the deletion is a deletion in nucleotide sequence of the 3′ untranslated region.

22. The pentavalent immunogenic composition of claim 21, wherein the deletion is selected from the group consisting of: a Δ30 deletion, a Δ31 deletion, a Δ30/31 deletion, and a Δ86 deletion.

23. The pentavalent immunogenic composition of claim 21, further comprising a mutation is at nucleotide 4891 of the NS3 gene and/or at nucleotide 4995 of the NS3 gene.

24. The pentavalent immunogenic composition of claim 18, further comprising an adjuvant.

25. A multivalent immunogenic composition comprising:
at least one first attenuated virus that is immunogenic against a flavivirus, and
a second attenuated virus that is immunogenic against ZIKV.

26. The multivalent immunogenic composition of claim 25, wherein the flavivirus is at least one of dengue virus serotype 1, dengue virus serotype 2, dengue virus serotype 3, dengue virus serotype 4, West Nile virus, yellow fever virus, Japanese encephalitis virus, and tick-borne encephalitis virus, or a combination thereof.

27. The multivalent immunogenic composition of claim 25, wherein the second attenuated virus is a Zika nucleic acid chimera comprising:
a first nucleotide sequence encoding at least one structural protein from a Zika virus (ZIKV),
a second nucleotide sequence encoding at least one nonstructural protein from a first flavivirus, and
a third nucleotide sequence of a 3′ untranslated region from a second flavivirus.

28. The multivalent immunogenic composition of claim 25, wherein the second attenuated virus is a ZIKV comprising one or more attenuating mutations and/or deletions in the genome.

29. A method of inducing an immune response in a subject comprising administering an effective amount of the Zika nucleic acid chimera of claim 1 to the subject.

30. A method of preventing or treating a ZIKV infection in a subject, wherein the method comprises administering to the subject an effective amount of the Zika nucleic acid chimera of claim 1.

31. A method of inducing an immune response in a subject comprising administering an effective amount of the composition of claim 25 to the subject.

32. A method of preventing or treating a ZIKV infection in a subject, wherein the method comprises administering to the subject an effective amount of the immunogenic composition of claim 25.
